SEQUENCE LISTING (465 sequences)

SEQ ID NO: 1 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 2 - protein, 598 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 3 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 895 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 8 - protein, 218 aa, synthetic construct
QVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 9 - protein, 1048 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 10 - protein, 1048 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPALFKSSFPPGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 721 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 12 - protein, 721 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPALFKSSFPPGSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - protein, 727 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 14 - protein, 727 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPALFKSSFPPGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 590 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 16 - protein, 586 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 17 - protein, 934 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 18 - protein, 930 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 19 - protein, 613 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 20 - protein, 609 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 21 - protein, 949 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 22 - protein, 945 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 23 - protein, 949 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 24 - protein, 945 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 25 - protein, 964 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 26 - protein, 960 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 27 - protein, 964 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGPALFKSSFPPGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 28 - protein, 960 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGPALFKSSFPPGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 29 - protein, 614 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSSGGPALFKSSFPPGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 30 - protein, 614 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSSGGPALFKSSFPPGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 31 - protein, 629 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSSGGPALFKSSFPPGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 32 - protein, 582 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 33 - protein, 586 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 34 - protein, 1042 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 35 - protein, 1042 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSSGGPALFKSSFPPGSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - protein, 538 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGPALFKSSFPPGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 217 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 221 aa, synthetic construct
QVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 40-143 - skipped (104 entries)

SEQ ID NO: 144 - protein, 141 aa, synthetic construct
QVQLQESGGGLVQAGGSLRLSCAASGRTFSSVYDMGWFRQAPGKDREFVARITESARNTRYADSVRGRFTISRDNAKNTVYLQMNNLELEDAAVYYCAADPQTVVVGTPDYWGQGTQVTVSSAAAYPYDVPDYGSHHHHHH

SEQ ID NO: 145 - protein, 247 aa, synthetic construct
QSVLTQPPSVSGAPGQRVTISCTGSSSNIGSNTVKWYQQLPGTAPKLLIYGNDQRPSGVPDRFSGSKSGTSASLAITGLQAEDEADYYCQSYDRYTHPAYVFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 146 - protein, 247 aa, synthetic construct
QSVLTQPPSVSGAPGQRVTISCTGSSSNIGSNTVKWYQQLPGTAPKLLIYYNDQRPSGVPDRFSGSKSGTSASLAITGLQAEDEADYYCQSYDRYTHPAYVFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 147 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARHGSHDNWGQGTMVTVSSHHHHHH

SEQ ID NO: 148 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSSHHHHHH

SEQ ID NO: 149 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYAESVKGRFTISRDNSKNTLYLQMNSLRAEDTAVYYCKTHGSHDNWGQGTMVTVSSHHHHHH

SEQ ID NO: 150 - protein, 247 aa, synthetic construct
QSVLTQPPSVSGAPGQRVTISCSGSRSNIGSQTVKWYQQLPGTAPKLLIYYNDQRPSGVPDRFSGSKSGTSASLAITGLQAEDEADYYCQSYERYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 151 - protein, 247 aa, synthetic construct
QSVLTQPPSVSGAPGQRVTISCSGSRSNIGSQTVKWYQQLPGTAPKLLIYYNDQRPSGVPDRFSGSKSGTSASLAITGLQAEDEADYYCQSYSRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 152 - protein, 247 aa, synthetic construct
QSVLTQPPSVSGAPGQRVTISCSGSESNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 153 - protein, 247 aa, synthetic construct
QSVLTQPPSVSGAPGQRVTISCSGSSSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 154 - protein, 247 aa, synthetic construct
QSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 155 - protein, 247 aa, synthetic construct
QSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 156 - protein, 247 aa, synthetic construct
QSVLTQPPSVSGAPGQRVTISCSGSRSNIGSDTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 157 - protein, 247 aa, synthetic construct
QSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 158 - protein, 247 aa, synthetic construct
QSVLTQPPSVSGAPGQRVTISCSGSRSNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 159 - protein, 247 aa, synthetic construct
QSVLTQPPSVSGAPGQRVTISCSGSRSNIGSNTVDWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 160 - protein, 247 aa, synthetic construct
QSVLTQPPSVSGAPGQRVTISCSGSRSNIGSNTVEWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 161 - protein, 247 aa, synthetic construct
QSVLTQPPSVSGAPGQRVTISCSGSRSNIGSNTVKWYQQLPGTAPKLLIYYNDQDPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 162 - protein, 247 aa, synthetic construct
QSVLTQPPSVSGAPGQRVTISCSGSRSNIGSNTVKWYQQLPGTAPKLLIYYNDQEPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 163 - protein, 247 aa, synthetic construct
QSVLTQPPSVSGAPGQRVTISCSGSRSNIGSNTVKWYQQLPGTAPKLLIYYNDQRPDGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 164 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDEYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 165 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTDPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 166 - protein, 247 aa, synthetic construct
QSVLTQPPSVSGAPGQRVTISCSGSESNIGSNTVKWYQQLPGTAPKLLIYYNDQEPSGVPDRFSGSKSGTSASLAITGLQAEDEADYYCQSYDEYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 167 - protein, 247 aa, synthetic construct
QSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 168 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFESYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 169 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSEYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 170 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSDYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 171 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVKGRFTISRDNSKNTLYLQMNSLRAEDTAVYYCKTHGSHDNWGQGTMVTVSSHHHHHH

SEQ ID NO: 172 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIDYDGSNKYYADSVKGRFTISRDNSKNTLYLQMNSLRAEDTAVYYCKTHGSHDNWGQGTMVTVSSHHHHHH

SEQ ID NO: 173 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNDYYADSVKGRFTISRDNSKNTLYLQMNSLRAEDTAVYYCKTHGSHDNWGQGTMVTVSSHHHHHH

SEQ ID NO: 174 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNEYYADSVKGRFTISRDNSKNTLYLQMNSLRAEDTAVYYCKTHGSHDNWGQGTMVTVSSHHHHHH

SEQ ID NO: 175 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVEGRFTISRDNSKNTLYLQMNSLRAEDTAVYYCKTHGSHDNWGQGTMVTVSSHHHHHH

SEQ ID NO: 176 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSEDNWGQGTMVTVSSHHHHHH

SEQ ID NO: 177 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVEGRFTISRDNSKNTLYLQMNSLRAEDTAVYYCKTHGSHDNWGQGTMVTVSSHHHHHH

SEQ ID NO: 178 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVEGRFTISRDNSKNTLYLQMNSLRAEDTAVYYCKTHGSEDNWGQGTMVTVSSHHHHHH

SEQ ID NO: 179 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 180 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 181 - protein, 247 aa, synthetic construct
QSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 182 - protein, 247 aa, synthetic construct
QSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 183 - protein, 247 aa, synthetic construct
QSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 184 - protein, 247 aa, synthetic construct
QSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 185 - protein, 247 aa, synthetic construct; identical to SEQ ID NO: 167

SEQ ID NO: 186 - protein, 247 aa, synthetic construct
QSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSHHHHHH

SEQ ID NO: 187 - protein, 247 aa, synthetic construct
QSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 188 - protein, 247 aa, synthetic construct
QSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSSHHHHHH

SEQ ID NO: 189 - protein, 218 aa, synthetic construct
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 190 - protein, 218 aa, synthetic construct; identical to SEQ ID NO: 8

SEQ ID NO: 191 - protein, 218 aa, synthetic construct
QVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 192 - protein, 217 aa, synthetic construct; identical to SEQ ID NO: 37

SEQ ID NO: 193 - protein, 217 aa, synthetic construct
QSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 194 - protein, 224 aa, synthetic construct
QVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCHHHHHH

SEQ ID NO: 195 - protein, 8 aa, synthetic construct
GPAGLYAQ

SEQ ID NO: 196 - protein, 8 aa, synthetic construct
GPAGMKGL

SEQ ID NO: 197 - protein, 8 aa, synthetic construct
PGGPAGIG

SEQ ID NO: 198 - protein, 8 aa, synthetic construct
ALFKSSFP

SEQ ID NO: 199 - protein, 8 aa, synthetic construct
ALFFSSPP

SEQ ID NO: 200 - protein, 8 aa, synthetic construct
LAQRLRSS

SEQ ID NO: 201 - protein, 8 aa, synthetic construct
LAQKLKSS

SEQ ID NO: 202 - protein, 30 aa, synthetic construct
GALFKSSFPSGGGPAGLYAQGGSGKGGSGK

SEQ ID NO: 203 - protein, 30 aa, synthetic construct
RGSGGGPAGLYAQGSGGGPAGLYAQGGSGK

SEQ ID NO: 204 - protein, 30 aa, synthetic construct
KGGGPAGLYAQGPAGLYAQGPAGLYAQGSR

SEQ ID NO: 205 - protein, 30 aa, synthetic construct
RGGPAGLYAQGGPAGLYAQGGGPAGLYAQK

SEQ ID NO: 206 - protein, 30 aa, synthetic construct
KGGALFKSSFPGGPAGIGPLAQKLKSSGGS

SEQ ID NO: 207 - protein, 30 aa, synthetic construct
SGGPGGPAGIGALFKSSFPLAQKLKSSGGG

SEQ ID NO: 208 - protein, 30 aa, synthetic construct
RGPLAQKLKSSALFKSSFPGGPAGIGGGGK

SEQ ID NO: 209 - protein, 30 aa, synthetic construct
GGGALFKSSFPLAQKLKSSPGGPAGIGGGR

SEQ ID NO: 210 - protein, 30 aa, synthetic construct
RGPGGPAGIGPLAQKLKSSALFKSSFPGGG

SEQ ID NO: 211 - protein, 30 aa, synthetic construct
RGGPLAQKLKSSPGGPAGIGALFKSSFPGK

SEQ ID NO: 212 - protein, 30 aa, synthetic construct
RSGGPAGLYAQALFKSSFPLAQKLKSSGGG

SEQ ID NO: 213 - protein, 30 aa, synthetic construct
GGPLAQKLKSSALFKSSFPGPAGLYAQGGR

SEQ ID NO: 214 - protein, 30 aa, synthetic construct
GGALFKSSFPGPAGLYAQPLAQKLKSSGGK

SEQ ID NO: 215 - protein, 30 aa, synthetic construct
RGGALFKSSFPLAQKLKSSGPAGLYAQGGK

SEQ ID NO: 216 - protein, 30 aa, synthetic construct
RGGGPAGLYAQPLAQKLKSSALFKSSFPGG

SEQ ID NO: 217 - protein, 30 aa, synthetic construct
SGPLAQKLKSSGPAGLYAQALFKSSFPGSK

SEQ ID NO: 218 - protein, 30 aa, synthetic construct
KGGPGGPAGIGPLAQRLRSSALFKSSFPGR

SEQ ID NO: 219 - protein, 30 aa, synthetic construct
KSGPGGPAGIGALFFSSPPLAQKLKSSGGR

SEQ ID NO: 220 - protein, 30 aa, synthetic construct
SGGFPRSGGSFNPRTFGSKRKRRGSRGGGG

SEQ ID NO: 221 - protein, 8 aa, synthetic construct
GPLGLKAQ

SEQ ID NO: 222 - protein, 8 aa, synthetic construct
LPLGLKAQ

SEQ ID NO: 223 - protein, 8 aa, synthetic construct
SPLGLKAQ

SEQ ID NO: 224 - protein, 8 aa, synthetic construct
QPLGLKAQ

SEQ ID NO: 225 - protein, 8 aa, synthetic construct
KPLGLKAQ

SEQ ID NO: 226 - protein, 8 aa, synthetic construct
FPLGLKAQ

SEQ ID NO: 227 - protein, 8 aa, synthetic construct
HPLGLKAQ

SEQ ID NO: 228 - protein, 8 aa, synthetic construct
PPLGLKAQ

SEQ ID NO: 229 - protein, 8 aa, synthetic construct
APLGLKAQ

SEQ ID NO: 230 - protein, 8 aa, synthetic construct
DPLGLKAQ

SEQ ID NO: 231 - protein, 8 aa, synthetic construct
GPHGLKAQ

SEQ ID NO: 232 - protein, 8 aa, synthetic construct
GPSGLKAQ

SEQ ID NO: 233 - protein, 8 aa, synthetic construct
GPQGLKAQ

SEQ ID NO: 234 - protein, 8 aa, synthetic construct
GPPGLKAQ

SEQ ID NO: 235 - protein, 8 aa, synthetic construct
GPEGLKAQ

SEQ ID NO: 236 - protein, 8 aa, synthetic construct
GPFGLKAQ

SEQ ID NO: 237 - protein, 8 aa, synthetic construct
GPRGLKAQ

SEQ ID NO: 238 - protein, 8 aa, synthetic construct
GPGGLKAQ

SEQ ID NO: 239 - protein, 8 aa, synthetic construct
GPAGLKAQ

SEQ ID NO: 240 - protein, 8 aa, synthetic construct
LPAGLKGA

SEQ ID NO: 241 - protein, 8 aa, synthetic construct
GPAGLYAQ

SEQ ID NO: 242 - protein, 8 aa, synthetic construct
GPANLVAQ

SEQ ID NO: 243 - protein, 8 aa, synthetic construct
GPAALVGA

SEQ ID NO: 244 - protein, 8 aa, synthetic construct
GPANLRAQ

SEQ ID NO: 245 - protein, 8 aa, synthetic construct
GPAGLRAQ

SEQ ID NO: 246 - protein, 8 aa, synthetic construct
GPAGLVAQ

SEQ ID NO: 247 - protein, 8 aa, synthetic construct
GPAGLRGA

SEQ ID NO: 248 - protein, 8 aa, synthetic construct
LPAGLVGA

SEQ ID NO: 249 - protein, 8 aa, synthetic construct
GPAGLKGA

SEQ ID NO: 250 - protein, 8 aa, synthetic construct
GPLALKAQ

SEQ ID NO: 251 - protein, 8 aa, synthetic construct
GPLNLKAQ

SEQ ID NO: 252 - protein, 8 aa, synthetic construct
GPLHLKAQ

SEQ ID NO: 253 - protein, 8 aa, synthetic construct
GPLYLKAQ

SEQ ID NO: 254 - protein, 8 aa, synthetic construct
GPLPLKAQ

SEQ ID NO: 255 - protein, 8 aa, synthetic construct
GPLELKAQ

SEQ ID NO: 256 - protein, 8 aa, synthetic construct
GPLRLKAQ

SEQ ID NO: 257 - protein, 8 aa, synthetic construct
GPLLLKAQ

SEQ ID NO: 258 - protein, 8 aa, synthetic construct
GPLSLKAQ

SEQ ID NO: 259 - protein, 8 aa, synthetic construct
GPLGLYAQ

SEQ ID NO: 260 - protein, 8 aa, synthetic construct
GPLGLFAQ

SEQ ID NO: 261 - protein, 8 aa, synthetic construct
GPLGLLAQ

SEQ ID NO: 262 - protein, 8 aa, synthetic construct
GPLGLHAQ

SEQ ID NO: 263 - protein, 8 aa, synthetic construct
GPLGLRAQ

SEQ ID NO: 264 - protein, 8 aa, synthetic construct
GPLGLAAQ

SEQ ID NO: 265 - protein, 8 aa, synthetic construct
GPLGLEAQ

SEQ ID NO: 266 - protein, 8 aa, synthetic construct
GPLGLGAQ

SEQ ID NO: 267 - protein, 8 aa, synthetic construct
GPLGLPAQ

SEQ ID NO: 268 - protein, 8 aa, synthetic construct
GPLGLQAQ

SEQ ID NO: 269 - protein, 8 aa, synthetic construct
GPLGLSAQ

SEQ ID NO: 270 - protein, 8 aa, synthetic construct
GPLGLVAQ

SEQ ID NO: 271 - protein, 8 aa, synthetic construct
GPLGLKLQ

SEQ ID NO: 272 - protein, 8 aa, synthetic construct
GPLGLKFQ

SEQ ID NO: 273 - protein, 8 aa, synthetic construct
GPLGLKEQ

SEQ ID NO: 274 - protein, 8 aa, synthetic construct
GPLGLKKQ

SEQ ID NO: 275 - protein, 8 aa, synthetic construct
GPLGLKQQ

SEQ ID NO: 276 - protein, 8 aa, synthetic construct
GPLGLKSQ

SEQ ID NO: 277 - protein, 8 aa, synthetic construct
GPLGLKGQ

SEQ ID NO: 278 - protein, 8 aa, synthetic construct
GPLGLKHQ

SEQ ID NO: 279 - protein, 8 aa, synthetic construct
GPLGLKPQ

SEQ ID NO: 280 - protein, 8 aa, synthetic construct
GPLGLKAG

SEQ ID NO: 281 - protein, 8 aa, synthetic construct
GPLGLKAF

SEQ ID NO: 282 - protein, 8 aa, synthetic construct
GPLGLKAP

SEQ ID NO: 283 - protein, 8 aa, synthetic construct
GPLGLKAL

SEQ ID NO: 284 - protein, 8 aa, synthetic construct
GPLGLKAE

SEQ ID NO: 285 - protein, 8 aa, synthetic construct
GPLGLKAA

SEQ ID NO: 286 - protein, 8 aa, synthetic construct
GPLGLKAH

SEQ ID NO: 287 - protein, 8 aa, synthetic construct
GPLGLKAK

SEQ ID NO: 288 - protein, 8 aa, synthetic construct
GPLGLKAS

SEQ ID NO: 289 - protein, 8 aa, synthetic construct
GPLGLFGA

SEQ ID NO: 290 - protein, 8 aa, synthetic construct
GPLGLQGA

SEQ ID NO: 291 - protein, 8 aa, synthetic construct
GPLGLVGA

SEQ ID NO: 292 - protein, 8 aa, synthetic construct
GPLGLAGA

SEQ ID NO: 293 - protein, 8 aa, synthetic construct
GPLGLLGA

SEQ ID NO: 294 - protein, 8 aa, synthetic construct
GPLGLRGA

SEQ ID NO: 295 - protein, 8 aa, synthetic construct
GPLGLYGA

SEQ ID NO: 296 - protein, 8 aa, synthetic construct
ALFKSSPP

SEQ ID NO: 297 - protein, 8 aa, synthetic construct
SPFRSSRQ

SEQ ID NO: 298 - protein, 8 aa, synthetic construct
KLFKSSPP

SEQ ID NO: 299 - protein, 8 aa, synthetic construct
HLFKSSPP

SEQ ID NO: 300 - protein, 8 aa, synthetic construct
SLFKSSPP

SEQ ID NO: 301 - protein, 8 aa, synthetic construct
QLFKSSPP

SEQ ID NO: 302 - protein, 8 aa, synthetic construct
LLFKSSPP

SEQ ID NO: 303 - protein, 8 aa, synthetic construct
PLFKSSPP

SEQ ID NO: 304 - protein, 8 aa, synthetic construct
FLFKSSPP

SEQ ID NO: 305 - protein, 8 aa, synthetic construct
GLFKSSPP

SEQ ID NO: 306 - protein, 8 aa, synthetic construct
VLFKSSPP

SEQ ID NO: 307 - protein, 8 aa, synthetic construct
ELFKSSPP

SEQ ID NO: 308 - protein, 8 aa, synthetic construct
AKFKSSPP

SEQ ID NO: 309 - protein, 8 aa, synthetic construct
AHFKSSPP

SEQ ID NO: 310 - protein, 8 aa, synthetic construct
AGFKSSPP

SEQ ID NO: 311 - protein, 8 aa, synthetic construct
APFKSSPP

SEQ ID NO: 312 - protein, 8 aa, synthetic construct
ANFKSSPP

SEQ ID NO: 313 - protein, 8 aa, synthetic construct
AFFKSSPP

SEQ ID NO: 314 - protein, 8 aa, synthetic construct
AAFKSSPP

SEQ ID NO: 315 - protein, 8 aa, synthetic construct
ASFKSSPP

SEQ ID NO: 316 - protein, 8 aa, synthetic construct
AEFKSSPP

SEQ ID NO: 317 - protein, 8 aa, synthetic construct
ALRKSSPP

SEQ ID NO: 318 - protein, 8 aa, synthetic construct
ALLKSSPP

SEQ ID NO: 319 - protein, 8 aa, synthetic construct
ALAKSSPP

SEQ ID NO: 320 - protein, 8 aa, synthetic construct
ALQKSSPP

SEQ ID NO: 321 - protein, 8 aa, synthetic construct
ALHKSSPP

SEQ ID NO: 322 - protein, 8 aa, synthetic construct
ALPKSSPP

SEQ ID NO: 323 - protein, 8 aa, synthetic construct
ALTKSSPP

SEQ ID NO: 324 - protein, 8 aa, synthetic construct
ALGKSSPP

SEQ ID NO: 325 - protein, 8 aa, synthetic construct
ALDKSSPP

SEQ ID NO: 326 - protein, 8 aa, synthetic construct
ALFFSSPP

SEQ ID NO: 327 - protein, 8 aa, synthetic construct
ALFHSSPP

SEQ ID NO: 328 - protein, 8 aa, synthetic construct
ALFTSSPP

SEQ ID NO: 329 - protein, 8 aa, synthetic construct
ALFASSPP

SEQ ID NO: 330 - protein, 8 aa, synthetic construct
ALFQSSPP

SEQ ID NO: 331 - protein, 8 aa, synthetic construct
ALFLSSPP

SEQ ID NO: 332 - protein, 8 aa, synthetic construct
ALFGSSPP

SEQ ID NO: 333 - protein, 8 aa, synthetic construct
ALFESSPP

SEQ ID NO: 334 - protein, 8 aa, synthetic construct
ALFPSSPP

SEQ ID NO: 335 - protein, 8 aa, synthetic construct
ALFKHSPP

SEQ ID NO: 336 - protein, 8 aa, synthetic construct
ALFKLSPP

SEQ ID NO: 337 - protein, 8 aa, synthetic construct
ALFKKSPP

SEQ ID NO: 338 - protein, 8 aa, synthetic construct
ALFKASPP

SEQ ID NO: 339 - protein, 8 aa, synthetic construct
ALFKISPP

SEQ ID NO: 340 - protein, 8 aa, synthetic construct
ALFKGSPP

SEQ ID NO: 341 - protein, 8 aa, synthetic construct
ALFKNSPP

SEQ ID NO: 342 - protein, 8 aa, synthetic construct
ALFKRSPP

SEQ ID NO: 343 - protein, 8 aa, synthetic construct
ALFKESPP

SEQ ID NO: 344 - protein, 8 aa, synthetic construct
ALFKFSPP

SEQ ID NO: 345 - protein, 8 aa, synthetic construct
ALFKPSPP

SEQ ID NO: 346 - protein, 8 aa, synthetic construct
ALFKSFPP

SEQ ID NO: 347 - protein, 8 aa, synthetic construct
ALFKSLPP

SEQ ID NO: 348 - protein, 8 aa, synthetic construct
ALFKSIPP

SEQ ID NO: 349 - protein, 8 aa, synthetic construct
ALFKSKPP

SEQ ID NO: 350 - protein, 8 aa, synthetic construct
ALFKSAPP

SEQ ID NO: 351 - protein, 8 aa, synthetic construct
ALFKSQPP

SEQ ID NO: 352 - protein, 8 aa, synthetic construct
ALFKSPPP

SEQ ID NO: 353 - protein, 8 aa, synthetic construct
ALFKSEPP

SEQ ID NO: 354 - protein, 8 aa, synthetic construct
ALFKSGPP

SEQ ID NO: 355 - protein, 8 aa, synthetic construct
ALFKSSFP

SEQ ID NO: 356 - protein, 8 aa, synthetic construct
ALFKSSLP

SEQ ID NO: 357 - protein, 8 aa, synthetic construct
ALFKSSGP

SEQ ID NO: 358 - protein, 8 aa, synthetic construct
ALFKSSSP

SEQ ID NO: 359 - protein, 8 aa, synthetic construct
ALFKSSVP

SEQ ID NO: 360 - protein, 8 aa, synthetic construct
ALFKSSHP

SEQ ID NO: 361 - protein, 8 aa, synthetic construct
ALFKSSAP

SEQ ID NO: 362 - protein, 8 aa, synthetic construct
ALFKSSNP

SEQ ID NO: 363 - protein, 8 aa, synthetic construct
ALFKSSKP

SEQ ID NO: 364 - protein, 8 aa, synthetic construct
ALFKSSEP

SEQ ID NO: 365 - protein, 8 aa, synthetic construct
ALFKSSPF

SEQ ID NO: 366 - protein, 8 aa, synthetic construct
ALFKSSPH

SEQ ID NO: 367 - protein, 8 aa, synthetic construct
ALFKSSPG

SEQ ID NO: 368 - protein, 8 aa, synthetic construct
ALFKSSPA

SEQ ID NO: 369 - protein, 8 aa, synthetic construct
ALFKSSPS

SEQ ID NO: 370 - protein, 8 aa, synthetic construct
ALFKSSPV

SEQ ID NO: 371 - protein, 8 aa, synthetic construct
ALFKSSPQ

SEQ ID NO: 372 - protein, 8 aa, synthetic construct
ALFKSSPK

SEQ ID NO: 373 - protein, 8 aa, synthetic construct
ALFKSSPL

SEQ ID NO: 374 - protein, 8 aa, synthetic construct
ALFKSSPD

SEQ ID NO: 375 - protein, 8 aa, synthetic construct
KRALGLPG

SEQ ID NO: 376 - protein, 40 aa, synthetic construct
DEDEDEDEDEDEDEDERPLALWRSDRDRDRDRDRDRDRDR

SEQ ID NO: 377 - skipped

SEQ ID NO: 378 - protein, 5 aa, synthetic construct
LEATA

SEQ ID NO: 379 - protein, 10 aa, synthetic construct
GGAANLVRGG

SEQ ID NO: 380 - protein, 10 aa, synthetic construct
SGRIGFLRTA

SEQ ID NO: 381 - protein, 6 aa, synthetic construct
PLGLAG

SEQ ID NO: 382 - protein, 6 aa, synthetic construct
PLGLAX

SEQ ID NO: 383 - protein, 6 aa, synthetic construct
  MOD_RES 4: note = s-methyl cysteine
PLGCAG

SEQ ID NO: 384 - protein, 8 aa, synthetic construct
ESPAYYTA

SEQ ID NO: 385 - protein, 6 aa, synthetic construct
RLQLKL

SEQ ID NO: 386 - protein, 7 aa, synthetic construct
RLQLKAC

SEQ ID NO: 387 - protein, 7 aa, synthetic construct
  MOD_RES 3: note = citrulline
  MOD_RES 5: note = homophenylalanine
EPXGXYL

SEQ ID NO: 388 - protein, 5 aa, synthetic construct
SGRSA

SEQ ID NO: 389 - protein, 4 aa, synthetic construct
DAFK

SEQ ID NO: 390 - protein, 5 aa, synthetic construct
GGGRR

SEQ ID NO: 391 - protein, 4 aa, synthetic construct
GFLG

SEQ ID NO: 392 - protein, 4 aa, synthetic construct
ALAL

SEQ ID NO: 393-394 - skipped (2 entries)

SEQ ID NO: 395 - protein, 5 aa, synthetic construct
  MOD_RES 3: note = s-ethyl cysteine
PICFF

SEQ ID NO: 396 - protein, 8 aa, synthetic construct
GGPRGLPG

SEQ ID NO: 397 - protein, 6 aa, synthetic construct
HSSKLQ

SEQ ID NO: 398 - protein, 7 aa, synthetic construct
HSSKLQL

SEQ ID NO: 399 - protein, 9 aa, synthetic construct
HSSKLQEDA

SEQ ID NO: 400 - protein, 10 aa, synthetic construct
LVLASSSFGY

SEQ ID NO: 401 - protein, 10 aa, synthetic construct
GVSQNYPIVG

SEQ ID NO: 402 - protein, 10 aa, synthetic construct
GVVQASCRLA

SEQ ID NO: 403 - skipped

SEQ ID NO: 404 - protein, 6 aa, synthetic construct
DPRSFL

SEQ ID NO: 405 - protein, 6 aa, synthetic construct
PPRSFL

SEQ ID NO: 406 - protein, 4 aa, synthetic construct
DEVD

SEQ ID NO: 407 - protein, 5 aa, synthetic construct
DEVDP

SEQ ID NO: 408 - protein, 8 aa, synthetic construct
KGSGDVEG

SEQ ID NO: 409 - protein, 6 aa, synthetic construct
GWEHDG

SEQ ID NO: 410 - protein, 7 aa, synthetic construct
EDDDDKA

SEQ ID NO: 411 - protein, 9 aa, synthetic construct
KQEQNPGST

SEQ ID NO: 412 - protein, 6 aa, synthetic construct
GKAFRR

SEQ ID NO: 413 - protein, 4 aa, synthetic construct
DAFK

SEQ ID NO: 414 - protein, 4 aa, synthetic construct
DVLK

SEQ ID NO: 415 - protein, 4 aa, synthetic construct
DAFK

SEQ ID NO: 416 - protein, 7 aa, synthetic construct
ALLLALL

SEQ ID NO: 417 - protein, 7 aa, synthetic construct
GPLGVRG

SEQ ID NO: 418 - protein, 8 aa, synthetic construct
IPVSLRSG

SEQ ID NO: 419 - protein, 8 aa, synthetic construct
VPLSLYSG

SEQ ID NO: 420 - protein, 10 aa, synthetic construct
SGESPAYYTA

SEQ ID NO: 421-446 - skipped (26 entries)

SEQ ID NO: 447 - protein, 7 aa, synthetic construct
PAGLYAQ

SEQ ID NO: 448 - protein, 7 aa, synthetic construct
LFKSSFP

SEQ ID NO: 449 - protein, 8 aa, synthetic construct
KSPWFTTL

SEQ ID NO: 450 - protein, 14 aa, synthetic construct
CAARDVGDNSKLIW

SEQ ID NO: 451 - protein, 15 aa, synthetic construct
CAMREGPGGNNKLTF

SEQ ID NO: 452 - protein, 13 aa, synthetic construct
CAMYGGSGNKLIF

SEQ ID NO: 453 - protein, 14 aa, synthetic construct
CAVSGGGSNYKLTF

SEQ ID NO: 454 - protein, 14 aa, synthetic construct
CAVSIGTGNYKYVF

SEQ ID NO: 455 - protein, 14 aa, synthetic construct
CAVSKGTGNYKYVF

SEQ ID NO: 456 - protein, 13 aa, synthetic construct
CVLGDSGYNKLTF

SEQ ID NO: 457 - protein, 14 aa, synthetic construct
CAVSMGTGNYKYVF

SEQ ID NO: 458 - protein, 11 aa, synthetic construct
CATTGNYKYVF

SEQ ID NO: 459 - protein, 14 aa, synthetic construct
CAMREGTNTGKLTF

SEQ ID NO: 460 - protein, 13 aa, synthetic construct
CAARTTGYQNFYF

SEQ ID NO: 461 - protein, 12 aa, synthetic construct
CAVPTGGYKVVF

SEQ ID NO: 462 - protein, 14 aa, synthetic construct
CAVSTGTGNYKYVF

SEQ ID NO: 463 - protein, 14 aa, synthetic construct
CAASGTQVVGQLTF

SEQ ID NO: 464 - protein, 13 aa, synthetic construct
CAVSASSGQKLVF

SEQ ID NO: 465 - protein, 13 aa, synthetic construct
CAARGSALGRLHF

(19) United States
(12) Patent Application Publication
(10) Pub. No.: US 2026/0125436 A1
(43) Pub. Date: May 7, 2026

(54) IL-12 PRODRUGS, METHODS OF USE AND PHARMACEUTICAL COMPOSITIONS

(71) Applicant: Werewolf Therapeutics, Inc., East Watertown, MA (US)

(72) Inventors: Randi ISAACS, East Hampton, NY (US); Sameer CHOPRA, Brookline, MA (US); Kulandayan SUBRAMANIAN, Boxborough, MA (US)

(21) Appl. No.: 19/326,578

(22) Filed: Sep. 11, 2025

Related U.S. Application Data
(63) Continuation of application No. PCT/US2024/023550, filed on Apr. 8, 2024.
(60) Provisional application No. 63/494,517, filed on Apr. 6, 2023.
(60) Provisional application No. 63/495,899, filed on Apr. 13, 2023.

(51) Int. Cl. C07K 14/54; A61K 38/00

(52) U.S. Cl. CPC C07K 14/5434; A61K 38/00

(57) ABSTRACT

This disclosure relates to methods and compositions for treating cancer including advanced solid tumor, a metastatic solid tumor or lymphoma using an inducible IL-12 prodrug.

The present application is a continuation of International Patent Application No. PCT/US2024/023550, filed Jun. 27, 2024, which designated the United States, which claims the benefit of U.S. Provisional Application No. 63/494,517, filed on Apr. 6, 2023, and U.S. Provisional Application No. 63/495,899, filed on Apr. 13, 2023, the entire contents of each of which are incorporated herein by reference in their entireties.

### 1. SEQUENCE LISTING

The instant application contains a Sequence Listing which has been submitted electronically in XML format and is hereby incorporated by reference in its entirety. Said XML copy, created on Apr. 5, 2024, is named 761146_292320_SL.xml and is 277 bytes in size.

### 2. BACKGROUND

Cancer immunotherapy has rapidly established itself as the fourth pillar of cancer treatment largely owing to the clinical success of checkpoint inhibitors. Despite the durable responses achieved by some patients using these new therapies, the proportion of responders is still relatively low and restricted to only some cancer types. Tumor mutational burden, the presence or absence of T cell infiltration in tumors, and the overall immunosuppressive microenvironment of tumors greatly influences the response to immunotherapies. Although immune checkpoint blockade can prevent the physiological stop-signal that arises in response to immune activation, other approaches can be used to positively stimulate the anti-tumor immune response. One approach involves the use of immune-activating cytokines. Numerous preclinical and clinical studies have demonstrated the promise of cytokine therapy to increase anti-tumor immunity. In fact, these were some of the first cancer immunotherapies approved for clinical use. However, systemic toxicity and poor pharmacokinetic profiles have limited their clinical application.

Interleukin-12 (IL-12) is a heterodimeric 70 kDa cytokine composed of two covalently linked glycosylated subunits (p35 and p40) (Lieschke et al., 1997; Jana et al., 2014). It is a potent immune agonist and has been considered a promising therapeutic agent for oncology. However, IL-12 has shown to have a narrow therapeutic window because they are highly potent and have a short serum half-life. Consequently, therapeutic administration of IL-12 produces undesirable systemic effects and toxicities. This is exacerbated by the need to administer large quantities of cytokines (i.e., IL-12) in order to achieve the desired levels of cytokine at the intended site of cytokine action (e.g., a tumor microenvironment). Unfortunately, due to the biology of cytokine and the inability to effectively target and control their activity, cytokines have not achieved the hoped for clinical advantages in the treatment in tumors.

Inducible forms of IL-12, that are conditionally activated in the tumor microenvironment through protease cleavage to release the fully active, native IL-12 cytokine within the tumor to stimulate a potent anti-tumor immune response, are described in International Application Nos. PCT/US2019/032320, PCT/US2019/032322, and PCT/US2021/033014. These IL-12 prodrugs include a native IL-12 molecule attached through a protease cleavable linker to a half-life extension domain (e.g., anti-human serum albumin antibody binding fragment such as a VH domain) and an IL-12 blocking element (e.g., anti-IL-12 antibody binding fragment, such as a Fab or scFv) to block binding of IL-12 to IL-12Rβ1 or IL-12Rβ2 receptors on normal tissue in the periphery. Upon cleavage of the protease cleavable linker, fully active native IL-12 is released within the tumor to stimulate a potent anti-tumor immune response.

### 3. SUMMARY

This disclosure relates to compositions and methods for treating cancer using an inducible IL-12 prodrug. The inducible IL-12 prodrug that contain an attenuated IL-12 and that have a long half-life in comparison to naturally occurring IL-12. If desired, the IL-12 can be a mutein. The IL-12 mutein can be aglycosylated or partially aglycosylated. The inducible IL-12 prodrugs disclosed herein comprise two or more polypeptide chains, and the inducible IL-12 prodrug includes IL-12 subunits p35 and p40, a half-life extension element, an IL-12 blocking element and a protease cleavable linker.

The inducible IL-12 prodrug can comprise two different polypeptides. The first polypeptide can comprise an IL-12 subunit, and optionally an IL-12 blocking element. The IL-12 blocking element when present is operably linked to the IL-12 subunit through a first protease cleavable linker. The second polypeptide chain can comprise an IL-12 subunit operably linked to a half-life extension element through a second protease cleavable linker, and optionally a IL-12 blocking element. The IL-12 blocking element when present can be operably linked to the IL-12 subunit through a protease cleavable linker or can be operably linked to the half-life extension element through a linker that is optionally protease cleavable. Only one of the first and second polypeptide contains the IL-12 blocking element. When the IL-12 subunit in the first polypeptide is p35, the IL-12 subunit in the second polypeptide is p40, and when the IL-12 subunit in the first polypeptide is p40, the IL-12 subunit in the second polypeptide is p35. A preferred blocking element of this inducible IL-12 prodrug is a single chain antibody that binds IL-12 or an antigen binding fragment thereof. The cleavable linkers in this inducible IL-12 prodrug can be the same or different.

The inducible IL-12 prodrug can comprise three different polypeptides. Typically, one polypeptide chain comprises either the p35 or p40 IL-12 subunit, but not both, and a second polypeptide comprises the other IL-12 subunit and the third polypeptide comprises at least a portion (component) of the blocking element. The first polypeptide can comprise an IL-12 subunit, and optionally a half-life extension element. The half-life extension element when present is operably linked to the IL-12 subunit through a protease cleavable linker.

The second polypeptide can comprise a IL-12 subunit, at least an antigen binding portion of an antibody light chain or an antigen binding portion of an antibody heavy chain, and optionally a half-life extension element. When the half-life extension element is present, it is operably linked to the IL-12 subunit through a protease cleavable linker and the antibody heavy chain or light chain is either a) operably linked to the IL-12 subunit through a second protease cleavable linker, or b) operably linked to the half-life extension element through an optionally cleavable linker.

The third polypeptide can comprise can an antigen binding portion of an antibody heavy chain that is complementary to the light chain in the second polypeptide, or an antibody light chain that is complementary to the heavy chain in the second polypeptide and together with said light chain forms an IL-12 binding site. When the IL-12 subunit in the first polypeptide is p35, the IL-12 subunit in the second polypeptide is p40, and when the IL-12 subunit in the first polypeptide is p40, the IL-12 subunit in the second polypeptide is p35. In this inducible IL-12 prodrug, the IL-12 blocking element is preferably an antigen binding fragment of an antibody. The antigen binding fragment comprises as separate components, at least an antigen-binding portion of an antibody light chain and at least an antigen-binding portion of a complementary antibody heavy chain. The protease cleavable linkers in this inducible IL-12 prodrug can be the same or different.

The inducible IL-12 prodrug can comprise two different polypeptides wherein p35 and p40 are located on the same polypeptide chain. A first polypeptide chain can comprise p35, p40, a half-life extension element and at least an antigen binding portion of an antibody light chain. p35 and p40 can be operably linked, and the half-life extension element can be operably linked to p40 through a first protease cleavable linker and the antigen binding portion of an antibody light chain can be operably linked to p35 through a protease cleavable linker. Alternatively, the half-life extension element can be operably linked to p35 through a protease cleavable linker and the antigen binding portion of an antibody light chain is operably linked to p40 through a protease cleavable linker. The second polypeptide comprises at least an antigen binding portion of an antibody heavy chain that is complementary to the light chain in the second polypeptide and together with said light chain forms and IL-12 binding site. The protease cleavable linkers in this inducible IL-12 prodrug can be the same or different.

In an alternative format, a first polypeptide chain can comprise p35, p40, a half-life extension element and at least an antigen binding portion of an antibody heavy chain. p35 and p40 can be operably linked, and the half-life extension element can be operably linked to p40 or through a protease cleavable linker and the antigen binding portion of an antibody heavy chain can be operably linked to p35 through a protease cleavable linker. Alternatively, the half-life extension element can be operably linked to p35 through a protease cleavable linker and the antigen binding portion of an antibody heavy chain can be operably linked to p40 through a second protease cleavable linker. A second polypeptide comprises at least an antigen binding portion of an antibody light chain that is complementary to the heavy chain in the second polypeptide and together with said light chain forms and IL-12 binding site. The protease cleavable linkers in this inducible IL-12 prodrug can be the same or different.

In one example, the inducible IL-12 prodrug comprises a first polypeptide does not comprise a blocking element and the second polypeptide has the formula: [A]-[L1]-[B]-[L3]-[D] or [D]-[L3]-[B]-[L1]-[A] or [B]-[L1]-[A]-[L2]-[D] or [D]-[L1]-[A]-[L2]-[B], wherein, A is the IL-12 subunit; L1 is the first protease-cleavable linker; L2 is the second protease cleavable linker; L3 is the optionally cleavable linker; B is the half-life extension element; and D is the blocking element.

In another example, the first polypeptide comprises the formula: [A]-[L1]-[D] or [D]-[L1]-[A]; and the second polypeptide has the formula: [A′]-[L2]-[B] or [B]-[L2]-[A′], wherein A is either p35 or p40, wherein when A is p35, A′ is p40 and when A is p40, A′ is p35; A′ is either p35 or p40; L1 is the first protease cleavable linker; L2 is the second protease cleavable linker; B is the half-life extension element; and D is the blocking element.

This disclosure relates to a method for treating an advanced solid tumor, a metastatic solid tumor or a lymphoma, comprising administering to a subject in need thereof an effective amount of an inducible IL-12 prodrug.

The inducible IL-12 prodrug administered can be Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, or an amino acid sequence variant of any of the foregoing.

The IL-12 prodrug can be administered orally, parenterally, intravenously, intraarticularly, intraperitoneally, intramuscularly, subcutaneously, intracavity, transdermally, intrahepatically, intracranially, nebulization/inhalation, by installation via bronchoscopy, or intratumorally.

Typically the inducible IL-12 prodrug is administered intravenously, and is administered about twice a week or less frequently, for example one every two weeks. The inducible IL-12 prodrug can be administered in a dose of about 0.016 mg/kg to about 500 mg/kg per administration, for example about 0.016 mg/kg, about 0.032 mg/kg, about 0.056 mg/kg, about 0.084 mg/kg, about 0.126 mg/kg, about 0.190 mg/kg, about 0.290 mg/kg or about 0.440 mg/kg, in each case per dose. A dose of about 1 mg to about 500 mg IL-12 prodrug can be administered at each administration. For example a dose of about 1 mg, about 3 mg, about 10 mg, about 20 mg, about 30 mg, about 40 mg, about 50 mg, about 60 mg, about 100 mg, or about 200 mg can be administered at each administration.

In some embodiments, the patient to be treated has failed to achieve a complete response to a prior treatment or to an ongoing treatment, typically treatment with an immune checkpoint inhibitor, such as anti-PD-1 or anti-PD-L1 or anti-CTLA4. In embodiments, the inducible IL-12 prodrug that is administered is Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, or Compound 36 or an amino acid sequence variant of any of the foregoing.

The method can be used to treat any desired advanced solid tumor, metastatic solid tumor, or lymphoma. For example, adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

The advanced solid tumor or the metastatic solid tumor can be colon cancer, lung cancer, melanoma, renal cell carcinoma, or breast cancer.

The advanced solid tumor, the metastatic solid tumor or the lymphoma can be melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), or oesophageal carcinoma.

The methods disclosed herein are particularly suitable for treating colorectal cancer.

This disclosure also relates to a pharmaceutical composition an inducible IL-12 prodrug, citric acid and/or a citrate salt, a disaccharide and a surfactant. In embodiments, the citrate salt is sodium citrate, magnesium citrate or potassium citrate; the disaccharide is sucrose, trehalose, lactose or maltose, and the surfactant is a nonionic surfactant selected from polysorbate 80, polysorbate 20, Span-80, castor oil, or a poloxamer. The pharmaceutical composition can be a liquid (e.g., an aqueous liquid for injection or infusion) or a solid (e.g., a lyophilizate or spray dried formulation). Exemplary pharmaceutical compositions comprises about 1 mg/mL to about 100 mg/mL inducible IL-12 prodrug; about 5 mM to about 500 mM sodium citrate; about 20 mM to about 500 mM sucrose, about 0.001% to about 2% polysorbate 80.

When the pharmaceutical composition is an aqueous liquid, it has a pH of about 5.0 to about 8.0.

### 4. BRIEF DESCRIPTION OF THE DRAWINGS

The drawings are not necessarily to scale or exhaustive. Instead, the emphasis is generally placed upon illustrating the principles of the inventions described herein. The accompanying drawings, which constitute part of the specification, illustrate several embodiments consistent with the disclosure and, together with the description, serve to explain the principles of the disclosure. In the drawings:

FIG. 1 is a graph showing the in vitro activity of chimeric Compound 1 in the IL-12 HEK-Blue reporter assay, comparing intact chimeric Compound 1 (squares) and cleaved chimeric Compound 1 (triangles) to chimeric IL-12 (circles).

FIGS. 2A-2B show that chimeric Compound 1 is well tolerated and induces tumor regression in a cleavage dependent manner. FIG. 2A is a graph showing anti-tumor activity of chimeric Compound 1 at various doses in the murine model. Chimeric Compound 1 was dosed intraperitonially twice a week for two weeks at 7 μg/dose and 43 μg/dose and a NC (Non-cleavable) version of chimeric Compound 1 was dosed at 43 μg/dose. FIG. 2B is a graphic depiction of the calculated therapeutic window for chimeric IL-12 and chimeric Compound 1 on a per molar basis using the same tumor model (MC38), based on the identification of the active and toxic dose level for both treatments.

FIGS. 3A-3G are graphs showing that chimeric Compound 1 generates anti-tumor immunity and protective memory in multiple syngeneic tumor models. FIGS. 3A-3E show anti-tumor activity of chimeric Compound 1 at various doses in various murine syngeneic tumor models, CT26 model (FIG. 3A), B16-F10 model (FIG. 3B), EMT-6 model (FIG. 3C), A20 model (FIG. 3D), and EG7.OVA model (FIG. 3E). Mice were dosed twice a week with the dose noted on the figure legends for a total of two weeks. FIGS. 3F and 3G are graphs showing tumor volume in the EMT6 (FIG. 3F) and MC38 (FIG. 3G) models re-challenged with the same tumor on the opposite flank over time, demonstrating that treatment with chimeric Compound 1 induces immunological memory against the same tumor type.

FIGS. 4A-4F show that chimeric Compound 1 treatment reshapes the tumor microenvironment and induces activation of intratumoral effector cells (NKs and CD8+ T cells) in the MC38 model. FIG. 4A is a heatmap of transcripts with statistically significant differences between the two treatments derived from nanostring analysis of bulk RNA from tumor samples. Transcripts were excluded from the heat map if they had average normalized counts below 50. Each lane represents an individual animal. FIG. 4B shows a volcano plot of transcripts differentially expressed between chimeric Compound 1 and vehicle treated mice. FIGS. 4C-4D show the frequency of tumor infiltrating NK cells producing IFNγ or Granzyme B. FIG. 4E are flow cytometry images showing the frequency of tetramer positive CD8+ T cells producing IFNγ and/or TNF. FIG. 4F are pie chart graphs showing the frequency of polyfunctional tetramer positive CD8+ T cells measured by examining co-expression of IFNγ, TNF, and Granzyme B.

FIGS. 5A-5F show that chimeric Compound 1 treatment reshapes the tumor microenvironment and activates B16-F10 tumor infiltrating NK cells and CD8+ T cells. FIG. 5A shows a heatmap of transcripts with statistically significant differences in expression between the two treatments derived from nanostring analysis of bulk RNA from tumor samples. Transcripts were excluded from the heatmap if they had average normalized counts below 50. Each lane represents an individual animal. FIG. 5B shows a volcano plot of transcripts differentially expressed between chimeric Compound 1 and vehicle-treated mice. FIG. 5C shows graphs of pathway scores for vehicle and chimeric Compound 1 for antigen processing, interferon, MHC, and NK Cell Functions. FIG. 5D are graphs showing normalized counts from individual transcripts for vehicle and chimeric Compound 1. FIG. 5E is a flow cytometry diagram showing the frequency of tetramer+ CD8+ T cells producing IFN gamma and/or Granzyme B. FIG. 5F are pie graphs showing the frequency of polyfunctional tetramer positive CD8+ T cells measured by examining co-expression of IFN gamma, TNF, and Granzyme B by flow cytometry.

FIG. 6 are pie graphs showing that chimeric Compound 1 treatment induces a sustained polyfunctional CD8+ T cells response. Mice were implanted with EMT6 cells and randomized into treatment groups. Mice were dosed twice weekly for two weeks, and tumors were harvested at the indicated timepoints. The frequency of polyfunctional tumor infiltrating CD8+ T cells was measured by examining co-expression of IFN gamma, TNF, and Granzyme B. All animals in the vehicle group were out of the study by day 21 due to tumor burden.

FIG. 7A-7D shows that systemic administration of chimeric Compound 1 results in CD8+ T cell infiltration and activation in the tumors assessed by immunofluorescence staining and the increase of IL-12 and IFN gamma signaling by tumor infiltrating CD8+ T cells. Mice were implanted with EMT6 cells and randomized into treatment groups. Mice were dosed twice weekly for two weeks, and tumors were harvested on Day 11. Nanostring GeoMX analysis was performed on FFPE tumor tissues. FIG. 7A are immunofluorescence images of tumor infiltrating CD8+ T cells in vehicle and chimeric Compound 1. FIG. 7B is a graph showing differential gene expression analysis of tumor infiltrating CD8+ T cells. FIGS. 7C-7D are heat maps showing genes associated with IL-12 (FIG. 7C) and IFN gamma (FIG. 7D) signaling.

FIGS. 8A-8B are graphs showing anti-tumor activity of chimeric Compound 1 in various studies of the murine syngeneic MC38 tumor model. FIG. 8A is a graph showing tumor growth over time in MC38 tumor bearing mice treated with chimeric Compound 1 (+/−) daily FTY720 treatment. FIG. 8B is a graph showing tumor growth over time in MC38 tumor bearing mice dosed twice a week with CD4, CD8, and NK cell depleting antibodies in conjunction with chimeric Compound 1.

FIGS. 9A-9C show that chimeric Compound 1 is preferentially activated within the TME and expands the therapeutic window compared to chimeric IL-12. FIGS. 9A-9B are graphs showing the presence of total chimeric Compound 1 or free chimeric IL-12 over time from plasma (FIG. 9A) or tumor (FIG. 9B) from MC38 tumor-bearing mice treated with chimeric Compound 1. The area under the curve was calculated, and the ratio of total chimeric Compound 1 to free chimeric IL-12 was calculated. FIG. 9C are pie chart graphs showing the frequency of polyfunctional CD8+ T cells in the tumor, peripheral blood, tumor draining or non-tumor draining lymph nodes in MC38 tumor bearing mice dosed twice with chimeric Compound 1. The frequency of polyfunctional CD8+ T cells was measured by examining co-expression of IFN gamma, TNF, and Granzyme B after PMA/ionomycin restimulation.

FIGS. 10A-10F show chimeric Compound 1 activates tumor infiltrating immune cell populations in the MC38 syngeneic tumor model. FIG. 10A are representative flow plots of CD11b+ and CD103+ tumor infiltrating dendritic cells. FIG. 10B is a graph showing the ratio of CD11b+ and CD103+ tumor infiltrating dendritic cells. FIG. 10C is a graph showing the frequency of CD4+ T conventional cells with a T_{H}1 phenotype (Tbet+ IFN gamma+TFN+). FIG. 10D are representative flow plots showing the frequency of tumor infiltrating FoxP3+ Tregs producing IFN gamma and TNF. FIGS. 10E-10F are graphs depicting the frequency of tumor infiltrating FoxP3+ Tregs producing IFN gamma and TNF (FIG. 10E) and Tbet (FIG. 10F). Unless otherwise stated, data are represented as the mean±SD, and P values are derived from t tests (**, p<0.01; ***, p<0.001; ****, p<0.00001).

FIGS. 11A-11E show that systemic treatment with chimeric Compound 1 expands novel TCR clones and increases overall clonality of the TCR repertoire. FIG. 11A is a heat map depicting intratumoral CD8+ T cells downstream TCR signaling following vehicle and chimeric Compound 1 treatment. FIGS. 11B-11C are graphs depicting the clone frequency of individual VDJ recombination on the TCR-beta chain in an EMT-6 tumor model treated with vehicle and chimeric Compound 1. Live T cells were isolated from EMT-6 tumors on Day 11 and TCR sequencing was performed. FIG. 11D is graph depicting the clonality index score for vehicle and chimeric Compound 1. FIG. 11E is a graph depicting the frequency of the top fifty TCR clones plotted for each animal.

FIGS. 12A-12V show that chimeric Compound 1 treatment drives increased mitochondrial respiration and fitness. FIG. 12A is a heatmap of tumor infiltrating CD8+ T cells depicting genes associated with glycolysis. FIGS. 12B-12C are graphs showing intake of 2-NDBG in EMT-6 TILs from either vehicle or chimeric Compound 1 treated animals. FIGS. 12D-12F are heatmaps of tumor infiltrating CD8+ T cells depicting genes associated with the TCA cycle (FIG. 12D), mitochondrial biogenesis (FIG. 12E), and mitochondrial translation (FIG. 12F). FIGS. 12G, 12H, 12K, 12L, 12O, 12P are graphs depicting EMT-6 infiltrating CD8+ T cells from either vehicle or chimeric Compound 1 treated animals stained with mitotracker red (FIGS. 12G and 12H), TMRM (FIGS. 12K, 12L), MitoSOX (FIGS. 12O-12P). FIGS. 12I, 12J, 12M, 12N, 12Q, 12R are graphs depicting EMT-6 infiltrating NK cells from either vehicle or chimeric Compound 1 treated animals stained with mitotracker red (FIGS. 12I and 12J), TMRM (FIGS. 12M-12N), MitoSOX (FIGS. 12Q-12R). Data are presented as mean±SD, and P values are derived from t tests (*, p<0.05, **p<0.01; ***, p<0.001, ****, p<0.0001). FIGS. 12S-12T show that chimeric Compound 1 preferentially expands new clones rather than previously present clones. FIG. 12S discloses SEQ ID NOS 450-457, respectively, in order of appearance. FIG. 12T discloses SEQ ID NOS 458-465, respectively, in order of appearance. FIGS. 17A-17B are graphs showing the percentage of TCR repertoire for the top 50 shared clones. FIGS. 12U-12V shows that chimeric Compound 1 treatment increases mitochondrial mass and fitness in tumor infiltrating immune cells.

FIGS. 13A-13B show that Compound 36 is inducible, stable in human serum, and selectively processed by dissociated primary human tumor samples. FIG. 13A depicts a western blot analysis of Compound 36 that was diluted into healthy human serum from n=6 donors and incubated at 37° C. for 24 or 72 hours before analysis. FIG. 13B is a graph of protein cleavage after incubation with primary human dissociated tumor samples (n=88) or primary human healthy cells (n=13) exposed to Compound 36 for 48 hours before protein cleavage was measured by activity in a human Tblast assay. Box plots represent the 25^{th} and 75^{th} percentile, while the line represents the median value for each indication. Whiskers represent the minimum and maximum values within a given indication.

FIGS. 14A-14B are graphs showing IFN gamma production by intracellular cytokine staining with or without ex vivo restimulation in TILs from mice treated with either vehicle or chimeric Compound 1.

FIGS. 15A-15B show selective activation of tumor infiltrating immune cells. FIG. 15A is a graph showing the frequency of conventional CD4+ T conventional cells (FoxP3−) producing IFNγ and TNF in the tumor tissue compared to peripheral tissue. FIG. 15B is a graph showing the frequency of NK cells producing IFNγ and TNF in the tumor tissue compared to peripheral tissue.

FIGS. 16A, 16C, 16E, 16G, 16I, 16K, 16M, 16O, 16Q, 16S, 16U, 16W, 16Y, 16ZA, 16ZC, 16ZE, 16ZG, 16ZI, 16ZK, 16ZM, 16ZO are graphs showing the activity of inducible IL-12 prodrugs in a HEK-Blue IL-12 reporter assay in the presence of human serum albumin (HSA). Squares depict activity of the intact inducible IL-12 prodrug and triangles depict the activity of the in vitro protease activated (cleaved) inducible IL-12 prodrug. Circles depict activity of the control chimeric IL-12. EC50 values for each are shown in the table (N.D.=not determined). Analysis was performed based on quantification of Secreted Alkaline Phosphatase (SEAP) activity using the reagent QUANTI-Blue® (InvivoGen). Results confirm that inducible IL-12 prodrugs are active and inducible. FIGS. 16B, 16D, 16F, 16H, 16J, 16L, 16N, 16P, 16R, 16T, 16V, 16X, 16Z, 16ZB, 16ZD, 16ZF, 16ZH, 16ZI, 16ZJ, 16ZL, 16ZN, 16ZP are images of SDS-PAGE gels showing the results of protein cleavage assays with elastase.

FIGS. 17A, 17C, 17E, 17G, and 17I are graphs showing results of analyzing inducible IL-12 prodrugs in a syngeneic MC38 mouse tumor model. They show average tumor volume over time in mice treated with 5 μg, 50 μg, and 500 μg of each inducible IL-12 prodrug dosed biweekly. Data show the tumor volume was inhibited over time in a dose-dependent manner. FIGS. 17B, 17D, 17F, 17H, and 17J are graphs showing body weight average of the groups over time.

FIGS. 18A-18N are schematic illustrations depicting various inducible IL-12 prodrugs.

### 5. DETAILED DESCRIPTION

This disclosure relates to compositions and methods for treating cancer using an inducible IL-12 prodrug.

### A. IL-12 Prodrugs

The disclosure relates to inducible IL-12 prodrugs that contain an attenuated IL-12 and that have a long half-life in comparison to naturally occurring IL-12. The inducible IL-12 prodrugs disclosed herein contain at least one polypeptide chain, and can contain two or more polypeptides, if desired. The two or more polypeptide chains disclosed herein are different, i.e., the complexes can be heterodimers, heterotrimers, and the like. The inducible IL-12 prodrugs comprises a p35 IL-12 subunit, a p40 IL-12 subunit, a half-life extension element, an IL-12 blocking element, and a protease cleavable linker. The p35 subunit and the p40 subunit associate to form the IL-12 heterodimer, which has intrinsic IL-12 receptor agonist activity. In the context of the inducible IL-12 prodrug, the IL-12 receptor agonist activity is attenuated and the circulating half-life is extended. The IL-12 receptor agonist activity is attenuated through the blocking element. The half-life extension element can also contribute to attenuation, for example through steric effects. The blocking element is capable of blocking the activity of all or some of the receptor agonist activity of IL-12 by sterically blocking and/or noncovalently binding to IL-12 (e.g., to p35, p40, or the p35p40 complex). Upon cleavage of the protease cleavable linker a form of IL-12 is released from the inducible IL-12 prodrug that is active (e.g., more active than the inducible IL-12 prodrugs). Typically, the released IL-12 is at least 10× more active than the inducible IL-12 prodrug. Preferably, the released IL-12 is at least 20×, at least 30×, at least 50×, at least 100×, at least 200×, at least 300×, at least 500×, at least 1000×, at least about 10,000× or more active than the inducible IL-12 prodrug.

The form of IL-12 that is released upon cleavage of the inducible IL-12 prodrug typically has a short half-life, which is often substantially similar to the half-life of naturally occurring IL-12. Even though the half-life of the inducible IL-12 prodrug is extended, toxicity is reduced or eliminated because the circulating inducible IL-12 prodrug is attenuated and active IL-12 is targeted to the desired site (e.g., tumor microenvironment).

It will be appreciated by those skilled in the art, that the number of polypeptide chains, and the location of the p35 and p40 subunits, the half-life extension element, the protease cleavable linker(s), and the blocking element (and components of such elements, such as a VH or VL domain) on the polypeptide chains can vary and is often a matter of design preference. All such variations are encompassed by this disclosure.

In embodiments, the inducible IL-12 prodrug comprises two different polypeptide chains. Typically, the first polypeptide chain comprises p35 and the second polypeptide chain comprises p40. The p35 and p40 subunits associate to form a biologically active heterodimer. The p35p40 heterodimer complex can be covalently linked, for example through a disulfide bond.

In embodiments, either the first of the second polypeptide can comprise an IL-12 blocking element (e.g., an 14zac that binds IL-12) that is operably linked to the IL-12 subunit through a protease cleavable linker. The other polypeptide chain can further comprise a half-life extension element that is operably linked to the IL-12 subunit through a protease cleavable linker. Preferably, the inducible IL-12 prodrug includes one functional blocking element and one functional half-life extension element. For example, when the first polypeptide chain comprises an IL-12 blocking element, the second polypeptide chain does not comprise an IL-12 blocking element. In other embodiments, one polypeptide chain includes either p35 or p40, and further includes a half-life extension element and a blocking element, each of which is operably linked to the p35 or p40 through a protease cleavable linker (e.g., one or more protease cleavable linker), and the other polypeptide include the complementary IL-12 subunit (e.g., either p40 or p35). The IL-12 blocking element on the second polypeptide can be operably linked to the IL-12 subunit through a protease cleavable linker. Alternatively, the IL-12 blocking element can be operably linked to the half-life extension element through an optional protease cleavable linker. The protease cleavable linkers on the first and second polypeptide chains can be the same or can be different. Preferably, the protease cleavable linkers on the first and second polypeptide chains are the same. The blocking element in this inducible IL-12 prodrug can be a single chain antibody. Any single chain antibody that has binding specificity for IL-12 can be a blocking element. Preferably, the blocking element is a scFv.

While the inducible IL-12 prodrugs disclosed herein preferably contain one half-life extension element and one blocking element, such elements can contain two or more components that are present on the same polypeptide chain or on different polypeptide chains. Illustrative of this, and as disclosed and exemplified herein, components of the blocking element can present on separate polypeptide chains. For example, a first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the inducible IL-12 prodrugs to form an antigen-binding site, such as a Fab that binds IL-12 and attenuates IL-12 activity.

In embodiments, the p35 and p40 subunit can be located on the same polypeptide chain, and linked through and optionally protease cleavable linker. In such embodiments of two or multichain prodrugs, at least one of the half-life extension element, the blocking element, or a component of the half-life extension or blocking element is on a separate polypeptide. For example, a first polypeptide can include p35 and p40, linked through an optionally cleavable polypeptide chain, and other elements of the inducible IL-12 prodrug are located on a second polypeptide chain. In another example, the first polypeptide chain comprises the p35 subunit, the p40 subunit, the half-life extension element, and a portion of an antibody light chain. The second polypeptide contains a portion of an antibody heavy chain that is complementary to the antibody light chain. The portion of the antibody light chain together with the complementary heavy chain associate in the inducible IL-12 prodrug to form a binding site for IL-12. In another example, the first polypeptide comprises the p35 subunit, the p40 subunit, the half-life extension element, and a portion of an antibody heavy chain. In this example the second polypeptide contains a portion of an antibody light chain that is complementary to the antibody heavy chain. The portion of the antibody heavy chain together with the complementary light chain associate in the inducible IL-12 prodrug to form a binding site for IL-12. In these inducible IL-12 prodrugs, the p35 subunit and p40 subunit can be operably linked through an optional protease cleavable linker. Preferably, the p35 subunit and the p40 subunit are operably linked by a non-cleavable linker.

In the inducible IL-12 prodrugs disclosed herein, the half-life extension element is preferably operably linked to either the p35 subunit or the p40 subunit through a protease cleavable linker. For example, the inducible IL-12 prodrug can include a first polypeptide in which p35 or p40 is operably linked to a half-life extension element through a protease cleavable linker. In another example, the inducible IL-12 prodrug can include a first polypeptide in which p35 or p40 is operably linked to a half-life extension element through a protease cleavable linker, and the half-life extension element is further operably linked to a blocking element (or component of a blocking element) through an optionally protease cleavable linker. In such exemplary embodiments, the inducible IL-12 prodrug comprises at least one additional polypeptide that includes the IL-12 subunit (p40 or p35) that is not present on the first polypeptide. Additional arrangements of the elements of the inducible IL-12 prodrug are envisioned and encompassed by this disclosure. For example, the blocking element can be operably linked to either the p35 subunit or the p40 subunit through a protease cleavable linker. One of the half-life extension element or the blocking element can be operably linked to the p35 subunit, and the other of the half-life or extension element or the blocking element can be operably linked to the p40 subunit. When the half-life extension element is operably linked to the p35 subunit, the blocking element can be operably linked to the p40 subunit. When the half-life extension element is operably linked the p40 subunit, the blocking element can be operably linked to the p35 subunit. The blocking element in this inducible IL-12 prodrug is preferably a Fab.

The inducible IL-12 prodrugs can comprise three polypeptide chains. Typically, one polypeptide chain comprises either the p35 or p40 IL-12 subunit, but not both, and a second polypeptide comprises the other IL-12 subunit and the third polypeptide comprises at least a portion (component) of the blocking element. When the IL-12 subunit on the first polypeptide is p35, the IL-12 subunit on the second polypeptide is p40. When the IL-12 subunit on the first polypeptide is p40, the IL-12 subunit on the second polypeptide is p35. When the polypeptides are expressed and folded, the p35 and p40 subunits can associate to form a biologically active heterodimer. The p35p40 heterodimer complex can be covalently linked, for example through a disulfide bond.

In some embodiments, the first polypeptide can additionally comprise a half-life extension element that when present is operably linked to the IL-12 subunit through a protease cleavable linker. The second polypeptide further comprises a portion of the blocking element, and the third polypeptide can comprise the remainder of the blocking element. In such a inducible IL-12 prodrug, the IL-12 blocking element can be antigen binding fragment of an antibody that is formed by the interaction of polypeptide two and polypeptide three, e.g. a Fab fragment. In embodiments, the second polypeptide can comprise at least an antigen binding portion of an antibody light chain. Alternatively, the second polypeptide can comprise at least an antigen binding portion of an antibody heavy chain. The antigen binding portion of an antibody light chain or the antigen binding portion of the heavy chain can be operably linked to the IL-12 subunit through a protease cleavable linker. In some embodiments, the second polypeptide can contain a half-life extension element. When the second polypeptide contains the half-life extension element, the first polypeptide does not contain the half-life extension element. The half-life extension element can be operably linked to the IL-12 subunit through a protease cleavable linker. Alternatively or in addition, the half-life extension element can be operably linked to a portion of the blocking element (e.g., an antigen binding portion of an antibody light chain or the antigen binding portion of the heavy chain) through an optional protease cleavable linker. When the half-life extension element is present and operably linked to the IL-12 subunit, the antibody heavy chain or light chain can be operably linked to the IL-12 subunit through a protease cleavable linker, Alternatively, when the half-life extension element is present and operably linked to the IL-12 subunit, the antibody heavy chain or light chain can be operably linked to the IL-12 subunit through an optionally cleavable linker. The protease cleavable linkers on the first, second, and/or polypeptide chains can be the same or can be different.

Compounds 1, 2, 3, 4, 5, and 6 are specific examples of inducible IL-12 prodrugs that comprise two polypeptide chains for use according to this disclosure. Compounds 1, 2, 3, 4, 5, and 6 and additional details regarding their activity is disclosed in International Application No.: PCT/US2021/33014.

Compounds 7, 8, 17, 18, 21-28, 34, and 35 are specific examples of inducible IL-12 prodrugs that comprise one polypeptide chain for use according to this disclosure. Compounds 9-13, 15, 19, 20, 29-31, and 36 are specific examples of inducible IL-12 prodrugs that comprise two polypeptide chains for use according to this disclosure. Compounds 14, 16, 32, and 33 are specific examples of inducible IL-12 prodrugs that comprise three polypeptide chains for use according to this disclosure.

TABLE 1
Inducible IL-12 prodrugs

| IL-12 | First | Second | Third |
|---|---|---|---|
| Prodrug | Polypeptide | Polypeptide | Polypeptide |
| Chimeric | SEQ ID NO: 1 | SEQ ID NO: 7 | N/A |
| Compound 1 | | | |
| Compound 2 | SEQ ID NO: 2 | SEQ ID NO: 7 | N/A |
| Compound 3 | SEQ ID NO: 3 | SEQ ID NO: 8 | SEQ ID NO: 7 |
| Compound 4 | SEQ ID NO: 4 | SEQ ID NO: 8 | SEQ ID NO: 7 |
| Compound 5 | SEQ ID NO: 5 | SEQ ID NO: 8 | SEQ ID NO: 7 |
| Compound 6 | SEQ ID NO: 6 | SEQ ID NO: 8 | N/A |
| Compound 7 | SEQ ID NO: 9 | N/A | N/A |
| Compound 8 | SEQ ID NO: 10 | N/A | N/A |
| Compound 9 | SEQ ID NO: 11 | SEQ ID NO: 7 | N/A |
| Compound 10 | SEQ ID NO: 12 | SEQ ID NO: 7 | N/A |
| Compound 11 | SEQ ID NO: 13 | SEQ ID NO: 7 | N/A |
| Compound 12 | SEQ ID NO: 14 | SEQ ID NO: 7 | N/A |
| Compound 13 | SEQ ID NO: 15 | SEQ ID NO: 36 | N/A |
| Compound 14 | SEQ ID NO: 15 | SEQ ID NO: 7 | SEQ ID NO: 37 |
| Compound 15 | SEQ ID NO: 16 | SEQ ID NO: 36 | N/A |
| Compound 16 | SEQ ID NO: 16 | SEQ ID NO: 7 | SEQ ID NO: 37 |
| Compound 17 | SEQ ID NO: 17 | N/A | N/A |
| Compound 18 | SEQ ID NO: 18 | N/A | N/A |
| Compound 19 | SEQ ID NO: 19 | SEQ ID NO: 7 | N/A |
| Compound 20 | SEQ ID NO: 20 | SEQ ID NO: 7 | N/A |
| Compound 21 | SEQ ID NO: 21 | N/A | N/A |
| Compound 22 | SEQ ID NO: 22 | N/A | N/A |
| Compound 23 | SEQ ID NO: 23 | N/A | N/A |
| Compound 24 | SEQ ID NO: 24 | N/A | N/A |
| Compound 25 | SEQ ID NO: 25 | N/A | N/A |
| Compound 26 | SEQ ID NO: 26 | N/A | N/A |
| Compound 27 | SEQ ID NO: 27 | N/A | N/A |
| Compound 28 | SEQ ID NO: 28 | N/A | N/A |
| Compound 29 | SEQ ID NO: 29 | SEQ ID NO: 7 | N/A |
| Compound 30 | SEQ ID NO: 30 | SEQ ID NO: 7 | N/A |
| Compound 31 | SEQ ID NO: 31 | SEQ ID NO: 7 | N/A |
| Compound 32 | SEQ ID NO: 32 | SEQ ID NO: 7 | SEQ ID NO: 38 |
| Compound 33 | SEQ ID NO: 33 | SEQ ID NO: 7 | SEQ ID NO: 38 |
| Compound 34 | SEQ ID NO: 34 | N/A | N/A |
| Compound 35 | SEQ ID NO: 35 | N/A | N/A |
| Compound 36 | SEQ ID NO: 39 | SEQ ID NO: 7 | N/A |

As described above, the IL-12 can be a mutein, if desired. The IL-12 mutein retains IL-12 activity, for example intrinsic IL-12 receptor agonist activity. IL-12 subunits, p35 and/or p40 can be muteins.

The invention also relates to certain single chain IL-12 inducible polypeptides. The single chain IL-12 polypeptides disclosed herein comprise IL-12, a blocking element, a half-life extension element, and a protease cleavable linker. IL-12 has receptor agonist activity for its cognate IL-12 receptor. IL-12 receptor activating activity is attenuated when the blocking element binds to IL-12. Upon cleavage of the protease cleavable linkers, active IL-12 polypeptide is released. Single chain inducible IL-12 polypeptides have been disclosed in International Application No.: PCT/US2019/032320 and International Application No.: PCT/US2019/032322.

### B. Half-Life Extension Element

Contemplated herein are domains which extend the half-life of the inducible IL-12 prodrug. Increasing the in vivo half-life of therapeutic molecules with naturally short half-lives allows for a more acceptable and manageable dosing regimen without sacrificing effectiveness.

The half-life extension element, increases the in vivo half-life and provides altered pharmacodynamics and pharmacokinetics of the inducible IL-12 prodrug. Without being bound by theory, the half-life extension element alters pharmacodynamics properties including alteration of tissue distribution, penetration, and diffusion of the inducible IL-12 prodrug. In some embodiments, the half-life extension element can improve tissue targeting, tissue penetration, diffusion within the tissue, and enhanced efficacy as compared with a protein without a half-life extension element. Without being bound by theory, an exemplary way to improve the pharmacokinetics of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins, or fragments retain FcRn binding and are routinely linked to other polypeptides to extend their serum half-life. HSA may also be directly bound to the pharmaceutical compositions or bound via a short linker. Fragments of HSA may also be used. HSA and fragments thereof can function as both a blocking element and a half-life extension element. Human IgGs and Fc fragments can also carry out a similar function.

The serum half-life extension element can also be antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocalin and CTLA4 scaffolds. Further examples of antigen-binding polypeptides include a ligand for a desired receptor, a ligand-binding portion of a receptor, a lectin, and peptides that binds to or associates with one or more target antigens.

The half-life extension element as provided herein is preferably a human serum albumin (HSA) binding domain, and antigen binding polypeptide that binds human serum albumin or an immunoglobulin Fc or fragment thereof.

The half-life extension element of a inducible IL-12 prodrug extends the half-life of inducible IL-12 prodrug or the by at least about two days, about three days, about four days, about five days, about six days, about seven days, about eight days, about nine days, about 10 days or more. In some embodiments, the half-life extension element extends the half-life of a inducible IL-12 prodrug to at least 2-3 days, 3-4 days, 4-5 days, 5-6 days, 6-7 days, 7-8 days or more.

### C. Blocking Element

The blocking element can be any element that binds to IL-12 and inhibits the ability of the inducible IL-12 prodrug to bind and activate its receptor. The blocking element can inhibit the ability of the IL-12 to bind and/or activate its receptor e.g., by sterically blocking and/or by noncovalently binding to the IL-12 prodrug. The blocking element disclosed herein can bind to p19, p35, p40, the p35p40 heterodimeric complex, or the p19p40 heterodimeric complex.

Examples of suitable blocking elements include the full length or an IL-12-binding fragment or mutein of the cognate receptor of IL-12. Antibodies and antigen-binding fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like that bind IL-12 can also be used. Other suitable antigen-binding domain that bind IL-12 can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocalin and CTLA4 scaffolds. Further examples of suitable blocking polypeptides include polypeptides that sterically inhibit or block binding of IL-12 to its cognate receptor. Advantageously, such moieties can also function as half-life extending elements. For example, a peptide that is modified by conjugation to a water-soluble polymer, such as PEG, can sterically inhibit or prevent binding of the cytokine to its receptor. Polypeptides, or fragments thereof, that have long serum half-lives can also be used, such as serum albumin (human serum albumin), immunoglobulin Fc, transferrin and the like, as well as fragments and muteins of such polypeptides.

Preferred IL-12 blocking elements are single chain variable fragments (scFv) or Fab fragments. The scFv blocking elements comprise the amino acid sequence as set forth in SEQ ID NOs: 144-188. Alternatively, the Fab blocking element comprises the amino acid sequence as set forth in SEQ ID NOs: 189-194. The IL-12 antibody fragments encompassed by SEQ ID NOs: 144-194 have been optimized to enhance the developability of the inducible IL-12 prodrug disclosed herein.

Preferred antibody light chain blocking elements comprise SEQ ID NOs: 192-193. These preferred components can be located on one polypeptide chain and the complementary antigen binding portion of the heavy chain can be located on a second polypeptide chain. Preferred heavy chain blocking elements comprise SEQ ID NOs: 189-191 and 194. These preferred components can be located on one polypeptide chain and the complementary light chain is located on a second polypeptide chain. The antibody light chain and the antibody heavy chain together form a binding site for IL-12.

In some embodiments, the IL-12 blocking element comprises an amino acid sequence that is at least about 70%, at least about 75%, at least about 80%, at least about 85%, at least about 90%, at least about 95%, at least about 96%, at least about 97%, at least about 98%, at least about 99% identical to SEQ ID NOs: 144-194, e.g., over the full length of SEQ ID Nos:144-194. Typically, the amino acid sequence of the CDRs in not altered, and amino acid substitutions are present in the framework regions.

The disclosure also relates to functional variants of IL-12 blocking elements comprising SEQ ID NOs: 144-194. The functional variants of IL-12 blocking elements comprising SEQ ID NOs: 144-194 generally differ from SEQ ID NOs: 144-194 by one or a few amino acids (including substitutions, deletions, insertions, or any combination thereof), and substantially retain their ability to bind to the IL-12 polypeptide (e.g., the p35 subunit, the p40 subunit, or the p35p40 complex) and inhibit binding of IL-12 to its cognate receptor.

The functional variant can contain at least one or more amino acid substitutions, deletions, or insertions relative to the IL-12 blocking element comprising SEQ ID NOs: 144-194. The functional variant can comprise 1, 2, 3, 4, 5, 6, 7, 8, 9, or 10 amino acid alterations compared to the IL-12 blocking element comprising SEQ ID NOs: 144-194. In some preferred embodiments, the functional variant differs from the IL-12 blocking element comprising SEQ ID NOs: 144-194 by less than 10, less, than 8, less than 5, less than 4, less than 3, less than 2, or one amino acid alterations, e.g., amino acid substitutions or deletions. In other embodiments, the functional variant may comprise 1, 2, 3, 4, 5, 6, 7, 8, 9, or 10 amino acid substitutions compared to SEQ ID NOs: 144-194. The amino acid substitution can be a conservative substitution or a non-conservative substitution, but preferably is a conservative substitution.

In other embodiments, the functional variants of the IL-12 blocking element may comprise 1, 2, 3, 4, or 5 or more non-conservative amino acid substitutions compared the IL-12 blocking elements comprising SEQ ID NOs: 144-194. Non-conservative amino acid substitutions could be recognized by one of skill in the art. The functional variant of the separation moiety preferably contains no more than 1, 2, 3, 4, or 5 amino acid deletions.

Also disclosed herein is an inducible IL-12 prodrug that contains a blocking element having specificity for IL-12 and contains a half-life extension element. The blocking element is an antibody or antigen binding fragment that has binding specificity for IL-12, specifically the IL-12 subunit beta precursor (p40) as defined by SEQ ID NO: 421, disclosed herein. The antibody or antigen binding fragment comprises an antigen binding domain that binds to the residues shown in Table 2 of SEQ ID NO: 421. This disclosure relates to an antibody or antigen-binding fragment that binds the IL-12 epitope defined by the amino acid residues shown in Table 2, and to an inducible IL-12 prodrug that contains such an antibody or antigen-binding fragment, and to the use of such an antibody or antigen-binding fragment for the preparation of an inducible IL-12 prodrug, or a medicament containing such an inducible IL-12 prodrug.

TABLE 2
Epitope binding residues in the IL-12 subunit beta precursor

| | # with | # without |
|---|---|---|
| | signal | signal |
| | sequence | sequence |
| ASP | 36 | 14 |
| TRP | 37 | 15 |
| TYR | 38 | 16 |
| PRO | 39 | 17 |
| ASP | 40 | 18 |
| LYS | 106 | 84 |
| LYS | 107 | 85 |
| GLU | 108 | 86 |
| ASP | 109 | 87 |
| GLY | 110 | 88 |
| ILE | 111 | 89 |
| THR | 114 | 92 |
| ASP | 115 | 93 |
| LYS | 124 | 102 |
| ASN | 125 | 103 |
| LYS | 126 | 104 |
| LYS | 219 | 197 |

### D. Protease Cleavable Linker

As disclosed herein, the inducible IL-12 prodrug comprises one or more linker sequences. A linker sequence serves to provide flexibility between the polypeptides, such that, for example, the blocking element is capable of inhibiting the activity of IL-12. The linker can be located between the IL-12 subunit, the half-life extension element, and/or the blocking element. As described herein the inducible IL-12 prodrug comprises a protease cleavable linker. The protease cleavable linker can comprise one or more cleavage sites for one or more desired protease. Preferably, the desired protease is enriched or selectively expressed at the desired target site of IL-12 (e.g., the tumor microenvironment). Thus, the inducible IL-12 prodrug is preferentially or selectively cleaved at the target site of desired IL-12 activity.

Suitable linkers are typically less than about 100 amino acids. Such linkers can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domain. In a preferred embodiment, the linker is cleavable by a cleaving agent, e.g., an enzyme. Preferably, the separation moiety comprises a protease cleavage site. In some cases, the separation moiety comprises one or more cleavage sites. The separation moiety can comprise a single protease cleavage site. The separation moiety can also comprise 2 or more protease cleavage sites. For example, 2 cleavage sites, 3 cleavage sites, 4, cleavage sites, 5 cleavage sites, or more. In cases the separation moiety comprises 2 or more protease cleavage sites, the cleavage sites can be cleaved by the same protease or different proteases. A separation moiety comprising two or more cleavage sites is referred to as a “tandem linker.” The two or more cleavage sites can be arranged in any desired orientation, including, but not limited tom one cleavage site adjacent to another cleavage site, one cleavage site overlapping another cleavage site, or one cleavage site following by another cleavage site with intervening amino acids between the two cleavage sites.

Of particular interest in the present invention are disease specific protease-cleavable linkers. Also preferred are protease-cleavable linkers that are preferentially cleaved at a desired location in the body, such as the tumor microenvironment, relative to the peripheral circulation. For example, the rate at which the protease-cleavable linker is cleaved in the tumor microenvironment can be at least about 10 times, at least about 100 times, at least about 1000 times or at least about 10,000 times faster in the desired location in the body, e.g., the tumor microenvironment, in comparison to in the peripheral circulation (e.g., in plasma).

Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin G, Cathepsin K, Cathepsin L, kallikreins, hKl, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAPα), dipeptidyl peptidase, meprins, granzymes and dipeptidyl peptidase IV (DPPIV/CD26). Proteases capable of cleaving linker amino acid sequences (which can be encoded by the chimeric nucleic acid sequences provided herein) can, for example, be selected from the group consisting of a prostate specific antigen (PSA), a matrix metalloproteinase (MMP), an A Disintigrin and a Metalloproteinase (ADAM), a plasminogen activator, a cathepsin, a caspase, a tumor cell surface protease, and an elastase. The MMP can, for example, be matrix metalloproteinase 2 (MMP2), matrix metalloproteinase 9 (MMP9), matrix metalloproteinase 14 (MMP14). In addition, or alternatively, the linker can be cleaved by a cathepsin, such as, Cathepsin B, Cathepsin C, Cathepsin D, Cathepsin E, Cathepsin G, Cathepsin K and/or Cathepsin L. Preferably, the linker can be cleaved by MMP14 or Cathepsin L.

Proteases useful for cleavage of linkers and for use in the inducible IL-12 prodrug disclosed herein are presented in Table 3, and exemplary proteases and their cleavage site are presented in Table 4.

TABLE 3
Proteases relevant to inflammation and cancer

| | | |
|---|---|---|
| Protease | Specificity | Other aspects |

Secreted by killer T cells:

| | | |
|---|---|---|
| Granzyme B (grB) | Cleaves after Asp residues | Type of serine protease; strongly implicated |
| | (asp-ase) | in inducing perforin-dependent target cell |
| | | apoptosis |
| Granzyme A (grA) | trypsin-like, cleaves after | Type of serine protease; |
| | basic residues | |
| Granzyme H (grH) | Unknown substrate | Type of serine protease; |
| | specificity | Other granzymes are also secreted by killer |
| | | T cells, but not all are present in humans |
| Caspase-8 | Cleaves after Asp residues | Type of cysteine protease; plays essential |
| | | role in TCR-induced cellular expansion- |
| | | exact molecular role unclear |
| Mucosa-associated | Cleaves after arginine | Type of cysteine protease; likely acts both |
| lymphoid tissue (MALT1) | residues | as a scaffold and proteolytically active |
| | | enzyme in the CBM-dependent signaling |
| | | pathway |
| Tryptase | Targets: angiotensin I, | Type of mast cell-specific serine protease; |
| | fibrinogen, prourokinase, | trypsin-like; resistant to inhibition by |
| | TGFβ; preferentially | macromolecular protease inhibitors |
| | cleaves proteins after | expressed in mammals due to their |
| | lysine or arginine residues | tetrameric structure, with all sites facing |
| | | narrow central pore; also associated with |
| | | inflammation |

Associated with inflammation:

| | | |
|---|---|---|
| Thrombin | Targets: FGF-2, | Type of serine protease; modulates activity |
| | HB-EGF, Osteo-pontin, | of vascular growth factors, chemokines and |
| | PDGF, VEGF | extracellular proteins; strengthens VEGF- |
| | | induced proliferation; induces cell |
| | | migration; angiogenic factor; regulates |
| | | hemostasis |
| Chymase | Exhibit chymotrypsin-like | Type of mast cell-specific serine protease |
| | specificity, cleaving | |
| | proteins after aromatic | |
| | amino acid residues | |
| Carboxypeptidase A (MC- | Cleaves amino acid | Type of zinc-dependent metalloproteinase |
| CPA) | residues from C-terminal | |
| | end of peptides and | |
| | proteins | |
| Kallikreins | Targets: high molecular | Type of serine protease; modulate relaxation |
| | weight | response; contribute to inflammatory |
| | kininogen, pro-urokinase | response; fibrin degradation |
| Elastase | Targets: E-cadherin, GM- | Type of neutrophil serine protease; degrades |
| | CSF, IL-1, IL-2, IL-6, IL8, | ECM components; regulates inflammatory |
| | p38^{MAPK}, TNFα, VE- | response; activates pro-apoptotic signaling |
| | cadherin | |
| Cathepsin G | Targets: EGF, ENA-78, | Type of serine protease; degrades ECM |
| | IL-8, MCP-1, MMP-2, | components; chemo-attractant of |
| | MT1-MMP, | leukocytes; regulates inflammatory |
| | PAI-1, RANTES, TGFβ, | response; promotes apoptosis |
| | TNFα | |
| PR-3 | Targets: ENA-78, IL-8, IL- | Type of serine protease; promotes |
| | 18, JNK, p38^{MAPK}, TNFα | inflammatory response; activates pro- |
| | | apoptotic signaling |
| Granzyme M (grM) | Cleaves after Met and | Type of serine protease; only expressed in |
| | other long, unbranched | NK cells |
| | hydrophobic residues | |
| Calpains | Cleave between Arg and | Family of cysteine proteases; calcium- |
| | Gly | dependent; activation is involved in the |
| | | process of numerous inflammation- |
| | | associated diseases |

TABLE 4
Exemplary Proteases and Protease Recognition
Sequences

| | Cleavage | SEQ |
|---|---|---|
| | Domain | ID |
| Protease | Sequence | NO: |
| MMP7 | KRALGLPG | 375 |
| MMP7 | (DE)₈RPLALWRS(DR)₈ | 376 |
| MMP9 | PR(S/T)(L/I)(S/T) | 377 |
| MMP9 | LEATA | 378 |
| MMP11 | GGAANLVRGG | 379 |
| MMP14 | SGRIGFLRTA | 380 |
| MMP | PLGLAG | 381 |
| MMP | PLGLAX | 382 |
| MMP | PLGC(me)AG | 383 |
| MMP | ESPAYYTA | 384 |
| MMP | RLQLKL | 385 |
| MMP | RLQLKAC | 386 |
| MMP2, MMP9, | EP(Cit)G(Hof)YL | 387 |
| MMP14 | | |
| Urokinase | SGRSA | 388 |
| plasminogen | | |
| activator (uPA) | | |
| Urokinase | DAFK | 389 |
| plasminogen | | |
| activator (uPA) | | |
| Urokinase | GGGRR | 390 |
| plasminogen | | |
| activator (uPA) | | |
| Lysosomal Enzyme | GFLG | 391 |
| Lysosomal Enzyme | ALAL | 392 |
| Lysosomal Enzyme | FK | 393 |
| Cathepsin B | NLL | 394 |
| Cathepsin D | PIC(Et)FF | 395 |
| Cathepsin K | GGPRGLPG | 396 |
| Prostate Specific | HSSKLQ | 397 |
| Antigen | | |
| Prostate Specific | HSSKLQL | 398 |
| Antigen | | |
| Prostate Specific | HSSKLQEDA | 399 |
| Antigen | | |
| Herpes Simplex | LVLASSSFGY | 400 |
| Virus Protease | | |
| HIV Protease | GVSQNYPIVG | 401 |
| CMV Protease | GVVQASCRLA | 402 |
| Thrombin | F(Pip)RS | 403 |
| Thrombin | DPRSFL | 404 |
| Thrombin | PPRSFL | 405 |
| Caspase-3 | DEVD | 406 |
| Caspase-3 | DEVDP | 407 |
| Caspase-3 | KGSGDVEG | 408 |
| Interleukin 1ß | GWEHDG | 409 |
| converting enzyme | | |
| Enterokinase | EDDDDKA | 410 |
| FAP | KQEQNPGST | 411 |
| Kallikrein 2 | GKAFRR | 412 |
| Plasmin | DAFK | 413 |
| Plasmin | DVLK | 414 |
| Plasmin | DAFK | 415 |
| TOP | ALLLALL | 416 |
| | GPLGVRG | 417 |
| | IPVSLRSG | 418 |
| | VPLSLYSG | 419 |
| | SGESPAYYTA | 420 |

Exemplary protease cleavable linkers include, but are not limited to kallikrein cleavable linkers, thrombin cleavable linkers, chymase cleavable linkers, carboxypeptidase A cleavable linkers, cathepsin cleavable linkers, elastase cleavable linkers, FAP cleavable linkers, ADAM cleavable linkers, PR-3 cleavable linkers, granzyme M cleavable linkers, a calpain cleavable linkers, a matrix metalloproteinase (MMP) cleavable linkers, a plasminogen activator cleavable linkers, a caspase cleavable linkers, a tryptase cleavable linkers, or a tumor cell surface protease. Specifically, MMP9 cleavable linkers, ADAM cleavable linkers, CTSL1 cleavable linkers, FAPα cleavable linkers, and cathepsin cleavable linkers. Some preferred protease-cleavable linkers are cleaved by a MMP and/or a cathepsin.

The separation moieties disclosed herein are typically less than 100 amino acids. Such separation moieties can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domains.

In some embodiments, the separation moiety comprises the sequence GPAGLYAQ (SEQ ID NO: 195); GPAGMKGL (SEQ ID NO: 196); PGGPAGIG (SEQ ID NO: 197); ALFKSSFP (SEQ ID NO: 198); ALFFSSPP (SEQ ID NO: 199); LAQRLRSS (SEQ ID NO: 200); LAQKLKSS (SEQ ID NO; 201); GALFKSSFPSGGGPAGLYAQGGSGKGGSGK (SEQ ID NO: 202); RGSGGGPAGLYAQGSGGGPAGLYAQGGSGK (SEQ ID NO: 203); KGGGPAGLYAQGPAGLYAQGPAGLYAQGSR (SEQ ID NO: 204); RGGPAGLYAQGGPAGLYAQGGGPAGLYAQK (SEQ ID NO: 205); KGGALFKSSFPGGPAGIGPLAQKLKSSGGS (SEQ ID NO: 206); SGGPGGPAGIGALFKSSFPLAQKLKSSGGG (SEQ ID NO: 207); RGPLAQKLKSSALFKSSFPGGPAGIGGGGK (SEQ ID NO: 208); GGGALFKSSFPLAQKLKSSPGGPAGIGGGR (SEQ ID NO: 209); RGPGGPAGIGPLAQKLKSSALFKSSFPGGG (SEQ ID NO: 210); RGGPLAQKLKSSPGGPAGIGALFKSSFPGK (SEQ ID NO: 211); RSGGPAGLYAQALFKSSFPLAQKLKSSGGG (SEQ ID NO: 212); GGPLAQKLKSSALFKSSFPGPAGLYAQGGR (SEQ ID NO: 213); GGALFKSSFPGPAGLYAQPLAQKLKSSGGK (SEQ ID NO: 214); RGGALFKSSFPLAQKLKSSGPAGLYAQGGK (SEQ ID NO: 215); RGGGPAGLYAQPLAQKLKSSALFKSSFPGG (SEQ ID NO: 216); SGPLAQKLKSSGPAGLYAQALFKSSFPGSK (SEQ ID NO: 217); KGGPGGPAGIGPLAQRLRSSALFKSSFPGR (SEQ ID NO: 218); KSGPGGPAGIGALFFSSPPLAQKLKSSGGR (SEQ ID NO: 219); or SGGFPRSGGSFNPRTFGSKRKRRGSRGGGG (SEQ ID NO: 220)

Certain preferred separation moieties comprises the sequence GPAGLYAQ (SEQ ID NO: 195) or ALFKSSFP (SEQ ID NO: 198). The separation moieties disclosed herein can comprise one or more cleavage motif or functional variants that are the same or different. The separation moieties can comprise 1, 2, 3, 4, 5, or more cleavage motifs or functional variants. Separation moieties comprising 30 amino acids can contain 2 cleavage motifs or functional variants, 3 cleavage motifs or functional variants or more. A “functional variant” of a separation moiety retains the ability to be cleaved with high efficiency at a target site (e.g., a tumor microenvironment that expresses high levels of the protease) and are not cleaved or cleaved with low efficiency in the periphery (e.g., serum). For example, the functional variants retain at least about 50%, about 55%, about 60%, about 70%, about 80%, about 85%, about 95% or more of the cleavage efficiency of a separation moiety comprising any one of SEQ ID NOs: 195-220 or 447-448.

The separation moieties comprising more than one cleavage motif can be selected from SEQ ID NOs: 195-201 or 447-448 and combinations thereof. Preferred separation moieties comprising more than one cleavage motif comprise the amino acids selected from SEQ ID NO: 202-220.

The separation moiety can comprise both ALFKSSFP (SEQ ID NO: 198) and GPAGLYAQ (SEQ ID NO: 195). The separation moiety can comprise two cleavage motifs that each have the sequence GPAGLYAQ (SEQ ID NO: 195). Alternatively or additionally, the separation moiety can comprise two cleavage motifs that each have the sequence ALFKSSFP (SEQ ID NO: 198). The separation moiety can comprise a third cleavage motif that is the same or different.

In some embodiments, the separation moiety comprises an amino acid sequence that is at least about 90%, at least about 95%, at least about 96%, at least about 97%, at least about 98%, or at least 99% identical to SEQ ID NOs: 195 to SEQ ID NO: 220 or 447-448 over the full length of SEQ ID NO: 195-220 or SEQ ID NOS 447-448.

The disclosure also relates to functional variants of separation moieties comprising SEQ ID NOs: 195-220 or 447-448. The functional variants of separation moieties comprising SEQ ID NOs: 195-220 or 447-448 generally differ from SEQ ID NOs: 195-220 or 447-448 by one or a few amino acids (including substitutions, deletions, insertions, or any combination thereof), and substantially retain their ability to be cleaved by a protease.

The functional variants can contain at least one or more amino acid substitutions, deletions, or insertions relative to the separation moieties comprising SEQ ID NOs: 195-220 or 447-448. The functional variant can comprise 1, 2, 3, 4, 5, 6, 7, 8, 9, or 10 amino acid alterations comparted to the separation moieties comprising SEQ ID NOs: 195-220 or 447-448. In some preferred embodiments, the functional variant differs from the separation moiety comprising SEQ ID NOs: 195-220 by less than 10, less, than 8, less than 5, less than 4, less than 3, less than 2, or one amino acid alterations, e.g., amino acid substitutions or deletions. In other embodiments, the functional variant may comprise 1, 2, 3, 4, 5, 6, 7, 8, 9, or 10 amino acid substitutions compared to SEQ ID NOs: 195-220 or 447-448. The amino acid substitution can be a conservative substitution or a non-conservative substitution, but preferably is a conservative substitution.

In other embodiments, the functional variants of the separation moieties may comprise 1, 2, 3, 4, or 5 or more non-conservative amino acid substitutions compared the separation moieties comprising SEQ ID NOs: 195-220 or 447-448. Non-conservative amino acid substitutions could be recognized by one of skill in the art. The functional variant of the separation moiety preferably contains no more than 1, 2, 3, 4, or 5 amino acid deletions.

The amino acid sequences disclosed in the separation moieties can be described by the relative linear position in the separation moiety with respect to the sissile bond. As will be well-understood by persons skilled in the art, separation moieties comprising 8 amino acid protease substrates (e.g., SEQ ID Nos: 195-201 or 447-448) contain amino acid at positions P4, P3, P2, P1, P1′, P2′, P3′, P4′, wherein the sissile bond is between P1 and P1′. For example, amino acid positions for the separation moiety comprising the sequence GPAGLYAQ (SEQ ID NO: 195) can be described as follows:

| G | P | A | G | L | Y | A | Q |
|---|---|---|---|---|---|---|---|
| P4 | P3 | P2 | P1 | P1′ | P2′ | P3′ | P4′ |

Amino acids positions for the separation moiety comprising the sequence ALFKSSFP (SEQ ID NO: 198) can be described as follows:

| A | L | F | K | S | S | F | P |
|---|---|---|---|---|---|---|---|
| P4 | P3 | P2 | P1 | P1′ | P2′ | P3′ | P4′ |

Preferably, the amino acids surrounding the cleavage site (e.g., positions P1 and P1′ for SEQ ID NOs: 195-201 or 447-448) are not substituted.

In embodiments, the separation moiety comprises the sequence GPAGLYAQ (SEQ ID NO: 195) or ALFKSSFP (SEQ ID NO: 198) or a functional variant of SEQ ID NO: 195 or a function variant of SEQ ID NO: 198. As described herein, a functional variant of PAGLYAQ (SEQ ID NO: 447) or ALFKSSFP (SEQ ID NO: 198) can comprise one or more amino acid substitutions, and substantially retain their ability to be cleaved by a protease. Specifically, the functional variants of GPAGLYAQ (SEQ ID NO: 195) is cleaved by MMP14, and the functional variant of ALFKSSFP (SEQ ID NO: 198) is cleaved by Capthepsin L (CTSL1). The functional variants also retain their ability to be cleaved with high efficiency at a target site (e.g., a tumor microenvironment that expresses high levels of the protease). For example, the functional variants of GPAGLYAQ (SEQ ID NO: 195) or ALFKSSFP (SEQ ID NO: 198) retain at least about 50%, about 55%, about 60%, about 70%, about 80%, about 85%, about 95% or more of the cleavage efficiency of a separation moiety comprising amino acid sequence GPAGLYAQ (SEQ ID NO: 195) or ALFKSSFP (SEQ ID NO: 198), respectively.

Preferably, the functional variant of GPAGLYAQ (SEQ ID NO: 195) or ALFKSSFP (SEQ ID NO: 198) comprise no more than 1, 2, 3, 4, or 5 conservative amino acid substitutions compared to GPAGLYAQ (SEQ ID NO: 195) or ALFKSSFP (SEQ ID NO: 198). Preferably, the amino acids at position P1 and P1′ are not substituted. The amino acids at positions P1 and P1′ in SEQ ID NO: 195 are G and L, and the amino acids at positions P1 and P1′ in SEQ ID NO: 198 are K and S.

The functional variant of GPAGLYAQ (SEQ ID NO: 195) can preferably comprise one or more of the following: a) an arginine amino acid substitution at position P4, b) a leucine, valine, asparagine, or proline amino acid substitution at position P3, c) a asparagine amino acid substitution at position P2, d) a histidine, asparagine, or glycine amino acid substitution at position P1, e) a asparagine, isoleucine, or leucine amino acid substitution at position P1′, f) a tyrosine or arginine amino acid substitution at position P2′, g) a glycine, arginine, or alanine amino acid substitution at position P3′, h) or a serine, glutamine, or lysine amino acid substitution at position P4′. The following amino acid substitutions are disfavored in functional variants of GPAGLYAQ (SEQ ID NO: 195): a) arginine or isoleucine at position P3, b) alanine at position P2, c) valine at position P1, d) arginine, glycine, asparagine, or threonine at position P1′, e) aspartic acid or glutamic acid at position P2′, f) isoleucine at position P3′, g) valine at position P4′. In some embodiments, the functional variant of GPAGLYAQ (SEQ ID NO: 195) does not comprise an amino acid substitution at position P1 and/or P1′.

The amino acid substitution of the functional variant of GPAGLYAQ (SEQ ID NO: 195) preferably comprises an amino acid substitution at position P4 and/or P4′. For example, the functional variant of GPAGLYAQ (SEQ ID NO: 195) can comprise a leucine at position P4, or serine, glutamine, lysine, or phenylalanine at position P4. Alternatively or additionally, the functional variant of GPAGLYAQ (SEQ ID NO: 195) can comprise a glycine, phenylalanine, or a proline at position P4′.

In some embodiments, the amino acid substitutions at position P2 or P2′ of GPAGLYAQ (SEQ ID NO: 195) are not preferred.

In some embodiments, the functional variant of GPAGLYAQ (SEQ ID NO: 195) comprises the amino acid sequence selected from SEQ ID NOs: 221-295. Specific functional variants of GPAGLYAQ (SEQ ID NO: 195) include GPLGLYAQ (SEQ ID NO: 259), and GPAGLKGA (SEQ ID NO: 249).

The functional variants of LFKSSFP (SEQ ID NO: 448) preferably comprises hydrophobic amino acid substitutions. The functional variant of LFKSSFP (SEQ ID NO: 448) can preferably comprise one or more of the following: (a) lysine, histidine, serine, glutamine, leucine, proline, or phenylalanine at position P4; (b) lysine, histidine, glycine, proline, asparagine, phenylalanine at position P3; (c) arginine, leucine, alanine, glutamine, or histatine at position P2; (d) phenylalanine, histidine, threonine, alanine, or glutamine at position P1; has histidine, leucine, lysine, alanine, isoleucine, arginine, phenylalanine, asparagine, glutamic acid, or glycine at position P1′, (f) phenylalanine, leucine, isoleucine, lysine, alanine, glutamine, or proline at position P2′; (g) phenylalanine, leucine, glycine, serine, valine, histidine, alanine, or asparagine at position P3′; and phenylalanine, histidine, glycine, alanine, serine, valine, glutamine, lysine, or leucine.

The inclusion of aspartic acid and/or glutamic acid in functional variants of SEQ ID NO: 448 are generally disfavored and avoided. The following amino acid substitutions are also disfavored in functional variants of LFKSSFP (SEQ ID NO: 448): (a) alanine, serine, or glutamic acid at position P3; (b) proline, threonine, glycine, or aspartic acid at position P2; (c) proline at position P1; (d) proline at position P1′; (e) glycine at position P2′; (f) lysine or glutamic acid at position P3′; (g) aspartic acid at position P4′.

The amino acid substitution of the functional variant of LFKSSFP (SEQ ID NO: 448) preferably comprises an amino acid substitution at position P4 and/or P1. In some embodiments, an amino acid substitution of the functional variant of LFKSSFP (SEQ ID NO: 448) at position P4′ is not preferred.

In some embodiments, the functional variant of LFKSSFP (SEQ ID NO: 448) comprises the amino acid sequence selected from SEQ ID NOs: 296-374. Specific functional variants of LFKSSFP (SEQ ID NO: 448) include ALFFSSPP (SEQ ID NO: 199), ALFKSFPP (SEQ ID NO: 346), ALFKSLPP (SEQ ID NO: 347); ALFKHSPP (SEQ ID NO: 335); ALFKSIPP (SEQ ID NO: 348); ALFKSSLP (SEQ ID NO: 356); or SPFRSSRQ (SEQ ID NO: 297).

The separation moieties disclosed herein can form a stable complex under physiological conditions with the amino acid sequences (e.g. domains) that they link, while being capable of being cleaved by a protease. For example, the separation moiety is stable (e.g., not cleaved or cleaved with low efficiency) in the circulation and cleaved with higher efficiency at a target site (i.e. a tumor microenvironment). Accordingly, fusion polypeptides that include the linkers disclosed herein can, if desired, have a prolonged circulation half-life and/or lower biological activity in the circulation in comparison to the components of the fusion polypeptide as separate molecular entities. Yet, when in the desired location (e.g., tumor microenvironment) the linkers can be efficiently cleaved to release the components that are joined together by the linker and restoring or nearly restoring the half-life and biological activity of the components as separate molecular entities.

The separation moiety desirably remains stable in the circulation for at least 2 hours, at least 5, hours, at least 10 hours, at least 15 hours, at least 20 hours, at least 24 hours, at least 30 hours, at least 35 hours, at least 40 hours, at least 45 hours, at least 50 hours, at least 60 hours, at least 65 hours, at least 70 hours, at least 80 hours, at least 90 hours, or longer.

In some embodiments, the separation moiety is cleaved by less than 90%, 80%, 70%, 60%, 50%, 40%, 30%, 20%, 20%, 5%, or 1% in the circulation as compared to the target location. The separation moiety is also stable in the absence of an enzyme capable of cleaving the linker. However, upon expose to a suitable enzyme (i.e., a protease), the separation moiety is cleaved resulting in separation of the linked domain.

### E. Pharmaceutical Compositions

Also provided herein, are pharmaceutical compositions comprising an inducible IL-12 prodrug described herein, a vector comprising the polynucleotide encoding the inducible IL-12 prodrug or a host cell transformed by this vector and at least one pharmaceutically acceptable carrier.

Provided herein are pharmaceutical formulations or compositions containing the inducible IL-12 prodrugs as described herein and a pharmaceutically acceptable carrier. Compositions comprising the inducible IL-12 prodrugs as described herein are suitable for administration in vitro or in vivo. The term “pharmaceutically acceptable carrier” includes, but is not limited to, any carrier that does not interfere with the effectiveness of the biological activity of the ingredients and that is not toxic to the subject to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic, although the formulate can be hypertonic or hypotonic if desired. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the IL-12 or nucleic acid sequences encoding the inducible IL-12 prodrugs to humans or other subjects.

This disclosure also relates to pharmaceutical formulations that contain an IL-12 prodrug as described herein. The formulation is preferably an aqueous liquid, more preferable an aqueous liquid suitable for inject or infusion. The formulation can also preferably be a dry solid formulation, such as a lyophilizate or spray dried formulation. In embodiments, the formulation is a lyophilizate (a lyophilized cake). Preferred formulations include an IL-12 prodrug as described herein, citric acid and/or a citrate salt, a disaccharide and a surfactant.

In such liquid formulations, the IL-12 prodrug can be present from about 1 mg/mL to about 100 mg/mL, for example about 50 mg/mL to about 100 mg/mL, about 50 mg/mL to about 75 mg/mL, about 75 mg/mL to about 100 mg/mL, about 25 mg/mL to about 50 mg/mL, about 1 mg/mL to about 50 mg/mL, about 1 mg/mL to about 25 mg/mL, about 1 mg/mL to about 20 mg/mL, about 1 mg/mL to about 15 mg/mL, about 1 mg/mL to about 10 mg/mL, about 5 mg/mL to about 25 mg/mL, about 5 mg/mL to about 20 mg/mL, about 5 mg/mL to about 15 mg/mL, about 5 mg/mL to about 10 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL or about 25 mg/mL. The aqueous liquid can have a pH between about 5.0 and about 8.0, for example about 5.0 to about 7.0, about 5.5 to about 7.0, about 5.0, about 5.5, about 6.0, about 6.5, about 7.0, about 7.5 or about 8.0.

In such liquid formulation, a citrate salt (e.g., monosodium citrate, disodium citrate and trisodium citrate) is present at a concentration of about 5 mM to about 500 mM, for example, 5 mM to about 300 mM, 5 mM to about 250 mM, 5 mM to about 200 mM, 5 mM to about 150 mM, 5 mM to about 100 mM, 10 mM to about 100 mM, 20 mM to about 100 mM, 20 mM to about 90 mM, 20 mM to about 80 mM, 30 mM to about 80 mM, 30 mM to about 70 mM, 40 mM to about 70 mM, 40 mM to about 60 mM, about 20 mM, about 30 mM, about 40 mM, about 50 mM, about 60 mM, about 70 mM, about 80 mM, about 90 mM, or about 100 mM.

In such liquid formulation, a disaccharide (e.g sucrose, trehalose, lactose, maltose) is present at a concentration of about 20 mM to about 500 mM, for example, about 20 mM to about 300 mM, 20 mM to about 250 mM, 100 mM to about 300 mM, 100 mM to about 250 mM, about 100 mM, about 120 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 370 mM, about 390 mM or about 400 mM.

In such liquid formulation, a surfactant (e.g polysorbate 80, polysorbate 20, span-80, poloxamer) is present at about 0.001% to about 2%, for example, about 0.001% to about 1%, about 0.001% to about 0.1%, about 0.01% to about 1%, about 0.01% to about 0.1%, about 0.002% to about 0.2%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, or about 0.2%.

Suitable citrate salts are known in the art and include, sodium citrate (such as monosodium citrate, disodium citrate and trisodium citrate), magnesium citrate, potassium citrate, and the like. Suitable disaccharides are known in the art and include, sucrose, trehalose, lactose, maltose and the like. Surfactants are known in the art and include ionic surfactants such as fatty acids and fatty acid salts (e.g., sodium stearate, magnesium stearate), alkyl sulfates and salts thereof (e.g. sodium dodecyl sulfate), certain water soluble quaternary ammonium salts, and the like. Suitable surfactants also include nonionic surfactants, such as polysorbate 20, polysorbate 80, Span-80, castor oil, poloxamers, and the like.

The formulation can include an IL-12 prodrug as described herein, citric acid and/or sodium citrate (e.g. monosodium citrate, disodium citrate and trisodium citrate), a disaccharide (e.g., sucrose, trehalose, lactose, maltose) and a non-ionic surfactant (polysorbate 20, polysorbate 80, Span-80, castor oil, poloxamers). The formulation can include an IL-12 prodrug as described herein, citric acid and/or sodium citrate (e.g. monosodium citrate, disodium citrate and trisodium citrate), sucrose and polysorbate 80.

In particular embodiments, the formulation is a aqueous liquid for injection of infusion and contains an IL-12 prodrug as described herein at a concentration of about 1 mg/mL to about 100 mg/mL, sodium citrate (e.g. monosodium citrate, disodium citrate and trisodium citrate) at a concentration of about 5 mM to about 500 mM, sucrose at a concentration of about 20 mM to about 500 mM, polysorbate 80 at a concentration of about 0.001% to about 2%, and a pH of about 5.0 and about 8.0. Such formulations also include water, for example water for injection, USP.

Preferably the IL-12 prodrug concentration is about 50 mg/mL to about 75 mg/mL, about 75 mg/mL to about 100 mg/mL, about 25 mg/mL to about 50 mg/mL, about 1 mg/mL to about 50 mg/mL, about 1 mg/mL to about 25 mg/mL, about 1 mg/mL to about 20 mg/mL, about 1 mg/mL to about 15 mg/mL, about 1 mg/mL to about 10 mg/mL, about 5 mg/mL to about 25 mg/mL, about 5 mg/mL to about 20 mg/mL, about 5 mg/mL to about 15 mg/mL, about 5 mg/mL to about 10 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL or about 25 mg/mL; the sodium citrate (e.g. monosodium citrate, disodium citrate and trisodium citrate) concentration is about 5 mM to about 300 mM, 5 mM to about 250 mM, 5 mM to about 200 mM, 5 mM to about 150 mM, 5 mM to about 100 mM, 10 mM to about 100 mM, 20 mM to about 100 mM, 20 mM to about 90 mM, 20 mM to about 80 mM, 30 mM to about 80 mM, 30 mM to about 70 mM, 40 mM to about 70 mM, 40 mM to about 60 mM, about 20 mM, about 30 mM, about 40 mM, about 50 mM, about 60 mM, about 70 mM, about 80 mM, about 90 mM, or about 100 mM; the sucrose concentration is about 20 mM to about 300 mM, 20 mM to about 250 mM, 100 mM to about 300 mM, 100 mM to about 250 mM, about 100 mM, about 120 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 370 mM, about 390 mM or about 400 mM; the polysorbate 80 concentration is about 0.001% to about 1%, about 0.001% to about 0.1%, about 0.01% to about 1%, about 0.01% to about 0.1%, about 0.002% to about 0.2%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, or about 0.2%, and the pH is about 5.0 to about 7.0, about 5.5 to about 7.0, about 5.0, about 5.5, about 6.0, about 6.5, about 7.0, about 7.5 or about 8.0.

The formulation can also be a dry solid formulation, such as a lyophilizate (lyophilized cake) or spray dried powder of any of the liquid formulations described herein. Such dry solid formulations can be reconstituted, for example using water for injection, USP, to produce a liquid formulation for injection or infusion. The dry solid formulations of this disclosure are not necessarily anhydrous and may include some water if desired.

In some embodiments of the pharmaceutical compositions, the inducible IL-12 prodrug described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the inducible IL-12 prodrug is attached to liposomes. In some instances, the inducible IL-12 prodrug are conjugated to the surface of liposomes. In some instances, the inducible IL-12 prodrugs are encapsulated within the shell of a liposome. In some instances, the liposome is a cationic liposome.

The inducible IL-12 prodrug described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

Optionally, the inducible IL-12 prodrug or nucleic acid sequences encoding the inducible IL-12 prodrug are administered by a vector. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein. Such compositions and methods can be used to transfect or transduce cells in vitro or in vivo, for example, to produce cell lines that express and preferably secrete the encoded chimeric polypeptide or to therapeutically deliver nucleic acids to a subject. The components of the IL-12 polypeptide disclosed herein are typically operably linked in frame to encode a fusion protein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general and methods of making them are described by Coffin et al., Retroviruses, Cold Spring Harbor Laboratory Press (1997). The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided inducible IL-12 prodrugs and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The inducible IL-12 prodrugs disclosed herein can be delivered by subviral dense bodies (DBs). DBs transport proteins into target cells by membrane fusion. Methods for making and using DBs are described in, for example, Pepperl-Klindworth et al., Gene Therapy 10:278-84 (2003). The provided polypeptides can be delivered by tegument aggregates. Methods for making and using tegument aggregates are described in International Publication No. WO 2006/110728.

Non-viral based delivery methods, can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Pal Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Such vectors can also be used to make the inducible IL-12 prodrugs by expression in a suitable host cell, such as CHO cells.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g., β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

### F. Therapeutic Applications

Also provided herein, are methods and uses for the treatment of a disease, disorder or condition associated with a target antigen comprising administering to a subject in need thereof a inducible IL-12 prodrug as described herein. Diseases, disorders, or conditions include, but are not limited to, cancer, inflammatory disease, an immunological disorder, autoimmune disease, infectious disease (i.e., bacterial, viral, or parasitic disease). Preferably, the disease, disorder, or condition is cancer.

Any suitable cancer may be treated with the inducible IL-12 prodrugs provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. In embodiments, the cancer is melanoma or breast cancer.

In some embodiments, provided herein is a method of enhancing an immune response in a subject in need thereof by administering an effective amount of an inducible IL-12 prodrugs provided herein to the subject. The enhanced immune response may prevent, delay, or treat the onset of cancer, a tumor, or a viral disease. Without being bound by theory, the inducible IL-12 prodrug enhances the immune response by activating the innate and adaptive immunities. In some embodiments, the methods described herein increase the activity of Natural Killer Cells and T lymphocytes. In some embodiments, the inducible IL-12 prodrug provided herein, can induce IFNγ release from Natural Killer cells as well as CD4+ and CD8+ T cells.

The IL-12 prodrug can be administered to a subject in need thereof in combination with an immune checkpoint inhibitor. Immune checkpoint proteins include, for example, PD-1 which binds ligands PD-L1 (B7-H1, CD274) and PD-L2 (B7-DC, CD273), CTLA-4 (CD152) which binds B7-1 (CD80) and B7-2 (CD86), LAG 3 (CD223) which binds Galectin3, LSECtin and FGL1; TIM3 (HAVCR2) which binds ligands Ceacam1 and Galectin9; TIGIT (VSTM3, WUCAM) which binds CD112 and CD155; BTLA (CD272) which binds HVEM (TNFRSF14), B7-H3 (CD276), B7-H4 (VTCN1), VISTA (B7-H5), KIR, CD44 (2B4), CD160 (BY55) which bind HVEM; CD134 (TNRFSR4, OX40) which binds CD252 (OX-40L). Therapeutic agents, such as antibodies, that bind immune checkpoint proteins and inhibit their immunosuppressive activity include the anti-PD1 antibodies pembrolizumab (KEYTRUDA), dostarlimab (JEMPERLI), cemiplimab-rwlc (LIBATYO), nivolumab (OPDIVO), camrelizumab, tislelizumab, toripalimab, and sintilimab (TYVYT); the anti-PD-L1 antibodies avelumab (BAVENCIO), durvalumab (IMFINZI), and atezolizumab (TECENTRIQ); the anti-CTLA-4 antibody ipilimumab (YERVOY).

The method can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-L1, anti-CTLA4, anti-PD-1, anti-CD47, anti-GD2), cellular therapies (e.g., CAR-T, T-cell therapy), oncolytic viruses and the like. Non-limiting examples of anti-cancer agents that can be used include acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacytidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-nl interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinzolidine sulfate; vinzolidine sulfate; vorozole; zeniplatin; zinostatin; zorubicin hydrochloride.

In some embodiments of the methods described herein, the inducible IL-12 prodrug is administered in combination with an agent for the treatment of the particular disease, disorder, or condition. Agents include, but are not limited to, therapies involving antibodies, small molecules (e.g., chemotherapeutics), hormones (steroidal, peptide, and the like), radiotherapies (γ-rays, C-rays, and/or the directed delivery of radioisotopes, microwaves, UV radiation and the like), gene therapies (e.g., antisense, retroviral therapy and the like) and other immunotherapies. In some embodiments, the inducible IL-12 prodrug is administered in combination with anti-diarrheal agents, anti-emetic agents, analgesics and/or non-steroidal anti-inflammatory agents.

The disclosure relates to methods for treating cancer using an inducible IL-12 prodrug as described herein. The methods disclosed herein comprise administering to a subject a therapeutically effective amount of an inducible IL-12 prodrug described herein.

The inducible IL-12 prodrug can be administered in a dose of about 0.016 mg/kg to about 500 mg/kg per administration. For example, about 0.016 mg/kg, about 0.017 mg/kg about 0.018 mg/kg, about 0.019 mg/kg, about 0.020 mg/kg, about 0.021 mg/kg, about 0.022 mg/kg, about 0.023 mg/kg, about 0.024 mg/kg, about 0.025 mg/kg, about 0.026 mg/kg, about 0.027 mg/kg, about 0.028 mg/kg, about 0.029 mg/kg, about 0.030 mg/kg, about 0.031 mg/kg, about 0.032 mg/kg, about 0.033 mg/kg, about 0.034 mg/kg, about 0.035 mg/kg, about 0.036 mg/kg, about 0.037 mg/kg, about 0.038 mg/kg, about 0.039 mg/kg, about 0.040 mg/kg, about 0.041 mg/kg, about 0.042 mg/kg, about 0.043 mg/kg, about 0.044 mg/kg, about 0.045 mg/kg, about 0.046 mg/kg, about 0.047 mg/kg, about 0.048 mg/kg, about 0.049 mg/kg, about 0.050 mg/kg, about 0.051 mg/kg, about 0.052 mg/kg, about 0.053 mg/kg, about 0.054 mg/kg, about 0.055 mg/kg, about 0.056 mg/kg, about 0.057 mg/kg, about 0.058 mg/kg, about 0.059 mg/kg, about 0.060 mg/kg, about 0.061 mg/kg, about 0.062 mg/kg, about 0.063 mg/kg, about 0.064 mg/kg, about 0.065 mg/kg, about 0.066 mg/kg, about 0.067 mg/kg, about 0.068 mg/kg, about 0.069 mg/kg, about 0.070 mg/kg, about 0.071 mg/kg, about 0.072 mg/kg, about 0.073 mg/kg, about 0.074 mg/kg, about 0.075 mg/kg, about 0.076 mg/kg, about 0.077 mg/kg, about 0.078 mg/kg, about 0.079 mg/kg, about 0.080 mg/kg, about 0.081 mg/kg, about 0.082 mg/kg, about 0.083 mg/kg, about 0.084 mg/kg, about 0.085 mg/kg, about 0.086 mg/kg, about 0.087 mg/kg, about 0.088 mg/kg, about 0.089 mg/kg, about 0.090 mg/kg, about 0.091 mg/kg, about 0.092 mg/kg, about 0.093 mg/kg, about 0.094 mg/kg, about 0.095 mg/kg, about 0.096 mg/kg, about 0.097 mg/kg, about 0.098 mg/kg, about 0.099 mg/kg, or about 0.100 mg/kg, in each case per administration.

For example, about 0.100 mg/kg, about 0.105 mg/kg, about 0.110 mg/kg, about 0.115 mg/kg, about 0.120 mg/kg, about 0.125 mg/kg, about 0.130 mg/kg, about 0.135 mg/kg, about 0.140 mg/kg, about 0.145 mg/kg, about 0.150 mg/kg, about 0.155 mg/kg, about 0.160 mg/kg, about 0.165 mg/kg, about 0.170 mg/kg, about 0.175 mg/kg, about 0.180 mg/kg, about 0.185 mg/kg, about 0.190 mg/kg, about 0.195 mg/kg, about 0.200 mg/kg, about 0.205 mg/kg, about 0.210 mg/kg, about 0.215 mg/kg, about 0.220 mg/kg, about 0.230 mg/kg, about 0.235 mg/kg, about 0.240 mg/kg, about 0.245 mg/kg, about 0.250 mg/kg, about 0.255 mg/kg, about 0.260 mg/kg, about 0.265 mg/kg, about 0.270 mg/kg, about 0.275 mg/kg, about 0.280 mg/kg, about 0.285 mg/kg, about 0.290 mg/kg, about 0.295 mg/kg, about 0.300 mg/kg, about 0.305 mg/kg, about 0.310 mg/kg, about 0.315 mg/kg, about 0.320 mg/kg, about 0.325 mg/kg, about 0.330 mg/kg, about 0.340 mg/kg, about 0.345 mg/kg, about 0.350 mg/kg, about 0.355 mg/kg, about 0.360 mg/kg, about 0.365 mg/kg, about 0.370 mg/kg, about 0.375 mg/kg, about 0.380 mg/kg, about 0.385 mg/kg, about 0.390 mg/kg, about 0.395 mg/kg, about 0.400 mg/kg, about 0.405 mg/kg, about 0.410 mg/kg, about 0.415 mg/kg, about 0.420 mg/kg, about 0.425 mg/kg, about 0.430 mg/kg, about 0.435 mg/kg, about 0.440 mg/kg, about 0.445 mg/kg, about 0.450 mg/kg, about 0.455 mg/kg, about 0.460 mg/kg, about 0.465 mg/kg, about 0.470 mg/kg, about 0.475 mg/kg, about 0.480 mg/kg, about 0.485 mg/kg, about 0.490 mg/kg, about 0.495 mg/kg, or about 0.500 mg/kg, in each case per administration.

Typically, the IL-12 prodrug is administered in an amount of about 0.032 mg/kg, about 0.056 mg/kg, about 0.084 mg/kg, about 0.126 mg/kg, about 0.190 mg/kg, about 0.290 mg/kg or about 0.440 mg/kg, in each case per administration.

In such methods, the IL-12 prodrug can be administered orally, parenterally, intravenously, intraarticularly, intraperitoneally, intramuscularly, subcutaneously, intracavity, transdermally, intrahepatically, intracranially, nebulization/inhalation, by installation via bronchoscopy, or intratumorally. Typically, the IL-12 prodrug is administered intravenously.

The inducible IL-12 prodrug can be administered about twice a week or less frequently, for example once every two weeks.

### G. Definitions

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

As used herein, the terms “activatable,” “activate,” “induce,” and “inducible” refers to a polypeptide complex that has an attenuated activity form (e.g., attenuated receptor binding and/or agonist activity) and an activated form. The polypeptide complex is activated by protease cleavage of the linker that causes the blocking element and half-life extension element to dissociate from the polypeptide complex. The induced/activated polypeptide complex can bind with increased affinity/avidity to the IL-12 receptor.

The terms “antibody” and “immunoglobulin” are used interchangeably herein. An antibody or immunoglobulin, as used herein, is intended to refer to immunoglobulin molecules comprised of two heavy (H) chains. Typically, antibodies in mammals (e.g., humans, rodents, and monkey's) comprise four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CHI, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Antibodies can include, for example, monoclonal antibodies, recombinantly produced antibodies, monospecific antibodies, multi specific antibodies (including bispecific antibodies), human antibodies, humanized antibodies, chimeric antibodies, immunoglobulins, synthetic antibodies, or tetrameric antibodies comprising two heavy chain and two light chain molecules. One of skill in the art would recognize that other forms of antibodies exist (e.g. camelid and shark antibodies).

The term “attenuated” as used herein is an IL-12 receptor agonist that has decreased receptor agonist activity as compared to the IL-12 receptor's naturally occurring agonist. An attenuated IL-12 agonist can have at least about 10×, at least about 50×, at least about 100×, at least about 250×, at least about 500×, at least about 1000× or less agonist activity as compared to the receptor's naturally occurring agonist. When a IL-12 polypeptide complex that contains IL-12 as described herein is described as “attenuated” or having “attenuated activity”, it is meant that the IL-12 polypeptide complex is an attenuated IL-12 receptor agonist.

The term “cancer” refers to the physiological condition in mammals in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such a leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer), osteosarcoma, melanoma, colon cancer, colorectal cancer, endometrial (e.g., serous) or uterine cancer, salivary gland carcinoma, kidney cancer, liver cancer, prostate cancer, vulval cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu.

A “conservative” amino acid substitution, as used herein, generally refers to substitution of one amino acid residue with another amino acid residue from within a recognized group which can change the structure of the peptide but biological activity of the peptide is substantially retained. Conservative substitutions of amino acids are known to those skilled in the art. Conservative substitutions of amino acids can include, but not limited to, substitutions made amongst amino acids within the following groups: (a) M, I, L, V; (b) F, Y, W; (c) K, R, H; (d) A, G; (e) S, T; (f) Q, N; and (g) E, D. For instance, a person of ordinary skill in the art reasonably expect that an isolated replacement of a leucine with an isoleucine or valine, an aspartate with a glutamate, a threonine with a serine, or a similar replacement of an amino acid with a structurally related amino acid will not have a major effect on the biological activity of the resulting molecule.

As used herein, the term “half-life extension element” in the context of the polypeptide complex disclosed herein, refers to a chemical element, preferable a polypeptide that increases the serum half-life and improve pK, for example, by altering its size (e.g., to be above the kidney filtration cutoff), shape, hydrodynamic radius, charge, or parameters of absorption, biodistribution, metabolism, and elimination.

As used herein, the term “operably linked” in the context of a polypeptide complex refers to the orientation of the components of a polypeptide complex that permits the components to function in their intended manner. For example, a polypeptide comprising an IL-12 subunit and an IL-12 blocking element are operably linked by a protease cleavable linker in a polypeptide complex when the IL-12 blocking element is capable of inhibiting the IL-12 receptor-activating activity of the IL-12 polypeptide, but upon cleavage of the protease cleavable linker the inhibition of the IL-12 receptor-activating activity of the IL-12 polypeptide by the IL-12 blocking element is decreased or eliminated, for example because the IL-12 blocking element can diffuse away from the IL-12.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

The term “subject” herein to refers to any animal, such as any mammal, including but not limited to, humans, non-human primates, rodents, and the like. In some embodiments, the mammal is a mouse. In some embodiments, the mammal is a human.

As used herein, the term “therapeutically effective amount” refers to an amount of a compound described herein (i.e., a IL-12 polypeptide complex) that is sufficient to achieve a desired pharmacological or physiological effect under the conditions of administration. For example, a “therapeutically effective amount” can be an amount that is sufficient to reduce the signs or symptoms of a disease or condition (e.g., a tumor). Those skilled in the art will appreciate that the therapeutic effects need not be complete or curative, as long as some benefit is provided to the subject. A therapeutically effective amount of a pharmaceutical composition can vary according to factors such as the disease state, age, sex, and weight of the individual, and the ability of the pharmaceutical composition to elicit a desired response in the individual. An ordinarily skilled clinician can determine appropriate amounts to administer to achieve the desired therapeutic benefit based on these and other considerations.

### 6. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 7. EXAMPLES

The following are examples of methods and compositions of the invention. It is understood that various other embodiments may be practiced, given the general description provided herein.

### Example 1

### 1.1 Materials and Methods

### 1. HEK-Blue IL-12 Reporter Assay

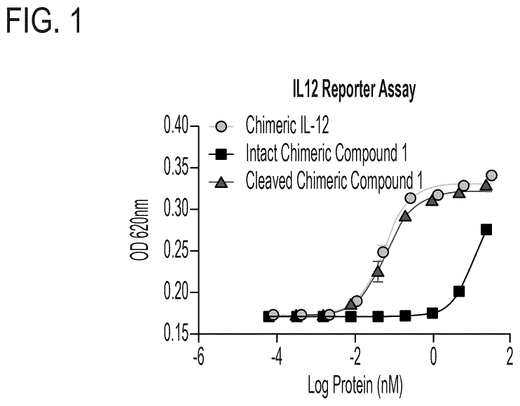

HEK-Blue IL-12 cells (InvivoGen) were plated in suspension at a density of 50,000 cells/well in culture media with or without 15 or 40 mg/ml human serum albumin (HSA) and stimulated with a dilution series of recombinant hIL-12, chimeric IL-12 (mouse p35/human p40), activatable chimeric IL-12, or activatable hIL-12 for 20-24 hours at 37° C. and 5% CO2. Activity of uncleaved and cleaved activatable hIL-12 was tested. Cleaved inducible hIL-12 was generated by incubation with active MMP9 or CTSL-1. IL-12 activity was assessed by quantification of Secreted Alkaline Phosphatase (SEAP) activity using the reagent QUANTI-Blue (InvivoGen), a colorimetric based assay. Results confirm that IL-12 fusion proteins are active and inducible. Results are shown in FIG. 1.

### 2. Cell Lines

All cell lines were grown and maintained by Charles River Laboratories (Morrisville, NC and Worcester, MA) according to ATCC guidelines and kept in culture for no longer than 2 weeks. Frozen cells were thawed and maintained for 1-3 passages before implantation. B16-F10, CT26, and EMT-6 cell lines were cultured in RPMI-1640 with L-Glutamine (Gibco, 11875-085) with 10% heat-inactivated fetal calf serum (Gibco, 35-015-CV) while MC38 was cultured in Dulbecco's Modified Eagle Medium (Gibco, 1966-025) supplemented with 10% heat-inactivated fetal calf serum (Gibco, 16000-044). Prior to tumor implantation, cells were washed twice with PBS and counted.

### 3. MC38 Model Tumor Implantation

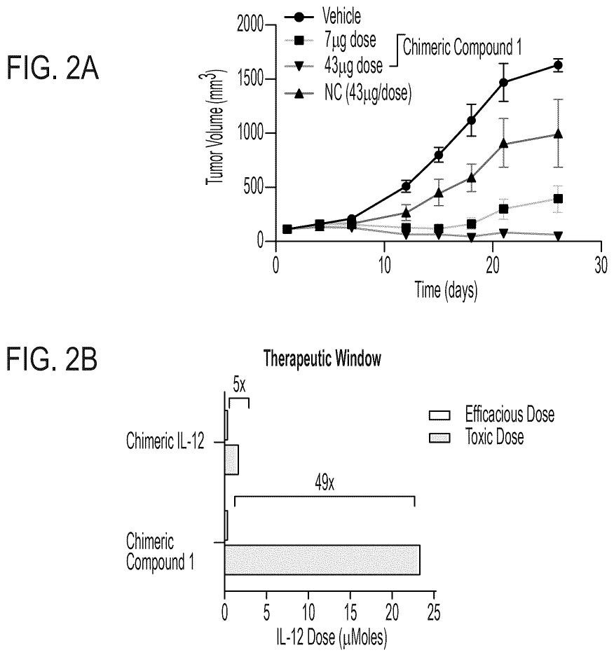

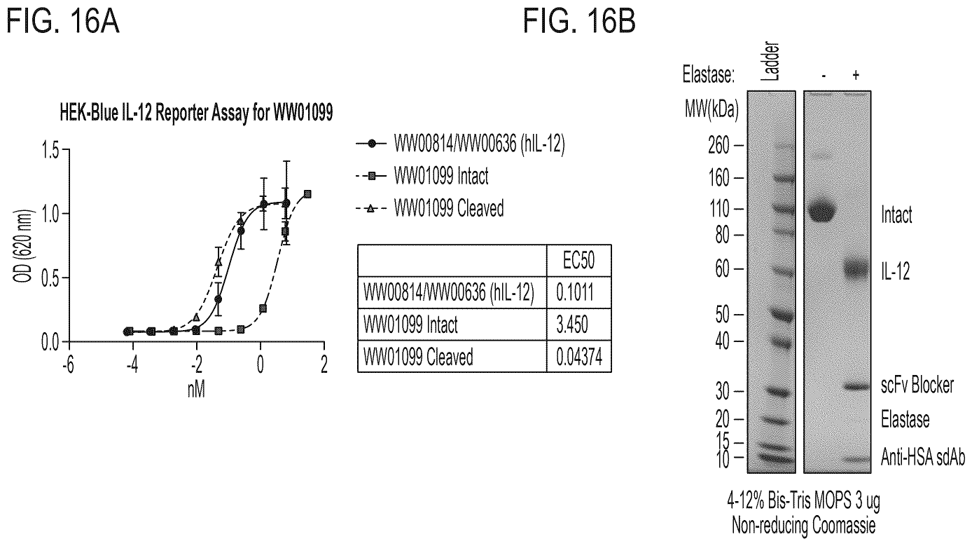

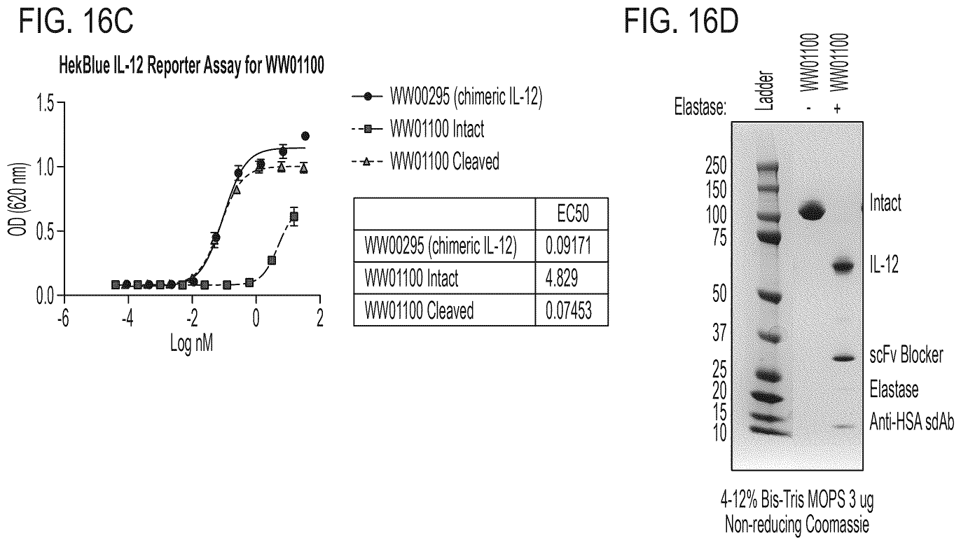

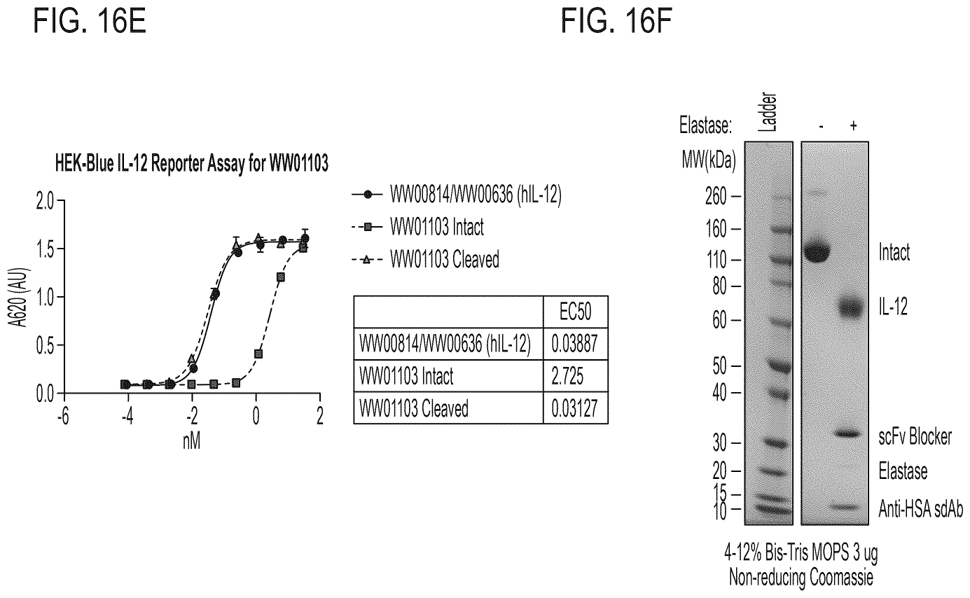

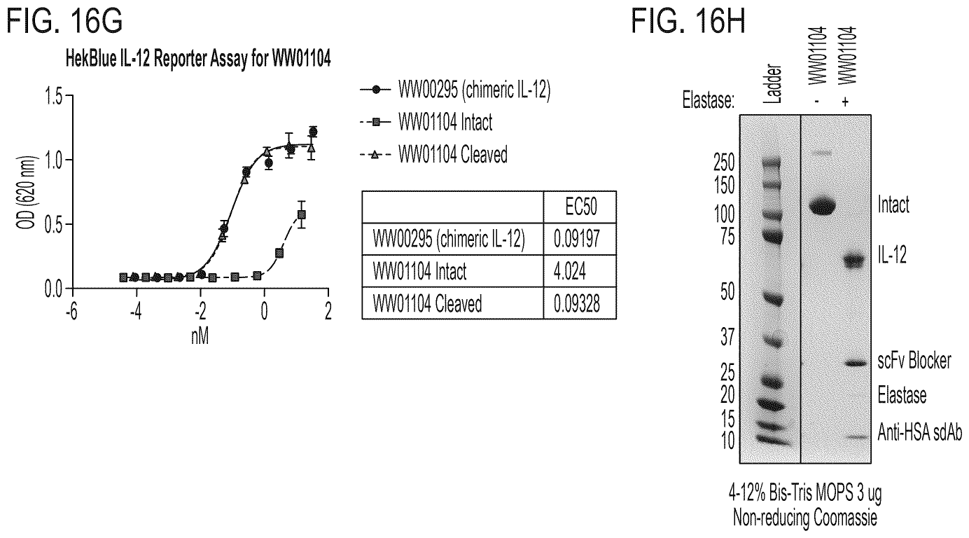

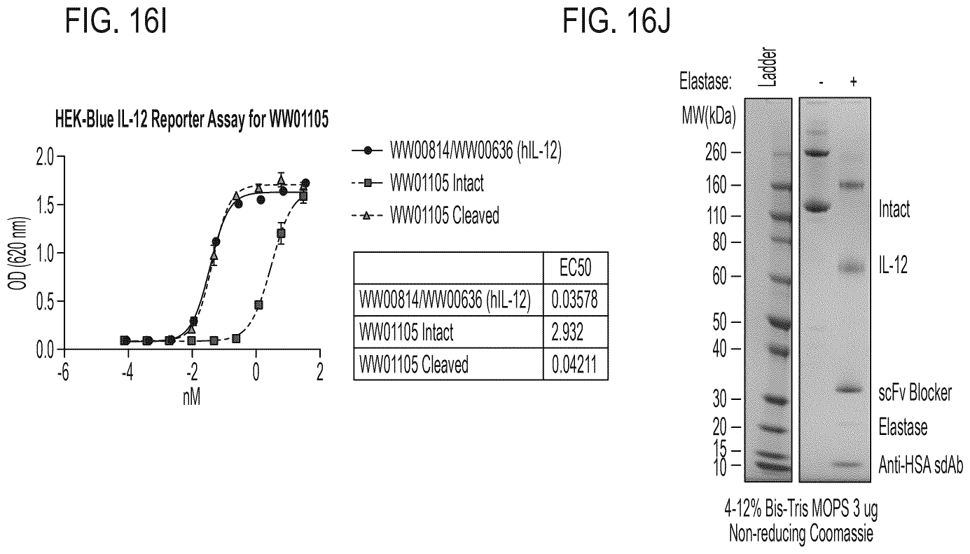

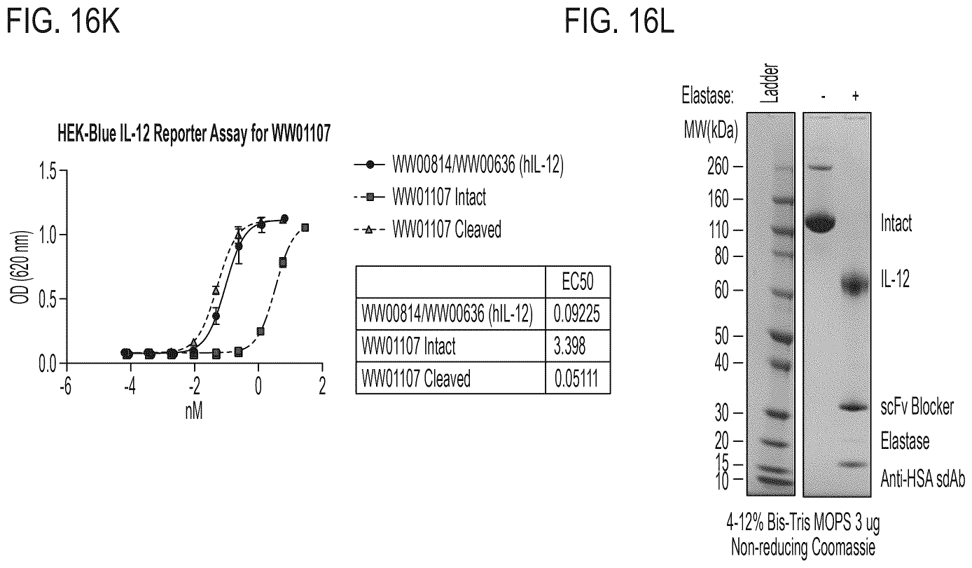

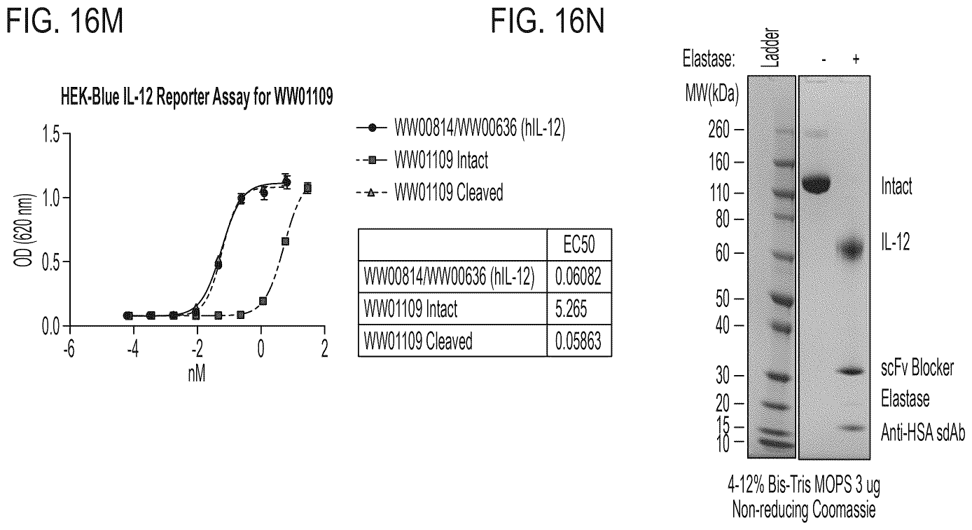

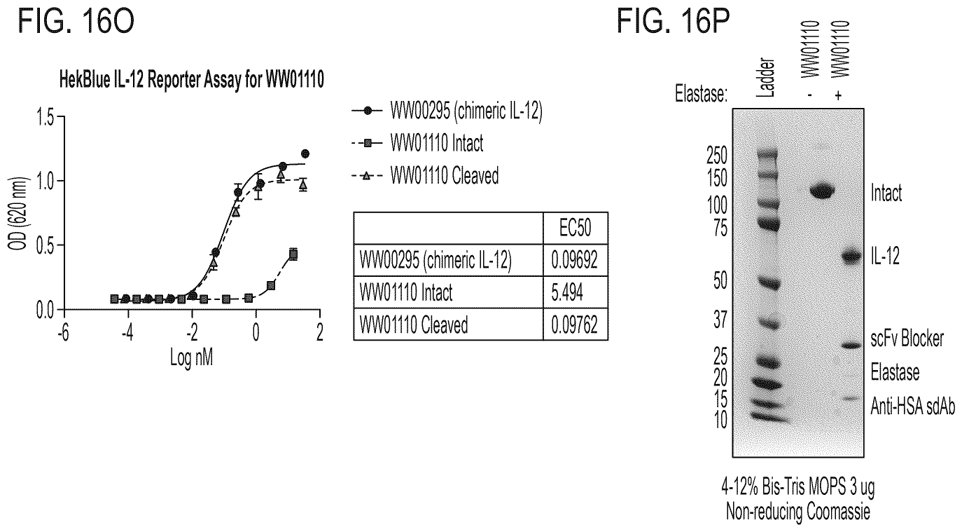

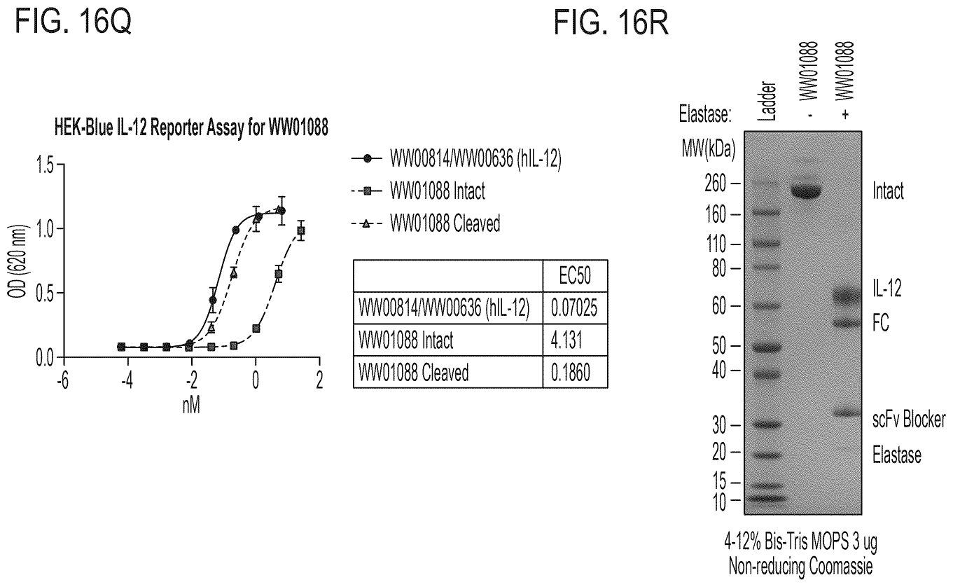

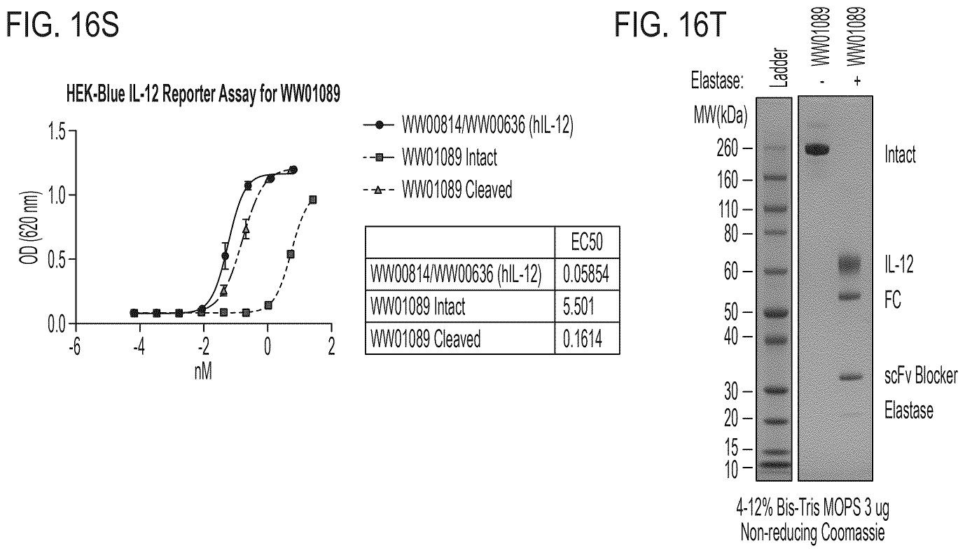

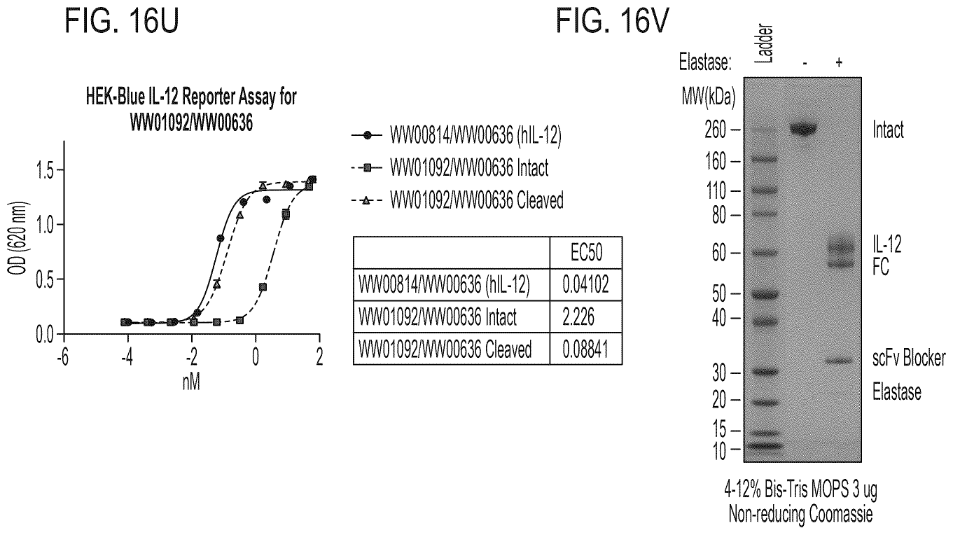

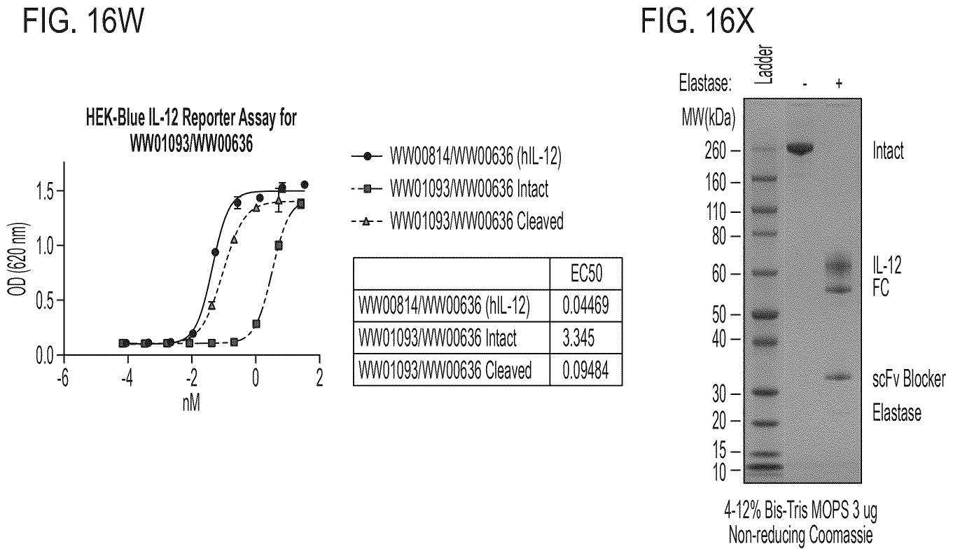

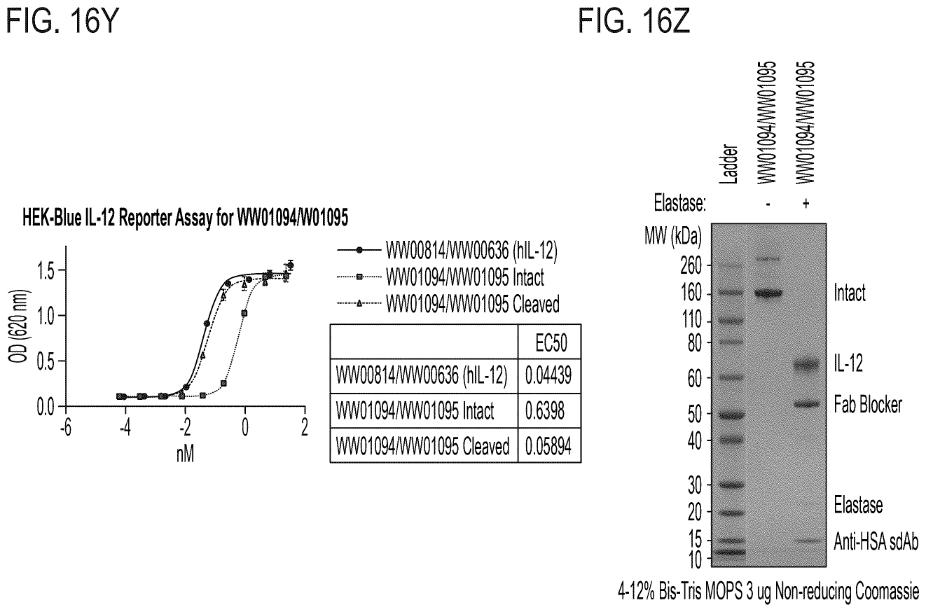

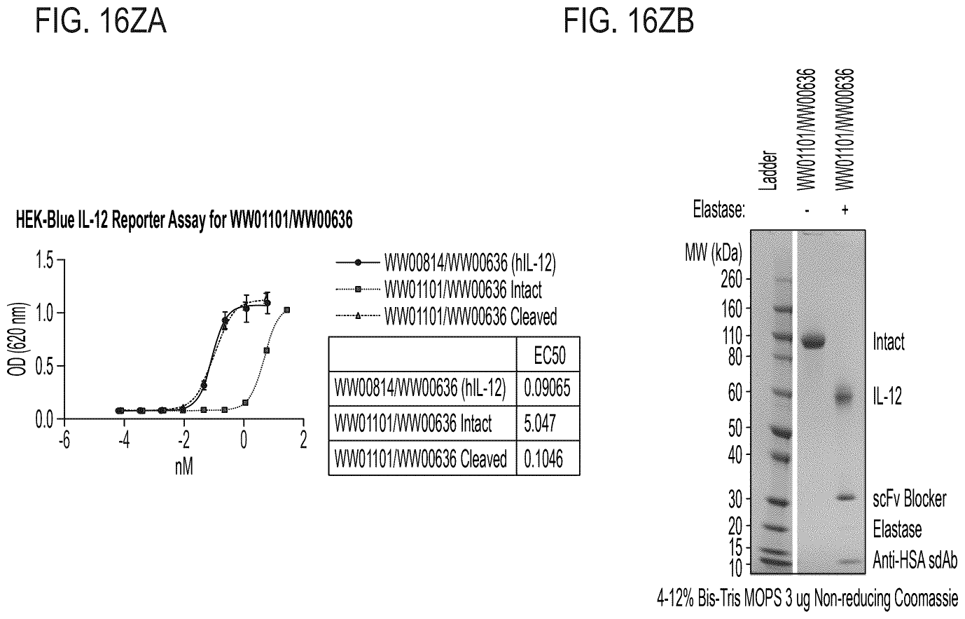

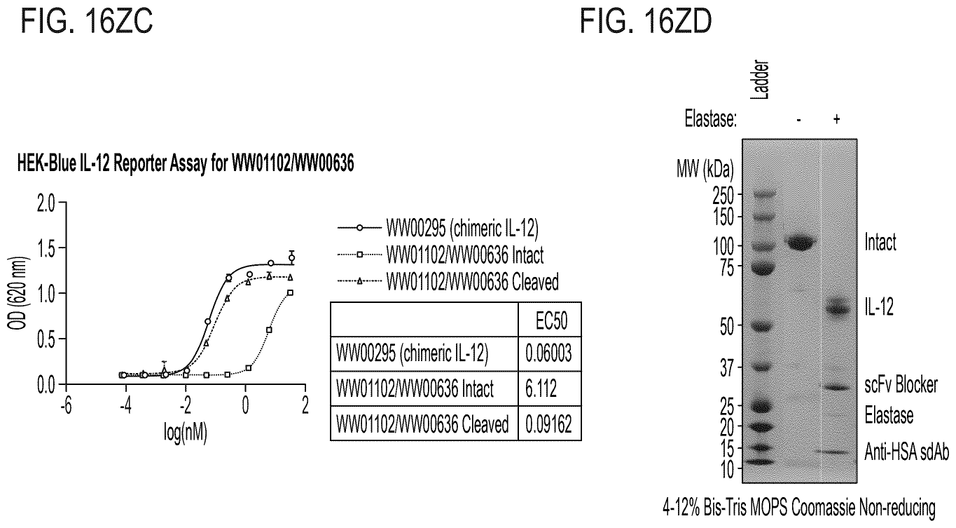

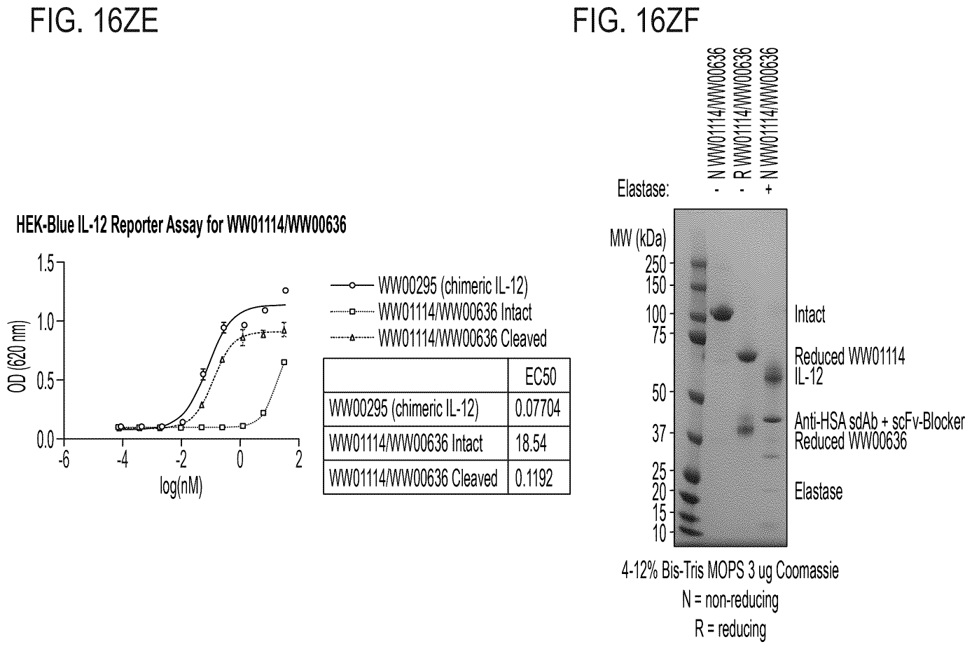

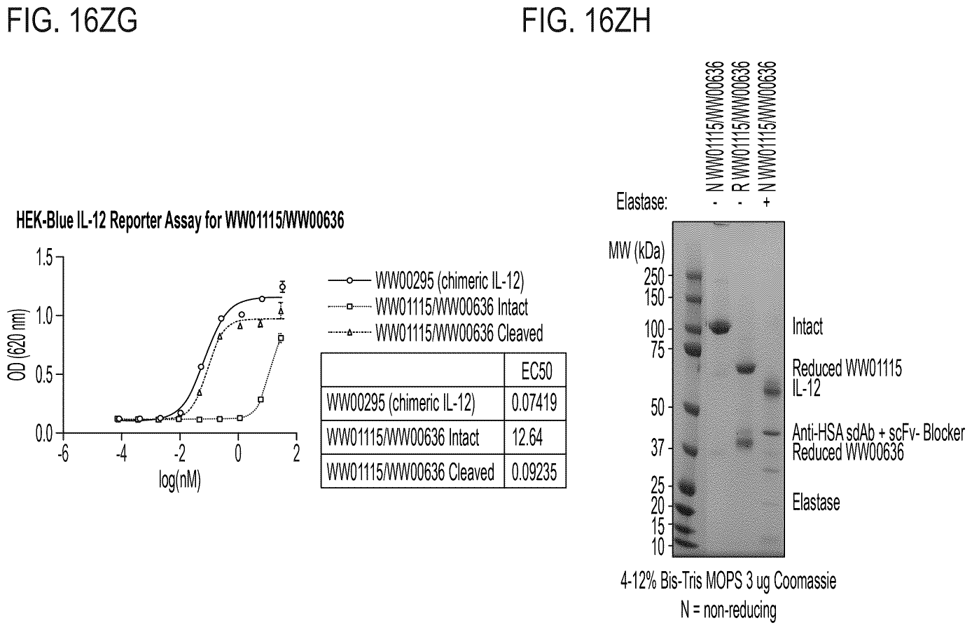

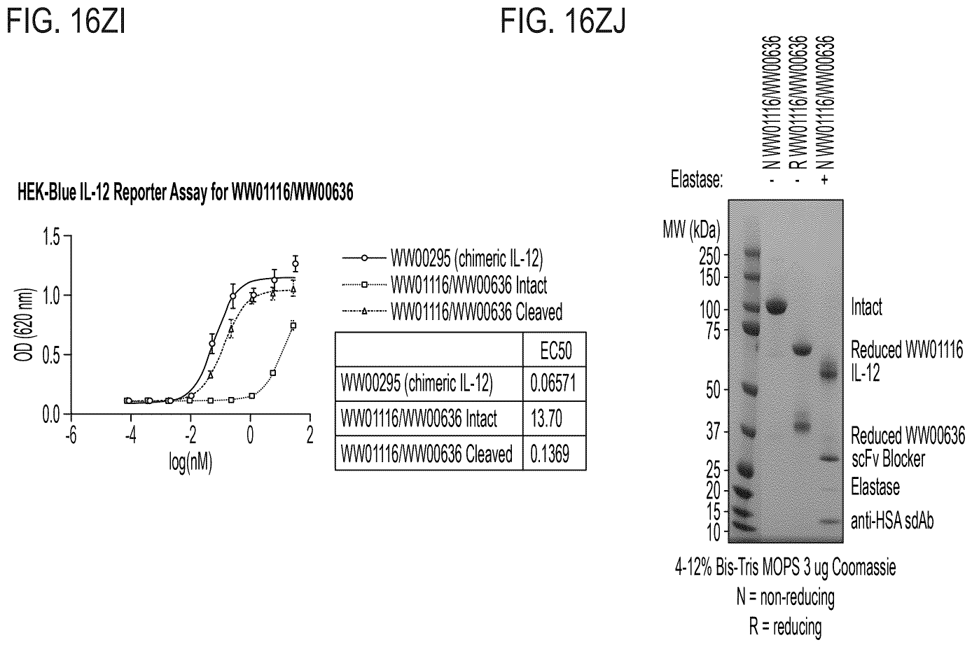

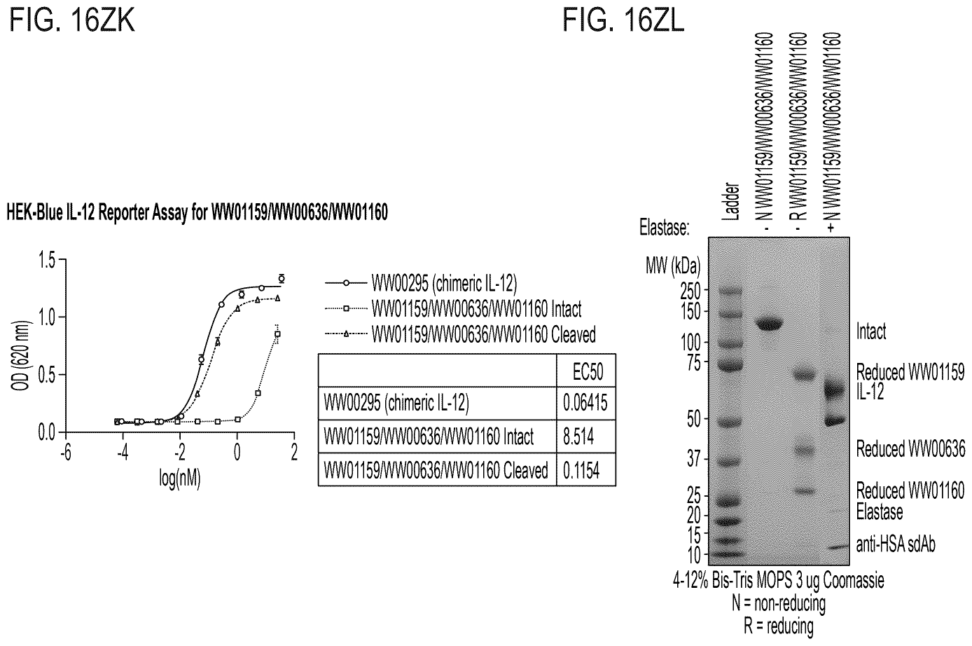

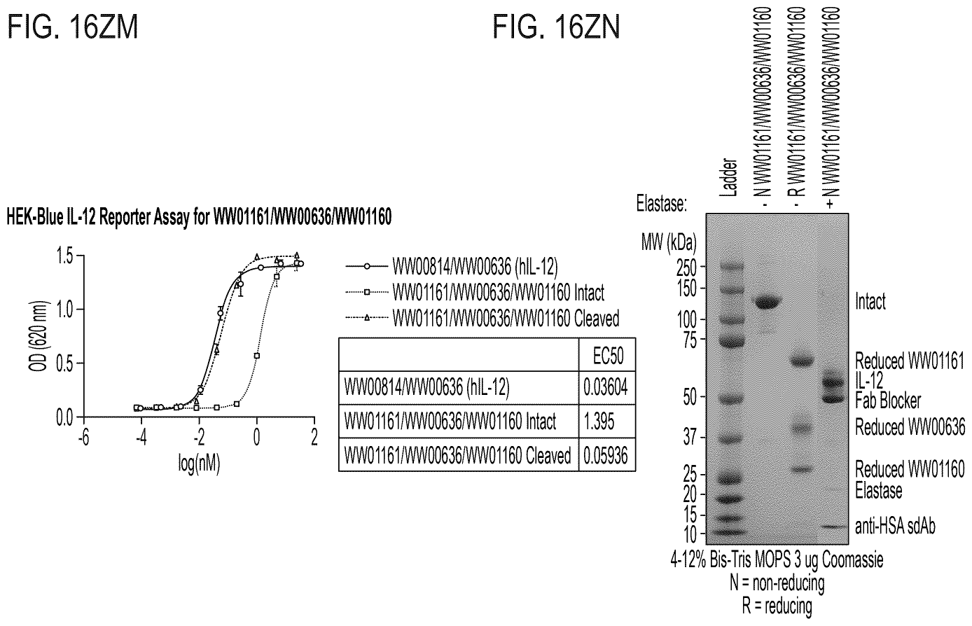

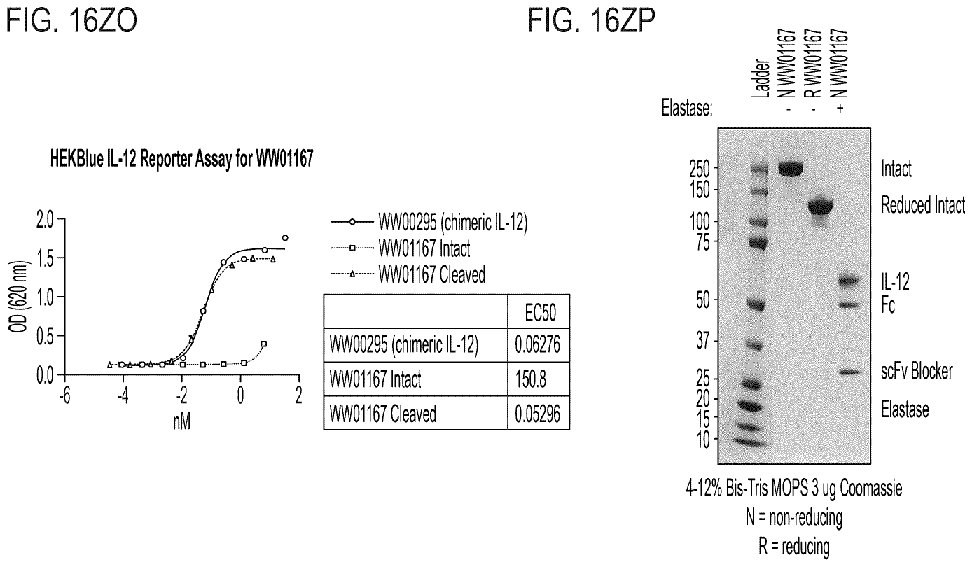

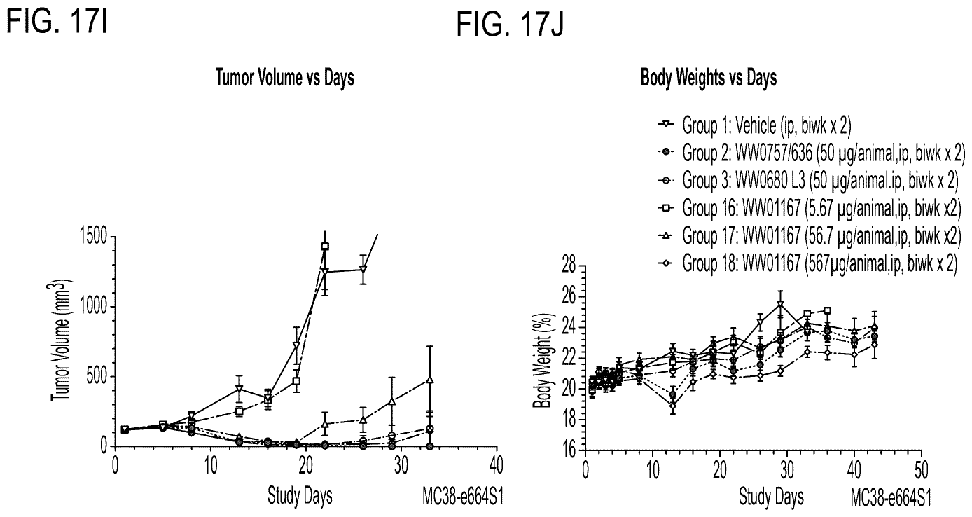

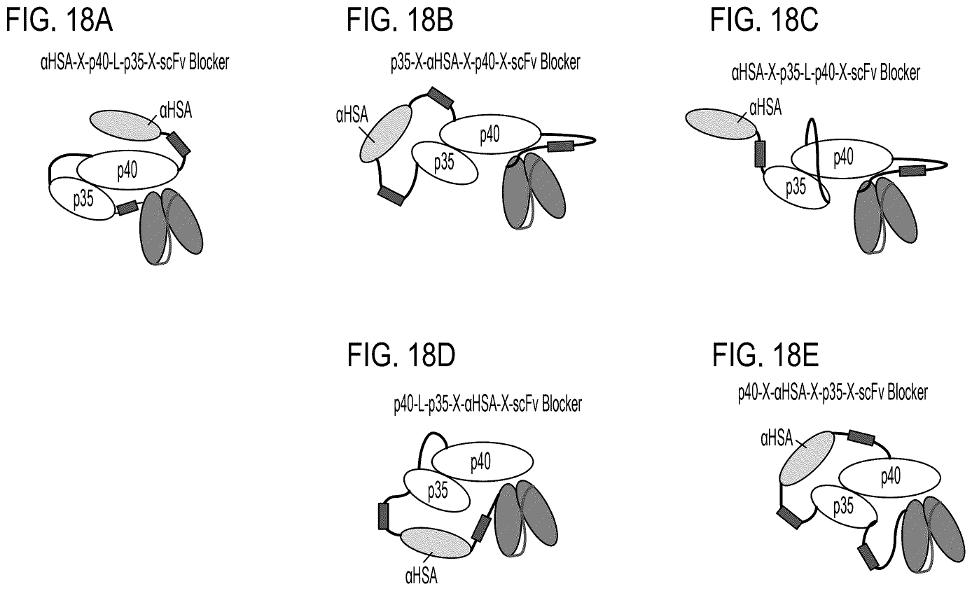

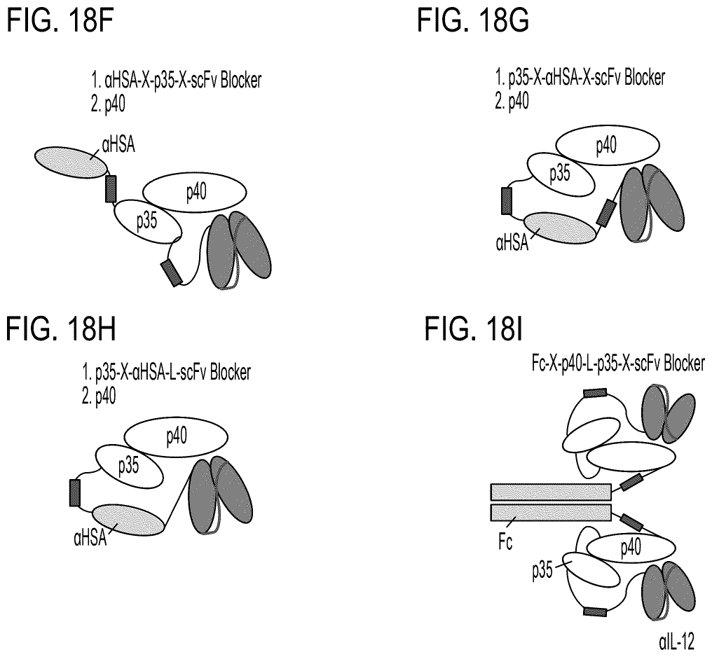

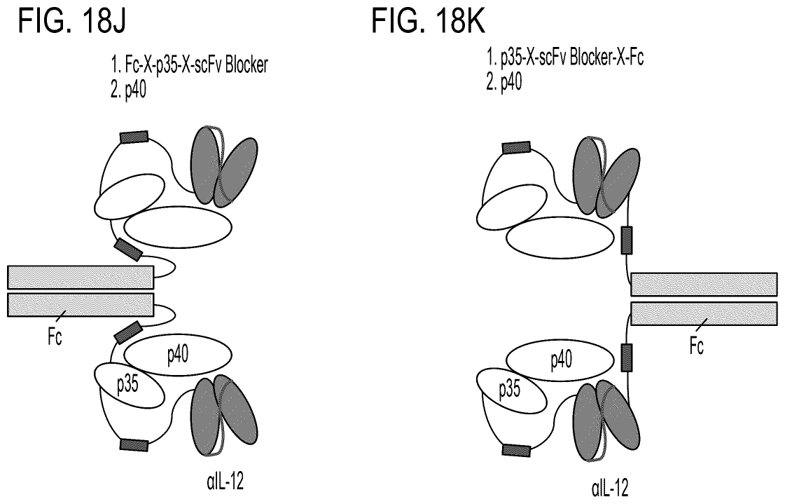

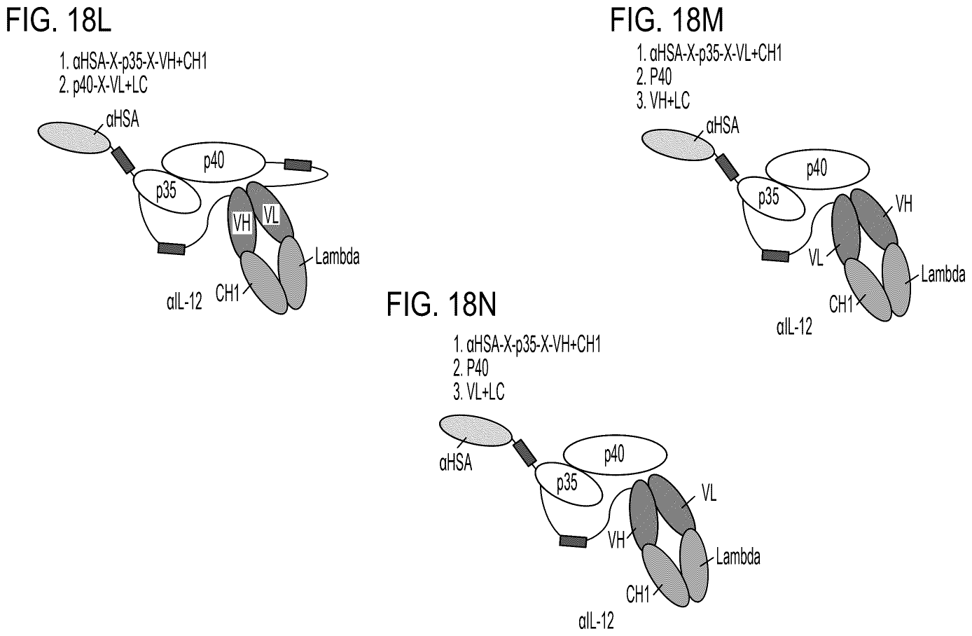

All mouse in vivo work was performed in accordance with current regulations and standards and the NIH at Charles River Laboratories (Morrisville, NC and Worcester, MA) with the approval of an Institutional Animal Care and Use Committee (IACUC). Female, 6-8 week-old C57Bl/6 mice from Charles River Laboratories were shaved on their flank 1 day prior to tumor cell implantation. A total of 5×10⁵ MC38 cells were injected subcutaneously and monitored for tumor growth. Extra mice were implanted in order to have sufficiently sized tumors for randomization. Tumor volume was monitored until the group average was 100-150 mm³, and mice were randomized into treatment groups on Day 0. Mice receiving chimeric Compound 1 were dosed twice a week for two weeks (Days 1, 4, 8, and 11) unless otherwise noted. Inducible IL-12 prodrugs used in these studies included chimeric Compound 1. Mice receiving recombinant chimeric IL-12 (chimeric IL-12 or WW0295) were dosed twice a day for 5 days before receiving a 2-day break (5/2 regimen) and the cycle was repeated for a total of two weeks. All treatments were administered by intraperitoneal injection. Body weight and tumor volume were both measured twice weekly for the duration of the study. Tumors were measured in two dimensions using calipers, and volume was calculated using the formula: Tumor volume (mm³)=[(w2×l)/2] where w=width and l=length, in mm, of a tumor. Mice were kept on study until tumors reached 1500 mm3, or the study reached the termination point at Day 45. See, FIGS. 2A-28, 3G, and 4A-4F.

### 4. B16-F10 Model

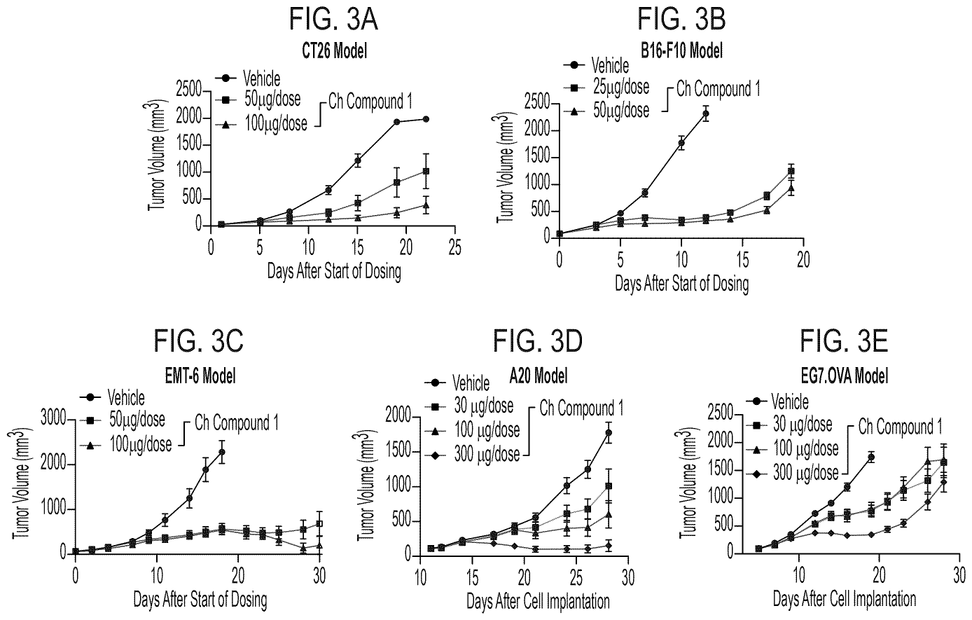

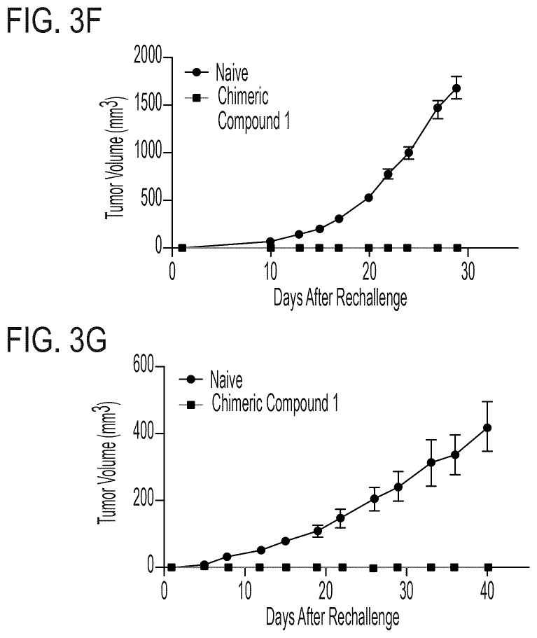

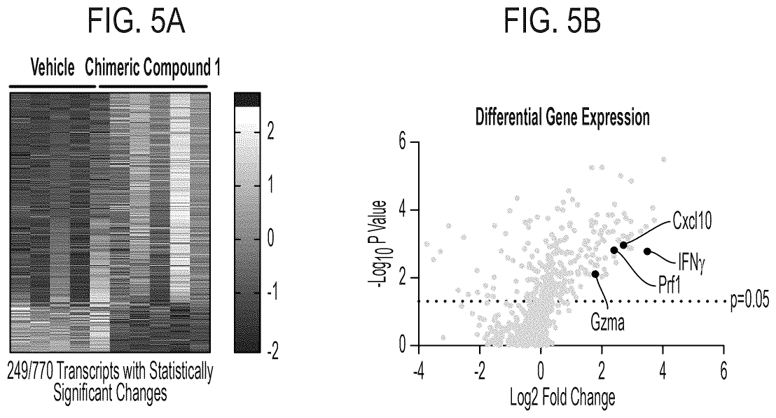

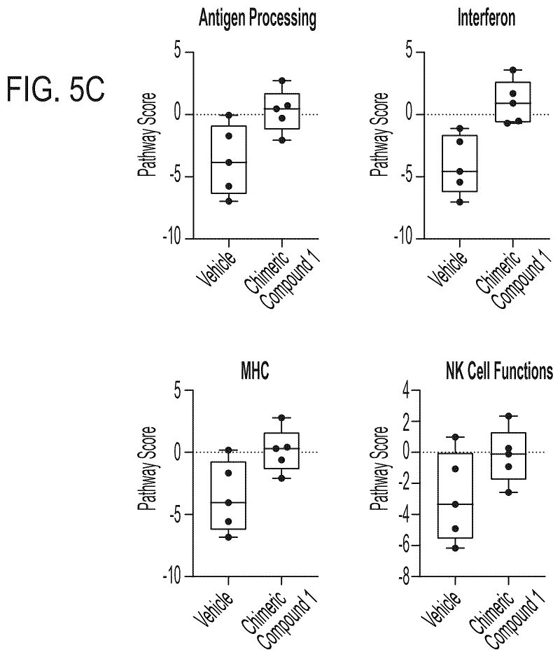

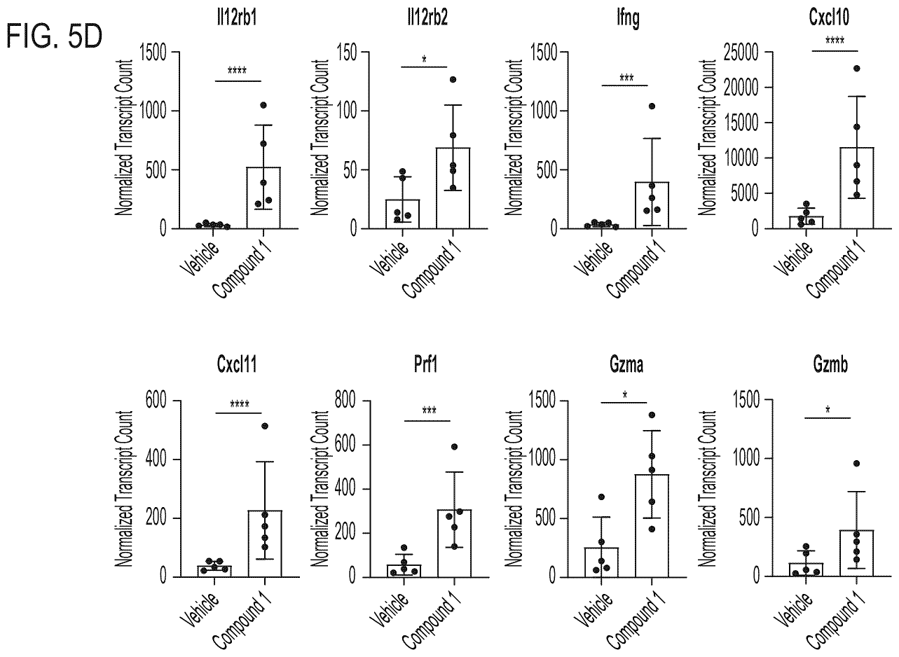

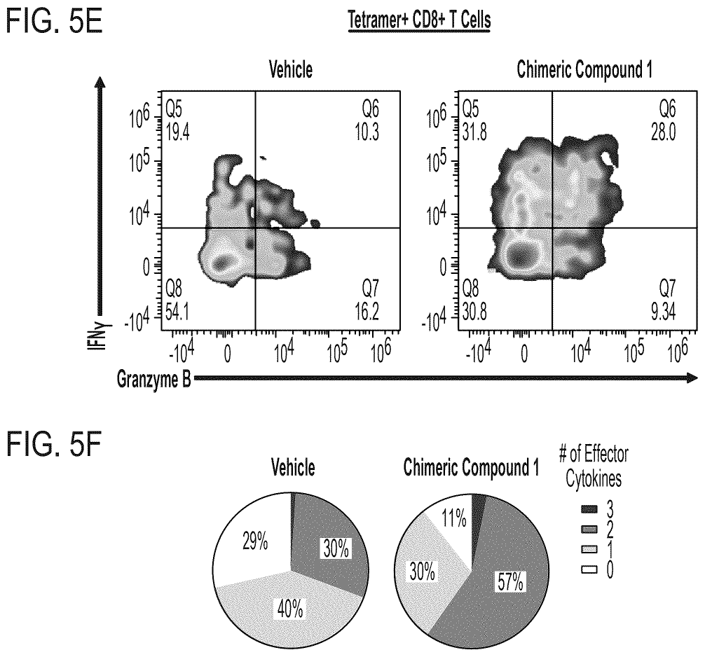

All mouse in vivo work was performed in accordance with current regulations and standards and the NIH at Charles River Laboratories (Morrisville, NC and Worcester, MA) with the approval of an Institutional Animal Care and Use Committee (IACUC). Female, 6-8 week-old C57Bl/6 mice from Charles River Laboratories were shaved on their flank 1 day prior to tumor cell implantation. A total of 1×10⁵ B16-F10 cells were injected subcutaneously and monitored for tumor growth. Extra mice were implanted in order to have sufficiently sized tumors for randomization. Tumor volume was monitored until the group average was 50-100 mm³, and mice were randomized into treatment groups on Day 0. Mice receiving chimeric Compound 1 were dosed on Days 1 and 4, and tumors were harvested 24 hours after the second dose (Day 5). Inducible IL-12 prodrugs used in these studies included chimeric Compound 1. All treatments were administered by intraperitoneal injection. See, FIG. 3B and FIG. 5A-5F.

### 5. EMT-6 Model

All mouse in vivo work was performed in accordance with current regulations and standards and the NIH at Charles River Laboratories (Morrisville, NC and Worcester, MA) with the approval of an Institutional Animal Care and Use Committee (IACUC). Female, 6-8 week-old C57Bl/6 mice from Charles River Laboratories were shaved on their flank 1 day prior to tumor cell implantation. A total of 1×10⁵ EMT6 cells were injected subcutaneously and monitored for tumor growth. Extra mice were implanted in order to have sufficiently sized tumors for randomization. Tumor volume was monitored until the group average was 50-100 mm³, and mice were randomized into treatment groups on Day 0. Mice receiving chimeric Compound 1 were dosed twice a week for two weeks (Days 1, 4, 8, and 11) unless otherwise noted. Inducible IL-12 prodrugs used in these studies included chimeric Compound 1. All treatments were administered by intraperitoneal injection. In some experiments, mice that rejected tumors previously were then rechallenged with 1×10⁵ EMT6 cells on the opposite flank four months after the initial rejection. In those experiments, age matched, tumor naïve animals were used as a control. In some experiments, tumor samples were harvested and incubated in 5-10 mLs of 10% neutral buffered formalin for at least 72 hours before being embedded in paraffin and mounted on slides. Unstained slides were submitted to Nanostring for immunofluorescence staining and geospatial transcriptional analysis using a Nanostring GeoMX DSP system. See, FIGS. 3C, 3F, 6, and 7A-7D.

### 6. CT26 Model

All mouse in vivo work was performed in accordance with current regulations and standards and the NIH at Charles River Laboratories (Morrisville, NC and Worcester, MA) with the approval of an Institutional Animal Care and Use Committee (IACUC). Female, 6-8 week-old Balb/C mice from Charles River Laboratories were shaved on their flank 1 day prior to tumor cell implantation. A total of 3×10⁵ CT26 cells were injected subcutaneously and monitored for tumor growth. Extra mice were implanted in order to have sufficiently sized tumors for randomization. Tumor volume was monitored until the group average was 30-60 mm³, and mice were randomized into treatment groups on Day 0. Mice receiving chimeric Compound 1 were dosed twice a week for two weeks (Days 1, 4, 8, and 11) unless otherwise noted. Inducible IL-12 prodrugs used in these studies included chimeric Compound 1. All treatments were administered by intraperitoneal injection. Body weight and tumor volume were both measured twice weekly for the duration of the study. Tumors were measured in two dimensions using calipers, and volume was calculated using the formula: Tumor volume (mm³)=[(w²×l)/2] where w=width and l=length, in mm, of a tumor. Mice were kept on study until tumors reached 1500 mm³, or the study reached the termination point at Day 45. See, FIG. 3A.

### 7. EG7.OVA Model

All mouse in vivo work was performed in accordance with current regulations and standards and the NIH at Covance (Ann Arbor, MI) with the approval of an Institutional Animal Care and Use Committee (IACUC). Female, 6-8 week-old C57Bl/6 mice from Charles River Laboratories were shaved on their flank 1 day prior to tumor cell implantation. A total of 1×10⁶ EG7. OVA cells were injected subcutaneously and monitored for tumor growth. Extra mice were implanted in order to have sufficiently sized tumors for randomization. Tumor volume was monitored until the group average was ˜93 mm³, and mice were randomized into treatment groups on Day 0. Mice receiving chimeric Compound 1 were dosed twice a week for two weeks (Days 1, 4, 8, and 11) unless otherwise noted. Inducible IL-12 prodrugs used in these studies included chimeric Compound 1. All treatments were administered by intraperitoneal injection. Body weight and tumor volume were both measured twice weekly for the duration of the study. Tumors were measured in two dimensions using calipers, and volume was calculated using the formula: Tumor volume (mm³)=[(w²×l)/2] where w=width and l=length, in mm, of a tumor. Mice were kept on study until tumors reached 1500 mm³, or the study reached the termination point at Day 45. See, FIG. 3E.

### 8. A20 Model

All mouse in vivo work was performed in accordance with current regulations and standards and the NIH at Covance (Ann Arbor, MI) with the approval of an Institutional Animal Care and Use Committee (IACUC). Female, 6-8 week-old Balb/C mice from Charles River Laboratories were shaved on their flank 1 day prior to tumor cell implantation. A total of 5×10⁵ A20 cells were injected subcutaneously and monitored for tumor growth. Extra mice were implanted in order to have sufficiently sized tumors for randomization. Tumor volume was monitored until the group average was ˜90-130 mm³, and mice were randomized into treatment groups on Day 0. Mice receiving chimeric Compound 1 were dosed twice a week for two weeks (Days 1, 4, 8, and 11) unless otherwise noted. Inducible IL-12 prodrugs used in these studies included chimeric Compound 1. All treatments were administered by intraperitoneal injection. Body weight and tumor volume were both measured twice weekly for the duration of the study. Tumors were measured in two dimensions using calipers, and volume was calculated using the formula: Tumor volume (mm³)=[(w²×l)/2] where w=width and l=length, in mm, of a tumor. Mice were kept on study until tumors reached 1500 mm³, or the study reached the termination point at Day 45. See, FIG. 3D.

### 9. Tumor Digestions and NanoString Analysis

MC38 and B16-F10 tumors were chopped into small pieces (<5 mm³) in phenol-free RPMI-1640 (ThermoFisher) before being enzymatically digested with Collagenase IV (3 mg/mL, Gibco, 17104019) at 37° C. for 35 minutes while shaking. After digestion, tumor samples were mechanically dissociated through a 70 μM cell strainer. EMT6 tumors were processed using gentleMACS™ C-Tubes from Miltenyi Biotech (130-093-237). Briefly, tumors were cut into small pieces (<5 mm³) in HBSS containing 1.25 mg/mL Collagenase Type IV (Gibco, 17104019), 0.0025 mg/mL Hyaluronidase (Sigma-Aldrich, H3506), and 0.01 mg/mL DNASE I (Worthington, LS002004). Samples were placed on a gentleMACS™ Octo Dissociator and processed using program 37C_m_TDK_1 before samples were passed through a 70 μM cell strainer to remove any undigested tumor pieces. Single cell suspensions were then counted and analyzed by flow cytometry. For NanoString analysis, 5×10⁵ cells were frozen in 100 μL of RLT Lysis buffer (Qiagen, 1053393). RNA samples were shipped to LakePharma and analyzed using the nCounter Mouse PanCancer Immune Profiling Codeset Panel with the nCounter FLEX analysis system. NanoString analysis was performed using nSolver™ Software with the Advanced Analysis module installed.

### 10. Flow Cytometry

All cell staining was performed in 96-well round bottom plates using FACs Buffer (PBS+0.5% BSA) or 1× Permeabilization Buffer (eBioscience, 00-5223-56) where appropriate. Cells were first treated with FC block (BioLegend, 101320) at room temperature before tetramer staining was performed for 20 minutes. After tetramer staining, cells were then stained with a master mix of extracellular antibodies for 20 minutes at 4° C. before being fixed/permeabilized overnight using the eBioscience™ Foxp3 Transcription Factor Staining Buffer Set according to the manufacturer's protocol. The next day, samples were washed with Perm Buffer and stained with intracellular markers for 20 minutes at 4° C. Cells were analyzed on a Cytek Aurora system. Fluorescence minus one (FMO) and single stain controls (cells or OneComp Ebeads™ (Thermofisher, 01-1111-42)) were stained alongside cells. Unless otherwise noted, when flow cytometry was used to assess effector cytokine production, cells were restimulated with PMA (50 ng/mL, Sigma-Aldrich, P1585) and Ionomycin (1 μg/mL, Sigma-Aldrich, IO634-1MG) in the presence of 1× Brefeldin A (Thermofisher Scientific, 00-4506-51) for 4 hours in complete media at 37° C. prior to staining. Cells used for 2-NDBG assay were starved in glucose free RPMI-1640 Media (Gibco, 11879-020) for 1 hour, then incubated with 2-NDBG (Cayman Chemical, #186689-07-6) for 1 hour at 37° C./5% CO2 before being stained for extracellular markers. MitoTracker Deep Red FM (ThermoFisher, #M46753), MitoTracker Green FM (ThermoFisher, M46750) MitoSOX Red (ThermoFisher, M36008), and TMRM (ThermoFisher, T668) staining was performed at 37° C./5% CO2 in RPMI 1640 media (Gibco, A10491-01) with 10% heat inactivated FBS (Gibco, 10082-147) and Penicillin/Streptomycin (Gibco, 15140-122) for 1 hour. Cells were then washed with FACs buffer and stained for extracellular markers. Specific antibody clones are detailed as follows. Fluorescent dye-conjugated antibodies specific for the following proteins were purchased from BioLegend: CD8α APC, clone 53-67; CD4 BV650, clone RM4-5; CD3 AF700, clone 17A2; CD45 BV605, clone 30-F11; CD49b APC/Cy7, clone DX5; CD25 BV421, clone PC61; CD25 APC/Fire 750, clone PC61; Ki67 PeCy7, clone 16A8; Ki67 AF700, clone 16A8; granzyme B FITC, clone GB11; IFNγ PE, clone XMG1.2; F4/80 Pe/Dazzle 594, clone BM8; CD3 Complex PeCy7, clone 17A2; FC Block, clone 93. Fluorescent dye-conjugated antibodies specific for the following proteins were purchased from eBioscience: CD45 BUV395, clon30-F11; CD4 BUV496, clone GK1.5; CD8 BUV563, 53.6-7; TNF BV750, clone MP6-XT22; CD49B Pe-Cy5, clone DX5, FoxP3 AF488, clone FJK-16s; FoxP3 eFlour450, clone FJK-16s. The fluorescent dye-conjugated tetramer against the MulV p15E peptide KSPWFTTL (SEQ ID NO: 449) was purchased from ThermoFisher Scientific (50-168-9385). The Live/Dead Blue Dye was also purchased from ThermoFisher Scientific (L23105).

### 11. Pharmacokinetic Analysis

Plasma and tumor samples were collected at indicated time points by Charles River Laboratories and stored at −80° C. MC38 tumor lysates were generated by homogenizing each tumor with a Qiagen TissueRuptor with disposable probes (Qiagen) in ice cold Lysis Buffer (1×Tris Buffered Saline (Sigma-Aldrich, T5912-1L), 1 mM EDTA (Sigma-Aldrich, 3690-100 mL), 1% Triton X-100 (Sigma-Aldrich, X100-1000 mL), with protease inhibitors (Sigma-Aldrich, P8340-1L) in diH2O). Plasma and tumor samples were analyzed using a sandwich ELISA on the MSD platform, which detects both intact chimeric Compound 1 and free/released IL-12. Free IL-12 level was quantified using an in-house developed ECLIA assay on MSD MESO™ QuickPlex SQ 120 system. Data acquisition and analysis were performed using MSD Workbench 4.0.12, and pharmacokinetic parameters were calculated using Phoenix WinNonlin Version 8.1.

### 12. Compound 36 Stability in Human Serum

Compound 36 was incubated in human serum (BioIVT) from healthy donors in duplicate for each timepoint. Time zero (T0) samples were immediately frozen at −80° C. The remaining samples were incubated at 37° C. for 24 (T24) or 72 (T72) hours before being stored at 80° C. Stability of Compound 36 was assessed by western blot analysis using the JESS system (Protein Simple, SM-W004) according to the manufacturer's general protocol. Input controls (intact and protease cleaved) were also analyzed. Samples and antibodies (anti-IL-12 primary (R&D Systems, AB-219-NA)) and secondary (Jackson Labs, 805-035-180) were loaded into a 12-230 kDA Jess separation module and run using a Jess system set to the standard settings for chemiluminescence, with modifications to the standard protocol for non-reducing conditions. Analysis of the resulting western blot was performed using Compass for Simple Western Software (v4.1.0).

### 13. Human Primary Cell Assays

Human PBMCs were isolated from whole blood (BioIVT) using Ficoll-Paque Plus (GE Healthcare, GE17-1440-03) according to the manufacturer's protocol and frozen in Recovery Cell Culture Freezing Media (Gibco, 12648010) for later use. To generate activated T cells (Tblasts), PBMCs were thawed, counted, and stimulated with 5 μg/mL of PHA (Sigma-Aldrich, L1668-5MG) for 72 hours before being frozen. Tblasts were thawed, counted, and plated in a 96-well round bottom plate, and incubated with titrated amounts of intact or protease-activated (cleaved) INDUKINE™ proteins or chimeric IL-12. After 72 hours, IFNγ production was measured using a Human IFNγ specific AlphaLisa Kit (Perkin Elmer, AL217C) according to the manufacturer's protocol with a Perkin Elmer Enspire Alpha Reader running Enspire Manager Software (V4.13.3005.1482).

### 14. Ex Vivo Inducible IL-12 Pro-Drug Protein Processing Assay

Primary human healthy cells were purchased from either ATCC, Lonza, or Zen-Bio, and cultured according to the manufacturers' protocols. Dissociated human tumor samples were purchased from Discovery Life Sciences. To examine inducible IL-12 prodrug processing, samples were thawed, washed, and counted. Cells were then resuspended in media containing either intact Compound 36, a non-cleavable variant of Compound 36, or pre-cut Compound 36 for 48 hours before cell culture supernatants were collected and frozen for later analysis. Cell culture supernatants were then used to stimulate previously activated human Tblasts using the assay detailed above. IFNγ production was normalized against the controls using the following equation to assess the level of processing.

$$Percent of Full Activity = ( 1 - ( ( Sample - Uncleavable Ctrl ) ) / ( ( Cleaved Ctrl - Uncleavable Ctrl ) ) ) * 100$$

### 15. Data Representation, Bioinformatic Analysis, and Statistics

Flow cytometry plots were generated with FlowJo Software (v10.5.30) and are representative samples. All the quantitative plots were generated using GraphPad Prism 8 Software for Windows (64-Bit) (San Diego, CA). For in vitro activity assays, data were analyzed using a non-linear sigmoidal, 4PL curve fit model without constraints. Statistical analysis was also performed using GraphPad Prism software (San Diego, CA). Two sample comparisons used a student's t-test while comparisons of more than two groups used an analysis of variance (ANOVA) test with multiple comparisons. Antitumor effects over time were analyzed by using a mixed-effects model. For the NanoString dataset, statistical analysis was performed using nSolver™ software with the Advanced Analysis Module installed. Pathway analysis was performed using Partek software (v 10.0.22.0428), based on transcripts that were significantly different following mWTX-330 with an FDR step-up of 0.05.

### 1.2 Results

### 1. Chimeric Compound 1 is a Selectively Activated Inducible IL-12 Prodrug that Generates a Robust, Cleavage Dependent Anti-Tumor Immune Response in Multiple Models

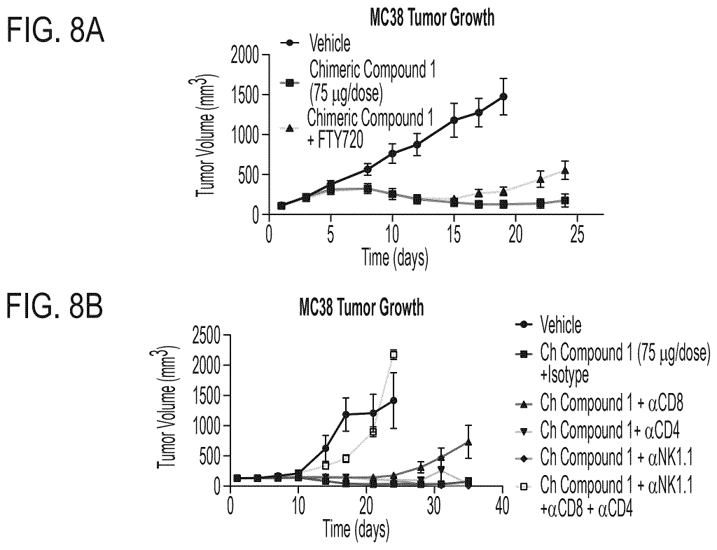

In order to address the clinical shortcomings of free cytokine therapy, a selectively inducible IL-12 prodrug was developed. To measure the difference in activity between intact and protease activated (cleaved) chimeric Compound 1, HEK-Blue IL-12 reporter cells were incubated with either intact or protease activated chimeric Compound 1, and IL-12 signaling was assessed. In this assay, intact chimeric Compound 1 had 175-fold less activity than either cleaved chimeric Compound 1 or chimeric IL-12. (FIG. 1). To test whether chimeric Compound 1 could generate anti-tumor immunity in vivo, MC38 tumor bearing animals were treated with titrated amounts of chimeric Compound 1, and tumor growth was monitored over time. In this study, a variant of chimeric Compound 1 with a non-cleavable linker (FIG. 1) was also included at the highest dose as a control. In this model, even the lowest tested dose of chimeric Compound 1 (7 μg/dose) generated statistically significant tumor growth inhibition, with 43 μg/dose being sufficient to generate complete tumor rejections (FIG. 2A). In contrast, the non-cleavable (NC) variant of chimeric Compound 1 had less activity than even the lowest dose of chimeric Compound 1 demonstrating that the full potency of chimeric Compound 1 is dependent on in vivo processing of the molecule. Chimeric Compound 1 treatment also generated robust anti-tumor immunity in less immune cell infiltrated (“colder”) syngeneic tumor models, including CT26 (FIG. 3A), B16-F10 (FIG. 3B), and EMT-6 (FIG. 3C), demonstrating the broad activity of this molecule in vivo. While the use of a non-cleavable control demonstrated the necessity of processing for full activity, these data did not directly indicate that processing was occurring in the TME. FTY720 is a small molecule inhibitor of Sphingosine-1-phosphate receptor-1 which prevents lymphocyte egress from secondary lymphoid tissues, and effectively isolates the TILs from the normal recirculating population of immune cells in vivo. Chimeric Compound 1/FTY720 co-treated animals retained the potent early anti-tumor activity associated with chimeric Compound 1 treatment, although tumor control was less complete after dosing with the INDUKINE™ molecule had stopped (FIG. 8A)

These data demonstrate that systemically administered chimeric Compound 1 is processed in the TME, and that this local release of IL-12 is sufficient to generate early tumor growth inhibition.

In order to identify the effector cell populations responsible for the chimeric Compound 1 induced anti-tumor immunity, MC38 tumor bearing mice were treated with chimeric Compound 1 in conjunction with antibody-based depletion of individual effector cell populations.

Interestingly, while depletion of the CD8+ T cell population did not inhibit early tumor control, chimeric Compound treated mice without CD8+ T cells were ultimately unable to control tumor growth (FIG. 8B). In contrast, depletion of either NK cells or total CD4+ T cells alone did not inhibit chimeric Compound 1 induced anti-tumor activity (FIG. 8B). However, in mice where all three populations were depleted, chimeric Compound 1 treatment had little antitumor activity, suggesting that these cell types work jointly to reject MC38 tumors in response to treatment. Furthermore, when chimeric Compound 1 treated mice that previously rejected either MC38 tumors (FIG. 3F) or EMT-6 tumors (FIG. 3G) were rechallenged, 100% of the animals were protected against tumor growth. Together, these data suggest that chimeric Compound 1 treatment generates a robust and durable anti-tumor immune response that is dependent on in vivo cleavage/activation of the molecule.

### 2. The Inducible IL-12 Prodrug Design of Chimeric Compound 1 Enhances its Pharmacokinetic Profile and Expands the Therapeutic Window of Chimeric IL-12

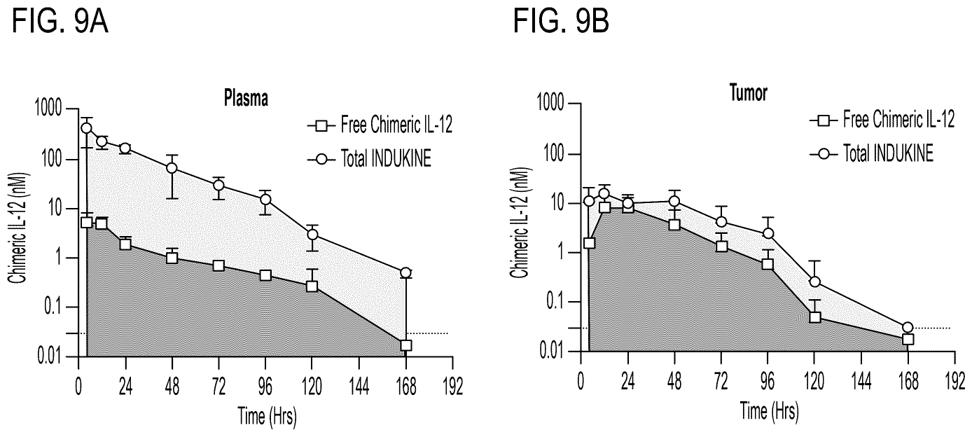

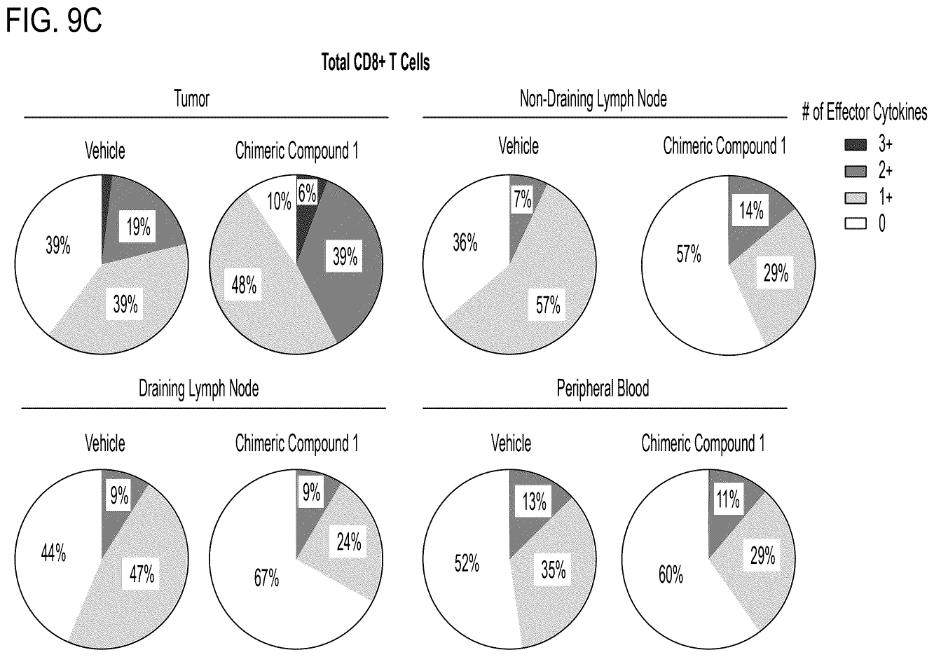

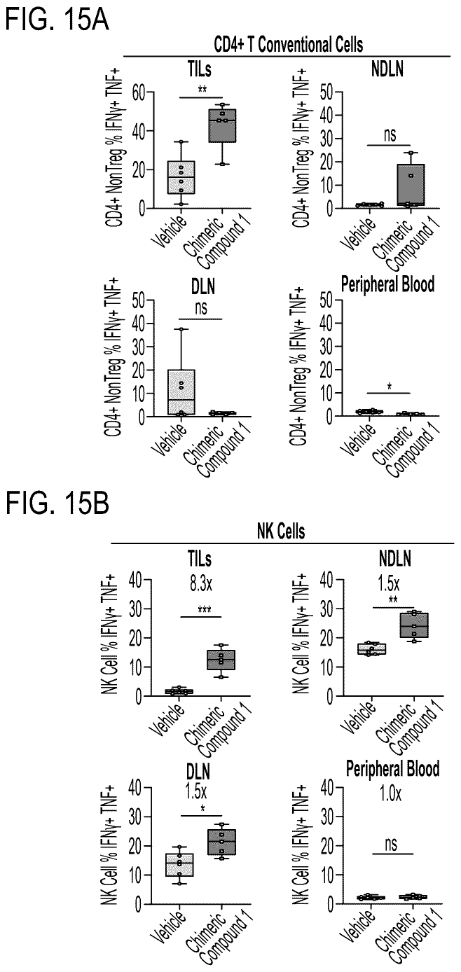

In the clinic, free cytokines have poor pharmacokinetic profiles with short half-lives, resulting in rapid clearance and poor exposure in patients, and leading to unreasonable dosing schedules. In order to examine whether the design of chimeric Compound 1 enhanced the half-life and exposure of the molecule, MC38 tumor bearing mice were dosed with a single dose of either chimeric IL-12 or chimeric Compound 1, and peripheral blood and tumor samples were collected over time. Using a unique set of detection reagents, it was possible to separately measure the amount of total IL-12 (blocked+unblocked) or to selectively measure the amount of unblocked IL-12 present. In the plasma of tumor bearing mice, chimeric IL-12 had a half-life of only 4 hours, while chimeric Compound 1 had a half-life of nearly 16 hours (FIG. 9A). Furthermore, only about 2% of the chimeric Compound 1 found in the plasma was in the form of the cleaved molecule. In contrast, when the same analysis was performed on tumor samples (FIG. 9B), nearly 45% of the molecule was unmasked IL-12 and inter-tumoral exposure was maintained far beyond what was achieved by treatment with chimeric IL-12. To further corroborate the selective processing of chimeric Compound 1 in the tumor, the activation status of tumor infiltrating CD8+ T cells, CD4+ T conventional cells, and NK cells was compared to those same populations in the tumor draining and non-draining lymph nodes, as well as the peripheral blood following chimeric Compound 1 treatment. While chimeric Compound 1 treatment resulted in a significant increase in the frequency of polyfunctional CD8+ T cells within the MC38 tumors, no such increase was observed in the lymph nodes or in the peripheral blood (FIG. 9C). Likewise, among CD4+ T conventional cells (FoxP3−) (FIG. 15A) and NK Cells (FIG. 15B), chimeric Compound 1 preferentially increased the frequency of cells producing effector cytokines in the tumor compared to the peripheral tissues.

### 3. Chimeric Compound 1 Treatment Activates Various TIL Populations in the MC38 Model

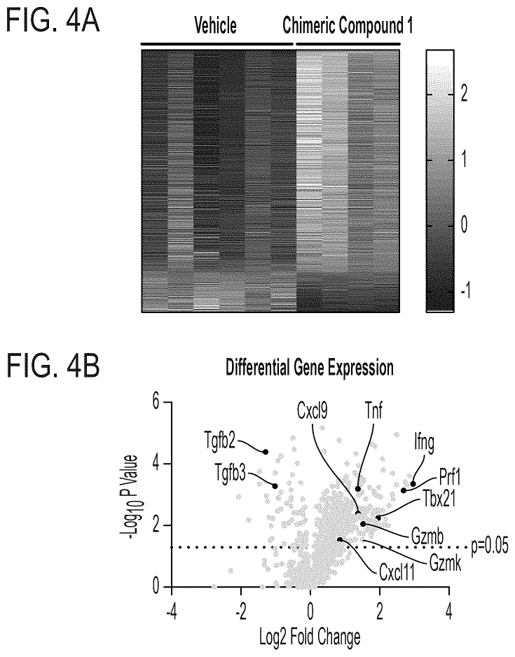

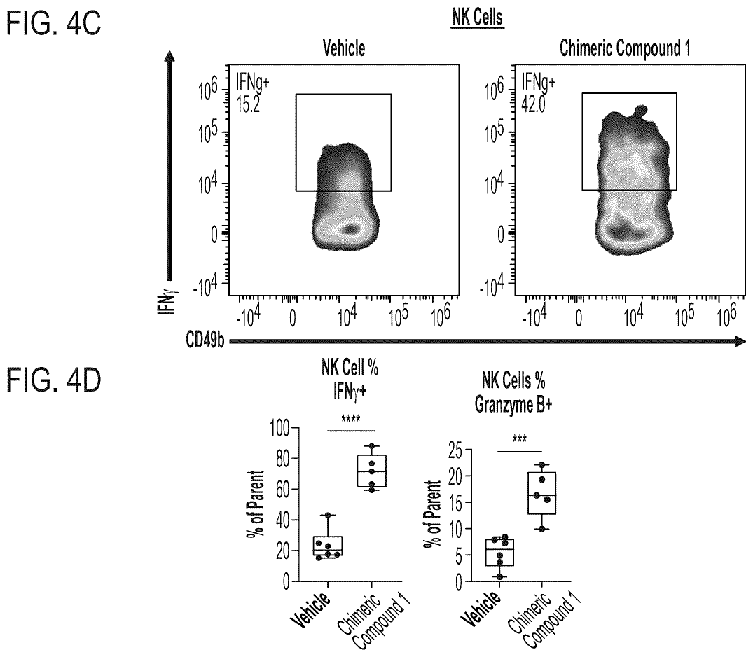

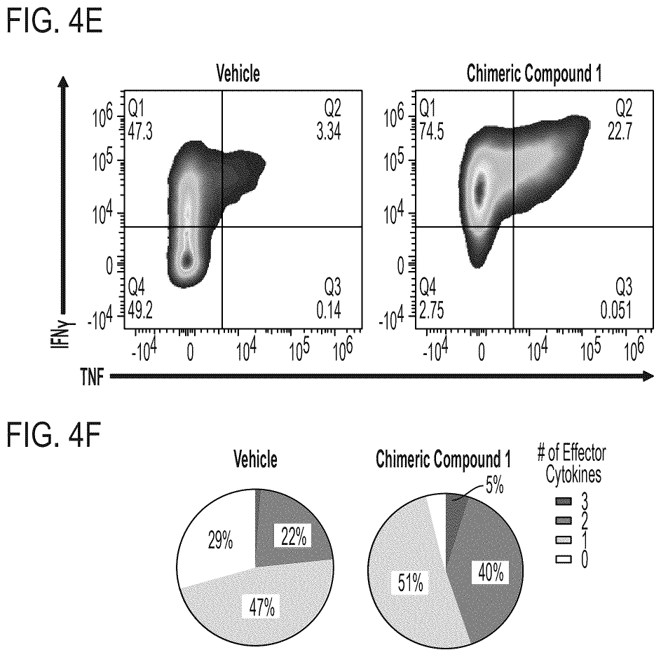

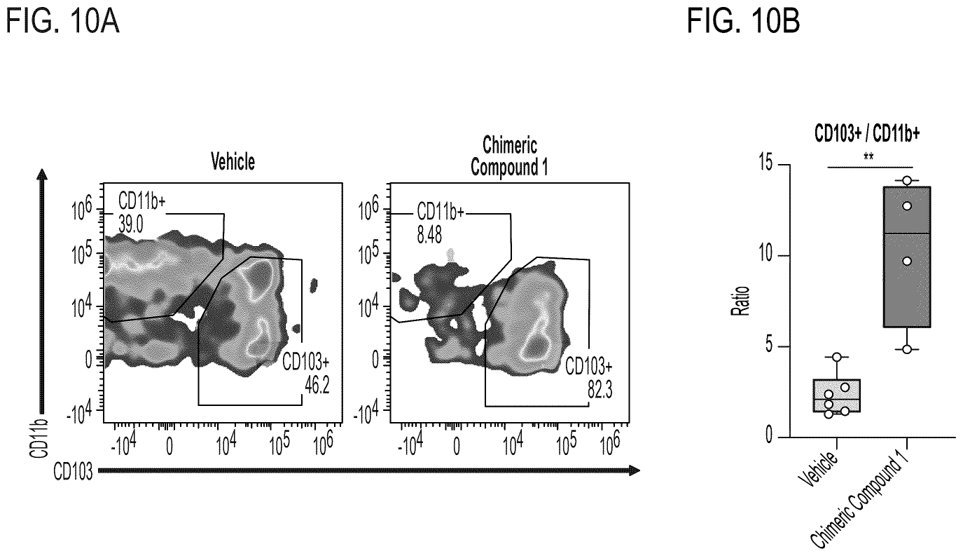

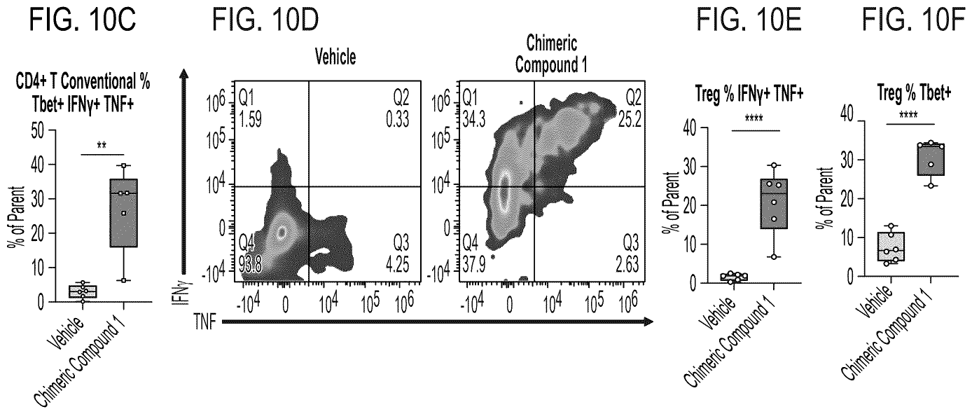

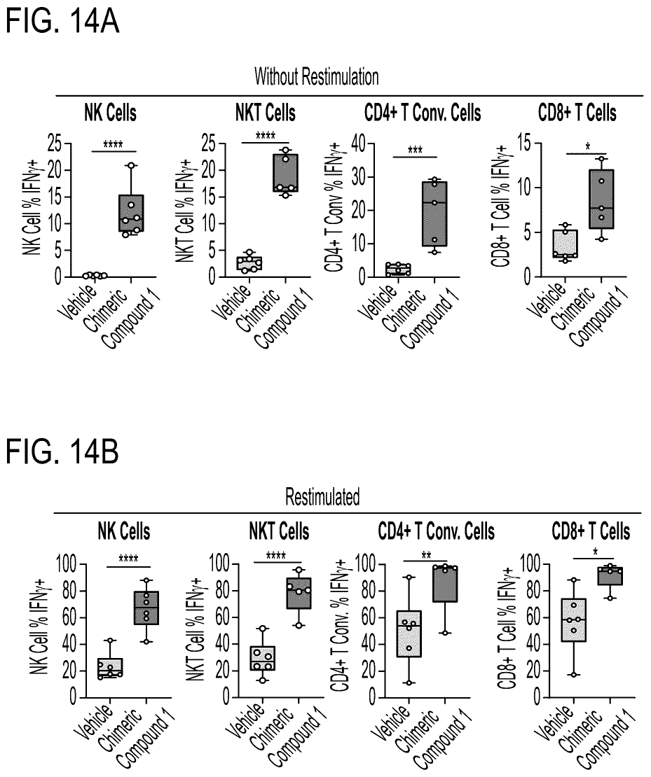

To better understand the mechanism by which chimeric Compound 1 treatment generates anti-tumor immunity, MC38 tumor-bearing mice were randomized into treatment groups on Day 0 and treated with either vehicle or chimeric Compound 1 on Day 1 and Day 4. Tumors were harvested 24 hours after the second dose and analyzed by flow cytometry or NanoString analysis using the PanCancer Mouse Immune Profiling Panel. Systemic treatment with chimeric Compound 1 had a striking effect on the transcriptional profile of the TME, with 364 of the 770 investigated transcripts having statistically significant differences in expression after treatment (FIG. 4A). Chimeric Compound 1 treatment resulted in significant enrichment of several immune related signaling pathways, including “PD-L1 Expression and PD-1 Checkpoint in Cancer”, “NK Cell Cytotoxicity”, and “TH1 and TH2 Differentiation”. In agreement with this analysis, chimeric Compound 1 treatment resulted in a significant increase in the frequency of tumor infiltrating NK cells producing IFNγ, TNF, and Granzyme B (FIG. 4D). Interestingly, chimeric Compound 1 treatment resulted in NK, NKT, CD4+ T conventional cells, and CD8+ T cells producing such elevated levels of IFNγ that it was measurable by intracellular cytokine staining without ex vivo restimulation (FIGS. 14A-14B). However, the signaling pathway with the highest enrichment score following treatment with chimeric Compound 1 was “Antigen Processing and Presentation.” Gene set enrichment analysis revealed that chimeric Compound 1 treatment significantly enriched several gene sets associated with antigen presentation of exogenous peptides and/or antigens in either MHC class I or MHC class II proteins. Presentation of exogenously derived antigens in MHC class I proteins is a phenomenon known as cross presentation, and it is exclusively mediated by a unique population of dendritic cells, identified by the expression of CD103(25). In agreement with the bioinformatic analysis, flow cytometric analysis demonstrated that chimeric Compound 1 treatment significantly increased the frequency of the cross presenting CD103+ DC population among total DCs (FIGS. 10A-10B).

Given the role of CD8+ T cells in chimeric Compound 1 mediated tumor rejections (FIG. 8A) and the finding that chimeric Compound 1 increased tumor infiltration by cross presenting DCs, it seemed likely that chimeric Compound 1 treatment also enhanced CD8+ T cell activation. Indeed, differential expression analysis of the total RNA demonstrated that chimeric Compound 1 treatment significantly increased the expression of many transcripts associated with activation of cytotoxic CD8+ T cells, including Ifnγ, Granzyme B, perforin, and Tnf, in addition to several chemoattractant molecules (FIG. 4B). While chimeric Compound 1 did not increase the frequency of tumor specific CD8+ T cells at this early time point, treatment did result in robust activation of the tumor specific CD8+ T cell population, as demonstrated by an increase in the frequency of tetramer+ polyfunctional CD8+ T cells (FIG. 4F), with nearly 100% of the tumor specific T cells producing IFNγ. Comparable results were seen when considering the entire tumor infiltrating CD8+ T cell population, and not just the tetramer positive ones. Additionally, among CD4+ T cells, chimeric Compound 1 treatment significantly increased the frequency of CD4+ T conventional cells with a TH1 phenotype (Tbet+ IFNγ+ TNF+) (FIG. 10C). Finally, recent publications have highlighted the role of IFNγ in driving Tregs away from regulatory activity and towards an effector phenotype, in a phenomenon known as Treg fragility. Chimeric Compound 1 treatment resulted in a significant subset of the FoxP3+ Treg population co-producing the effector cytokines TNF and IFNγ (FIGS. 10D-10F) and expressing Tbet (FIG. 10F), demonstrating that systemic treatment with chimeric Compound 1 can induce Treg instability in the TME. Altogether, these data demonstrate that systemic administration of chimeric Compound 1 results in the transcriptional reprogramming of the TME, and the subsequent activation of various tumor infiltrating effector cell populations.

### 4. Chimeric Compound 1 Treatment Expands Unique TCR Clones and Increases TCR Clonality in the TME

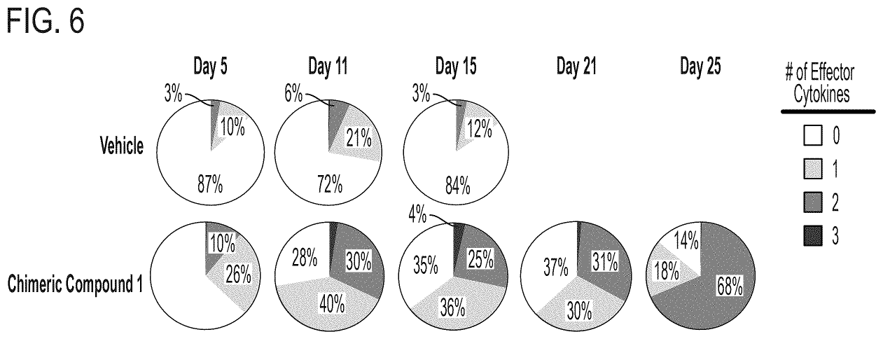

In the MC38 tumor model, chimeric Compound 1 treatment results in rapid tumor rejection, making it technically challenging to fully investigate the kinetics of immune activation. In contrast, chimeric Compound 1 treatment of the EMT-6 tumor model generates complete rejections over a longer period, which is favorable to a more thorough analysis of an ongoing CD8+ T cell response (FIG. 3C). Therefore, mice bearing established EMT-6 tumors were randomized into treatment groups and dosed twice a week for two weeks with either vehicle or chimeric Compound 1. Tumors and plasma were then harvested at various timepoints. Interestingly, in the control animals, the frequency of polyfunctional CD8+ T cells did expand over the course of the experiment, but eventually retracted in line with eventual tumor growth. In contrast, chimeric Compound 1 treatment increased the frequency of polyfunctional CD8+ T cells over that of the control animals as soon as Day 5 after the start of treatment (FIG. 6), and this frequency continued to expand even after exposure to chimeric Compound 1 was undetectable.

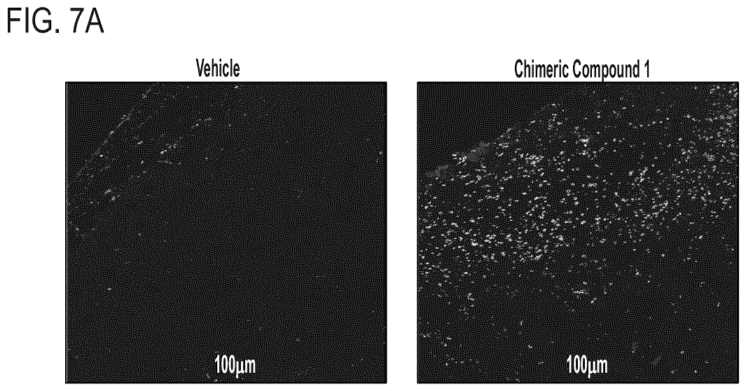

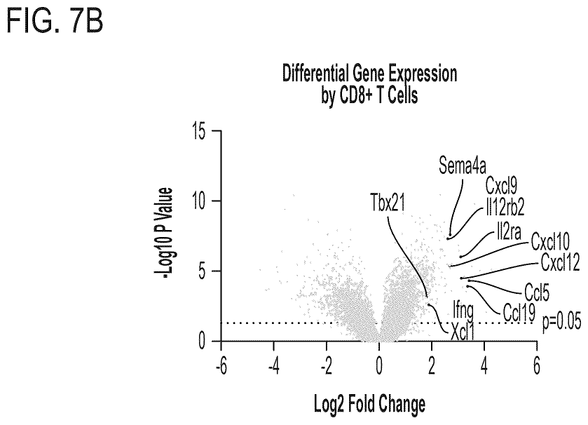

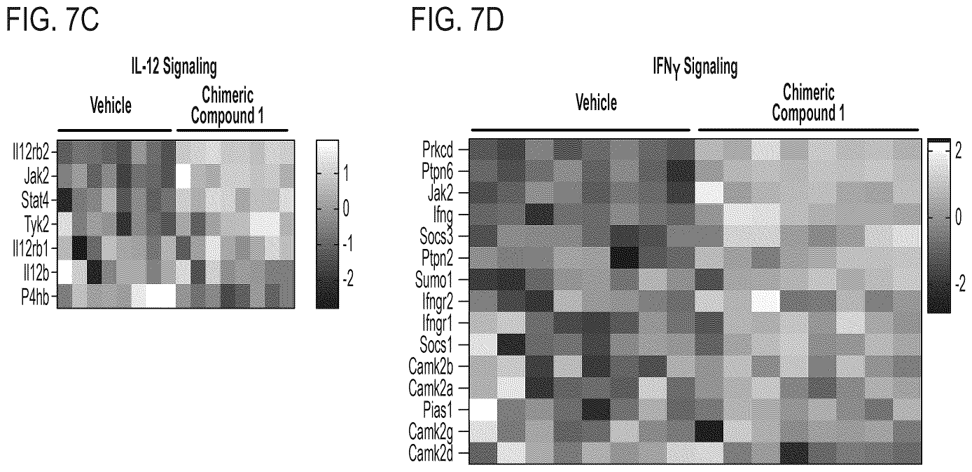

To better understand the transcriptional effects of chimeric Compound 1 treatment specifically on the tumor infiltrating CD8+ T cell population, Geospatial NanoString analysis was performed on tumor samples from Day 11. This technology merges immunofluorescence with whole transcriptome analysis of specific cells, allowing for transcriptional analysis of a specific cell population while maintaining spatial information that would otherwise be lost during tissue dissociation. In the control group, CD8+ T cells were largely confined to the outer margins of the tumor (FIG. 7A). In contrast, chimeric Compound 1 treatment induced significant infiltration of the EMT-6 tumors, with CD8+ T cells penetrating deeply into the tumor tissue (FIG. 7A). Whole transcriptome analysis of the tumor infiltrating CD8+ T cells demonstrated that chimeric Compound 1 treatment resulted in substantial transcriptional reprogramming of these cells, including upregulation of many genes associated with T cell activation such as Tbet, IFNγ, Cd25, and chemoattractants known to be responsible for the recruitment of additional immune cells (FIG. 7B).

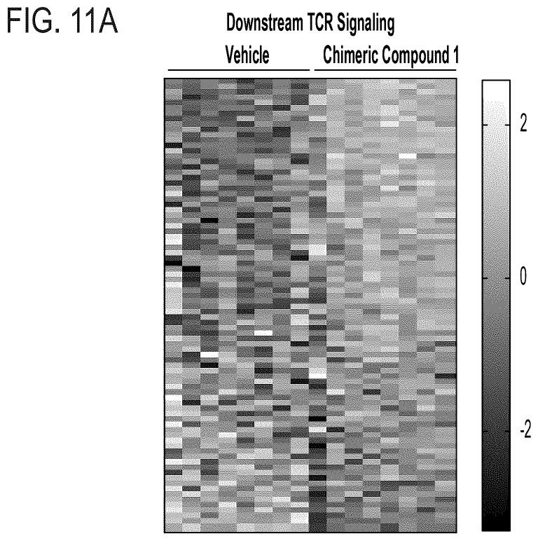

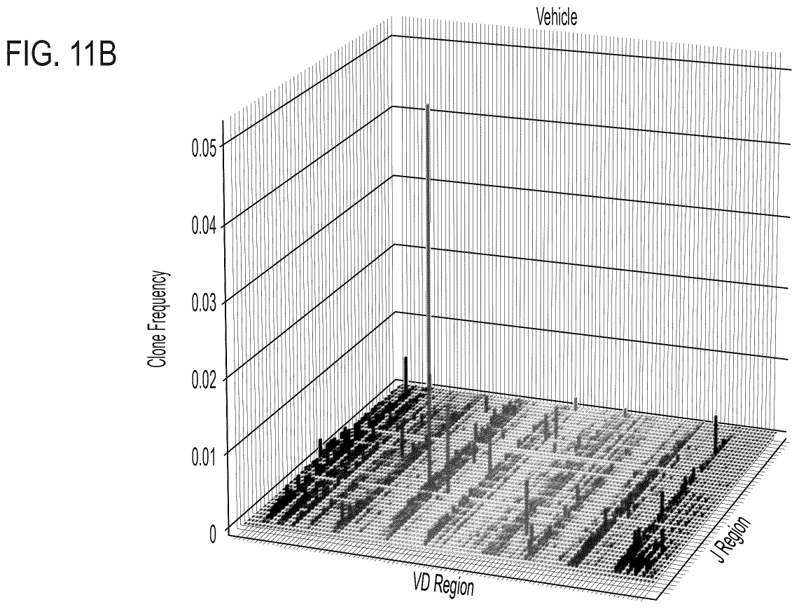

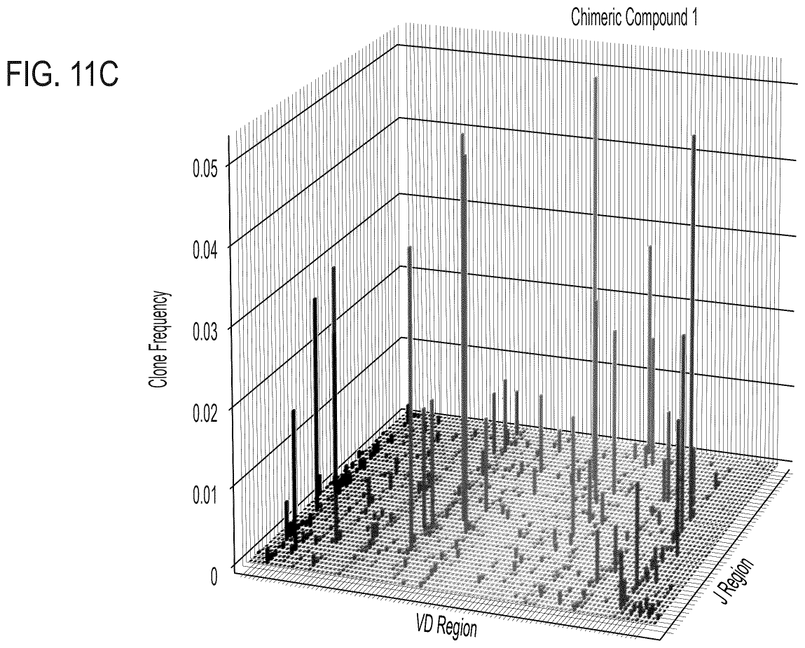

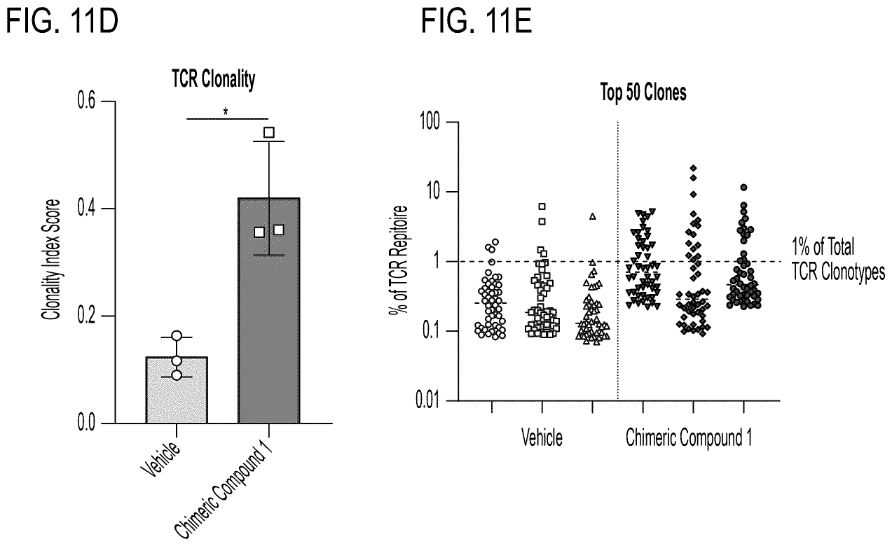

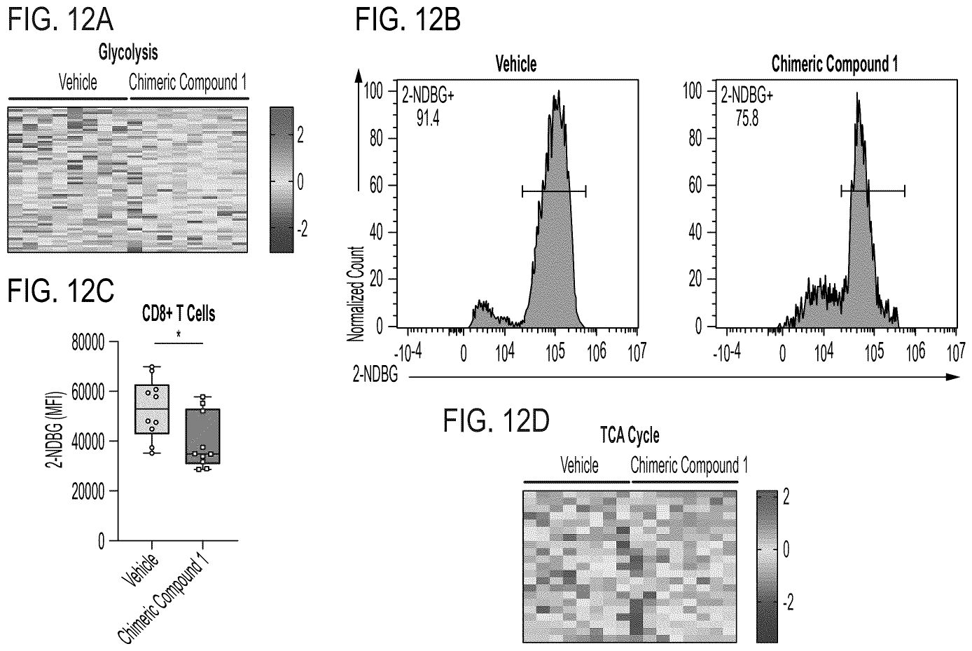

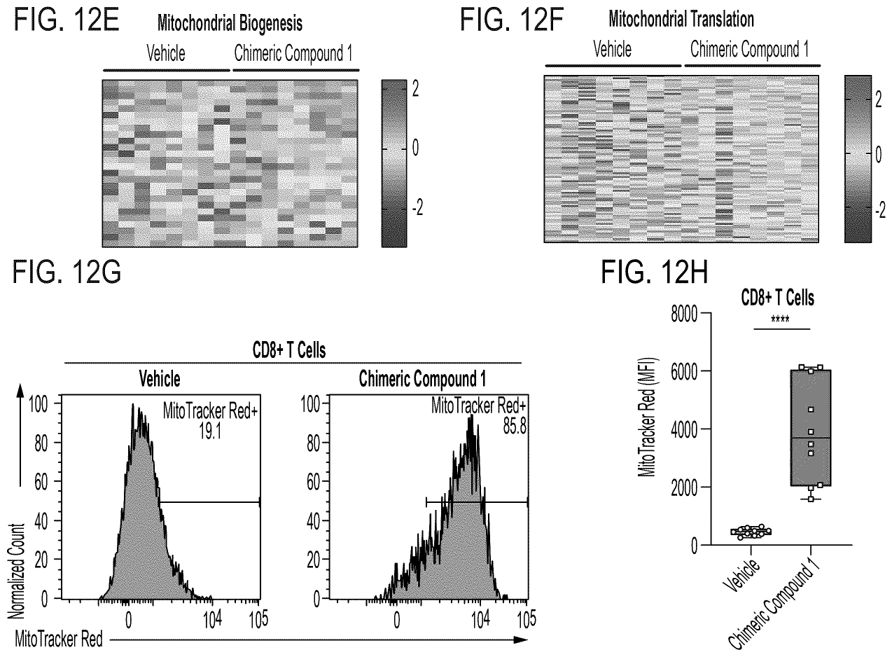

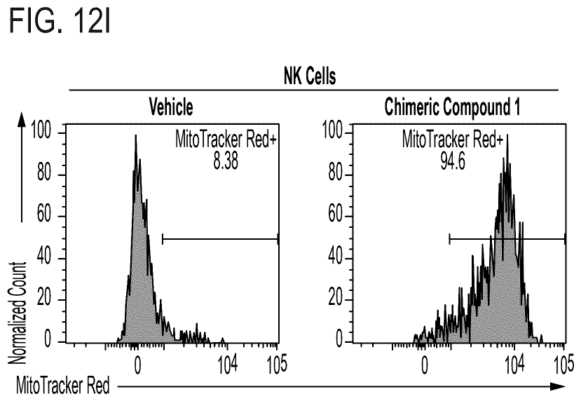

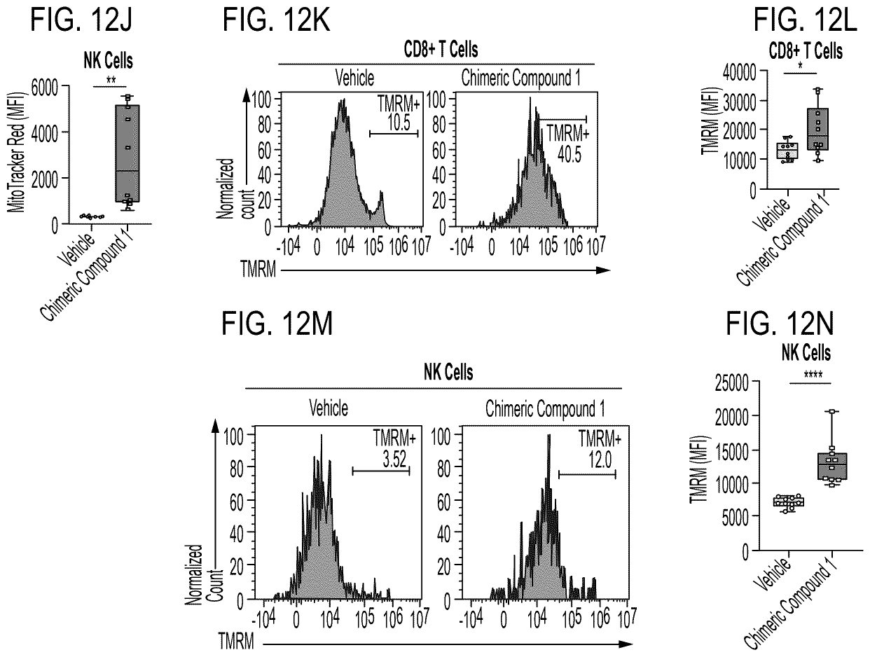

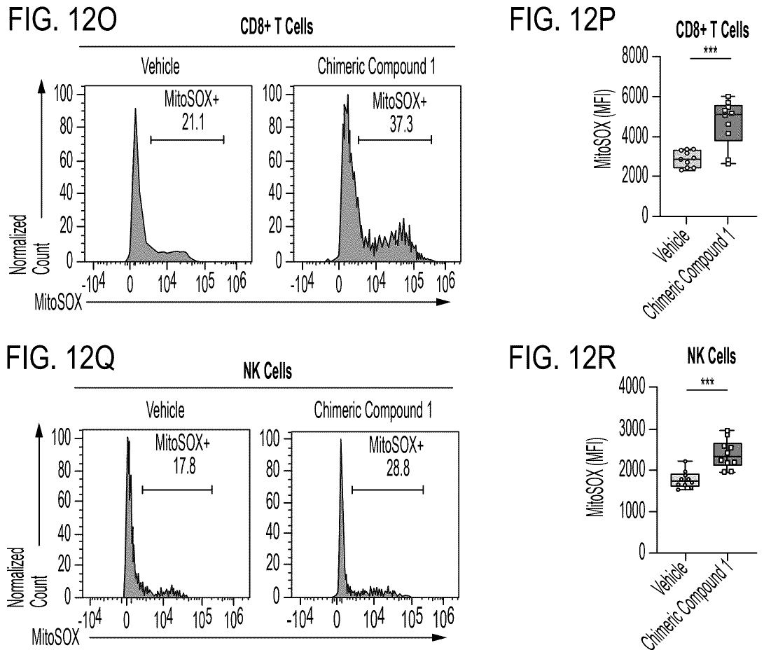

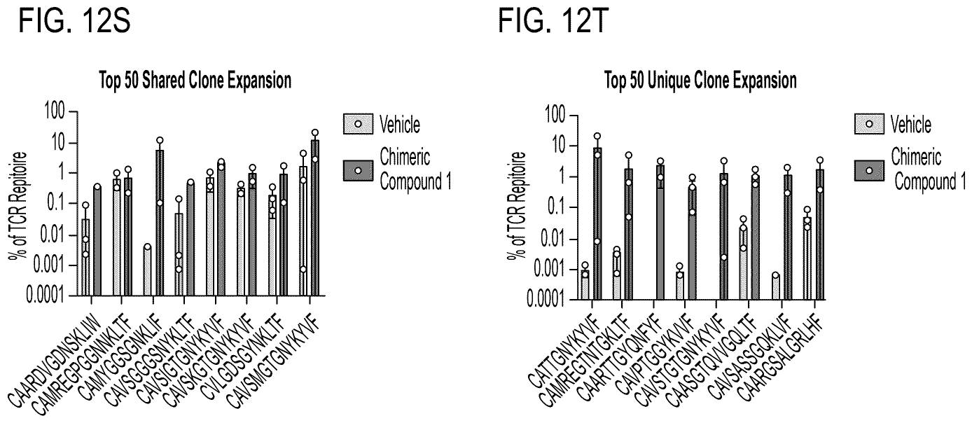

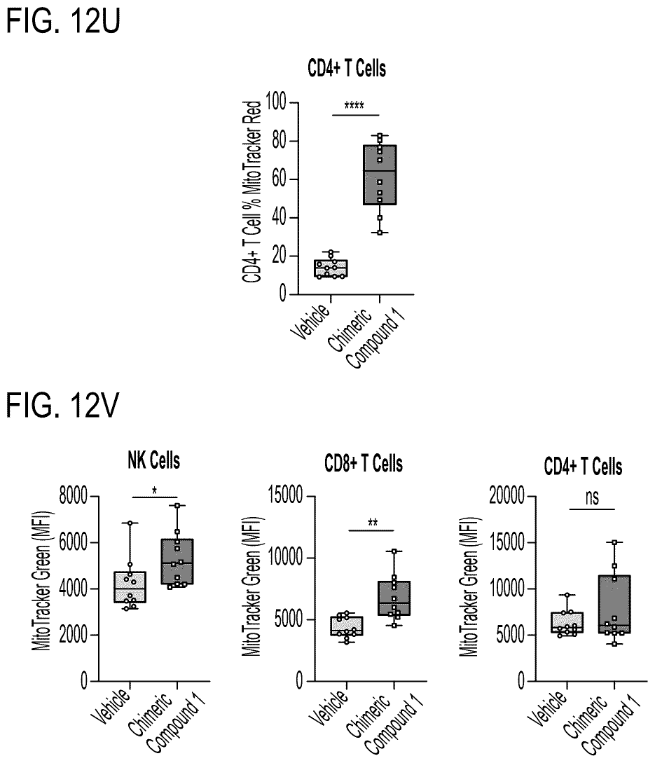

Among the pathways activated in tumor infiltrating CD8+ T cells by chimeric Compound 1 treatment, IL-12 (FIG. 7C) and IFNγ signaling (FIG. 7D) were both significantly upregulated, confirming the local release of unmasked IL-12 and subsequent production of IFNγ within the TME. Chimeric Compound 1 treatment also increased expression of transcripts downstream of TCR signaling (FIG. 11A). Given the effects on cross presenting DCs, we hypothesized that chimeric Compound 1 treatment may result in the activation of new T cell clones to generate anti-tumor immunity. In order to test this, T cells were isolated from EMT-6 tumors following chimeric Compound 1 or vehicle treatment and sent for TCR sequencing. While the tumor infiltrating TCR repertoire of control animals was dominated by many low frequency clones, chimeric Compound 1 treatment drove a robust expansion of several TCR clones (FIG. 11B), resulting in a significant increase in the overall clonality of the tumor infiltrating TCR repertoire (FIG. 11C). Indeed, when examining the overall frequency of the top fifty clones in each group, chimeric Compound 1 treatment resulted in a substantial increase in the number of clones making up more than 1% of the total repertoire across multiple animals (FIG. 11D). Further analysis of this subset revealed that only 1 of the 8 clones common to both treatment groups expanded at least 10-fold with chimeric Compound 1 treatment (FIG. 12SA). In contrast, of the clones unique to the chimeric Compound 1 treated group, all of them expanded over 10-fold compared to the control group (FIG. 12T), suggesting that chimeric Compound 1 treatment is increasing the clonality of T cell populations primarily by expanding clones that were previously underrepresented, rather than increasing the frequency of already dominant clones.

### 5. Chimeric Compound 1 Substantially Increases Mitochondrial Activity in Tumor Infiltrating CD8+ T Cells and NK Cells

Recently activated CD8+ T cells have substantial energy requirements and rely heavily on glucose uptake and glycolysis to quickly generate the energy necessary to perform their effector functions, before transitioning towards mitochondria dependent oxidative phosphorylation as they develop into long-lived memory cells. However, recent publications have demonstrated that tumor infiltrating CD8+ T cells often fail to induce significant mitochondrial respiration compared to those activated in the spleen or lymph nodes, suggesting that the TME negatively impacts the metabolic health of effector cells. Among tumor infiltrating CD8+ T cells, chimeric Compound 1 treatment resulted in a significant enrichment of transcripts associated with glycolysis (FIG. 12A). Therefore, we hypothesized chimeric Compound 1 treatment may increase glucose uptake by tumor infiltrating CD8+ T cells, and thereby lead to increased glycolysis. However, when these cells were incubated with a non-metabolizable fluorescent glucose analog (2-NDBG), tumor infiltrating CD8+ T cells from chimeric Compound 1 treated animals actually had slightly less glucose uptake than those from vehicle animals (FIGS. 12B-12C). Therefore, rather than simply increasing glucose uptake by tumor infiltrating CD8+ T cells, chimeric Compound 1 treatment was instead reprogramming those cells to utilize glucose more efficiently than those from vehicle treated animals.

In addition to driving increased glycolysis, chimeric Compound 1 treatment also enriched for transcripts associated with the TCA cycle, mitochondrial biogenesis, and mitochondrial translation, suggesting that chimeric Compound 1 treatment may enhance the mitochondrial activity and health of tumor infiltrating effector cells (FIGS. 12D-12F). To test this, TILs were isolated from vehicle or chimeric Compound 1 treated animals and mitochondrial phenotyping was performed by flow cytometry. Mitotracker Red is a dye that specifically stains actively respirating mitochondria due to its pH sensitivity. While tumor infiltrating CD8+ T cells from vehicle treated animals had limited evidence of ongoing active mitochondrial respiration, those from chimeric Compound 1 treated animals had significantly increased levels of active respiration (FIGS. 12G-12H). Interestingly, this finding also extended to NK cells (FIGS. 12I-12J) and total CD4+ T cells (FIG. 12U). This increase was primarily due to increased mitochondrial activity, rather than simply an increase in total mitochondrial mass, as chimeric Compound 1 treatment only slightly increased the total mitochondrial mass of tumor infiltrating NK cells, CD8+ T cells, and total CD4+ T cells (FIG. 12V). Furthermore, TMRM staining also revealed that chimeric Compound 1 treatment significantly increased the mitochondrial membrane potential in both CD8+ T cells (FIGS. 12K-12L) as well as NK cells (FIGS. 12M-12N). Mitochondrial reactive oxygen species (ROS) have previously been linked both to NFAT signaling and subsequent production of IL-2(31), as well as IFNγ production by memory CD4+ T cells, and can be detected using the dye MitoSOX Red. Chimeric Compound 1 treatment increased the production of mitochondrial ROS species in both CD8+ T cells (FIGS. 12O-12P) as well as NK cells (FIGS. 12Q-12R).

Oxidative phosphorylation is the primary energy source for memory T cells, and increased dependence on this pathway has been associated with superior anti-tumor immunity and a “stem-cell like” phenotype. Tumor infiltrating CD8+ T cells from chimeric Compound 1 treated mice also significantly upregulated expression of genes associated with T cell stemness, including Tcf7, Cxcr3, and Il2rγ while significantly downregulating expression of several genes associated with CD8+ T cell exhaustion, including Pdcd1, Havcr2, and Lag3. Together, these data demonstrate that systemic administration of chimeric Compound 1 is sufficient to restore the mitochondrial respiration of tumor infiltrating CD8+ T and NK cells and pushes the CD8+ T cell population to adopt a more “stem-cell like” phenotype, which may translate into superior anti-tumor immunity.

### 6. Compound 36, a Fully Human Inducible IL-12 Prodrug, is Stable in Human Serum and Preferentially Activated by Primary Human Tumor Samples

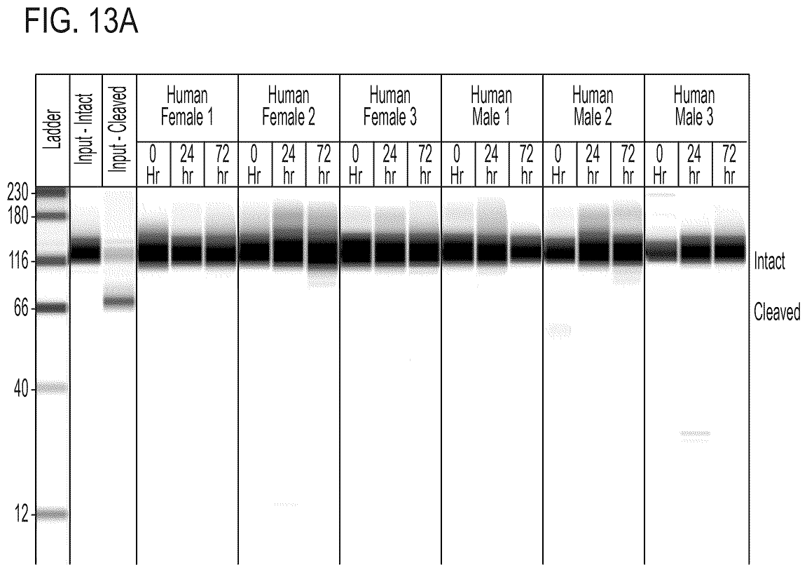

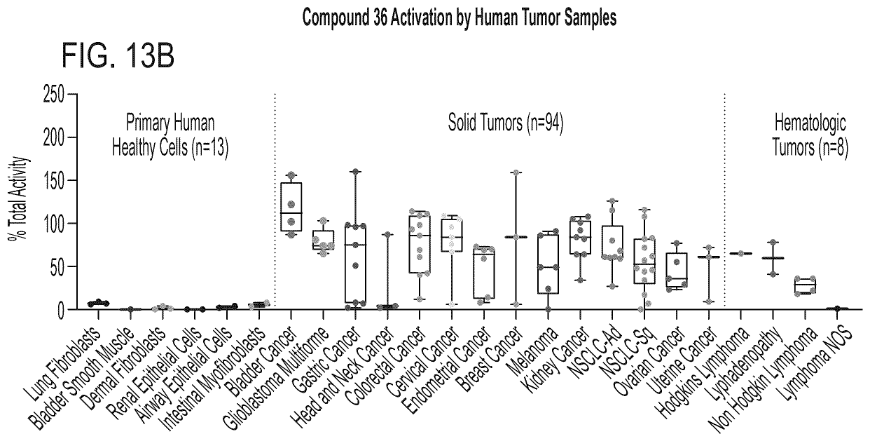

For preclinical murine studies, it was important to use a surrogate molecule that was active in mice. However, for clinical development, a fully human IL-12 payload will be used. Compound 36 is identical to chimeric Compound 1 except that it contains fully human IL-12 as the payload. As with the murine surrogate molecule, intact Compound 36 had substantially less activity than either cleaved Compound 36 or recombinant human IL-12 in a HEK-Blue IL-12 reporter assay. Likewise, when exposed to stimulated primary human Tblasts from multiple donors, intact Compound 36 was 61-fold less active on average than the cleaved molecule. In both of these in vitro assays, cleaved Compound 36 had activity similar to recombinant human IL-12. Furthermore, when Compound 36 incubated in serum from healthy human donors (n=6), no free IL-12 was detected after 72 hours at 37° C. (FIG. 13A), confirming the stability of the molecule. In order to examine whether Compound 36 would be selectively processed by primary human tumor samples, an in vitro cleavage assay was developed. Briefly, primary human dissociated tumor samples from various indications, or primary human cells from healthy tissues were incubated for 48 hours with either Compound 36, pre-cut Compound 36, or an uncleavable variant of Compound 36 before the cell culture supernatants containing the processed inducible IL-12 prodrug were collected. Since human Tblasts can differentiate between intact and cleaved Compound 36, primary human Tblasts were exposed to the cell culture supernatants and the production of IFNγ was used as a surrogate marker for the processing of Compound 36. The results of this assay were then normalized to the uncleavable negative control (0% processed) and the positive pre-cleaved control (100% processed). Among the n=88 primary human tumor samples evaluated; Compound 36 was efficiently processed across all tested indications (FIG. 13B). In contrast, incubation of Compound 36 with primary human cells from various healthy tissues (n=13) resulted in no evidence of processing. These data suggest that Compound 36 is efficiently and selectively processed by primary human tumor samples and support the continued clinical development of this molecule.

### 1.3 Discussion

IL-12 has long been a cytokine of great interest for oncology due to its potential to induce innate and adaptive immune responses (9,11) and its promising anti-tumor preclinical data(12,18,20,34,35). Nevertheless, despite this interest, the poor pharmacokinetic properties of this cytokine and the unacceptable levels of toxicity associated with its systemic administration have prevented its use in clinical settings(9,10,24,36). To address these concerns, we developed an inducible IL-12 prodrug, Compound 36. Compound 36 a prodrug molecule, designed to be an infrequently administered, systemically delivered therapy with targeted intra-tumoral activation that releases native IL-12 into the tumor microenvironment. Our data with chimeric Compound 1 demonstrated anti-tumor activity in the MC38 tumor model that was dependent on in vivo cleavage of the inducible IL-12 prodrug by the tumor. Furthermore, chimeric Compound 1 was a very potent monotherapy in several mouse tumor models with varying levels of baseline infiltration, including complete responses in a model refractory to anti-PD-1 treatment (EMT-6). These complete responses translated into robust immune memory against subsequent rechallenge with the same tumor cell line, highlighting the role of the immune system in tumor rejection. In the MC38 model, long term efficacy was dependent on the presence of CD8+ T cells, but overall tumor growth inhibition was driven by contributions from three main effector cell types, CD8+ T cells, CD4+ T conventional cells, and NK cells. The inducible IL-12 prodrug design also resulted in increased exposure and a favorable ratio of active IL-12 versus the blocked pro-drug molecule in the tumor tissue compared to the plasma, which correlated with selectively localized pharmacodynamic changes (effector cell polyfunctionality) observed in the tumor versus peripheral tissues. Importantly, chimeric Compound 1 proved to be well-tolerated in mice compared with recombinant chimeric IL-12 treatment, while maintaining the potential to induce complete tumor regressions, resulting in an almost 10-fold improvement of the therapeutic window compared to the unblocked cytokine. Improvement of the therapeutic window is a key feature of inducible IL-12 prodrugs and is necessary to facilitate clinical development of potent cytokines for oncology treatment.

Chimeric Compound 1 treatment robustly activated various tumor infiltrating innate and adaptive effector cell populations, supporting a mechanism of action where infiltration and activation of multiple effector cells plays a fundamental role in initial tumor control. The tumor specific delivery of active IL-12 and subsequent induction of intratumoral IFNγ also induced Treg fragility, which likely contributes to the potent efficacy delivered by chimeric Compound 1 treatment. However, of equal importance, is the effect that chimeric Compound 1 treatment had on antigen processing and presentation and the observed increase in tumor infiltration by cross-presenting dendritic cells. These cells are responsible for the de novo generation of new T cell responses to novel tumor antigens, and several publications have identified their importance in the generation of pre-clinical anti-tumor immunity(37,38). The dual role of IL-12 as a direct activator of effector cell populations and as a driver of cross presenting dendritic cell activation will likely set apart therapeutics based on this cytokine compared to other treatments when it comes to triggering efficacy in “cold” tumors. Indeed, we observed this effect using a model of a “cold” tumor, the poorly infiltrated EMT-6 model. NanoString Digital Spatial Profiling demonstrated that systemic treatment with chimeric Compound 1 enhanced deep infiltration of EMT-6 tumors by CD8+ T cells and confirmed the intratumoral increase in IL-12 and IFNγ signaling, as well as the significant upregulation of transcripts associated with robust CD8+ T cell activation. Treatment also significantly increased the clonality of the TCR repertoire among tumor infiltrating T cells and drove the expansion of several novel clones, suggesting that systemic chimeric Compound 1 treatment resulted in the activation of a de novo T cell response to unique tumor antigens, which may be key to the CD8+ T cell dependent tumor rejection observed earlier.

Finally, systemic chimeric Compound 1 treatment had a substantial effect on the metabolism of the tumor infiltrating effector cells, transforming the metabolic status of not just the activated, tumor infiltrating CD8+ T cells but also that of the intratumoral NK cells. The TME is known to have several distinct characteristics when compared to a typical cellular environment, including a lower pH, hypoxic conditions, and significant competition for extracellular glucose, all of which may impair effector cell activity. Despite the evidence for increased glycolysis, we observed a slight reduction in extracellular glucose uptake following chimeric Compound 1 treatment, suggesting that chimeric Compound 1 treatment is not simply driving greater glucose uptake by tumor infiltrating cells, but is instead facilitating an increase in metabolic efficiency and increasing oxidative phosphorylation. Recent studies have demonstrated that tumor infiltrating T cells often fail to robustly activate mitochondrial respiration compared to those that have been activated in the spleen or lymph nodes, suggesting that the TME impairs metabolic health of effector cells(27,28,30). In agreement with these studies, EMT-6 tumor infiltrating CD8+ T cells and NK cells from vehicle treated animals had very little evidence of ongoing mitochondrial respiration, despite efficiently taking up 2-NDBG in vitro. In contrast, effector cells from Compound 36 treated animals robustly upregulated active mitochondrial respiration, mitochondrial membrane potential, and mitochondrial reactive oxygen species. The transition of these cells strongly towards oxidative phosphorylation could be important in the context of the highly dysregulated metabolic environment within the tumor, where there is stiff competition for glucose, and every molecule must be used to its fullest extent to support effector cell activation.

In conjunction with the metabolic effects of the TME, effector cells also have to contend with another mechanism of immune regulation, exhaustion. This state is characterized by high co-expression of checkpoint proteins (such as PD-1, LAG-3, TIGIT, and TIM-3)(39), the loss of effector cytokine production, and the failure to proliferate following restimulation. However, recent publications have demonstrated that some tumor specific T cells maintain a more stem-cell like phenotype, termed increased “stemness,” and have identified a crucial role for the transcription factor TCF1 in maintaining that phenotype(40). Furthermore, increased “stemness” has been associated with greater anti-tumor immunity. In addition to the metabolic reinvigoration of the effector cells, treatment with chimeric Compound 1 likely increased the stemness of the tumor infiltrating CD8+ T cells, with upregulation of TCF7 (the mRNA transcript associated with TCF1, the protein) and decreased expression of PD-1 and Tim-3 by these cells. Together, these data suggest that chimeric Compound 1 treatment results in a robust and all-encompassing re-programming of the tumor infiltrating CD8+ T cell response.

The mechanistic studies described herein not only identified the cell types responsible for the anti-tumor efficacy of chimeric Compound 1 treatment, but also provide insight on how these effector cells are able to overcome the suppressive microenvironment within the tumor. Furthermore, these data demonstrate that the inducible IL-12 prodrug design of chimeric Compound 1 significantly increased the half-life of the molecule compared to recombinant IL-12 and allowed for selective activation in the TME following systemic administration, resulting in a significant expansion of the therapeutic window. Additional studies with fully human Compound 36 demonstrated that the inducible IL-12 prodrug was highly inducible and was cleaved by a majority of human tumor samples in vitro while demonstrating stability when incubated with normal primary cells and serum. Together, these data provide clear evidence for the continued preclinical development of this therapeutic molecule and support moving Compound 36 towards human clinical testing.

### Example 2. MC38 Experiments (Study MC38-e52)

The MC38 cell line, a rapidly growing colon adenocarcinoma cell line, was used. Using this tumor model, the ability of IL-12 prodrugs to affect tumor growth and body weight was examined.

TABLE 5
Agents and treatment regimen

| | | | Formulation | | |
|---|---|---|---|---|---|
| Gr. | N | Number | dose | Route | Schedule |
| 1^{#} | 8 | n/a | — | ip | biwk × 2 |
| 2 | 8 | WW00757/WW00636 | 50 μg/animal | ip | biwk × 2 |
| 3 | 8 | WW00680 | 50 μg/animal | ip | biwk × 2 |
| 4 | 8 | WW01102/WW00636 | 5 μg/animal | ip | biwk × 2 |
| 5 | 8 | WW01102/WW00636 | 50 μg/animal | ip | biwk × 2 |
| 6 | 8 | WW01102/WW00636 | 500 μg/animal | ip | biwk × 2 |
| 7 | 8 | WW01100 | 5 μg/animal | ip | biwk × 2 |
| 8 | 8 | WW01100 | 50 μg/animal | ip | biwk × 2 |
| 9 | 8 | WW01100 | 500 μg/animal | ip | biwk × 2 |
| 10 | 8 | WW01104 | 5 μg/animal | ip | biwk × 2 |
| 11 | 8 | WW01104 | 50 μg/animal | ip | biwk × 2 |
| 12 | 8 | WW01104 | 500 μg/animal | ip | biwk × 2 |
| 13 | 8 | WW01110 | 5 μg/animal | ip | biwk × 2 |
| 14 | 8 | WW01110 | 50 μg/animal | ip | biwk × 2 |
| 15 | 8 | WW01110 | 500 μg/animal | ip | biwk × 2 |
| 16 | 8 | WW01167 | 5.67 μg/animal | ip | biwk × 2 |
| 17 | 8 | WW01167 | 56.7 μg/animal | ip | biwk × 2 |
| 18 | 8 | WW01167 | 567 μg/animal | ip | biwk × 2 |

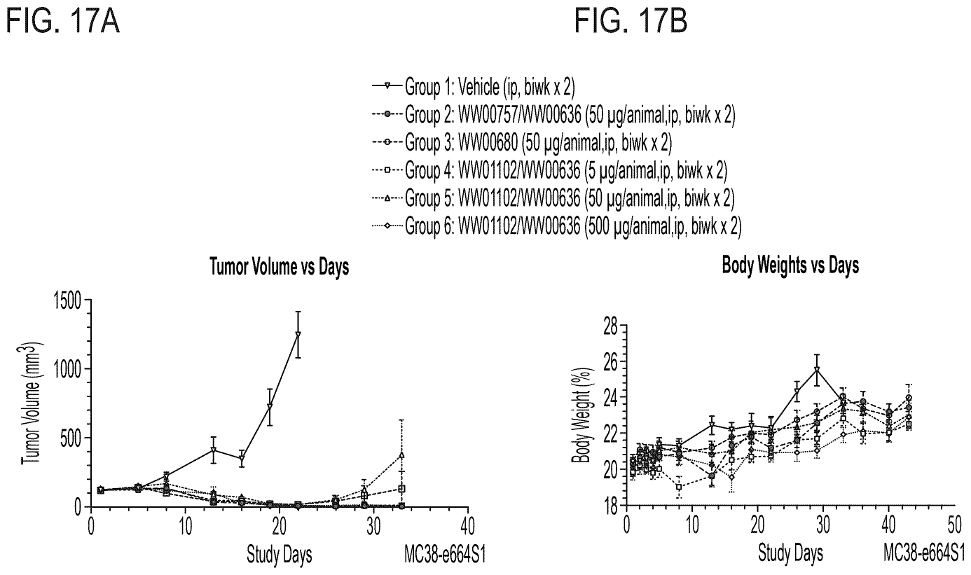

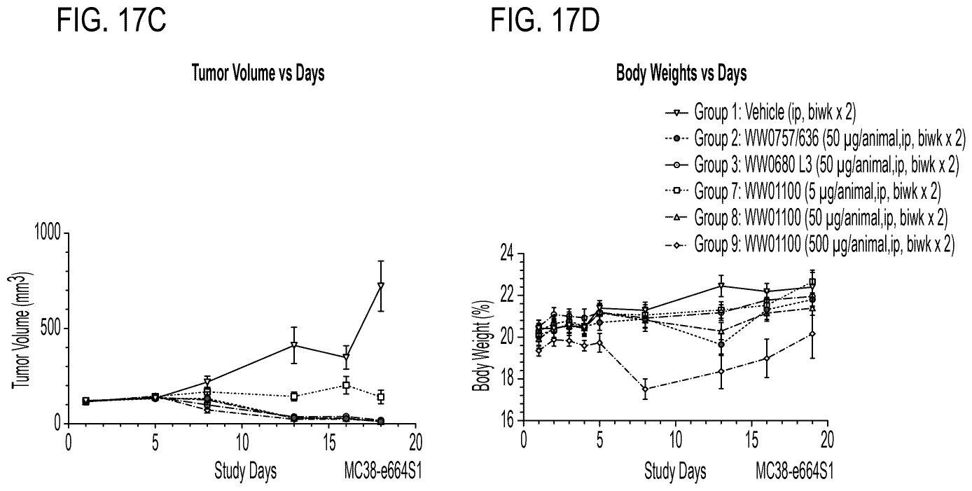

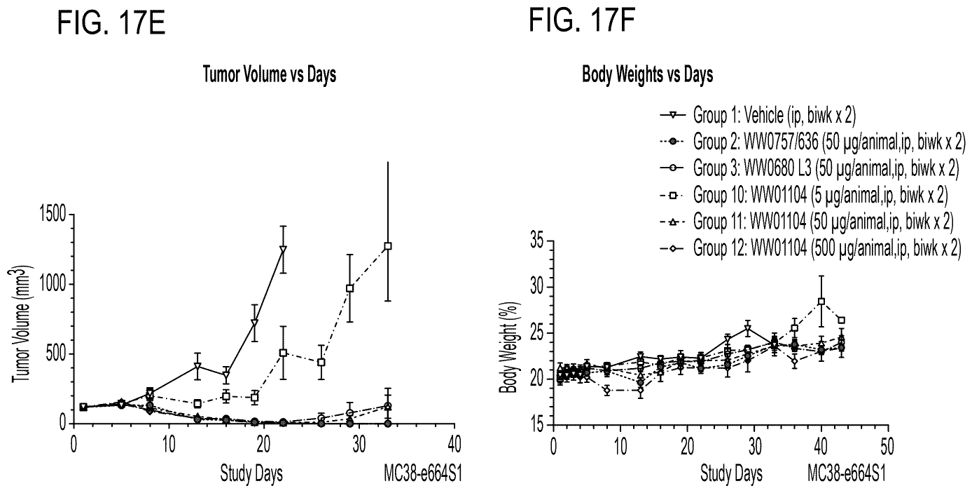

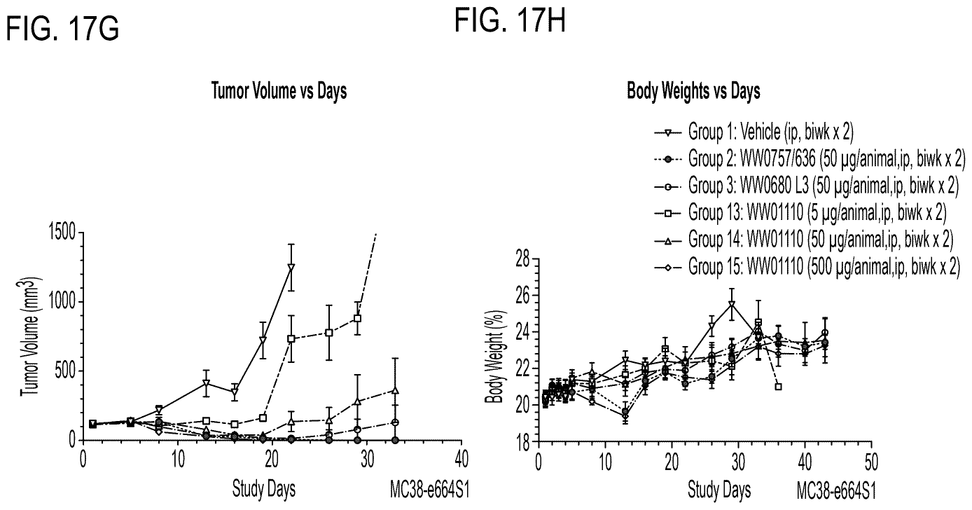

Mice were anaesthetized with isoflurane for implantation of cells to reduce the ulcerations. Female C57BL/6 mice were set up with 5×10⁵ MC38 tumor cells in 0% Matrigel sc in flank. Cell injection volume was 0.1 mL/mouse. Mouse age at start date was 8 to 12 weeks. Pair matches were performed when tumors reach an average size of 100-150 mm³ and began treatment. This was Day 1 of the study. Body weights were taken at initiation and then biweekly to the end. Caliper measurements were taken biweekly to the end. Any adverse reactions were reported immediately. Any individual animal with a single observation of >than 25% body weight loss or three consecutive measurements of >20% body weight loss was euthanized. Any group with a mean body weight loss of >20% or >10% mortality stopped dosing; the group was not euthanized, and recovery was allowed. Within a group with >20% weight loss, individuals hitting the individual body weight loss endpoint were euthanized. If the group treatment related body weight loss was recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 40 days, whichever came first. When the endpoint was reached, the animals were euthanized. Results are shown in FIGS. 17A-17J.

### REFERENCES FOR EXAMPLES 1 AND 2

1. Propper D J, Balkwill F R. Harnessing cytokines and chemokines for cancer therapy. Nat. Rev. Clin. Oncol. 2022. page 237-53.
2. Berraondo P, Sanmamed M F, Ochoa M C, Etxeberria I, Aznar M A, Pérez-Gracia J L, et al. Cytokines in clinical cancer immunotherapy. Br. J. Cancer. Nature Publishing Group; 2019. page 6-15.
3. Conlon K C, Miljkovic M D, Waldmann T A. Cytokines in the Treatment of Cancer. J Interf Cytokine Res. Mary Ann Liebert, Inc.; 2019; 39:6-21.
4. Nirschl C J, Brodkin H R, Hicklin D J, Ismail N, Morris K, Seidel-Dugan C, et al. Discovery of a Conditionally Activated IL-2 that Promotes Antitumor Immunity and Induces Tumor Regression. Cancer Immunol Res. American Association for Cancer Research (AACR); 2022; 10:581-96.
5. Charych D H, Hoch U, Langowski J L, Lee S R, Addepalli M K, Kirk P B, et al. NKTR-214, an Engineered Cytokine with Biased IL2 Receptor Binding, Increased Tumor Exposure, and Marked Efficacy in Mouse Tumor Models. Clin Cancer Res. American Association for Cancer Research Inc.; 2016; 22:680-90.
6. Klein C, Waldhauer I, Nicolini V G, Freimoser-Grundschober A, Nayak T, Vugts D J, et al. Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines. Oncoimmunology. Taylor and Francis Inc.; 2017; 6.
7. Lopes J E, Fisher J L, Flick H L, Wang C, Sun L, Ernstoff M S, et al. ALKS 4230: A novel engineered IL-2 fusion protein with an improved cellular selectivity profile for cancer immunotherapy. J Immunother Cancer. BMJ Publishing Group; 2020; 8:673.
8. Sharma M, Khong H, Fa'ak F, Bentebibel S E, Janssen L M E, Chesson B C, et al. Bempegaldesleukin selectively depletes intratumoral Tregs and potentiates T cell-mediated cancer therapy. Nat Commun. Nature Research; 2020; 11.
9. Waldmann T A. Cytokines in cancer immunotherapy. Cold Spring Harb Perspect Biol. Cold Spring Harbor Laboratory Press; 2018; 10.
10. Nguyen K G, Vrabel M R, Mantooth S M, Hopkins J J, Wagner E S, Gabaldon T A, et al. Localized Interleukin-12 for Cancer Immunotherapy. Front. Immunol. 2020.
11. Del Vecchio M, Bajetta E, Canova S, Lotze M T, Wesa A, Parmiani G, et al. Interleukin-12: biological properties and clinical application. Clin Cancer Res. 2007; 13:4677-85.
12. Lasek W, Zagożdżon R, Jakobisiak M. Interleukin 12: Still a promising candidate for tumor immunotherapy? Cancer Immunol. Immunother. 2014.
13. Tait Wojno E D, Hunter C A, Stumhofer J S. The Immunobiology of the Interleukin-12 Family: Room for Discovery. Immunity. 2019.
14. Glassman C R, Mathiharan Y K, Jude K M, Su L, Panova O, Lupardus P J, et al. Structural basis for IL-12 and IL-23 receptor sharing reveals a gateway for shaping actions on T versus NK cells. Cell. 2021;
15. Wang P, Li X, Wang J, Gao D, Li Y, Li H, et al. Re-designing Interleukin-12 to enhance its safety and potential as an anti-tumor immunotherapeutic agent. Nat Commun. Nature Publishing Group; 2017; 8:1395.
16. Wittrup K D, Kaufman H L, Schmidt M M, Irvine D J. Intratumorally anchored cytokine therapy. https://doi.org/101080/1742524720222084070. Taylor & Francis; 2022; 19:1-8.
17. Mirlekar B, Pylayeva-Gupta Y. IL-12 family cytokines in cancer and immunotherapy. Cancers (Basel). 2021.
18. Hewitt S L, Bailey D, Zielinski J, Apte A, Musenge F, Karp R, et al. Intratumoral IL12 mRNA therapy promotes TH1 transformation of the tumor microenvironment. Clin Cancer Res. American Association for Cancer Research (AACR); 2020; 26:6284-98.
19. Strauss J, Heery C R, Kim J W, Jochems C, Donahue R N, Montgomery A S, et al. First-in-human phase I trial of a tumor-targeted cytokine (NHS-IL12) in subjects with metastatic solid tumors. Clin Cancer Res. American Association for Cancer Research Inc.; 2019; 25:99-109.
20. Kueberuwa G, Kalaitsidou M, Cheadle E, Hawkins R E, Gilham D E. CD19 CAR T Cells Expressing IL-12 Eradicate Lymphoma in Fully Lymphoreplete Mice through Induction of Host Immunity. Mol Ther—Oncolytics. 2018;
21. Schoenhaut D S, Chua a O, Wolitzky a G, Quinn P M, Dwyer C M, McComas W, et al. Cloning and expression of murine IL-12. J Immunol. 1992;
22. Chang C H, Qiu J, O'Sullivan D, Buck M D, Noguchi T, Curtis J D, et al. Metabolic Competition in the Tumor Microenvironment Is a Driver of Cancer Progression. Cell. 2015;
23. Matloubian M, Lo C G, Cinamon G, Lesneski M J, Xu Y, Brinkmann V, et al. Lymphocyte egress from thymus and peripheral lymphoid organs is dependent on S1P receptor 1. Nature. Nature; 2004; 427:355-60.
24. Cohen J. IL-12 Deaths: Explanation and a Puzzle. Science (80-). 1995;
25. Bedoui S, Whitney P G, Waithman J, Eidsmo L, Wakim L, Caminschi I, et al. Cross-presentation of viral and self antigens by skin-derived CD103+ dendritic cells. Nat Immunol. 2009; 10:488-95.
26. Overacre-Delgoffe A E, Chikina M, Dadey R E, Yano H, Brunazzi E A, Shayan G, et al. Interferon-γ Drives T reg Fragility to Promote Anti-tumor Immunity. Cell. 2017; 169:1130-1141.e11.
27. Pearce E L, Poffenberger M C, Chang C H, Jones R G. Fueling immunity: Insights into metabolism and lymphocyte function. Science (80-). 2013.
28. Scharping N E, Menk A V, Moreci R S, Whetstone R D, Dadey R E, Watkins S C, et al. The Tumor Microenvironment Represses T Cell Mitochondrial Biogenesis to Drive Intratumoral T Cell Metabolic Insufficiency and Dysfunction. Immunity. Elsevier; 2016; 45:374-88.
29. McLane L M, Abdel-Hakeem M S, Wherry E J. CD8 T Cell Exhaustion During Chronic Viral Infection and Cancer. Annu. Rev. Immunol. 2019.
30. Siska P J, Beckermann K E, Mason F M, Andrejeva G, Greenplate A R, Sendor A B, et al. Mitochondrial dysregulation and glycolytic insufficiency functionally impair CD8 T cells infiltrating human renal cell carcinoma. JCI Insight. 2017;
31. Sena L A, Li S, Jairaman A, Prakriya M, Ezponda T, Hildeman D A, et al. Mitochondria Are Required for Antigen-Specific T Cell Activation through Reactive Oxygen Species Signaling. Immunity. 2013;
32. Rackov G, Tavakoli Zaniani P, Colomo del Pino S, Shokri R, Monserrat J, Alvarez-Mon M, et al. Mitochondrial reactive oxygen is critical for IL-12/IL-18-induced IFN-γ production by CD4+ T cells and is regulated by Fas/FasL signaling. Cell Death Dis 2022 136. Nature Publishing Group; 2022; 13:1-14.
33. Mo F, Yu Z, Li P, Oh J, Spolski R, Zhao L, et al. An engineered IL-2 partial agonist promotes CD8+ T cell stemness. Nature. 2021;
34. Hong Y, Robbins Y, Yang X, Mydlarz W K, Sowers A, Mitchell J B, et al. Cure of syngeneic carcinomas with targeted IL-12 through obligate reprogramming of lymphoid and myeloid immunity. JCI Insight. 2022;
35. Xue D, Moon B, Liao J, Guo J, Zou Z, Han Y, et al. A tumor-specific pro-IL-12 activates preexisting cytotoxic T cells to control established tumors. Sci Immunol. American Association for the Advancement of Science; 2022; 7.
36. Leonard J P, Sherman M L, Fisher G L, Buchanan L J, Larsen G, Atkins M B, et al. Effects of single-dose interleukin-12 exposure on interleukin-12 associated toxicity and interferon-γ production. Blood. 1997;
37. Hildner K, Edelson B T, Purtha W E, Diamond M, Matsushita H, Kohyama M, et al. Batf3 deficiency reveals a critical role for CD8alpha+ dendritic cells in cytotoxic T cell immunity. Science. 2008; 322:1097-100.
38. Broz M L, Binnewies M, Boldajipour B, Nelson A E, Pollack J L, Erle D J, et al. Dissecting the Tumor Myeloid Compartment Reveals Rare Activating Antigen-Presenting Cells Critical for T Cell Immunity. Cancer Cell. Elsevier; 2014; 26:638-52.
39. Nirschl C J, Drake C G. Molecular pathways: coexpression of immune checkpoint molecules: signaling pathways and implications for cancer immunotherapy. Clin Cancer Res. 2013; 19:4917-24.
40. Siddiqui I, Schaeuble K, Chennupati V, Fuertes Marraco S A, Calderon-Copete S, Pais Ferreira D, et al. Intratumoral Tcfl+PD-1+CD8+ T Cells with Stem-like Properties Promote Tumor Control in Response to Vaccination and Checkpoint Blockade Immunotherapy. Immunity. 2019.

### Example 3. Phase I, First in Human, Multi-Site, Dose Escalation and Expansion Study of an Inducible IL-12 Prodrug

### 3.1 Introduction and Background

### Interleukin-12

Cytokines are small, secreted proteins that act via both autocrine and paracrine mechanisms to regulate host immunity (Waldmann et al., 2018). Interleukin-12 (IL-12) has long been studied for its capacity to elicit antitumor immune responses (Waldmann et al., 2018; Del Vecchio et al., 2007). The active IL-12 (p70) molecule is a heterodimer comprised of a 35 kD subunit (p35; encoded by IL12A) covalently linked to a 40 kD subunit (p40; encoded by IL12B) and is structurally similar to IL23 and IL27 (Tait Wojno et al., 2019). Antigen presenting cells such as dendritic cells, macrophages, and monocytes produce IL-12 upon activation by pathogen-associated molecular patterns, damage associated molecular patterns, cytokines, and/or cell-cell interactions (Del Vecchio et al., 2007). Binding of IL-12 to its high-affinity heterodimeric receptor (IL-12Rβ1/IL-12Rβ2) on activated T cells, natural killer (NK) cells, and natural killer T (NKT) cells initiates an intracellular signal transduction cascade via tyrosine kinase 2 (TYK2), JAK2, and STAT proteins (particularly STAT4), which induces changes in gene expression (Tait Wojno et al., 2019; Bacon et al., 1995).

IL-12 signaling is proinflammatory and can drive productive immune responses against malignant cells via multiple mechanisms. IL-12 was first purified from the supernatant of EBV-transformed B cells and termed “natural killer cell stimulatory factor” for its capacity to augment NK cell-mediated cytotoxicity (Kobayashi et al., 1989). This activity of IL-12 is in part mediated by increased transcription of genes encoding granzyme B and perforin (Aste-Amezaga et al., 1994). IL-12 also enhances T cell cytotoxic activity and stimulates proliferation of activated NK cells and T cells, even in the presence of other cytokines and mitogens (Stern et al., 1990; Gately et al., 1992; Perussia et al., 1992; Trinchieri et al., 1994). IL-12-stimulated interferon gamma (IFNγ) release from NK cells, CD4+ T cells, CD8+ T cells, and plasmacytoid dendritic cells leverages several additional antitumor mechanisms (Trinchieri et al., 1994; Berraondo et al., 2018). IFNγ is directly cytostatic/cytotoxic to tumor cells, enhances MHC I/II expression on both immune and tumor cells, induces expression of immune trafficking chemokines (CXCL9, 10, 11), promotes tumoricidal M1 macrophage polarization, and inhibits angiogenesis, among other beneficial activities (Castro et al., 2018; Berraondo et al., 2018). IL-12 can also direct T cell fate by promoting differentiation of naïve CD4+ T cells to T helper type 1 (Th1) cells (Hsieh et al., 1993). Moreover, IL-12 provides a critical third signal to naïve CD8+ T cells to promote effector and memory differentiation and clonal expansion in the presence of antigen (signal 1) and co-stimulation (signal 2) (Curtsinger et al., 1999, Curtsinger et al., 2003; Mescher et al., 2006; Chowdhury et al., 2011). IL-12 also has unique signaling activities in dendritic cells that can act to prime additional IL-12 release and to enhance antigen presentation to T cells (Grohmann et al., 1998, Bianchi et al., 1999).

To try to leverage these favorable immune activities for patient benefit, recombinant IL-12 was developed and investigated as an anticancer agent. In multiple mouse models, recombinant murine IL-12 administered systemically as a monotherapy caused regression of subcutaneous and metastatic tumors and prolonged survival at tolerable doses (Curtsinger et al., 1993, Curtsinger et al., 1996; Nastala et al., 1994; Zou et al., 1995). Mice cured by IL-12 treatment were protected against rechallenge with the same tumor cells, providing evidence of antitumor immune memory (Zou et al., 1995; Brunda et al., 1996). Mechanistically, the antitumor activity of IL-12 in mice required T cells and IFNγ but not NK cells (Brunda et al., 1993, 1996; Nastala et al., 1994; Zou et al., 1995). Overall, the nonclinical experience showed that recombinant IL-12 could be delivered via intravenous (IV), intraperitoneal, and intratumoral routes and at tolerable doses improves the survival of transplantable, carcinogen-induced and genetically engineered mouse tumor models (Tugues et al., 2014).

Despite promising findings in mice, studies of recombinant human IL-12 (rhIL-12) in patients have proved far more challenging. In a Phase I dose escalation study (N=40), rhIL-12 was administered by IV bolus injection to patients with advanced renal cell carcinoma (RCC), melanoma and colon cancer. This first-in-human (FIH) study identified a tolerable dose and schedule, detected activities consistent with the biology of IL-12 (e.g., increased circulating IFNγ, NK cytolytic activity, and T-cell proliferation), and observed preliminary evidence of antitumor activity (Atkins et al., 1997; Robertson et al., 1999). However, in a subsequent Phase 2 study (N=17), administration of the identical dose to patients with advanced RCC unexpectedly caused severe toxicities leading to 12 hospitalizations and 2 deaths (Leonard et al., 1997). The profound difference in toxicity was attributed to omission of a single “test dose” in the Phase 2 trial prior to initiation of successive daily doses (Leonard et al., 1997). Exploration of a modified schedule of twice weekly IV dosing in patients with metastatic melanoma and RCC (N=28) demonstrated improved safety and tolerability but produced only modest antitumor activity, with one PR observed in a patient with RCC (Gollob et al., 2000). Subcutaneous (SC) administration of rhIL-12 was also explored in melanoma and RCC but did not substantially improve the therapeutic index (TI) in these indications (Bajetta et al., 1998; Motzer et al., 1998, Motzer et al., 2001). In contrast, SC rhIL-12 was both tolerable and active in lymphomas, producing clinical responses as a monotherapy in >50% of patients with cutaneous T-cell lymphoma (CTCL; N=9) and in combination with rituximab in nearly 70% of patients with B-cell non-Hodgkin lymphomas (NHLs; N=43) (Rook et al., 1999; Ansell et al., 2002). rhIL-12 also showed activity as a single agent in patients with relapsed/refractory NHL (N=32), but the response rate was substantially higher for IV versus SC administration (Younes et al., 2004). Ultimately, development of systemically administered rhIL-12 (IV or SC) was discontinued, and the therapy is not presently approved for any indication.

### Interleukin-12 Prodrug

The recent development of immuno-oncology agents that enhance antitumor immunity is rapidly changing the treatment of cancer. However, these agents are not effective in all tumor types or in all patients with a certain tumor type. This gap represents an unmet medical need for novel immunotherapy approaches.

The IL-12 prodrug is a conditionally activated IL-12 prodrug that was designed to address the multiple shortcomings of rhIL-12. The IL-12 prodrug is engineered with a native IL-12 molecule attached via protease cleavable linkers to an inactivation domain to inhibit binding of IL-12 to its receptor in the periphery and to a half-life extension domain to enhance tumor exposure. The prodrug is activated in the TME via proteolytic cleavage of the linkers, thereby releasing the fully active IL-12 cytokine to stimulate a potent antitumor immune response. The preferential activation of the IL-12 prodrug in tumors is designed to both reduce systemic toxicity and enhance antitumor efficacy, thereby maximizing potential clinical benefit for patients. A preferred IL-12 prodrug for evaluation is Compound 36.

### 3.2 Study Rationale

This Phase 1 first in human dose escalation and dose expansion study will investigate the IL-12 prodrug as a monotherapy for patients with relapsed/refractory (r/r) advanced or metastatic solid tumors and lymphomas. The patients enrolled in this study will include those demonstrating primary or secondary resistance to immune checkpoint inhibitor (CPI) therapy as well as patients with tumor types for which CPIs are not approved. The first in human study will characterize the clinical safety, tolerability, pharmacokinetics (PK), pharmacodynamics (PD) and preliminary antitumor efficacy of the IL-12 prodrug.

Patients enrolled in this study will be those with metastatic malignancies who have limited treatment options.

An initial clinical dose of 0.016 mg/kg administered IV every 2 weeks (Q2W) has been selected based on an integrated analysis of the available nonclinical safety and pharmacology data for the IL-12 prodrug, as well as prior knowledge from clinical studies with rhIL-12.

The HNSTD in the IND-enabling GLP toxicity study in NHP is 0.3 mg/kg. Considering a human equivalent dose (HED) of 0.3/3.1=0.0968 mg/kg, and incorporating a 6-fold safety factor, the proposed starting dose in patients for IL-12 prodrug is 0.0968/6=0.016 mg/kg. The anticipated PK for the IL-12 prodrug in humans has been predicted using allometric scaling of NHP PK, with typical scaling coefficients for clearance and volume. Predicted exposure margins for Cmax and AUC relative to the 0.3 mg/kg HNSTD dose in NHP are currently estimated to be 20-fold and 19 fold, respectively, for the FIH starting dose.

The anticipated average concentration in patients at the starting dose of 0.016 mg/kg IV is ˜2.7 fold lower than the average concentration that caused ˜50% tumor regression relative to vehicle control in the MC38 mouse model (i.e., minimum anticipated biological effect level [MABEL] based on pharmacology). The exposure associated with complete regression in the MC38 model is anticipated in patients above 0.29 mg/kg Q2W, ˜18 fold higher than the starting dose.

The nonclinical data demonstrate low systemic exposure to free IL-12 after administration of the IL-12 prodrug. In the NHP GLP toxicity study, free IL-12 (i.e., IL-12 and/or anti-HSA-IL-12) was detected immediately after IV bolus dosing of 0.3 mg/kg the IL-12 prodrug, but the maximum concentration was ˜580-fold lower than the Cmax for the IL-12 prodrug. At the starting dose of 0.016 mg/kg, the maximum free IL-12 exposure anticipated in patients is approximately 7 pM, which is ˜20-fold lower than the maximum free IL-12 exposure in NHP at the HNSTD (˜0.13 nM) and 27-fold lower than the level which has been shown to be tolerated after IV bolus dosing at 500 ng/kg rhIL-12 (Cmax 0.19 nM; Atkins et al., 1997).

In summary, the IL-12 prodrug is designed to minimize exposure to free IL-12 in the systemic circulation. At the starting dose, free IL-12 is predicted to be significantly below the level previously associated with systemic toxicity after repeated administration of rhIL-12 to cancer patients. In this Phase 1 FIH study, the initial dose and dose escalation strategy utilizing the Bayesian Logistic Regression Model (BLRM) with Escalation with Overdose Control (EWOC) is designed to reach the anticipated therapeutic range safely, minimizing exposure to potentially ineffective dosing regimens. Emerging safety and biomarker data will be used throughout this Phase 1 FIH study to further guide the dosing strategy for the IL-12 prodrug

### 3.5 Study Objectives

### Primary Objectives

### The Primary Objectives of the Dose Escalation.

To evaluate the safety and tolerability of the IL-12 prodrug; and
To determine the maximum tolerated dose (MTD) and/or recommended dose for expansion (RDE) of the inducible IL-12 prodrug; and
To evaluate antitumor activity.
   The Primary Objectives of the Dose Expansion Part of this Study.
To further characterize the safety and tolerability of the IL-12 prodrug; and
To evaluate the antitumor activity of the IL-12 prodrug as measured by overall response rate (ORR; complete response [CR]+partial response [PR]) by Response Evaluation Criteria in Solid Tumors (RECIST) 1.1, immune ORR (immune-ORR [iORR]; immune CR [iCR]+immune PR [iPR]) by immune RECIST (iRECIST), or Lugano classification (Cheson et al., 2014) for lymphomas.

### The Primary Endpoints.

Frequency, severity, and relatedness of treatment-emergent adverse events (TEAEs) and serious adverse events (SAEs), changes in safety laboratory parameters, and DLTs (if observed)
Overall response rate (ORR) (complete response [CR]+partial response [PR]), duration of response (DOR) by Response Evaluation Criteria in Solid Tumors (RECIST) 1.1 and immune-overall response rate (iORR) (immune-complete response [iCR]+immune-partial response [iPR]) by immune-RECIST (iRECIST), or response by Lugano criteria (for lymphoma only; Cheson et al., 2014)

### Secondary Objectives

The Secondary Objectives of this Study are the Following:
To characterize the PK profile of the IL-12 prodrug (i.e., both parent compound and free IL-12);
To evaluate changes in key immunological biomarkers in blood and in baseline and on-treatment tumor biopsies after administration of the IL-12 prodrug;
To evaluate the antitumor activity of the IL-12 prodrug as measured by duration of response (DOR) and progression-free survival (PFS) by RECIST 1.1, iRECIST, or Lugano classification (Cheson et al., 2014) for lymphomas;
To evaluate the immunogenicity of the IL-12 prodrug (i.e., the potential to generate an anti-drug antibody [ADA] response); and
To determine the impact of the IL-12 prodrug on overall survival (OS).

### The Secondary Endpoints are:

Plasma concentrations versus time profiles for the IL-12 prodrug and free IL-12 and calculated PK parameters
Characterization of changes in peripheral immune cells including T cell subsets from baseline in response to the IL-12 prodrug
Changes in immunological biomarkers in baseline and on-treatment tumor biopsies in response to the IL-12 prodrug as determined by immunohistochemistry (IHC), lymphocyte density and/or activation state in tumor biopsies
Incidence and titers of ADAs to the IL-12 prodrug
ORR (CR+PR) based on best overall response (BOR)
DOR
Disease control rate at month 3, 6 and 9
Progression free survival (PFS) per RECIST v1.1 (solid tumor) and iRECIST or Lugano classification (lymphoma; Cheson et al., 2014) assessed by Investigator Overall survival (OS)

### Exploratory Objectives

### The Exploratory Objectives are:

To evaluation pharmacodynamics;
To investigate immunological biomarkers (blood, tumor) that may correlate with treatment outcome; and
To assess tumor biopsies for potential biomarkers of target engagement and immune activation.

### The Exploratory Endpoints are:

Modulation of cytokines including but not limited to IL-2, IL-4, IL-5, IL-6, IL-8, IL10, IL-13, IL-15, IFNγ, -IFNγ-induced protein 10 (IP-10), transforming growth factor β (TGFβ), tumor necrosis factor α (TNFα), and C-reactive protein (CRP);
Changes in levels of lymphocytes (including lymphocyte subsets) in peripheral blood
Characterization of intra-tumoral immune cells, including T-cell subsets and immune cell frequency
Changes in gene expression profiles of immune response in baseline and posttreatment tumor biopsies in response to the IL-12 prodrug
Assessment of free IL-12 in ex-vivo cleavage assay with tumor biopsy

### 3.6 Study Description

This is a Phase 1, first in human, multi-site study starting with dose escalation of the IL-12 prodrug (Part 1) followed by dose expansion of the IL-12 prodrug (Part 2) in two arms (A and B).

### Dose Escalation Phase (Part 1)

The dose escalation part of the study will be conducted in patients with relapsed/refractory (r/r) advanced and/or metastatic solid tumors. Patients with primary CNS malignancies are ineligible. Patients with castrate-resistant prostate cancer (CRPC) and non-Hodgkin lymphoma (NHL) will be eligible for Dose Expansion (see Arm B below) but are not eligible for Dose Escalation. During dose escalation, the IL-12 prodrug will be administered as a single agent on Days 1 and 15 (i.e., every 2 weeks, Q2W) of 28-day treatment cycle.

The starting dose of IL-12 prodrug is 0.016 mg/kg. Selection of the starting dose in this FIH clinical study was informed by safety and PK data from non-Good Laboratory Practice (GLP) and GLP toxicological studies in cynomolgus monkeys, pharmacology studies in tumor-bearing mice, and the prior clinical experience with rhIL-12. A total of eight provisional dose levels are defined for the dose escalation. A dose level with a 50% lower dose than the starting dose is also included if the first dose level is not tolerated.

To minimize the number of patients treated at potentially subtherapeutic dose levels, cohorts will enroll a minimum of 3 patients and up to 6 patients. In each dose cohort, a minimum of 3 patients are required to have completed the dose-limiting toxicity (DLT) observation period before initiating enrollment of the subsequent cohort.

To help ensure patient safety, dosing of the first 2 patients at each dose level will be staggered by at least 7 days and dosing of the second and all following patients will be staggered by at least 2 days. The DLT assessment period for a dose cohort must be completed and the data reviewed by the Dose Escalation Committee (DEC) prior to the recruitment of patients into the next dose cohort.

Patients may receive the IL-12 prodrug for as long as they continue to show clinical benefit, as assessed by the Investigator, or until disease progression or other treatment discontinuation criteria are met. No intrapatient dose escalation is allowed.

A Bayesian Logistic Regression Model (BLRM) incorporating escalation with overdose control (EWOC) will be used to guide the dose escalation. Data from patients satisfying the requirements for inclusion in the dose-determining set (DDS) will be included in the model. After completion of a dose cohort, or at any time the BLRM is updated, a decision to escalate and the actual dose and schedule selected will be determined by the DEC based on a review of all available clinical, PK and laboratory data and on the recommendation of the BLRM regarding the highest admissible dose according to the EWOC principle.

Dose escalation will continue until determination of the MTD and/or RDE. Once the MTD/RDE is identified, the Dose Expansion part of the study (Part 2) will open. Determination of the RDE and selection of an optimal dosage will be informed not only by clinical PK, PD, antitumor activity and safety data, but also by nonclinical pharmacology and toxicology data.

The dose escalation and determination of the MTD and/or recommended dose will be guided by a BLRM with overdose control (EWOC). Dose escalation meetings will be conducted after all patients in a cohort complete one cycle of study treatment. Safety assessments including AEs and laboratory values will be closed monitored for all enrolled patients in order to identify any DLTs. Prior to the determination of the MTD and/or recommended dose for each group, a minimum of 6 patients must have been treated at the MTD and/or recommended dose with the IL-12 prodrug.

The starting dose and some of the provisional dose levels that could be evaluated during this study are described in Table 5. The doses to be investigated are not limited to the provisional dose level listed in the table. The proposed dose escalation scheme for the IL-12 prodrug includes a dose level −1 with a 50% lower dose than the first dose level, in the event that the first dose level is not tolerated. Doses are selected based on patient safety data and are subject to satisfying the EWOC criteria under BLRM.

TABLE 5
Provisional Doses for Dose Escalation (Part 1)

| Dose Level | Dose ^{b} | Increment from Previous Dose (%) |
|---|---|---|
| −1^{a} | 0.008 mg/kg | (50% decrease) |
| 1 | 0.016 mg/kg | Starting dose |
| 2 | 0.032 mg/kg | 100% |
| 3 | 0.056 mg/kg | 75% |
| 4 | 0.084 mg/kg | 50% |
| 5 | 0.126 mg/kg | 50% |
| 6 | 0.190 mg/kg | 50% |
| 7 | 0.290 mg/kg | 50% |
| 8^{c} | 0.440 mg/kg | 50% |

^{a}Dose level −1 represents a dose that may be evaluated if dose level 1 is poorly tolerated. No dose de-escalation below this level is planned for this study. If dose level −1 is poorly tolerated the study will be terminated.
^{b} It is possible for some dose levels to be skipped or additional dose levels to be added during course of the study.
^{c}IL-12 prodrug does higher than 0.440 mg/kg may be allowed depending on the observed safety, pharmacokinetics, pharmacodynamics, and based on the recommendations of the 2-parameter Bayesian Logistic Regression Model (BLRM).

### Dose Escalation and Determination of Maximum-Tolerated Dose (MTD)/Recommended Dose

The MTD is defined as the highest dose of study drug where the posterior probability of the true DLT rate in the target interval (0.16-0.33) is above 0.50, and the dose at which at least 6 patients have been treated in a confirmatory cohort of patients for the duration of the DLT observation period (i.e., safety review period) of study drug. AEs and laboratory abnormalities considered to be DLTs.

An adaptive 2-parameter BLRM incorporating EWOC will be used during the Dose Escalation (Part 1) to select dose levels and to estimate the MTD. Each cohort will consist of newly enrolled patients who will receive escalating doses of the IL-12 prodrug until the MTD is reached.

Determination of the MTD during dose escalation will be based upon an estimation of the probability of a DLT in Cycle 1 in the dose-determining set (DDS). If the MTD is not reached during dose escalation, the RDE will be determined based on review of DLTs, AEs and SAEs, laboratory, PK and PD data by the DEC as the dose with the optimal therapeutic window for the IL-12 prodrug.

At all decision timepoints, the adaptive BLRM permits alterations in the dose increments based on the observed toxicities. It is therefore possible for some dose levels to be skipped or additional intermediate dose levels or schedules to be added during the study. The BLRM will recommend the dose that may not be exceeded at any decision point during escalation and the maximum increase in dose allowed by the protocol. Dose escalation will not exceed a 100% increase from the current dose being administered. Cohorts may be expanded at any dose level below doses deemed unacceptable to further characterize safety, tolerability, PK, or PD. Dose escalation may be terminated at any time based on emerging safety concerns (i.e., without establishing the MTD).

### Dose Expansion Phase (Part 2)

Patients in Dose Expansion will have a confirmed diagnosis of a r/r locally advanced or metastatic solid tumor or lymphoma for which the patient has progressed or is intolerant of standard therapy, or for whom no standard therapy with proven benefit exists. Dose Expansion (Part 2) will be conducted in 2 arms that will enroll the following patient populations:
Arm A: Patients with indications for which a CPI is indicated/approved (e.g., cutaneous malignant melanoma, RCC, non-small cell lung cancer [NSCLC], head and neck squamous cell carcinoma [HNSCC], urothelial carcinoma, high microsatellite instability (MSI-H) tumors, etc.) who have been treated with a CPI regimen and who demonstrate primary or secondary resistance to CPI therapy. Primary resistance is defined as disease progression or stable disease (SD)<6 months as the best response after at least 6 weeks of exposure to inhibitors of PD-(L)1. Secondary resistance is defined as disease progression ≥6 months after initiation of PD-(L)1 inhibitors in patients who have received clinical benefit (i.e., CR or PR or SD >6 months). Patients who discontinue CPI therapy (e.g., anti-PD-(L)1) for toxicity or other reasons and who don't demonstrate primary or secondary resistance to CPIs as defined here are not eligible. Patients with Hodgkin lymphoma are also ineligible.
Arm B: Patients with tumor types for which CPI therapy is not indicated/approved (e.g., pancreatic cancer, microsatellite-stable [MSS] colorectal carcinoma, castrate-resistant prostate cancer [CRPC], NHL) and who are CPI naïve. Patients with NHL should have either follicular lymphoma or diffuse large B-cell lymphoma (DLBCL), though other subtypes of NHL including T-cell lymphomas may be considered. All patients with NHL must have received at least 2 prior systemic therapies. Patients with primary CNS malignancies, or who received anti-PD-(L)1 in a clinical trial or off label are not eligible.

Additional arms may be added in the Dose Expansion for specific indications of interest.

Patients will remain on study treatment until they experience unacceptable toxicity, progressive disease per RECIST (Appendix D; Eisenhauer et al., 2009) or iRECIST (Appendix E) for solid tumors, or confirmed progressive disease for NHL (dose expansion part) according to Lugano classification (Cheson et al., 2014), and/or treatment discontinued at discretion of the Investigator, or the patient withdraws consent.

The study consists of a screening period, a treatment period with either Dose Escalation (Part 1) or Dose Expansion (Part 2), an End-of-Treatment (EOT) visit, and a safety follow-up period. The Safety Follow-Up Visit should occur either 30 days after the last dose of study drug or prior to the start of a new cancer regimen, whichever comes first. Overall survival status will be evaluated every 12 weeks (+/−2 weeks) for patients who are on Dose Expansion (Part 2). Patients will be contacted via telephone to evaluate overall survival until initiation of a new therapy or death, whichever comes first.

The end of the study is defined as when 80% of patients have either discontinued the study or have completed follow-up. Throughout the study, patients will be assessed for efficacy, PK, PD and safety of the IL12 prodrug.

### Study Stopping

Patients may receive the IL-12 prodrug for as long as they continue to show clinical benefit as assessed by the Investigator, or until disease progression or other treatment discontinuation criteria are met.

Beyond Cycle 1 in Dose Escalation (Part 1) and during Dose Expansion (Part 2), the study will continue to use the DLT criteria. If toxicities that meet these DLT criteria are observed in >33% of patients at any point in time, the DEC will be convened to review the available safety data and determine appropriate next steps, which may include refinement of dose, modification of study assessments, study closure, etc. Enrollment may be paused while such a review is undertaken.

### 3.7 Selection of Study Population

### Inclusion Criteria

Each patient must meet all the following criteria to participate in the study:
1. Age ≥18 years, able to understand and voluntarily sign a written informed consent form (ICF) and willing and able to comply with protocol requirements.
2. For all patients in Dose Escalation (Part 1): A confirmed diagnosis of a r/r locally advanced or metastatic solid tumor for which the patient has progressed on or is intolerant of standard therapy, or for whom no standard therapy with proven benefit exists. Patients with castrate-resistant prostate cancer (CRPC) and non-Hodgkin lymphoma (NHL) will not be enrolled in dose escalation.
3. For all patients in Dose Expansion (Part 2): A confirmed diagnosis of r/r locally advanced or metastatic solid tumors and lymphomas for which the patient has progressed on or is intolerant of standard therapy, or for whom no standard therapy with proven benefit exists.

Dose Expansion (Part 2) consists of two arms, Arm A and Arm B. The following additional criteria must be met for patients to be eligible for Arm A or Arm B:
Arm A: Patients with indications for which a CPI is indicated/approved, (e.g., cutaneous malignant melanoma, RCC, NSCLC, HNSCC, urothelial carcinoma, etc.) who demonstrate primary or secondary resistance to CPI therapy, as defined above in Study Design. Patients who discontinue CPI therapy for toxicity or other reasons and who haven't demonstrated either primary or secondary resistance to CPIs are ineligible, as are patients with Hodgkin lymphoma.
Arm B: Patients with tumor types for which CPI therapy is not indicated/approved (e.g., pancreatic cancer, MSS colorectal carcinoma, CRPC, NHL, etc.) and who are CPI naïve. Patients with NHL should have either follicular lymphoma or DLBCL. Other subtypes of NHL including T-cell lymphoma may be considered. All patients with NHL must have received at least 2 prior systemic therapies. In Arm B, patients with CRPC must be status post bilateral orchiectomy or medical castration via ongoing treatment with a LHRH agonist/antagonist and should have serum testosterone levels <50 ng/dL documented within 4 weeks prior to the start of the study drug.
4. Eastern Cooperative Oncology Group (ECOG) Performance Status of 0 or 1.
5. At least one measurable lesion per RECIST 1.1 (Appendix D; Eisenhauer et al., 2009) or evaluable lesion per Lugano classification (Cheson et al., 2014). CRPC patients (i.e., in Arm B) may not have bone-only disease.
6. Agrees to undergo a pre-treatment and on-treatment biopsy of a primary or metastatic solid tumor or lymphoma lesion.
7. Human immunodeficiency virus (HIV) infected patients must be on antiretroviral therapy (ART). Patients on ART must have well-controlled HIV infection/disease as evidenced by the following:
a. A CD4+ T-cell count >350 cells/mm3 at time of screening.
b. Achieved and maintained virologic suppression defined as confirmed HIV RNA level below 50 copies/mL or the lower limit of qualification (below the limit of detection) using the locally available assay at the time of screening and for at least 12 weeks prior to screening.
c. Been on a stable regimen, without changes in drugs or dose modification, for at least 4 weeks prior to study entry (Day 1).
8. Has adequate organ and bone marrow function defined by:
a. Absolute neutrophil count (ANC) ≥1.5×109/L (≥1500/mm3).
b. Hemoglobin ≥9.0 g/dL or equivalent. Criteria must be met without packed red blood cell transfusion within the prior 2 weeks.
c. Platelet count ≥100×109/L (100,000/mm3).
d. Total bilirubin ≤1.5× upper limit of normal (ULN) in the absence of Gilbert's syndrome and ≤3×ULN if the patient has Gilbert's syndrome.
e. Measured or calculated creatinine clearance (estimated glomerular filtration rate) ≥30 mL/min.
f. Alanine aminotransferase (ALT) and aspartate aminotransferase (AST) ≤2.5×ULN or ≤5×ULN for patients with hepatic metastases.
9. Willingness of men and women of reproductive potential to agree to highly effective birth control for the duration of treatment and for 4 months following the last dose of study drug.

Male study participants should refrain from sperm donation during study treatment and up to 6 months following the last dose of study drug.

A woman of childbearing potential is a woman who is fertile following menarche and until becoming post-menopausal unless permanently sterile. Permanent sterilization methods include hysterectomy, bilateral salpingectomy, and bilateral oophorectomy.

A man is considered fertile after puberty unless permanently sterile by bilateral orchidectomy.

### Exclusion Criteria

Patients who meet any of the following criteria will be excluded from participating in the study:
1. Have a history of another active malignancy (a second cancer) within the previous 2 years, except for localized cancers that are not related to the current cancer being treated, is considered cured, and, in the opinion of the Investigator, presents a low risk of recurrence. These exceptions include, but are not limited to, basal or squamous cell skin cancer, superficial bladder cancer, or carcinoma in situ of the prostate, cervix, or breast.
2. Have received prior treatment with IL-12 (including by intratumoral injection).
3. Has primary CNS malignancies are excluded from all parts of the trial. Patients with CRPC and NHL are excluded from Dose Escalation.
4. Has symptomatic CNS metastases or CNS metastases that require local CNS directed therapy (such as XRT or surgery) or increasing doses of corticosteroids within 2 weeks prior to the first dose of study drug. Patients with treated brain metastases should be neurologically stable and receiving ≤10 mg per day of prednisone or equivalent prior to study entry. Patients with previously diagnosed brain metastases are eligible if they have completed their treatment, have recovered from the acute effects of radiation therapy or surgery prior to enrollment, have fulfilled the steroid requirement for these metastases, and are neurologically stable and asymptomatic.
5. Have significant cardiovascular disease, including myocardial infarction, arterial thromboembolism, or cerebrovascular thromboembolism, within 6 months prior to the first dose of study drug; symptomatic dysrhythmias or unstable dysrhythmias requiring medical therapy; angina requiring therapy; symptomatic peripheral vascular disease; New York Heart Association Class 3 or 4 congestive heart failure; or history of congenital prolonged QT syndrome.
6. Have significant electrocardiogram (ECG) abnormalities at Screening, including unstable cardiac arrhythmia requiring medication, left bundle branch block, second-degree atrioventricular (AV) block type II, third-degree AV block, ≥Grade 2 bradycardia, or QT interval corrected for heart rate using Fridericia's formula (QTcF) >470 msec.
7. Have active autoimmune disease requiring systemic treatment in the past 2 years (i.e., with use of disease-modifying antirheumatic agents or immunosuppressive drugs). Note: Replacement therapy (e.g., thyroxine, insulin, or physiologic corticosteroid replacement therapy for adrenal, thyroid, or pituitary insufficiency) is permitted.
8. Diagnosis of immunodeficiency, on immunosuppressive therapy, or receiving chronic systemic or enteric steroid therapy (dose >10 mg/day of prednisone or equivalent) or any other form of immunosuppressive therapy within 7 days prior to the first dose of study drug. At Screening and during study participation, patients may be using systemic corticosteroids (dose ≤10 mg/day of prednisone or equivalent) or topical or inhaled corticosteroids. 9. In patients with NHL, prior receipt of an allogeneic stem cell transplant or prior allogeneic CAR-T cell therapy (autologous stem cell transplantation and autologous CAR-T cell therapy are allowed).
10. Major surgery (excluding placement of vascular access) within 2 weeks prior to the first dose of study drug.
11. Investigational agent or anticancer therapy (including chemotherapy, biologic therapy, immunotherapy, anticancer Chinese medicine, or other anticancer herbal remedy) within 5 half-lives or 4 weeks (whichever is shorter) prior to the first dose of study drug. In addition, no concurrent investigational anticancer therapy is permitted during the study.
12. Has received radiotherapy within 2 weeks of start of study treatment. Participants must have recovered from all radiation-related toxicities, not require corticosteroids, and not have had radiation pneumonitis. A 1-week washout is permitted for palliative radiation (≤2 weeks of radiotherapy) to non-CNS disease.
13. Any unresolved toxicities from prior therapy greater than National Cancer Institute Common Terminology Criteria for Adverse Events (NCI-CTCAE) version 5.0 Grade 1 at the time of starting study drug with the exception of alopecia and Grade 2 platinum therapy related neuropathy.
14. Use of sensitive substrates of major CYP450 isozymes.
15. Any illness, medical condition, organ system dysfunction, or social situation, including mental illness or substance abuse, deemed by the Investigator to be likely to interfere with a patient's ability to sign the ICF, adversely affect the patient's ability to cooperate and participate in the study, or compromise the interpretation of study results.
16. Received a live vaccine within 30 days of the first dose of study drug.
17. Active, uncontrolled systemic bacterial, viral, or fungal infection.
18. HIV-infected participants with a history of Kaposi sarcoma and/or Multicentric Castleman Disease.
19. Active infection as determined by hepatitis B surface antigen and hepatitis B core antibody, or hepatitis B virus deoxyribonucleic acid (DNA) by quantitative polymerase chain reaction (qPCR) testing.
20. Active infection as determined by hepatitis C virus (HCV) antibody or HCV RNA by qPCR testing.
21. Pregnant or lactating.
22. History of hypersensitivity to any of the study drug components.

### 3.8 Study Treatment

### Investigational Product

The IL-12 prodrug drug is filled in 6R glass vials, lyophilized, and closed with a 20 mm rubber stopper and aluminum crimp with a plastic cap. The sterile lyophilizate is reconstituted with sterile water-for-injection to form a clear, colorless to slightly yellowish solution for IV administration. If initially reconstituted with 2.1 mL of sterile water-for-injection, the solution concentration is 5 mg/mL IL-12 prodrug, 50 mM sodium citrate, 240 mM sucrose, 0.02% polysorbate 80, pH 5.5. Diluted IL-12 prodrug solution in the syringe is stable for up to 8 hours at ambient temperature and for another 16 hours at 2-8° C. Total hold time of dose solutions in the syringe should not exceed 24 hours.

### 3.9 Study Procedures

### General Dosing

The IL-12 prodrug will be dosed based on body weight and administered as a 15-minute IV infusion via syringe pump on Days 1 and 15 (Q2W) of 28-day cycles until progressive disease by RECIST 1.1 (Eisenhauer et al., 2009), unacceptable toxicity, withdrawal of consent by the patient, discontinuation of the patient by the Investigator, decision to terminate the study or treatment, or death. Infusion duration may be prolonged in the event of an infusion-related reaction.

The IL-12 prodrug dose solutions were shown compatible with the following contact materials: polypropylene (syringe), polyamide (3-way stopcock), polyethylene (infusion line), polyvinyl chloride (infusion line), polyurethane (catheter) and stainless steel (needle). Non-siliconized syringes should be used to prepare and administer The IL-12 prodrug.

During the Dose Escalation Phase, a stagger of at least 7 days is required between dosing of the first and second patients at each new untested dose level, and a stagger of 2 days is required between dosing of the second and all subsequent patients in that cohort.

All infusions of the IL-12 prodrug should be administered in a monitored setting where there is immediate access to trained personnel and adequate equipment and medicine to manage potentially serious or life-threatening reactions. All patients will remain in a monitored/hospitalized setting for 24 hours following the first dose of the IL-12 prodrug and for 8 hours for all subsequent study drug administrations, except for patients who experience Grade 2 CRS with their first administration of study drug. Those patients will continue to be monitored for 24 hours following all subsequent administrations of study drug.

All details of study drug administration including dose, volume of infusion, duration, and interruptions or changes to study drug administration must be recorded on the Dose Administration Record electronic case report form (eCRF).

### Dose Modification

If a patient experiences TEAEs that may be related to the IL-12 prodrug, including immune-related adverse events (irAEs), additional doses may be held, modified, or permanently discontinued. During Cycle 1, dose modification should be avoided if a patient has not experienced a Grade 2 or higher TEAE related to study drug. Prolonged delay (>2 weeks) in administering the IL-12 prodrug doses during the first 28 days of treatment or in initiating Cycle 2 due to treatment-related toxicity will be considered a DLT. In subsequent cycles, dose interruptions and/or modifications are permissible. When the IL-12 prodrug is withheld for toxicity, if the toxicities do resolve, the IL-12 prodrug should be restarted within 4 weeks (28 days) of the originally scheduled dose or within 6 weeks (42 days) of the last administered dose. However, if a toxicity does not resolve and/or the IL-12 prodrug is held for >28 days from the originally scheduled dose or >42 days from the last administered dose, the study drug should be permanently discontinued for that patient.

### 3.10 Test and Evaluations

### Vital Signs and Physical Examination

Vital signs will be measured and will include measurements of systolic and diastolic blood pressure, heart rate, and body temperature.

Physical examinations will be performed. A physical examination and review of relevant systems, body weight, and height will occur at screening. Height will be measured at the Screening Visit only but may be measured later if missed. An abbreviated physical examination that is directed by disease site and symptoms will be performed on Day 1 of subsequent cycles after Cycle 1.

### Pharmacokinetics

Blood will be collected for measurement of total IL-12 (i.e., the IL-12 prodrug plus free IL-12) and free IL-12. At certain timepoints, blood will also be collected for assessment of ADA to determine the immunogenicity of the IL-12 prodrug. If supported by the data, additional supportive or exploratory analyses may be conducted using samples collected for ADA assessments.

If a dose of study drug is missed on a PK sample collection day, the PK sampling should be skipped and performed on the next dosing day.

PK parameters such as AUC, Cmax, minimum observed plasma concentration (Cmin), time to maximum observed plasma concentration (Tmax), terminal-elimination half-life (t½) clearance, volume of distribution (Vd), and volume of distribution at steady state (Vss), will be determined. PK sampling timepoints may be reduced or removed based on emerging data.

Additional PK assessments may be conducted when considered necessary by the Investigator to better understand the relationship between drug exposure and AEs or treatment response, for example.

### Electrocardiograms

Twelve-lead ECGs will be performed. Serial triplicate 12-lead ECGs separated by ≥1 minute will be performed throughout the Dose Escalation Phase. Single ECGs are sufficient for assessment of patient eligibility and for safety management. Scheduled ECG measurements coincide with PK collection and will be independently reviewed by a central laboratory. All eligibility and safety management decisions should be made based on the local reading of the ECG. Instructions for the collection and transmission of ECGs to the central laboratory will be provided in the ECG Manual.

In the Dose Expansion Phase, single 12-lead ECGs will be performed and read locally. Single ECGs should be repeated if an anomaly or abnormality is observed. When the ECG measurements coincide with blood sample draws for PK, the ECG assessment should be timed sufficiently prior to blood sample collection to not impact the PK sample collection time. ECGs will be recorded after the patient has been in a resting (semi-recumbent or semi-supine) position breathing quietly for 5 minutes. All pre-dose ECGs will be performed 15 to 30 minutes prior to study drug administration. Equipment for ECG measurements in Dose Escalation phase will be provided by dMed-CliniPace and ECG images will be stored centrally.

If emerging data suggest reduction in the number of PK time points post-dose, the ECG assessments performed post-dose to match the PK collection time points may also be reduced if no significant changes have been observed.

### Tumor Measurements

### Solid Tumors

Assessment of solid tumors will be based on RECIST 1.1 and iRECIST. Disease assessments will include radiographic measurements of tumors in the chest, abdomen, pelvis, and at any other known sites of disease using computed tomography (CT), magnetic resonance imaging (MRI), or positron emission tomography (PET)/CT. Oral and IV contrasts should be used unless there is a clear contraindication such as decreased renal function or an allergy, which cannot be addressed with standard prophylactic treatments. If a patient has a medical contraindication to iodinated contrast used for CT, a non-contrast chest CT and an MRI abdomen/pelvis with contrast may be performed as a substitute. Patients with CNS metastases must undergo brain imaging (MRI preferred; CT with contrast is acceptable if MRI imaging contraindicated) during Screening.

### Lymphomas

Assessment of lymphomas (NHL) in Dose Expansion Part B will be based on Lugano classification. At Screening, lymphoma patients with FDG-avid disease will undergo fluorodeoxyglucose (FDG) PET/CT imaging. All subsequent disease assessments should also be performed with FDG PET/CT, using the Deauville 5-point scale. The avidity of up to six of the largest dominant nodes, nodal masses and/or extranodal lesions, selected to be clearly measurable in two diameters, should be assessed. Nodes should preferably be from disparate regions of the body and should include, where applicable, mediastinal and retroperitoneal areas. Non-nodal lesions include those in solid organs, the GI tract, cutaneous lesions, or those noted on palpation. Non-measured lesions are any disease not selected as dominant or measurable. For patients with non-FDG-avid or variably FDG-avid tumors, a CT scan of the chest/abdomen/pelvis and any additional known sites of disease will be performed with IV contrast. For patients staged with CT, up to six of the largest target nodes, nodal masses, or other lymphomatous lesions that are measurable in two diameters (longest diameter [LDi] and shortest diameter) should be identified from different body regions representative of the patient's overall disease burden and include mediastinal and retroperitoneal disease, if involved. A measurable node must have an LDi greater than 15 mm. Measurable extranodal disease may be included in the 6 representative, target lesions. A measurable extranodal lesion should have an LDi greater than 10 mm. All other lesions (including nodal, extranodal, and assessable disease) should be followed as non-target disease.

### Frequency of Assessments

Baseline tumor assessments will be taken during Screening within 28 days of Cycle 1 Day 1. On-study scans should be performed every 8 weeks (±7 days) from Cycle 1 Day 1 for the first 6 cycles and every 12 weeks (±7 days) thereafter, and include imaging of the chest, abdomen, pelvis, and any other areas of known disease at baseline, using the same modality as used for screening/baseline imaging until progressive disease per RECIST 1.1 and iRECIST for solid tumors, or Lugano classification for NHL, withdrawal of consent, or initiation of a new anticancer therapy.

The results of all scans are to be recorded in the eCRF. A copy of all scans is to be sent to central imaging for storage.

### Pharmacodynamics and Biomarkers

Pharmacodynamic and biomarker assessments will be performed on blood samples and freshly biopsied and/or archival tumor tissue.

TABLE 6
Biomarker Sample Collection Plan for Inducible IL-12 Prodrug

| | | Approximate | | |
|---|---|---|---|---|
| | Visit/Time | volume (tumor or | | |
| Sample Type | point | blood samples) | Markers | Purpose |
| Fresh tumor | Baseline | Optimally 5 to 6 | Characterization of | Evaluation of target |
| biopsy (and | | passes of a core | intratumoral immune | engagement, immune |
| archival tumor | | needle biopsy | cells, including T cell | cell activation, and |
| sample, if | | | subsets (flow cytometry). | changes in tumor |
| available) | | | Changes in tumor | immune contexture. |
| Fresh tumor | C1D 22-D 28 | Optimally 5 to 6 | microenvironment RNA | Assessment of whether |
| biopsy | | passes of a core | expression profile | the patient's tumor can |
| | | needle biopsy | (NanoString). | activate IL-12 prodrug |
| | | | Changes in tumor | to release free IL-12. |
| | | | immune contexture | |
| | | | and/or spatial immune | |
| | | | profile (IHC/IF). | |
| | | | Cleavage of IL-12 | |
| | | | prodrug ex vivo with | |
| | | | release of free IL-12 | |
| | | | (baseline tumor sample | |
| | | | only). | |
| Blood (Serum or | Dose | 1 mL | Cytokines including, but | To investigate target |
| Plasma) | escalation | | not limited to, IL-2, IL-4, | engagement and assess |
| | and | | IL-5, IL-6, IL-8, IL-10, | systemic cytokine |
| | expansion: | | IL-13, IL-15, IFNγ, IFNγ- | modulation |
| | C1D 1 | | induced protein 10 (IP- | |
| | (pre-dose) | | 10), transforming growth | |
| | C1D 2 (24 h | | factor β (TGFβ), TNFα, | |
| | post-dose) | | and CRP. | |
| | C1D 3 (48 h | | | |
| | post-dose) | | | |
| | Dose | | | |
| | escalation | | | |
| | only: C2D 1 | | | |
| | (pre-dose) | | | |
| | C2D 2 (24 h | | | |
| | post-dose) | | | |
| | C2D 3 (48 h | | | |
| | post-dose) | | | |
| Blood | C1D 1 (pre- | — | Peripheral lymphocyte | Indicator of target |
| | dose) | | counts | engagement |
| | C1D 2 (24 h | | | |
| | post-dose) | | | |
| | C1D 8 (168 h | | | |
| | post-dose) | | | |
| Blood (collect in | Baseline, | 2 mL | Immune cell profile | To characterize changes |
| cytochex tube) | C1D 8 (168 h | | | in peripheral immune |
| | post-dose) | | | cells, including T cell |
| | | | | subsets |

C = cycle; CRP = C-reactive protein; D = day; IFNγ = interferon gamma; IL-12R = interleukin-12 receptor, TNF = tumor necrosis factor.

### Fresh Tumor Biopsies and Archival Tumor Samples

Pre- and on-treatment biopsies are required for immunophenotyping (flow cytometry), cancer gene expression analysis (NanoString), and evaluation of tumor immune contexture (IHC/IF). At baseline, a fresh biopsy ideally retrieving 5-6 cores and an archival tumor sample are both required. The fresh tumor biopsy may be collected any time after enrollment during the 28-day Screening period. Two fresh cores are required for immunophenotyping by flow cytometry, and the remaining fresh cores will be used for cancer gene expression analysis (NanoString), tumor spatial immune profiling (IHC/IF), and assessment of tumoral IL-12 prodrug cleavage ex vivo. In the event that collection of a fresh biopsy is medically infeasible, or the number of cores obtained inadequate, the patient may continue on study and archival tumor tissue will be used as the baseline sample (tumor block is preferred). However, archival tumor samples cannot be used for certain biomarker analyses such as immunophenotyping by flow cytometry and the IL-12 prodrug cleavage assay.

A second fresh tumor biopsy (again optimally retrieving 5-6 cores) will be collected during treatment between Day 22 and Day 28 of Cycle 1 for immunophenotyping (flow cytometry), cancer gene expression analysis (NanoString) and spatial immune profiling (IHC/IF). The IL-12 prodrug cleavage assay will not be performed using the on-treatment tumor sample. The timing of the second biopsy may shift based on evolving biomarker data.

### 3.11. References for Example 3

Ansell S M, Witzig T E, Kurtin P J, et al. Phase 1 study of interleukin-12 in combination with rituximab in patients with B-cell non-Hodgkin lymphoma. Blood 2002; 99(1):67-74.
Aste-Amezaga M, D'Andrea A, Kubin M, Trinchieri G. Cooperation of natural killer cell stimulatory factor/interleukin-12 with other stimuli in the induction of cytokines and cytotoxic cell-associated molecules in human T and NK cells. Cell Immunol 1994; 156(2):480-92.
Atkins M B, Robertson M J, Gordon M, Lotze M T, DeCoste M, DuBois J S, Ritz J, Sandler A B, Edington H D, Garzone P D, Mier J W, Canning C M, Battiato L, Tahara H, Sherman M L. Phase I evaluation of intravenous recombinant human interleukin-12 in patients with advanced malignancies. Clin Cancer Res 1997; 3(3):409-17.
Bacon C M, Petricoin E F 3rd, Ortaldo J R, et al. Interleukin-12 induces tyrosine phosphorylation and activation of STAT4 in human lymphocytes. Proc Natl Acad Sci USA 1995; 92(16):7307-11.
Bajetta E, Del Vecchio M, Mortarini R, et al. Pilot study of subcutaneous recombinant human interleukin-12 in metastatic melanoma. Clin Cancer Res 1998; 4(1):75-85.
Berraondo P, Etxeberria I, Ponz-Sarvise M, Melero I. Revisiting interleukin-12 as a Cancer Immunotherapy Agent. Clin Cancer Res 2018; 24(12):2716-8.
Bianchi R, Grohmann U, Vacca C, Belladonna M L, Fioretti M C, Puccetti P. Autocrine IL-12 is involved in dendritic cell modulation via CD40 ligation. J Immunol 1999; 163(5):2517-21.
Brunda M J, Luistro L, Warrier R R, et al. Antitumor and antimetastatic activity of interleukin-12 against murine tumors. J Exp Med 1993; 178(4):1223-30.
Brunda M J, Luistro L, Rumennik L, et al. Antitumor activity of interleukin-12 in preclinical models. Cancer Chemother Pharmacol 1996; 38 Suppl:S16-21.
Broz M L, Binnewies M, Boldajipour B, Nelson A E, Pollack J L, Erle D J, et al. Dissecting the Tumor Myeloid Compartment Reveals Rare Activating Antigen-Presenting Cells Critical for T Cell Immunity. Cancer Cell 2014; 26:638-52.
Car B D, Eng V M, Lipman J M, Anderson T D. The toxicology of interleukin-12: a review. Toxicol Pathol 1999; 27(1):58-63.
Castro F, Cardoso A P, Gonçalves R M, Serre K, Oliveira M J. Interferon-gamma at the crossroads of tumor immune surveillance or evasion. Front Immunol 2018; 9:847.
Cheson B D, Fisher R I, Barrington S F, et al. Recommendations for initial evaluation, staging, and response assessment of Hodgkin and non-Hodgkin lymphoma: the Lugano classification. J Clin Oncol 2014; 32(27):3059-68.
Chowdhury F Z, Ramos H J, Davis L S, Forman J, Farrar J D. IL-12 selectively programs effector pathways that are stably expressed in human CD8+ effector memory T cells in vivo. Blood 2011; 118(14):3890-900.
Curtsinger J M, Schmidt C S, Mondino A, et al. Inflammatory cytokines provide a third signal for activation of naive CD4+ and CD8+ T cells. J Immunol 1999; 162(6):3256-62.
Curtsinger J M, Lins D C, Mescher M F. Signal 3 determines tolerance versus full activation of naive CD8 T cells: dissociating proliferation and development of effector function. J Exp Med 2003; 197(9):1141-51.
Dallas S, Chattopadhyay S, Sensenhauser C, et al. Interleukins-12 and -23 do not alter expression or activity of multiple cytochrome P450 enzymes in cryopreserved human hepatocytes. Drug Metab Dispos 2013; 41(4):689-93.
de Jong L M, Jiskoot W, Swen J J, Manson M L. Distinct effects of inflammation on cytochrome P450 regulation and drug metabolism: lessons from experimental models and a potential role for pharmacogenetics. Genes 2020; 11(12):1509.
Del Vecchio M, Bajetta E, Canova S, et al. Interleukin-12: biological properties and clinical application. Clin Cancer Res 2007; 13(16):4677-85.
Eisenhauer E A, Therasse P, Bogaerts J, Schwartz L H, Sargent D, Ford R, et al. New response evaluation criteria in solid tumors: revised RECIST guideline (version 1.1). Eur J Cancer 2009; 45:228-47.
European Medicines Agency (EMA). Clinical trials in small populations. 2006. https://www.ema.europa.eu/en/clinical-trials-small-populations. Accessed 26 Aug. 2021.
Gately M K, Wolitzky A G, Quinn P M, Chizzonite R. Regulation of human cytolytic lymphocyte responses by interleukin-12. Cell Immunol 1992; 143(1):127-42.
Gollob J A, Mier J W, Veenstra K, et al. Phase I trial of twice-weekly intravenous interleukin-12 in patients with metastatic renal cell cancer or malignant melanoma: ability to maintain IFN-gamma induction is associated with clinical response. Clin Cancer Res 2000; 6(5):1678-92.
Grohmann U, Belladonna M L, Bianchi R, et al. IL-12 acts directly on DC to promote nuclear localization of NF-kappaB and primes DC for IL-12 production. Immunity 1998; 9(3):315-23.
Hachad H, Ragueneau-Mejlessi I, Levy R H. A useful tool for drug interaction evaluation: the University of Washington Metabolism and Transport Drug Interaction Database. Hum Genomics 2010; 5:1-12.
Hsieh C S, Macatonia S E, Tripp C S, Wolf S F, O'Garra A, Murphy K M. Development of TH1 CD4+ T cells through IL-12 produced by Listeria-induced macrophages. Science 1993; 260(5107):547-9.
Kobayashi M, Fitz L, Ryan M, et al. Identification and purification of natural killer cell stimulatory factor (NKSF), a cytokine with multiple biologic effects on human lymphocytes. J Exp Med 1989; 170(3):827-45.
Lee D W, Santomasso B D, Locke F L, et al. ASTCT Consensus grading for cytokine release syndrome and neurologic toxicity associated with immune effector cells. Biol Blood Marrow Transplant 2019; 25(4):625-38.
Lenzi R, Rosenblum M, Verschraegen C, et al. Phase I study of intraperitoneal recombinant human interleukin-12 in patients with Müllerian carcinoma, gastrointestinal primary malignancies, and mesothelioma. Clin Cancer Res 2002; 8(12):3686-95.
Leonard J P, Sherman M L, Fisher G L, Buchanan L J, Larsen G, Atkins M B, Sosman J A, Dutcher J P, Vogelzang N J, Ryan J L. Effects of single-dose interleukin-12 exposure on interleukin-12-associated toxicity and interferon-gamma production. Blood 1997; 90(7):2541-8.
Mescher M F, Curtsinger J M, Agarwal P, et al. Signals required for programming effector and memory development by CD8+ T cells. Immunol Rev 2006; 211:81-92.
Motzer R J, Rakhit A, Schwartz L H, et al. Phase I trial of subcutaneous recombinant human interleukin-12 in patients with advanced renal cell carcinoma. Clin Cancer Res 1998; 4(5):1183-91.
Motzer R J, Rakhit A, Thompson J A, et al. Randomized multicenter phase II trial of subcutaneous recombinant human interleukin-12 versus interferon-alpha 2a for patients with advanced renal cell carcinoma. J Interferon Cytokine Res 2001; 21(4):257-63.
Nastala C L, Edington H D, McKinney T G, et al. Recombinant IL-12 administration induces tumor regression in association with IFN-gamma production. J Immunol 1994; 153(4):1697-706.
Nguyen K G, Vrabel M R, Mantooth S M, et al. Localized Interleukin-12 for Cancer Immunotherapy. Front Immunol 2020; 11:575597.
Perussia B, Chan S H, D'Andrea A, et al. Natural killer (NK) cell stimulatory factor or IL-12 has differential effects on the proliferation of TCR-alpha beta+, TCR-gamma delta+ T lymphocytes, and NK cells. J Immunol 1992; 149(11):3495-502.
Robertson M J, Cameron C, Atkins M B, et al. Immunological effects of interleukin-12 administered by bolus intravenous injection to patients with cancer. Clin Cancer Res 1999; 5(1):9-16.
Rogatko A, Schoeneck D, Jonas W, Tighiouart M, Khuri F R, Porter A. Translation of innovative designs into Phase I trials. J Clin Oncol 2007; 25:4982-86.
Rook A H, Wood G S, Yoo E K, et al. Interleukin-12 therapy of cutaneous T-cell lymphoma induces lesion regression and cytotoxic T-cell responses. Blood 1999; 94(3):902-8.
Santomasso B D, Nastoupil L J, Adkins S, et al. Management of immune-related adverse events in patients treated with chimeric antigen receptor T-cell therapy: ASCO Guideline. J Clin Oncol 2021; 39(35):3978-92.
Sigal L J, Crotty S, Andino R, Rock K L. Cytotoxic T-cell immunity to virus-infected non-haematopoietic cells requires presentation of exogenous antigen. Nature 1999; 398:77-80.
Stern A S, Podlaski F J, Hulmes J D, et al. Purification to homogeneity and partial characterization of cytotoxic lymphocyte maturation factor from human B-lymphoblastoid cells. Proc Natl Acad Sci USA 1990; 87(17):6808-12.
Tait Wojno E D, Hunter C A, Stumhofer J S. The immunobiology of the interleukin-12 family: room for discovery. Immunity 2019; 50(4):851-70.
Trinchieri G. Interleukin-12: a cytokine produced by antigen-presenting cells with immunoregulatory functions in the generation of T-helper cells type 1 and cytotoxic lymphocytes. Blood 1994; 84(12):4008-27.
Tugues S, Burkhard S H, Ohs I, et al. New insights into IL-12-mediated tumor suppression. Cell Death Differ 2014; 22(2):237-46.
Van Herpen, Huijbens, Looman, et al. Pharmacokinetics and immunological aspects of a Phase Ib study with intratumoral administration of recombinant human interleukin-12 in patients with head and neck squamous cell carcinoma: a decrease of T-bet in peripheral blood mononuclear cells. Clin Cancer Res 2003; 9(8):2950-6.
Waldmann T A. Cytokines in Cancer Immunotherapy. Cold Spring Harb Perspect Biol [Internet] 2018; 10(12).
Weiss G R, O'Donnell M A, Loughlin K, Zonno K, Laliberte R J, Sherman M L. Phase 1 study of the intravesical administration of recombinant human interleukin-12 in patients with recurrent superficial transitional cell carcinoma of the bladder. J Immunother 2003; 26(4):343-8.
Wherry E J, Blattman J N, Murali-Krishna K, van der Most R, Ahmed R. Viral persistence alters CD8 T-cell immunodominance and tissue distribution and results in distinct stages of functional impairment. J Virol 2003; 77(8):4911-4927.
Younes A, Pro B, Robertson M J, et al. Phase II clinical trial of interleukin-12 in patients with relapsed and refractory non-Hodgkin's lymphoma and Hodgkin's disease. Clin Cancer Res 2004; 10(16):5432-8.
Zou J P, Yamamoto N, Fujii T, et al. Systemic administration of rIL-12 induces complete tumor regression and protective immunity: response is correlated with a striking reversal of suppressed IFN-gamma production by anti-tumor T cells. Int Immunol 1995; 7(7):1135-45.

1. SEQUENCE DISCLOSURE

| SEQ ID | | |
|---|---|---|
| NO: | Description | Sequence |
| 1 | Chimeric | EVQLVESGGGLVQPGNSLRLSCAASGFTFSKFGMSWVRQA |
| | Compound 1 | PGKGLEWVSSISGSGRDTLYAESVKGRFTISRDNAKTTLYL |
| | (WW0757) | QMNSLRPEDTAVYYCTIGGSLSVSSQGTLVTVSSsggpALFK |
| | Heterodimeric IL- | SSFPpgsrvipvsgparclsqsrnllkttddmvktareklkhysctaedidheditrdqtst |
| | 12 polypeptide, | lktclplelhknesclatretssttrgsclppqktslmmtlclgsiyedlkmyqtefqainaal |
| | anti-HSA sdAb, | qnhnhqqiildkgmlvaidelmqslnhngetlrqkppvgeadpyrvkmklcillhafstr |
| | scFv Blocker, 2 | vvtinrvmgylssasggpALFKSSFPpgsggggsggggsggggsggggsggggs |
| | cleavage sites | ggggsQSVLTQPPSVSGAPGQRVTISCSGSRSNIGSNTVKWY |
| | | QQLPGTAPKLLIYYNDQRPSGVPDRFSGSKSGTSASLAITG |
| | | LQAEDEADYYCQSYDRYTHPALLFGTGTKVTVLggggsgggg |
| | | sggggsQVQLVESGGGVVQPGRSLRLSCAASGFTFSSYGMH |
| | | WVRQAPGKGLEWVAFIRYeGSNKYYAeSVKGRFTISRDNS |
| | | KNTLYLQMNSLRAEDTAVYYCKTHGSHDNWGQGTMVTV |
| | | SS** |
| 2 | WW0805 | rnlpvatpdpgmfpclhhsqnllravsnmlqkarqtlefypctseeidheditkdktstvea |
| | Heterodimeric IL- | clpleltknesclnsretsfitngsclasrktsfmmalclssiyedlkmyqvefktmnakll |
| | 12 polypeptide, | mdpkrqifldqnmlavidelmqalnfnsetvpqkssleepdfyktkiklcillhafriravti |
| | anti-HSA sdAb, | drvmsylnassggpALFKSSFPpgsEVQLVESGGGLVQPGNSLRLS |
| | scFv Blocker, 1 | CAASGFTFSKFGMSWVRQAPGKGLEWVSSISGSGRDTLYA |
| | cleavage site | ESVKGRFTISRDNAKTTLYLQMNSLRPEDTAVYYCTIGGSL |
| | | SVSSQGTLVTVSSggggsggggsggggsggggsggggsggggsQSVLTQ |
| | | PPSVSGAPGQRVTISCSGSRSNIGSNTVKWYQQLPGTAPKL |
| | | LIYYNDQRPSGVPDRFSGSKSGTSASLAITGLQAEDEADYY |
| | | CQSYDRYTHPALLFGTGTKVTVLggggsggggsggggsQVQLVE |
| | | SGGGVVQPGRSLRLSCAASGFTFSSYGMHWVRQAPGKGL |
| | | EWVAFIRYeGSNKYYAeSVKGRFTISRDNSKNTLYLQMNSL |
| | | RAEDTAVYYCKTHGSHDNWGQGTMVTVSS** |
| 3 | WW0754 | EVQLVESGGGLVQPGNSLRLSCAASGFTFSKFGMSWVRQA |
| | Heterodimeric IL- | PGKGLEWVSSISGSGRDTLYAESVKGRFTISRDNAKTTLYL |
| | 12 polypeptide, | QMNSLRPEDTAVYYCTIGGSLSVSSQGTLVTVSSsggpGPAG |
| | anti-HSA sdAb, | LYAQpgsrnlpvatpdpgmfpclhhsqnllravsnmlqkarqtlefypctseeidhedi |
| | Fab Blocker, 2 | tkdktstveaclpleltknesclnsretsfitngsclasrktsfmmalclssiyedlkmyqvef |
| | cleavage sites | ktmnakllmdpkrqifldqnmlavidelmqalnfnsetvpqkssleepdfyktkiklcill |
| | | hafriravtidrvmsylnassggpGPAGLYAQpgsggggsggggsggggsggggs |
| | | ggggsggggsQSVLTQPPSVSGAPGQRVTISCSGSRSNIGSNTV |
| | | KWYQQLPGTAPKLLIYYNDQRPSGVPDRFSGSKSGTSASL |
| | | AITGLQAEDEADYYCQSYDRYTHPALLFGTGTKVTVLgqpk |
| | | aapsvtlfppsseelqankatlvclisdfypgavtvawkadsspvkagvetttpskqsnnk |
| | | yaassylsltpeqwkshrsyscqvthegstvektvaptecs** |
| 4 | WW0756 | EVQLVESGGGLVQPGNSLRLSCAASGFTFSKFGMSWVRQA |
| | Heterodimeric IL- | PGKGLEWVSSISGSGRDTLYAESVKGRFTISRDNAKTTLYL |
| | 12 polypeptide, | QMNSLRPEDTAVYYCTIGGSLSVSSQGTLVTVSSsggpGPAG |
| | anti-HSA sdAb, | LYAQpgsrnlpvatpdpgmfpclhhsqnllravsnmlqkarqtlefypctseeidhedi |
| | Fab Blocker, 2 | tkdktstveaclpleltknesclnsretsfitngsclasrktsfmmalclssiyedlkmyqvef |
| | cleavage sites | ktmnakllmdpkrqifldqnmlavidelmqalnfnsetvpqkssleepdfyktkiklcill |
| | | hafriravtidrvmsylnassggpGPAGLYAQpgsggggsggggsggggsggggs |
| | | ggggsggggsQSVLTQPPSVSGAPGQRVTISCSGSRSNIGdeTVK |
| | | WYQQLPGTAPKLLIYYNDQRPSGVPDRFSGSKSGTSASLAI |
| | | TGLQAEDEADYYCQSYDRYTHPALLFGTGTKVTVLgqpkaa |
| | | psvtlfppsseelqankatlvclisdfypgavtvawkadsspvkagvetttpskqsnnkya |
| | | assylsltpeqwkshrsyscqvthegstvektvaptecs** |
| 5 | WW0762 | EVQLVESGGGLVQPGNSLRLSCAASGFTFSKFGMSWVRQA |
| | Heterodimeric IL- | PGKGLEWVSSISGSGRDTLYAESVKGRFTISRDNAKTTLYL |
| | 12 polypeptide, | QMNSLRPEDTAVYYCTIGGSLSVSSQGTLVTVSSsggpALFK |
| | anti-HSA sdAb, | SSFPpgsrnlpvatpdpgmfpclhhsqnllravsnmlqkarqtlefypctseeidhedit |
| | Fab Blocker, 2 | kdktstveaclpleltknesclnsretsfitngsclasrktsfmmalclssiyedlkmyqvef |
| | cleavage sites | ktmnakllmdpkrqifldqnmlavidelmqalnfnsetvpqkssleepdfyktkiklcill |
| | | hafriravtidrvmsylnassggpALFKSSFPpgsggggsggggsggggsggggsg |
| | | gggsggggsQSVLTQPPSVSGAPGQRVTISCSGSRSNIGSNTVK |
| | | WYQQLPGTAPKLLIYYNDQRPSGVPDRFSGSKSGTSASLAI |
| | | TGLQAEDEADYYCQSYDRYTHPALLFGTGTKVTVLgqpkaa |
| | | psvtlfppsseelqankatlvclisdfypgavtvawkadsspvkagvetttpskqsnnkya |
| | | assylsltpeqwkshrsyscqvthegstvektvaptecs** |
| 6 | WW0770 | iwelkkdvyvveldwypdapgemvvltcdtpeedgitwtldqssevlgsgktltiqvkef |
| | Monomeric IL-12 | gdagqytchkggevlshsllllhkkedgiwstdilkdqkepknktflrceaknysgrftcw |
| | polypeptide, anti- | wlttistdltfsvkssrgssdpqgvtcgaatlsaervrgdnkeyeysvecqedsacpaaeesl |
| | HSA sdAb, Fab | pievmvdavhklkyenytssffirdiikpdppknlqlkplknsrqvevsweypdtwstp |
| | Blocker, 1 | hsyfsltfcvqvqgkskrekkdrvftdktsatvicrknasisvraqdryyssswsewasvp |
| | cleavage site | csggggsggggsggggsrnlpvatpdpgmfpclhhsqnllravsnmlqkarqtlefypct |
| | | seeidheditkdktstveaclpleltknesclnsretsfitngsclasrktsfmmalclssiyed |
| | | lkmyqvefktmnakllmdpkrqifldqnmlavidelmqalnfnsetvpqkssleepdfy |
| | | ktkiklcillhafriravtidrvmsylnassggpGPAGLYAQpgsEVQLVESG |
| | | GGLVQPGNSLRLSCAASGFTFSKFGMSWVRQAPGKGLEW |
| | | VSSISGSGRDTLYAESVKGRFTISRDNAKTTLYLQMNSLRP |
| | | EDTAVYYCTIGGSLSVSSQGTLVTVSSggggsggggsggggsgggg |
| | | sggggsggggsQSVLTQPPSVSGAPGQRVTISCSGSRSNIGSNTV |
| | | KWYQQLPGTAPKLLIYYNDQRPSGVPDRFSGSKSGTSASL |
| | | AITGLQAEDEADYYCQSYDRYTHPALLFGTGTKVTVLgqpk |
| | | aapsvtlfppsseelqankatlvclisdfypgavtvawkadsspvkagvetttpskqsnnk |
| | | yaassylsltpeqwkshrsyscqvthegstvektvaptecs** |
| 7 | WW0636 | iwelkkdvyvveldwypdapgemvvltcdtpeedgitwtldqssevlgsgktltiqvkef |
| | | gdagqytchkggevlshsllllhkkedgiwstdilkdqkepknktflrceaknysgrftcw |
| | | wlttistdltfsvkssrgssdpqgvtcgaatlsaervrgdnkeyeysvecqedsacpaaeesl |
| | | pievmvdavhklkyenytssffirdiikpdppknlqlkplknsrqvevsweypdtwstp |
| | | hsyfsltfcvqvqgkskrekkdrvftdktsatvicrknasisvraqdryyssswsewasvp |
| | | cs** |
| 8 | WW0727 | QVQLVESGGGVVQPGRSLRLSCAASGFTFSSYGMHWVRQ |
| | Monomeric IL-12 | APGKGLEWVAFIRYeGSNKYYAeSVKGRFTISRDNSKNTLY |
| | polypeptide, anti- | LQMNSLRAEDTAVYYCKTHGSHDNWGQGTMVTVSSastkg |
| | HSA sdAb, Fab | psvfplapsskstsggtaalgclvkdyfpepvtvswnsgaltsgvhtfpavlqssglyslss |
| | Blocker, 2 | vvtvpssslgtqtyicnvnhkpsntkvdkrvepksc** |
| | cleavage sites | |
| 9 | WW01088 | eskygppcppcpapeflggpsvflfppkpkdtlmisrtpevtcvvvdvsqedpevqf |
| | | nwyvdgvevhnaktkpreeqfnstyrvvsvltvlhqdwlngkeykckvsnkglps |
| | | siektiskakgqprepqvytlppsqeemtknqvsltclvkgfypsdiavewesngqp |
| | | ennykttppvldsdgsfflysrltvdksrwqegnvfscsvmhealhnhytqkslslslg |
| | | ksggpALFKSSFPpgsiwelkkdvyvveldwypdapgemvvltcdtpeedgi |
| | | twtldqssevlgsgktltiqvkefgdagqytchkggevlshsllllhkkedgiwstdilk |
| | | dqkepknktflrceaknysgrftcwwlttistdltfsvkssrgssdpqgvtcgaatlsaer |
| | | vrgdnkeyeysvecqedsacpaaeeslpievmvdavhklkyenytssffirdiikpd |
| | | ppknlqlkplknsrqvevsweypdtwstphsyfsltfcvqvqgkskrekkdrvftdk |
| | | tsatvicrknasisvraqdryyssswsewasvpcsggggsggggsggggsrnlpvat |
| | | pdpgmfpclhhsqnllravsnmlqkarqtlefypctseeidheditkdktstveaclpl |
| | | eltknesclnsretsfitngsclasrktsfmmalclssiyedlkmyqvefktmnakllm |
| | | dpkrqifldqnmlavidelmqalnfnsetvpqkssleepdfyktkiklcillhafrirav |
| | | tidrvmsylnassggpALFKSSFPpgsggggsggggsggggsggggsggggs |
| | | ggggsQSVLTQPPSVSGAPGQRVTISCSGSRSNIGSNTVKW |
| | | YQQLPGTAPKLLIYYNDQRPSGVPDRFSGSKSGTSASLA |
| | | ITGLQAEDEADYYCQSYDRYTHPALLFGTGTKVTVLggg |
| | | gsggggsggggsQVQLVESGGGVVQPGRSLRLSCAASGFTFS |
| | | SYGMHWVRQAPGKGLEWVAFIRYDGSNKYYADSVKG |
| | | RFTISRDNSKNTLYLQMNSLRAEDTAVYYCKTHGSHDN |
| | | WGQGTMVTVSS** |
| 10 | WW01089 | iwelkkdvyvveldwypdapgemvvltcdtpeedgitwtldqssevlgsgktltiqv |
| | | kefgdagqytchkggevlshsllllhkkedgiwstdilkdqkepknktflrceaknys |
| | | grftcwwlttistdltfsvkssrgssdpqgvtcgaatlsaervrgdnkeyeysvecqeds |
| | | acpaaeeslpievmvdavhklkyenytssffirdiikpdppknlqlkplknsrqvevs |
| | | weypdtwstphsyfsltfcvqvqgkskrekkdrvftdktsatvicrknasisvraqdry |
| | | yssswsewasvpcsggggsggggsggggsrnlpvatpdpgmfpclhhsqnllrav |
| | | snmlqkarqtlefypctseeidheditkdktstveaclpleltknesclnsretsfitngsc |
| | | lasrktsfmmalclssiyedlkmyqvefktmnakllmdpkrqifldqnmlavidel |
| | | mqalnfnsetvpqkssleepdfyktkiklcillhafriravtidrvmsylnassggpAL |
| | | FKSSFPpgsggggsggggsggggsggggsggggsggggsQSVLTQPPS |
| | | VSGAPGQRVTISCSGSRSNIGSNTVKWYQQLPGTAPKLL |
| | | IYYNDQRPSGVPDRFSGSKSGTSASLAITGLQAEDEADY |
| | | YCQSYDRYTHPALLFGTGTKVTVLggggsggggsggggsQVQ |
| | | LVESGGGVVQPGRSLRLSCAASGFTFSSYGMHWVRQAP |
| | | GKGLEWVAFIRYDGSNKYYADSVKGRFTISRDNSKNTL |
| | | YLQMNSLRAEDTAVYYCKTHGSHDNWGQGTMVTVSSs |
| | | ggpALFKSSFPpgseskygppcppcpapeflggpsvflfppkpkdtlmisrtpe |
| | | vtcvvvdvsqedpevqfnwyvdgvevhnaktkpreeqfnstyrvvsvltvlhqdw |
| | | lngkeykckvsnkglpssiektiskakgqprepqvytlppsqeemtknqvsltclvk |
| | | gfypsdiavewesngqpennykttppvldsdgsfflysrltvdksrwqegnvfscsv |
| | | mhealhnhytqkslslslgk** |
| 11 | WW01090 | vprdcgckpcictvpevssvfifppkpkdvltitltpkvtcvvvdiskddpevqfswf |
| | | vddvevhtaqtqpreeqfnstfrsvselpimhqdwlngkefkcrvnsaafpapiekti |
| | | sktkgrpkapqvytipppkeqmakdkvsltcmitdffpeditvewqwngqpaeny |
| | | kntqpimdtdgsyfvysklnvqksnweagntftcsvlheglhnhhtekslshspgks |
| | | ggpALFKSSFPpgsrvipvsgparclsqsrnllkttddmvktareklkhysctae |
| | | didheditrdqtstlktclplelhknesclatretssttrgsclppqktslmmtlclgsiyed |
| | | lkmyqtefqainaalqnhnhqqiildkgmlvaidelmqslnhngetlrqkppvgea |
| | | dpyrvkmklcillhafstrvvtinrvmgylssasggpALFKSSFPpgsggggsg |
| | | gggsggggsggggsggggsggggsQSVLTQPPSVSGAPGQRVTISC |
| | | SGSRSNIGSNTVKWYQQLPGTAPKLLIYYNDQRPSGVPD |
| | | RFSGSKSGTSASLAITGLQAEDEADYYCQSYDRYTHPAL |
| | | LFGTGTKVTVLggggsggggsggggsQVQLVESGGGVVQPGR |
| | | SLRLSCAASGFTFSSYGMHWVRQAPGKGLEWVAFIRYD |
| | | GSNKYYADSVKGRFTISRDNSKNTLYLQMNSLRAEDTA |
| | | VYYCKTHGSHDNWGQGTMVTVSS** |
| 12 | WW01091 | rvipvsgparclsqsrnllkttddmvktareklkhysctaedidheditrdqtstlktclpl |
| | | elhknesclatretssttrgsclppqktslmmtlclgsiyedlkmyqtefqainaalqnh |
| | | nhqqiildkgmlvaidelmqslnhngetlrqkppvgeadpyrvkmklcillhafstrv |
| | | vtinrvmgylssasggpALFKSSFPpgsggggsggggsggggsggggsgggg |
| | | sggggsQSVLTQPPSVSGAPGQRVTISCSGSRSNIGSNTVK |
| | | WYQQLPGTAPKLLIYYNDQRPSGVPDRFSGSKSGTSASL |
| | | AITGLQAEDEADYYCQSYDRYTHPALLFGTGTKVTVLg |
| | | gggsggggsggggsQVQLVESGGGVVQPGRSLRLSCAASGFT |
| | | FSSYGMHWVRQAPGKGLEWVAFIRYDGSNKYYADSVK |
| | | GRFTISRDNSKNTLYLQMNSLRAEDTAVYYCKTHGSHD |
| | | NWGQGTMVTVSSsggpALFKSSFPpgsvprdcgckpcictvpevss |
| | | vfifppkpkdvltitltpkvtcvvvdiskddpevqfswfvddvevhtaqtqpreeqfn |
| | | stfrsvselpimhqdwlngkefkcrvnsaafpapiektisktkgrpkapqvytipppk |
| | | eqmakdkvsltcmitdffpeditvewqwngqpaenykntqpimdtdgsyfvyskl |
| | | nvqksnweagntftcsvlheglhnhhtekslshspgk** |
| 13 | WW01092 | eskygppcppcpapeflggpsvflfppkpkdtlmisrtpevtcvvvdvsqedpevqf |
| | | nwyvdgvevhnaktkpreeqfnstyrvvsvltvlhqdwlngkeykckvsnkglps |
| | | siektiskakgqprepqvytlppsqeemtknqvsltclvkgfypsdiavewesngqp |
| | | ennykttppvldsdgsfflysrltvdksrwqegnvfscsvmhealhnhytqkslslslg |
| | | ksggpALFKSSFPpgsrnlpvatpdpgmfpclhhsqnllravsnmlqkarqtle |
| | | fypctseeidheditkdktstveaclpleltknesclnsretsfitngsclasrktsfmmal |
| | | clssiyedlkmyqvefktmnakllmdpkrqifldqnmlavidelmqalnfnsetvp |
| | | qkssleepdfyktkiklcillhafriravtidrvmsylnassggpALFKSSFPpgsg |
| | | gggsggggsggggsggggsggggsggggsQSVLTQPPSVSGAPGQR |
| | | VTISCSGSRSNIGSNTVKWYQQLPGTAPKLLIYYNDQRP |
| | | SGVPDRFSGSKSGTSASLAITGLQAEDEADYYCQSYDRY |
| | | THPALLFGTGTKVTVLggggsggggsggggsQVQLVESGGGV |
| | | VQPGRSLRLSCAASGFTFSSYGMHWVRQAPGKGLEWV |
| | | AFIRYDGSNKYYADSVKGRFTISRDNSKNTLYLQMNSL |
| | | RAEDTAVYYCKTHGSHDNWGQGTMVTVSS** |
| 14 | WW01093 | rnlpvatpdpgmfpclhhsqnllravsnmlqkarqtlefypctseeidheditkdktst |
| | | veaclpleltknesclnsretsfitngsclasrktsfmmalclssiyedlkmyqvefktm |
| | | nakllmdpkrqifldqnmlavidelmqalnfnsetvpqkssleepdfyktkiklcillh |
| | | afriravtidrvmsylnassggpALFKSSFPpgsggggsggggsggggsgggg |
| | | sggggsggggsQSVLTQPPSVSGAPGQRVTISCSGSRSNIGSN |
| | | TVKWYQQLPGTAPKLLIYYNDQRPSGVPDRFSGSKSGT |
| | | SASLAITGLQAEDEADYYCQSYDRYTHPALLFGTGTKV |
| | | TVLggggsggggsggggsQVQLVESGGGVVQPGRSLRLSCAA |
| | | SGFTFSSYGMHWVRQAPGKGLEWVAFIRYDGSNKYYA |
| | | DSVKGRFTISRDNSKNTLYLQMNSLRAEDTAVYYCKTH |
| | | GSHDNWGQGTMVTVSSsggpALFKSSFPpgseskygppcppcp |
| | | apeflggpsvflfppkpkdtlmisrtpevtcvvvdvsqedpevqfnwyvdgvevhn |
| | | aktkpreeqfnstyrvvsvltvlhqdwlngkeykckvsnkglpssiektiskakgqpr |
| | | epqvytlppsqeemtknqvsltclvkgfypsdiavewesngqpennykttppvlds |
| | | dgsfflysrltvdksrwqegnvfscsvmhealhnhytqkslslslgk** |
| 15 | WW01094 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsrnlpvatpdpgmfpclhhsqnllravsnmlqkarqtlefyp |
| | | ctseeidheditkdktstveaclpleltknesclnsretsfitngsclasrktsfmmalclss |
| | | iyedlkmyqvefktmnakllmdpkrqifldqnmlavidelmqalnfnsetvpqkss |
| | | leepdfyktkiklcillhafriravtidrvmsylnassggpALFKSSFPpgsggggs |
| | | ggggsggggsggggsggggsggggsQVQLVESGGGVVQPGRSLRL |
| | | SCAASGFTFSSYGMHWVRQAPGKGLEWVAFIRYeGSNK |
| | | YYAeSVKGRFTISRDNSKNTLYLQMNSLRAEDTAVYYC |
| | | KTHGSHDNWGQGTMVTVSSastkgpsvfplapsskstsggtaalgclv |
| | | kdyfpepvtvswnsgaltsgvhtfpavlqssglyslssvvtvpssslgtqtyicnvnhk |
| | | psntkvdkrvepksc** |
| 16 | WW01096 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsrvipvsgparclsqsrnllkttddmvktareklkhysctaedi |
| | | dheditrdqtstlktclplelhknesclatretssttrgsclppqktslmmtlclgsiyedlk |
| | | myqtefqainaalqnhnhqqiildkgmlvaidelmqslnhngetlrqkppvgeadp |
| | | yrvkmklcillhafstrvvtinrvmgylssasggpALFKSSFPpgsggggsggg |
| | | gsggggsggggsggggsggggsQVQLVESGGGVVQPGRSLRLSC |
| | | AASGFTFSSYGMHWVRQAPGKGLEWVAFIRYeGSNKY |
| | | YAeSVKGRFTISRDNSKNTLYLQMNSLRAEDTAVYYCK |
| | | THGSHDNWGQGTMVTVSSastkgpsvfplapsskstsggtaalgclvk |
| | | dyfpepvtvswnsgaltsgvhtfpavlqssglyslssvvtvpssslgtqtyicnvnhkp |
| | | sntkvdkrvepksc** |
| 17 | WW01099 | iwelkkdvyvveldwypdapgemvvltcdtpeedgitwtldqssevlgsgktltiqv |
| | | kefgdagqytchkggevlshsllllhkkedgiwstdilkdqkepknktflrceaknys |
| | | grftcwwlttistdltfsvkssrgssdpqgvtcgaatlsaervrgdnkeyeysvecqeds |
| | | acpaaeeslpievmvdavhklkyenytssffirdiikpdppknlqlkplknsrqvevs |
| | | weypdtwstphsyfsltfcvqvqgkskrekkdrvftdktsatvicrknasisvraqdry |
| | | yssswsewasvpcsggggsggggsggggsrnlpvatpdpgmfpclhhsqnllrav |
| | | snmlqkarqtlefypctseeidheditkdktstveaclpleltknesclnsretsfitngsc |
| | | lasrktsfmmalclssiyedlkmyqvefktmnakllmdpkrqifldqnmlavidel |
| | | mqalnfnsetvpqkssleepdfyktkiklcillhafriravtidrvmsylnassggpAL |
| | | FKSSFPpgsEVQLVESGGGLVQPGNSLRLSCAASGFTFSK |
| | | FGMSWVRQAPGKGLEWVSSISGSGRDTLYAESVKGRFT |
| | | ISRDNAKTTLYLQMNSLRPEDTAVYYCTIGGSLSVSSQG |
| | | TLVTVSSsggpALFKSSFPpgsggggsggggsggggsggggsggggsgg |
| | | ggsQSVLTQPPSVSGAPGQRVTISCSGSRSNIGSNTVKWY |
| | | QQLPGTAPKLLIYYNDQRPSGVPDRFSGSKSGTSASLAIT |
| | | GLQAEDEADYYCQSYDRYTHPALLFGTGTKVTVLggggs |
| | | ggggsggggsQVQLVESGGGVVQPGRSLRLSCAASGFTFSS |
| | | YGMHWVRQAPGKGLEWVAFIRYeGSNKYYAeSVKGRF |
| | | TISRDNSKNTLYLQMNSLRAEDTAVYYCKTHGSHDNW |
| | | GQGTMVTVSS** |
| 18 | WW01100 | iwelkkdvyvveldwypdapgemvvltcdtpeedgitwtldqssevlgsgktltiqv |
| | | kefgdagqytchkggevlshsllllhkkedgiwstdilkdqkepknktflrceaknys |
| | | grftcwwlttistdltfsvkssrgssdpqgvtcgaatlsaervrgdnkeyeysvecqeds |
| | | acpaaeeslpievmvdavhklkyenytssffirdiikpdppknlqlkplknsrqvevs |
| | | weypdtwstphsyfsltfcvqvqgkskrekkdrvftdktsatvicrknasisvraqdry |
| | | yssswsewasvpcsggggsggggsggggsrvipvsgparclsqsrnllkttddmvk |
| | | tareklkhysctaedidheditrdqtstlktclplelhknesclatretssttrgsclppqkts |
| | | Immtlclgsiyedlkmyqtefqainaalqnhnhqqiildkgmlvaidelmqslnhn |
| | | getlrqkppvgeadpyrvkmklcillhafstrvvtinrvmgylssasggpALFKSS |
| | | FPpgsEVQLVESGGGLVQPGNSLRLSCAASGFTFSKFGMS |
| | | WVRQAPGKGLEWVSSISGSGRDTLYAESVKGRFTISRD |
| | | NAKTTLYLQMNSLRPEDTAVYYCTIGGSLSVSSQGTLV |
| | | TVSSsggpALFKSSFPpgsggggsggggsggggsggggsggggsggggs |
| | | QSVLTQPPSVSGAPGQRVTISCSGSRSNIGSNTVKWYQQ |
| | | LPGTAPKLLIYYNDQRPSGVPDRFSGSKSGTSASLAITGL |
| | | QAEDEADYYCQSYDRYTHPALLFGTGTKVTVLggggsggg |
| | | gsggggsQVQLVESGGGVVQPGRSLRLSCAASGFTFSSYG |
| | | MHWVRQAPGKGLEWVAFIRYeGSNKYYAeSVKGRFTIS |
| | | RDNSKNTLYLQMNSLRAEDTAVYYCKTHGSHDNWGQ |
| | | GTMVTVSS** |
| 19 | WW01101 | rnlpvatpdpgmfpclhhsqnllravsnmlqkarqtlefypctseeidheditkdktst |
| | | veaclpleltknesclnsretsfitngsclasrktsfmmalclssiyedlkmyqvefktm |
| | | nakllmdpkrqifldqnmlavidelmqalnfnsetvpqkssleepdfyktkiklcillh |
| | | afriravtidrvmsylnassggpALFKSSFPpgsEVQLVESGGGLVQP |
| | | GNSLRLSCAASGFTFSKFGMSWVRQAPGKGLEWVSSIS |
| | | GSGRDTLYAESVKGRFTISRDNAKTTLYLQMNSLRPED |
| | | TAVYYCTIGGSLSVSSQGTLVTVSSsggpALFKSSFPpgsgg |
| | | ggsggggsggggsggggsggggsggggsQSVLTQPPSVSGAPGQRV |
| | | TISCSGSRSNIGSNTVKWYQQLPGTAPKLLIYYNDQRPS |
| | | GVPDRFSGSKSGTSASLAITGLQAEDEADYYCQSYDRY |
| | | THPALLFGTGTKVTVLggggsggggsggggsQVQLVESGGGV |
| | | VQPGRSLRLSCAASGFTFSSYGMHWVRQAPGKGLEWV |
| | | AFIRYeGSNKYYAeSVKGRFTISRDNSKNTLYLQMNSLR |
| | | AEDTAVYYCKTHGSHDNWGQGTMVTVSS** |
| 20 | WW01102 | rvipvsgparclsqsrnllkttddmvktareklkhysctaedidheditrdqtstlktclpl |
| | | elhknesclatretssttrgsclppqktslmmtlclgsiyedlkmyqtefqainaalqnh |
| | | nhqqiildkgmlvaidelmqslnhngetlrqkppvgeadpyrvkmklcillhafstrv |
| | | vtinrvmgylssasggpALFKSSFPpgsEVQLVESGGGLVQPGNS |
| | | LRLSCAASGFTFSKFGMSWVRQAPGKGLEWVSSISGSG |
| | | RDTLYAESVKGRFTISRDNAKTTLYLQMNSLRPEDTAV |
| | | YYCTIGGSLSVSSQGTLVTVSSsggpALFKSSFPpgsggggsgg |
| | | ggsggggsggggsggggsggggsQSVLTQPPSVSGAPGQRVTISCS |
| | | GSRSNIGSNTVKWYQQLPGTAPKLLIYYNDQRPSGVPD |
| | | RFSGSKSGTSASLAITGLQAEDEADYYCQSYDRYTHPAL |
| | | LFGTGTKVTVLggggsggggsggggsQVQLVESGGGVVQPGR |
| | | SLRLSCAASGFTFSSYGMHWVRQAPGKGLEWVAFIRYe |
| | | GSNKYYAeSVKGRFTISRDNSKNTLYLQMNSLRAEDTA |
| | | VYYCKTHGSHDNWGQGTMVTVSS** |
| 21 | WW01103 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsiwelkkdvyvveldwypdapgemvvltcdtpeedgitwt |
| | | ldqssevlgsgktltiqvkefgdagqytchkggevlshsllllhkkedgiwstdilkdq |
| | | kepknktflrceaknysgrftcwwlttistdltfsvkssrgssdpqgvtcgaatlsaervr |
| | | gdnkeyeysvecqedsacpaaeeslpievmvdavhklkyenytssffirdiikpdpp |
| | | knlqlkplknsrqvevsweypdtwstphsyfsltfcvqvqgkskrekkdrvftdktsa |
| | | tvicrknasisvraqdryyssswsewasvpcsggggsggggsggggsggggsggg |
| | | gsggggsrnlpvatpdpgmfpclhhsqnllravsnmlqkarqtlefypctseeidhed |
| | | itkdktstveaclpleltknesclnsretsfitngsclasrktsfmmalclssiyedlkmy |
| | | qvefktmnakllmdpkrqifldqnmlavidelmqalnfnsetvpqkssleepdfykt |
| | | kiklcillhafriravtidrvmsylnassggpALFKSSFPpgsggggsggggsgg |
| | | ggsggggsggggsggggsQSVLTQPPSVSGAPGQRVTISCSGSR |
| | | SNIGSNTVKWYQQLPGTAPKLLIYYNDQRPSGVPDRFSG |
| | | SKSGTSASLAITGLQAEDEADYYCQSYDRYTHPALLFGT |
| | | GTKVTVLggggsggggsggggsQVQLVESGGGVVQPGRSLRL |
| | | SCAASGFTFSSYGMHWVRQAPGKGLEWVAFIRYeGSNK |
| | | YYAeSVKGRFTISRDNSKNTLYLQMNSLRAEDTAVYYC |
| | | KTHGSHDNWGQGTMVTVSS** |
| 22 | WW01104 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsiwelkkdvyvveldwypdapgemvvltcdtpeedgitwt |
| | | ldqssevlgsgktltiqvkefgdagqytchkggevlshsllllhkkedgiwstdilkdq |
| | | kepknktflrceaknysgrftcwwlttistdltfsvkssrgssdpqgvtcgaatlsaervr |
| | | gdnkeyeysvecqedsacpaaeeslpievmvdavhklkyenytssffirdiikpdpp |
| | | knlqlkplknsrqvevsweypdtwstphsyfsltfcvqvqgkskrekkdrvftdktsa |
| | | tvicrknasisvraqdryyssswsewasvpcsggggsggggsggggsggggsggg |
| | | gsggggsrvipvsgparclsqsrnllkttddmvktareklkhysctaedidheditrdqt |
| | | stlktclplelhknesclatretssttrgsclppqktslmmtlclgsiyedlkmyqtefqai |
| | | naalqnhnhqqiildkgmlvaidelmqslnhngetlrqkppvgeadpyrvkmklci |
| | | llhafstrvvtinrvmgylssasggpALFKSSFPpgsggggsggggsggggsgg |
| | | ggsggggsggggsQSVLTQPPSVSGAPGQRVTISCSGSRSNIGS |
| | | NTVKWYQQLPGTAPKLLIYYNDQRPSGVPDRFSGSKSG |
| | | TSASLAITGLQAEDEADYYCQSYDRYTHPALLFGTGTK |
| | | VTVLggggsggggggggsQVQLVESGGGVVQPGRSLRLSCA |
| | | ASGFTFSSYGMHWVRQAPGKGLEWVAFIRYeGSNKYY |
| | | AeSVKGRFTISRDNSKNTLYLQMNSLRAEDTAVYYCKT |
| | | HGSHDNWGQGTMVTVSS** |
| 23 | WW01105 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsrnlpvatpdpgmfpclhhsqnllravsnmlqkarqtlefyp |
| | | ctseeidheditkdktstveaclpleltknesclnsretsfitngsclasrktsfmmalclss |
| | | iyedlkmyqvefktmnakllmdpkrqifldqnmlavidelmqalnfnsetvpqkss |
| | | leepdfyktkiklcillhafriravtidrvmsylnasggggsggggsg |
| | | gggsggggsiwelkkdvyvveldwypdapgemvvltcdtpeedgitwtldqssev |
| | | lgsgktltiqvkefgdagqytchkggevlshsllllhkkedgiwstdilkdqkepknkt |
| | | flrceaknysgrftcwwlttistdltfsvkssrgssdpqgvtcgaatlsaervrgdnkeye |
| | | ysvecqedsacpaaeeslpievmvdavhklkyenytssffirdiikpdppknlqlkpl |
| | | knsrqvevsweypdtwstphsyfsltfcvqvqgkskrekkdrvftdktsatvicrkna |
| | | sisvraqdryyssswsewasvpcssggpALFKSSFPpgsggggsggggsggg |
| | | gsggggsggggsggggsQSVLTQPPSVSGAPGQRVTISCSGSRS |
| | | NIGSNTVKWYQQLPGTAPKLLIYYNDQRPSGVPDRFSGS |
| | | KSGTSASLAITGLQAEDEADYYCQSYDRYTHPALLFGT |
| | | GTKVTVLggggsggggsggggsQVQLVESGGGVVQPGRSLRL |
| | | SCAASGFTFSSYGMHWVRQAPGKGLEWVAFIRYeGSNK |
| | | YYAeSVKGRFTISRDNSKNTLYLQMNSLRAEDTAVYYC |
| | | KTHGSHDNWGQGTMVTVSS** |
| 24 | WW01106 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsrvipvsgparclsqsrnllkttddmvktareklkhysctaedi |
| | | dheditrdqtstlktclplelhknesclatretssttrgsclppqktslmmtlclgsiyedlk |
| | | myqtefqainaalqnhnhqqiildkgmlvaidelmqslnhngetlrqkppvgeadp |
| | | yrvkmklcillhafstrvvtinrvmgylssaggggsggggsggggsggggsggggs |
| | | ggggsiwelkkdvyvveldwypdapgemvvltcdtpeedgitwtldqssevlgsg |
| | | ktltiqvkefgdagqytchkggevlshsllllhkkedgiwstdilkdqkepknktflrce |
| | | aknysgrftcwwlttistdltfsvkssrgssdpqgvtcgaatlsaervrgdnkeyeysve |
| | | cqedsacpaaeeslpievmvdavhklkyenytssffirdiikpdppknlqlkplknsr |
| | | qvevsweypdtwstphsyfsltfcvqvqgkskrekkdrvftdktsatvicrknasisv |
| | | raqdryyssswsewasvpcssggpALFKSSFPpgsggggsggggsggggsg |
| | | gggsggggsggggsQSVLTQPPSVSGAPGQRVTISCSGSRSNIG |
| | | SNTVKWYQQLPGTAPKLLIYYNDQRPSGVPDRFSGSKS |
| | | GTSASLAITGLQAEDEADYYCQSYDRYTHPALLFGTGT |
| | | KVTVLggggsggggsggggsQVQLVESGGGVVQPGRSLRLSC |
| | | AASGFTFSSYGMHWVRQAPGKGLEWVAFIRYeGSNKY |
| | | YAeSVKGRFTISRDNSKNTLYLQMNSLRAEDTAVYYCK |
| | | THGSHDNWGQGTMVTVSS** |
| 25 | WW01107 | rnlpvatpdpgmfpclhhsqnllravsnmlqkarqtlefypctseeidheditkdktst |
| | | veaclpleltknesclnsretsfitngsclasrktsfmmalclssiyedlkmyqvefktm |
| | | nakllmdpkrqifldqnmlavidelmqalnfnsetvpqkssleepdfyktkiklcillh |
| | | afriravtidrvmsylnassggpALFKSSFPpgsggggsggggsggggsEVQ |
| | | LVESGGGLVQPGNSLRLSCAASGFTFSKFGMSWVRQAP |
| | | GKGLEWVSSISGSGRDTLYAESVKGRFTISRDNAKTTLY |
| | | LQMNSLRPEDTAVYYCTIGGSLSVSSQGTLVTVSSggggs |
| | | ggggsggggssggpALFKSSFPpgsiwelkkdvyvveldwypdapgemvv |
| | | ltcdtpeedgitwtldqssevlgsgktltiqvkefgdagqytchkggevlshsllllhkk |
| | | edgiwstdilkdqkepknktflrceaknysgrftcwwlttistdltfsvkssrgssdpqg |
| | | vtcgaatlsaervrgdnkeyeysvecqedsacpaaeeslpievmvdavhklkyenyt |
| | | ssffirdiikpdppknlqlkplknsrqvevsweypdtwstphsyfsltfcvqvqgksk |
| | | rekkdrvftdktsatvicrknasisvraqdryyssswsewasvpcssggpALFKS |
| | | SFPpgsggggsggggsggggsggggsggggsggggsQSVLTQPPSVSG |
| | | APGQRVTISCSGSRSNIGSNTVKWYQQLPGTAPKLLIYY |
| | | NDQRPSGVPDRFSGSKSGTSASLAITGLQAEDEADYYCQ |
| | | SYDRYTHPALLFGTGTKVTVLggggsggggsggggsQVQLVE |
| | | SGGGVVQPGRSLRLSCAASGFTFSSYGMHWVRQAPGK |
| | | GLEWVAFIRYeGSNKYYAeSVKGRFTISRDNSKNTLYLQ |
| | | MNSLRAEDTAVYYCKTHGSHDNWGQGTMVTVSS** |
| 26 | WW01108 | rvipvsgparclsqsrnllkttddmvktareklkhysctaedidheditrdqtstlktclpl |
| | | elhknesclatretssttrgsclppqktslmmtlclgsiyedlkmyqtefqainaalqnh |
| | | nhqqiildkgmlvaidelmqslnhngetlrqkppvgeadpyrvkmklcillhafstrv |
| | | vtinrvmgylssasggpALFKSSFPpgsggggsggggsggggsEVQLVE |
| | | SGGGLVQPGNSLRLSCAASGFTFSKFGMSWVRQAPGKG |
| | | LEWVSSISGSGRDTLYAESVKGRFTISRDNAKTTLYLQM |
| | | NSLRPEDTAVYYCTIGGSLSVSSQGTLVTVSSggggsggggs |
| | | ggggssggpALFKSSFPpgsiwelkkdvyvveldwypdapgemvvltcdtp |
| | | eedgitwtldqssevlgsgktltiqvkefgdagqytchkggevlshsllllhkkedgiw |
| | | stdilkdqkepknktflrceaknysgrftcwwlttistdltfsvkssrgssdpqgvtcga |
| | | atlsaervrgdnkeyeysvecqedsacpaaeeslpievmvdavhklkyenytssffir |
| | | diikpdppknlqlkplknsrqvevsweypdtwstphsyfsltfcvqvqgkskrekkd |
| | | rvftdktsatvicrknasisvraqdryyssswsewasvpcssggpALFKSSFPpg |
| | | sggggsggggsggggsggggsggggsggggsQSVLTQPPSVSGAPGQ |
| | | RVTISCSGSRSNIGSNTVKWYQQLPGTAPKLLIYYNDQR |
| | | PSGVPDRFSGSKSGTSASLAITGLQAEDEADYYCQSYDR |
| | | YTHPALLFGTGTKVTVLggggsggggsggggsQVQLVESGGG |
| | | VVQPGRSLRLSCAASGFTFSSYGMHWVRQAPGKGLEW |
| | | VAFIRYeGSNKYYAeSVKGRFTISRDNSKNTLYLQMNSL |
| | | RAEDTAVYYCKTHGSHDNWGQGTMVTVSS** |
| 27 | WW01109 | iwelkkdvyvveldwypdapgemvvltcdtpeedgitwtldqssevlgsgktltiqv |
| | | kefgdagqytchkggevlshsllllhkkedgiwstdilkdqkepknktflrceaknys |
| | | grftcwwlttistdltfsvkssrgssdpqgvtcgaatlsaervrgdnkeyeysvecqeds |
| | | acpaaeeslpievmvdavhklkyenytssffirdiikpdppknlqlkplknsrqvevs |
| | | weypdtwstphsyfsltfcvqvqgkskrekkdrvftdktsatvicrknasisvraqdry |
| | | yssswsewasvpcssggpALFKSSFPpgsggggsggggsggggsEVQLV |
| | | ESGGGLVQPGNSLRLSCAASGFTFSKFGMSWVRQAPGK |
| | | GLEWVSSISGSGRDTLYAESVKGRFTISRDNAKTTLYLQ |
| | | MNSLRPEDTAVYYCTIGGSLSVSSQGTLVTVSSggggsggg |
| | | gsggggssggpALFKSSFPpgsrnlpvatpdpgmfpclhhsqnllravsnmlq |
| | | karqtlefypctseeidheditkdktstveaclpleltknesclnsretsfitngsclasrkts |
| | | fmmalclssiyedlkmyqvefktmnakllmdpkrqifldqnmlavidelmqalnf |
| | | nsetvpqkssleepdfyktkiklcillhafriravtidrvmsylnassggpALFKSS |
| | | FPpgsggggsggggsggggsggggsggggsggggsQSVLTQPPSVSGA |
| | | PGQRVTISCSGSRSNIGSNTVKWYQQLPGTAPKLLIYYN |
| | | DQRPSGVPDRFSGSKSGTSASLAITGLQAEDEADYYCQS |
| | | YDRYTHPALLFGTGTKVTVLggggsggggsggggsQVQLVES |
| | | GGGVVQPGRSLRLSCAASGFTFSSYGMHWVRQAPGKG |
| | | LEWVAFIRYeGSNKYYAeSVKGRFTISRDNSKNTLYLQM |
| | | NSLRAEDTAVYYCKTHGSHDNWGQGTMVTVSS** |
| 28 | WW01110 | iwelkkdvyvveldwypdapgemvvltcdtpeedgitwtldqssevlgsgktltiqv |
| | | kefgdagqytchkggevlshsllllhkkedgiwstdilkdqkepknktflrceaknys |
| | | grftcwwlttistdltfsvkssrgssdpqgvtcgaatlsaervrgdnkeyeysvecqeds |
| | | acpaaeeslpievmvdavhklkyenytssffirdiikpdppknlqlkplknsrqvevs |
| | | weypdtwstphsyfsltfcvqvqgkskrekkdrvftdktsatvicrknasisvraqdry |
| | | yssswsewasvpcssggpALFKSSFPpgsggggsggggsggggsEVQLV |
| | | ESGGGLVQPGNSLRLSCAASGFTFSKFGMSWVRQAPGK |
| | | GLEWVSSISGSGRDTLYAESVKGRFTISRDNAKTTLYLQ |
| | | MNSLRPEDTAVYYCTIGGSLSVSSQGTLVTVSSggggsggg |
| | | gsggggssggpALFKSSFPpgsrvipvsgparclsqsrnllkttddmvktarekl |
| | | khysctaedidheditrdqtstlktclplelhknesclatretssttrgsclppqktslmmt |
| | | lclgsiyedlkmyqtefqainaalqnhnhqqiildkgmlvaidelmqslnhngetlrq |
| | | kppvgeadpyrvkmklcillhafstrvvtinrvmgylssasggpALFKSSFPpg |
| | | sggggsggggsggggsggggsggggsggggsQSVLTQPPSVSGAPGQ |
| | | RVTISCSGSRSNIGSNTVKWYQQLPGTAPKLLIYYNDQR |
| | | PSGVPDRFSGSKSGTSASLAITGLQAEDEADYYCQSYDR |
| | | YTHPALLFGTGTKVTVLggggsggggsggggsQVQLVESGGG |
| | | VVQPGRSLRLSCAASGFTFSSYGMHWVRQAPGKGLEW |
| | | VAFIRYeGSNKYYAeSVKGRFTISRDNSKNTLYLQMNSL |
| | | RAEDTAVYYCKTHGSHDNWGQGTMVTVSS** |
| 29 | WW01114 | rvipvsgparclsqsrnllkttddmvktareklkhysctaedidheditrdqtstlktclpl |
| | | elhknesclatretssttrgsclppqktslmmtlclgsiyedlkmyqtefqainaalqnh |
| | | nhqqiildkgmlvaidelmqslnhngetlrqkppvgeadpyrvkmklcillhafstrv |
| | | vtinrvmgylssaggggsggggssggpALFKSSFPpgsggggsggggsEVQ |
| | | LVESGGGLVQPGNSLRLSCAASGFTFSKFGMSWVRQAP |
| | | GKGLEWVSSISGSGRDTLYAESVKGRFTISRDNAKTTLY |
| | | LQMNSLRPEDTAVYYCTIGGSLSVSSQGTLVTVSSggggs |
| | | ggggsggggsggggsggggsggggsQSVLTQPPSVSGAPGQRVTIS |
| | | CSGSRSNIGSNTVKWYQQLPGTAPKLLIYYNDQRPSGVP |
| | | DRFSGSKSGTSASLAITGLQAEDEADYYCQSYDRYTHP |
| | | ALLFGTGTKVTVLggggsggggsggggsQVQLVESGGGVVQP |
| | | GRSLRLSCAASGFTFSSYGMHWVRQAPGKGLEWVAFIR |
| | | YeGSNKYYAeSVKGRFTISRDNSKNTLYLQMNSLRAEDT |
| | | AVYYCKTHGSHDNWGQGTMVTVSS** |
| 30 | WW01115 | rvipvsgparclsqsrnllkttddmvktareklkhysctaedidheditrdqtstlktclpl |
| | | elhknesclatretssttrgsclppqktslmmtlclgsiyedlkmyqtefqainaalqnh |
| | | nhqqiildkgmlvaidelmqslnhngetlrqkppvgeadpyrvkmklcillhafstrv |
| | | vtinrvmgylssaggggsggggssggpALFKSSFPpgsggggsggggsEVQ |
| | | LVESGGGLVQPGNSLRLSCAASGFTFSKFGMSWVRQAP |
| | | GKGLEWVSSISGSGRDTLYAESVKGRFTISRDNAKTTLY |
| | | LQMNSLRPEDTAVYYCTIGGSLSVSSQGTLVTVSSggggs |
| | | ggggsggggsggggsggggsggggsQSVLTQPPSVSGAPGQRVTIS |
| | | CSGSRSNIGdeTVKWYQQLPGTAPKLLIYYNDQRPSGVP |
| | | DRFSGSKSGTSASLAITGLQAEDEADYYCQSYDRYTHP |
| | | ALLFGTGTKVTVLggggsggggsggggsQVQLVESGGGVVQP |
| | | GRSLRLSCAASGFTFSSYGMHWVRQAPGKGLEWVAFIR |
| | | YeGSNKYYAeSVKGRFTISRDNSKNTLYLQMNSLRAEDT |
| | | AVYYCKTHGSHDNWGQGTMVTVSS** |
| 31 | WW01116 | rvipvsgparclsqsrnllkttddmvktareklkhysctaedidheditrdqtstlktclpl |
| | | elhknesclatretssttrgsclppqktslmmtlclgsiyedlkmyqtefqainaalqnh |
| | | nhqqiildkgmlvaidelmqslnhngetlrqkppvgeadpyrvkmklcillhafstrv |
| | | vtinrvmgylssaggggsggggssggpALFKSSFPpgsggggsggggsEVQ |
| | | LVESGGGLVQPGNSLRLSCAASGFTFSKFGMSWVRQAP |
| | | GKGLEWVSSISGSGRDTLYAESVKGRFTISRDNAKTTLY |
| | | LQMNSLRPEDTAVYYCTIGGSLSVSSQGTLVTVSSsggpA |
| | | LFKSSFPpgsggggsggggsggggsggggsggggsggggsQSVLTQPPS |
| | | VSGAPGQRVTISCSGSRSNIGSNTVKWYQQLPGTAPKLL |
| | | IYYNDQRPSGVPDRFSGSKSGTSASLAITGLQAEDEADY |
| | | YCQSYDRYTHPALLFGTGTKVTVLggggsggggsggggsQVQ |
| | | LVESGGGVVQPGRSLRLSCAASGFTFSSYGMHWVRQAP |
| | | GKGLEWVAFIRYeGSNKYYAeSVKGRFTISRDNSKNTLY |
| | | LQMNSLRAEDTAVYYCKTHGSHDNWGQGTMVTVSS** |
| 32 | WW01159 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsrvipvsgparclsqsrnllkttddmvktareklkhysctaedi |
| | | dheditrdqtstlktclplelhknesclatretssttrgsclppqktslmmtlclgsiyedlk |
| | | myqtefqainaalqnhnhqqiildkgmlvaidelmqslnhngetlrqkppvgeadp |
| | | yrvkmklcillhafstrvvtinrvmgylssasggpALFKSSFPpgsggggsggg |
| | | gsggggsggggsggggsggggsQSVLTQPPSVSGAPGQRVTISCS |
| | | GSRSNIGSNTVKWYQQLPGTAPKLLIYYNDQRPSGVPD |
| | | RFSGSKSGTSASLAITGLQAEDEADYYCQSYDRYTHPAL |
| | | LFGTGTKVTVLastkgpsvfplapsskstsggtaalgclvkdyfpepvtvswn |
| | | sgaltsgvhtfpavlqssglyslssvvtvpssslgtqtyicnvnhkpsntkvdkrvepks |
| | | c** |
| 33 | WW01161 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsrnlpvatpdpgmfpclhhsqnllravsnmlqkarqtlefyp |
| | | ctseeidheditkdktstveaclpleltknescInsretsfitngsclasrktsfmmalclss |
| | | iyedlkmyqvefktmnakllmdpkrqifldqnmlavidelmqalnfnsetvpqkss |
| | | leepdfyktkiklcillhafriravtidrvmsylnassggpALFKSSFPpgsggggs |
| | | ggggsggggsggggsggggsggggsQSVLTQPPSVSGAPGQRVTIS |
| | | CSGSRSNIGSNTVKWYQQLPGTAPKLLIYYNDQRPSGVP |
| | | DRFSGSKSGTSASLAITGLQAEDEADYYCQSYDRYTHP |
| | | ALLFGTGTKVTVLastkgpsvfplapsskstsggtaalgclvkdyfpepvtv |
| | | swnsgaltsgvhtfpavlqssglyslssvvtvpssslgtqtyicnvnhkpsntkvdkrv |
| | | epksc** |
| 34 | WW01166 | vprdcgckpcictvpevssvfifppkpkdvltitltpkvtcvvvdiskddpevqfswf |
| | | vddvevhtaqtqpreeqfnstfrsvselpimhqdwlngkefkcrvnsaafpapiekti |
| | | sktkgrpkapqvytipppkeqmakdkvsltcmitdffpeditvewqwngqpaeny |
| | | kntqpimdtdgsyfvysklnvqksnweagntftcsvlheglhnhhtekslshspgks |
| | | ggpALFKSSFPpgsiwelkkdvyvveldwypdapgemvvltcdtpeedgit |
| | | wtldqssevlgsgktltiqvkefgdagqytchkggevlshsllllhkkedgiwstdilk |
| | | dqkepknktflrceaknysgrftcwwlttistdltfsvkssrgssdpqgvtcgaatlsaer |
| | | vrgdnkeyeysvecqedsacpaaeeslpievmvdavhklkyenytssffirdiikpd |
| | | ppknlqlkplknsrqvevsweypdtwstphsyfsltfcvqvqgkskrekkdrvftdk |
| | | tsatvicrknasisvraqdryyssswsewasvpcsggggsggggsggggsrvipvsg |
| | | parclsqsrnllkttddmvktareklkhysctaedidheditrdqtstlktclplelhknes |
| | | clatretssttrgsclppqktslmmtlclgsiyedlkmyqtefqainaalqnhnhqqiil |
| | | dkgmlvaidelmqslnhngetlrqkppvgeadpyrvkmklcillhafstrvvtinrv |
| | | mgylssasggpALFKSSFPpgsggggsggggsggggsggggsggggsgggg |
| | | sQSVLTQPPSVSGAPGQRVTISCSGSRSNIGSNTVKWYQ |
| | | QLPGTAPKLLIYYNDQRPSGVPDRFSGSKSGTSASLAITG |
| | | LQAEDEADYYCQSYDRYTHPALLFGTGTKVTVLggggsg |
| | | gggggggsQVQLVESGGGVVQPGRSLRLSCAASGFTFSSY |
| | | GMHWVRQAPGKGLEWVAFIRYeGSNKYYAeSVKGRFTI |
| | | SRDNSKNTLYLQMNSLRAEDTAVYYCKTHGSHDNWG |
| | | QGTMVTVSS** |
| 35 | WW01167 | iwelkkdvyvveldwypdapgemvvltcdtpeedgitwtldqssevlgsgktltiqv |
| | | kefgdagqytchkggevlshsllllhkkedgiwstdilkdqkepknktflrceaknys |
| | | grftcwwlttistdltfsvkssrgssdpqgvtcgaatlsaervrgdnkeyeysvecqeds |
| | | acpaaeeslpievmvdavhklkyenytssffirdiikpdppknlqlkplknsrqvevs |
| | | weypdtwstphsyfsltfcvqvqgkskrekkdrvftdktsatvicrknasisvraqdry |
| | | yssswsewasvpcsggggsggggsggggsrvipvsgparclsqsrnllkttddmvk |
| | | tareklkhysctaedidheditrdqtstlktclplelhknesclatretssttrgsclppqkts |
| | | lmmtlclgsiyedlkmyqtefqainaalqnhnhqqiildkgmlvaidelmqslnhn |
| | | getlrqkppvgeadpyrvkmklcillhafstrvvtinrvmgylssasggpALFKSS |
| | | FPpgsggggsggggsggggsggggsggggsggggsQSVLTQPPSVSGA |
| | | PGQRVTISCSGSRSNIGSNTVKWYQQLPGTAPKLLIYYN |
| | | DQRPSGVPDRFSGSKSGTSASLAITGLQAEDEADYYCQS |
| | | YDRYTHPALLFGTGTKVTVLggggsggggsggggsQVQLVES |
| | | GGGVVQPGRSLRLSCAASGFTFSSYGMHWVRQAPGKG |
| | | LEWVAFIRYeGSNKYYAeSVKGRFTISRDNSKNTLYLQM |
| | | NSLRAEDTAVYYCKTHGSHDNWGQGTMVTVSSsggpAL |
| | | FKSSFPpgsvprdcgckpcictvpevssvfifppkpkdvltitltpkvtcvvvdisk |
| | | ddpevqfswfvddvevhtaqtqpreeqfnstfrsvselpimhqdwlngkefkcrvn |
| | | saafpapiektisktkgrpkapqvytipppkeqmakdkvsltcmitdffpeditvew |
| | | qwngqpaenykntqpimdtdgsyfvysklnvqksnweagntftcsvlheglhnhh |
| | | tekslshspgk** |
| 36 | WW01095 | iwelkkdvyvveldwypdapgemvvltcdtpeedgitwtldqssevlgsgktltiqv |
| | | kefgdagqytchkggevlshsllllhkkedgiwstdilkdqkepknktflrceaknys |
| | | grftcwwlttistdltfsvkssrgssdpqgvtcgaatlsaervrgdnkeyeysvecqeds |
| | | acpaaeeslpievmvdavhklkyenytssffirdiikpdppknlqlkplknsrqvevs |
| | | weypdtwstphsyfsltfcvqvqgkskrekkdrvftdktsatvicrknasisvraqdry |
| | | yssswsewasvpcssggpALFKSSFPpgsQSVLTQPPSVSGAPGQ |
| | | RVTISCSGSRSNIGSNTVKWYQQLPGTAPKLLIYYNDQR |
| | | PSGVPDRFSGSKSGTSASLAITGLQAEDEADYYCQSYDR |
| | | YTHPALLFGTGTKVTVLgqpkaapsvtlfppsseelqankatlvclisdfy |
| | | pgavtvawkadsspvkagvetttpskqsnnkyaassylsltpeqwkshrsyscqvth |
| | | egstvektvaptecs** |
| 37 | WW00793 | QSVLTQPPSVSGAPGQRVTISCSGSRSNIGSNTVKWYQQ |
| | | LPGTAPKLLIYYNDQRPSGVPDRFSGSKSGTSASLAITGL |
| | | QAEDEADYYCQSYDRYTHPALLFGTGTKVTVLgqpkaaps |
| | | vtlfppsseelqankatlvclisdfypgavtvawkadsspvkagvetttpskqsnnky |
| | | aassylsltpeqwkshrsyscqvthegstvektvaptecs** |
| 38 | WW01160 | QVQLVESGGGVVQPGRSLRLSCAASGFTFSSYGMHWV |
| | | RQAPGKGLEWVAFIRYeGSNKYYAeSVKGRFTISRDNSK |
| | | NTLYLQMNSLRAEDTAVYYCKTHGSHDNWGQGTMVT |
| | | VSSgqpkaapsvtlfppsseelqankatlvclisdfypgavtvawkadsspvkagv |
| | | etttpskqsnnkyaassylsltpeqwkshrsyscqvthegstvektvaptecs** |
| 39 | WW0758 | EVQLVESGGGLVQPGNSLRLSCAASGFTFSKFGMSWVR |
| | | QAPGKGLEWVSSISGSGRDTLYAESVKGRFTISRDNAKT |
| | | TLYLQMNSLRPEDTAVYYCTIGGSLSVSSQGTLVTVSSsg |
| | | gpALFKSSFPpgsrnlpvatpdpgmfpclhhsqnllravsnmlqkarqtlefyp |
| | | ctseeidheditkdktstveaclpleltknesclnsretsfitngsclasrktsfmmalclss |
| | | iyedlkmyqvefktmnakllmdpkrqifldqnmlavidelmqalnfnsetvpqkss |
| | | leepdfyktkiklcillhafriravtidrvmsylnassggpALFKSSFPpgsggggs |
| | | ggggsggggsggggsggggsggggsQSVLTQPPSVSGAPGQRVTIS |
| | | CSGSRSNIGSNTVKWYQQLPGTAPKLLIYYNDQRPSGVP |
| | | DRFSGSKSGTSASLAITGLQAEDEADYYCQSYDRYTHP |
| | | ALLFGTGTKVTVLggggsggggggggsQVQLVESGGGVVQP |
| | | GRSLRLSCAASGFTFSSYGMHWVRQAPGKGLEWVAFIR |
| | | YeGSNKYYAeSVKGRFTISRDNSKNTLYLQMNSLRAEDT |
| | | AVYYCKTHGSHDNWGQGTMVTVSS** |
| 144 | Blocker 1 | QVQLQESGGGLVQAGGSLRLSCAASGRTFSSVYDMGWFR |
| | | QAPGKDREFVARITESARNTRYADSVRGRFTISRDNAKNT |
| | | VYLQMNNLELEDAAVYYCAADPQTVVVGTPDYWGQGTQ |
| | | VTVSSAAAYPYDVPDYGSHHHHHH** |
| 145 | Blocker 2 | QSVLTQPPSVSGAPGQRVTISCtGSsSNIGSNTVKWYQQLPG |
| | | TAPKLLIYgNDQRPSGVPDRFSGSKSGTSASLAITGLQAEDE |
| | | ADYYCQSYDRYTHPAyvFGTGTKVTVLggggsggggsggggsQV |
| | | QLVESGGGVVQPGRSLRLSCAASGFTFSSYGMHWVRQAP |
| | | GKGLEWVAFIRYDGSNKYYADSVKGRFTISRDNSKNTLYL |
| | | QMNSLRAEDTAVYYCKTHGSHDNWGQGTMVTVSSHHHH |
| | | HH** |
| 146 | Blocker 3 | QSVLTQPPSVSGAPGQRVTISCtGSsSNIGSNTVKWYQQLPG |
| | | TAPKLLIYYNDQRPSGVPDRFSGSKSGTSASLAITGLQAED |
| | | EADYYCQSYDRYTHPAyvFGTGTKVTVLggggsggggsggggsQ |
| | | VQLVESGGGVVQPGRSLRLSCAASGFTFSSYGMHWVRQA |
| | | PGKGLEWVAFIRYDGSNKYYADSVKGRFTISRDNSKNTLY |
| | | LQMNSLRAEDTAVYYCKTHGSHDNWGQGTMVTVSSHHH |
| | | HHH** |
| 147 | Blocker 4 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYaMHWVRQ |
| | | APGKGLEWVAvIsYDGSNKYYADSVKGRFTISRDNSKNTL |
| | | YLQMNSLRAEDTAVYYCarHGSHDNWGQGTMVTVSSHHH |
| | | HHH** |
| 148 | Blocker 5 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYeGSNKYYADSVKGRFTISRDNSKNTL |
| | | YLQMNSLRAEDTAVYYCKTHGSHDNWGQGTMVTVSSHH |
| | | HHHH** |
| 149 | Blocker 6 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYAeSVKGRFTISRDNSKNTL |
| | | YLQMNSLRAEDTAVYYCKTHGSHDNWGQGTMVTVSSHH |
| | | HHHH** |
| 150 | Blocker 7 | QSVLTQPPSVSGAPGQRVTISCSGSRSNIGSqTVKWYQQLP |
| | | GTAPKLLIYYNDQRPSGVPDRFSGSKSGTSASLAITGLQAE |
| | | DEADYYCQSYeRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 151 | Blocker 8 | QSVLTQPPSVSGAPGQRVTISCSGSRSNIGSqTVKWYQQLP |
| | | GTAPKLLIYYNDQRPSGVPDRFSGSKSGTSASLAITGLQAE |
| | | DEADYYCQSYSRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 152 | Blocker 9 | QSVLTQPPSVSGAPGQRVTISCSGSeSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 153 | Blocker 10 | QSVLTQPPSVSGAPGQRVTISCSGSsSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 154 | Blocker 11 | QSVLTQPPSVSGAPGQRVTISCSGSRSNIGdNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 155 | Blocker 12 | QSVLTQPPSVSGAPGQRVTISCSGSRSNIGeNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 156 | Blocker 13 | QSVLTQPPSVSGAPGQRVTISCSGSRSNIGSdTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 157 | Blocker 14 | QSVLTQPPSVSGAPGQRVTISCSGSRSNIGSeTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 158 | Blocker 15 | QSVLTQPPSVSGAPGQRVTISCSGSRSNIGSNdVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 159 | Blocker 16 | QSVLTQPPSVSGAPGQRVTISCSGSRSNIGSNTVdWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 160 | Blocker 17 | QSVLTQPPSVSGAPGQRVTISCSGSRSNIGSNTVeWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 161 | Blocker 18 | QSVLTQPPSVSGAPGQRVTISCSGSRSNIGSNTVKWYQQLP |
| | | GTAPKLLIYYNDQdPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 162 | Blocker 19 | QSVLTQPPSVSGAPGQRVTISCSGSRSNIGSNTVKWYQQLP |
| | | GTAPKLLIYYNDQePSGVPDRFSGSKSGTSASLAITGLQAED |
| | | EADYYCQSYDRYTHPALLFGTGTKVTVLggggsggggsggggsQ |
| | | VQLVESGGGVVQPGRSLRLSCAASGFTFSSYGMHWVRQA |
| | | PGKGLEWVAFIRYDGSNKYYADSVKGRFTISRDNSKNTLY |
| | | LQMNSLRAEDTAVYYCKTHGSHDNWGQGTMVTVSSHHH |
| | | HHH** |
| 163 | Blocker 20 | QSVLTQPPSVSGAPGQRVTISCSGSRSNIGSNTVKWYQQLP |
| | | GTAPKLLIYYNDQRPdGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 164 | Blocker 21 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDeYTHPALLFGTGTKVTVLggggsggggsgggg |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 165 | Blocker 22 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTdPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 166 | Blocker 23 | QSVLTQPPSVSGAPGQRVTISCSGSeSNIGSNTVKWYQQLP |
| | | GTAPKLLIYYNDQePSGVPDRFSGSKSGTSASLAITGLQAED |
| | | EADYYCQSYDeYTHPALLFGTGTKVTVLggggsggggsggggsQ |
| | | VQLVESGGGVVQPGRSLRLSCAASGFTFSSYGMHWVRQA |
| | | PGKGLEWVAFIRYDGSNKYYADSVKGRFTISRDNSKNTLY |
| | | LQMNSLRAEDTAVYYCKTHGSHDNWGQGTMVTVSSHHH |
| | | HHH** |
| 167 | Blocker 24 | QSVLTQPPSVSGAPGQRVTISCSGSeSNIGSNdVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 168 | Blocker 25 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFeSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 169 | Blocker 26 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSeYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 170 | Blocker 27 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSdYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 171 | Blocker 28 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIeYDGSNKYYADSVKGRFTISRDNSKNTL |
| | | YLQMNSLRAEDTAVYYCKTHGSHDNWGQGTMVTVSSHH |
| | | HHHH** |
| 172 | Blocker 29 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIdYDGSNKYYADSVKGRFTISRDNSKNTL |
| | | YLQMNSLRAEDTAVYYCKTHGSHDNWGQGTMVTVSSHH |
| | | HHHH** |
| 173 | Blocker 30 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNdYYADSVKGRFTISRDNSKNTL |
| | | YLQMNSLRAEDTAVYYCKTHGSHDNWGQGTMVTVSSHH |
| | | HHHH** |
| 174 | Blocker 31 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNeYYADSVKGRFTISRDNSKNTL |
| | | YLQMNSLRAEDTAVYYCKTHGSHDNWGQGTMVTVSSHH |
| | | HHHH** |
| 175 | Blocker 32 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVeGRFTISRDNSKNTL |
| | | YLQMNSLRAEDTAVYYCKTHGSHDNWGQGTMVTVSSHH |
| | | HHHH** |
| 176 | Blocker 33 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSeDNWGQGTMVTVSSHH |
| | | HHHH** |
| 177 | Blocker 34 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIeYDGSNKYYADSVeGRFTISRDNSKNTLY |
| | | LQMNSLRAEDTAVYYCKTHGSHDNWGQGTMVTVSSHHH |
| | | HHH** |
| 178 | Blocker 35 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIeYDGSNKYYADSVeGRFTISRDNSKNTLY |
| | | LQMNSLRAEDTAVYYCKTHGSeDNWGQGTMVTVSSHHH |
| | | HHH** |
| 179 | Blocker 36 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 180 | Blocker 37 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYeGSNKYYAeSVKGRFTISRDNSKNTLY |
| | | LQMNSLRAEDTAVYYCKTHGSHDNWGQGTMVTVSSHHH |
| | | HHH** |
| 181 | Blocker 38 | QSVLTQPPSVSGAPGQRVTISCSGSRSNIGSeTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYeGSNKYYAeSVKGRFTISRDNSKNTLY |
| | | LQMNSLRAEDTAVYYCKTHGSHDNWGQGTMVTVSSHHH |
| | | HHH** |
| 182 | Blocker 39 | QSVLTQPPSVSGAPGQRVTISCSGSRSNIGdNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYeGSNKYYAeSVKGRFTISRDNSKNTLY |
| | | LQMNSLRAEDTAVYYCKTHGSHDNWGQGTMVTVSSHHH |
| | | HHH** |
| 183 | Blocker 40 | QSVLTQPPSVSGAPGQRVTISCSGSRSNIGdeTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 184 | Blocker 41 | QSVLTQPPSVSGAPGQRVTISCSGSRSNIGdeTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYeGSNKYYAeSVKGRFTISRDNSKNTLY |
| | | LQMNSLRAEDTAVYYCKTHGSHDNWGQGTMVTVSSHHH |
| | | HHH** |
| 185 | Blocker 42 | QSVLTQPPSVSGAPGQRVTISCSGSeSNIGSNdVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSHH |
| | | HHHH** |
| 186 | Blocker 43 | QSVLTQPPSVSGAPGQRVTISCSGSRSNIGeNTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYeGSNKYYAeSVKGRFTISRDNSKNTLY |
| | | LQMNSLRAEDTAVYYCKTHGSHDNWGQGTMVTVSSHHH |
| | | HHH** |
| 187 | Blocker 44 | QSVLTQPPSVSGAPGQRVTISCSGSRSNIGeeTVKWYQQLPG |
| | | TAPKLLIYYNDQRPSGVPDRFSGSKSGTSASLAITGLQAED |
| | | EADYYCQSYDRYTHPALLFGTGTKVTVLggggsggggsggggsQ |
| | | VQLVESGGGVVQPGRSLRLSCAASGFTFSSYGMHWVRQA |
| | | PGKGLEWVAFIRYDGSNKYYADSVKGRFTISRDNSKNTLY |
| | | LQMNSLRAEDTAVYYCKTHGSHDNWGQGTMVTVSSHHH |
| | | HHH** |
| 188 | Blocker 45 | QSVLTQPPSVSGAPGQRVTISCSGSRSNIGSeTVKWYQQLP |
| | | GTAPKLLIYYNDQRPSGVPDRFSGSKSGTSASLAITGLQAE |
| | | DEADYYCQSYDRYTHPALLFGTGTKVTVLggggsggggsggggs |
| | | QVQLVESGGGVVQPGRSLRLSCAASGFTFSSYGMHWVRQ |
| | | APGKGLEWVAFIRYeGSNKYYADSVKGRFTISRDNSKNTL |
| | | YLQMNSLRAEDTAVYYCKTHGSHDNWGQGTMVTVSSHH |
| | | HHHH** |
| 189 | Blocker 46 | QVQLVESGGGVVQPGRSLRLSCAASGFTFSSYGMHWVRQ |
| | | APGKGLEWVAFIRYDGSNKYYADSVKGRFTISRDNSKNTL |
| | | YLQMNSLRAEDTAVYYCKTHGSHDNWGQGTMVTVSSastk |
| | | gpsvfplapsskstsggtaalgclvkdyfpepvtvswnsgaltsgvhtfpavlqssglysls |
| | | svvtvpssslgtqtyicnvnhkpsntkvdkrvepksc** |
| 190 | Blocker 47 | QVQLVESGGGVVQPGRSLRLSCAASGFTFSSYGMHWVRQ |
| | | APGKGLEWVAFIRYeGSNKYYAeSVKGRFTISRDNSKNTLY |
| | | LQMNSLRAEDTAVYYCKTHGSHDNWGQGTMVTVSSastkg |
| | | psvfplapsskstsggtaalgclvkdyfpepvtvswnsgaltsgvhtfpavlqssglyslss |
| | | vvtvpssslgtqtyicnvnhkpsntkvdkrvepksc** |
| 191 | Blocker 48 | QVQLVESGGGVVQPGRSLRLSCAASGFTFSSYGMHWVRQ |
| | | APGKGLEWVAFIRYeGSNKYYADSVKGRFTISRDNSKNTL |
| | | YLQMNSLRAEDTAVYYCKTHGSHDNWGQGTMVTVSSastk |
| | | gpsvfplapsskstsggtaalgclvkdyfpepvtvswnsgaltsgvhtfpavlqssglysls |
| | | svvtvpssslgtqtyicnvnhkpsntkvdkrvepksc** |
| 192 | Blocker 49 | QSVLTQPPSVSGAPGQRVTISCSGSRSNIGSNTVKWYQQLP |
| | | GTAPKLLIYYNDQRPSGVPDRFSGSKSGTSASLAITGLQAE |
| | | DEADYYCQSYDRYTHPALLFGTGTKVTVLgqpkaapsvtlfppss |
| | | eelqankatlvclisdfypgavtvawkadsspvkagvetttpskqsnnkyaassylsltpe |
| | | qwkshrsyscqvthegstvektvaptecs** |
| 193 | Blocker 50 | QSVLTQPPSVSGAPGQRVTISCSGSRSNIGdeTVKWYQQLP |
| | | GTAPKLLIYYNDQRPSGVPDRFSGSKSGTSASLAITGLQAE |
| | | DEADYYCQSYDRYTHPALLFGTGTKVTVLgqpkaapsvtlfppss |
| | | eelqankatlvclisdfypgavtvawkadsspvkagvetttpskqsnnkyaassylsltpe |
| | | qwkshrsyscqvthegstvektvaptecs** |
| 194 | Blocker 51 | QVQLVESGGGVVQPGRSLRLSCAASGFTFSSYGMHWVRQ |
| | | APGKGLEWVAFIRYeGSNKYYAeSVKGRFTISRDNSKNTLY |
| | | LQMNSLRAEDTAVYYCKTHGSHDNWGQGTMVTVSSastkg |
| | | psvfplapsskstsggtaalgclvkdyfpepvtvswnsgaltsgvhtfpavlqssglyslss |
| | | vvtvpssslgtqtyicnvnhkpsntkvdkrvepkscHHHHHH** |
| 221 | MMP14 substrate | GPLGLKAQ |
| | motif sequence | |
| 222 | MMP14 substrate | LPLGLKAQ |
| | motif sequence | |
| 223 | MMP14 substrate | SPLGLKAQ |
| | motif sequence | |
| 224 | MMP14 substrate | QPLGLKAQ |
| | motif sequence | |
| 225 | MMP14 substrate | KPLGLKAQ |
| | motif sequence | |
| 226 | MMP14 substrate | FPLGLKAQ |
| | motif sequence | |
| 227 | MMP14 substrate | HPLGLKAQ |
| | motif sequence | |
| 228 | MMP14 substrate | PPLGLKAQ |
| | motif sequence | |
| 229 | MMP14 substrate | APLGLKAQ |
| | motif sequence | |
| 230 | MMP14 substrate | DPLGLKAQ |
| | motif sequence | |
| 231 | MMP14 substrate | GPHGLKAQ |
| | motif sequence | |
| 232 | MMP14 substrate | GPSGLKAQ |
| | motif sequence | |
| 233 | MMP14 substrate | GPQGLKAQ |
| | motif sequence | |
| 234 | MMP14 substrate | GPPGLKAQ |
| | motif sequence | |
| 235 | MMP14 substrate | GPEGLKAQ |
| | motif sequence | |
| 236 | MMP14 substrate | GPFGLKAQ |
| | motif sequence | |
| 237 | MMP14 substrate | GPRGLKAQ |
| | motif sequence | |
| 238 | MMP14 substrate | GPGGLKAQ |
| | motif sequence | |
| 239 | MMP14 substrate | GPAGLKAQ |
| | motif sequence | |
| 240 | MMP14 substrate | LPAGLKGA |
| | motif sequence | |
| 241 | MMP14 substrate | GPAGLYAQ |
| | motif sequence | |
| 242 | MMP14 substrate | GPANLVAQ |
| | motif sequence | |
| 243 | MMP14 substrate | GPAALVGA |
| | motif sequence | |
| 244 | MMP14 substrate | GPANLRAQ |
| | motif sequence | |
| 245 | MMP14 substrate | GPAGLRAQ |
| | motif sequence | |
| 246 | MMP14 substrate | GPAGLVAQ |
| | motif sequence | |
| 247 | MMP14 substrate | GPAGLRGA |
| | motif sequence | |
| 248 | MMP14 substrate | LPAGLVGA |
| | motif sequence | |
| 249 | MMP14 substrate | GPAGLKGA |
| | motif sequence | |
| 250 | MMP14 substrate | GPLALKAQ |
| | motif sequence | |
| 251 | MMP14 substrate | GPLNLKAQ |
| | motif sequence | |
| 252 | MMP14 substrate | GPLHLKAQ |
| | motif sequence | |
| 253 | MMP14 substrate | GPLYLKAQ |
| | motif sequence | |
| 254 | MMP14 substrate | GPLPLKAQ |
| | motif sequence | |
| 255 | MMP14 substrate | GPLELKAQ |
| | motif sequence | |
| 256 | MMP14 substrate | GPLRLKAQ |
| | motif sequence | |
| 257 | MMP14 substrate | GPLLLKAQ |
| | motif sequence | |
| 258 | MMP14 substrate | GPLSLKAQ |
| | motif sequence | |
| 259 | MMP14 substrate | GPLGLYAQ |
| | motif sequence | |
| 260 | MMP14 substrate | GPLGLFAQ |
| | motif sequence | |
| 261 | MMP14 substrate | GPLGLLAQ |
| | motif sequence | |
| 262 | MMP14 substrate | GPLGLHAQ |
| | motif sequence | |
| 263 | MMP14 substrate | GPLGLRAQ |
| | motif sequence | |
| 264 | MMP14 substrate | GPLGLAAQ |
| | motif sequence | |
| 265 | MMP14 substrate | GPLGLEAQ |
| | motif sequence | |
| 266 | MMP14 substrate | GPLGLGAQ |
| | motif sequence | |
| 267 | MMP14 substrate | GPLGLPAQ |
| | motif sequence | |
| 268 | MMP14 substrate | GPLGLQAQ |
| | motif sequence | |
| 269 | MMP14 substrate | GPLGLSAQ |
| | motif sequence | |
| 270 | MMP14 substrate | GPLGLVAQ |
| | motif sequence | |
| 271 | MMP14 substrate | GPLGLKLQ |
| | motif sequence | |
| 272 | MMP14 substrate | GPLGLKFQ |
| | motif sequence | |
| 273 | MMP14 substrate | GPLGLKEQ |
| | motif sequence | |
| 274 | MMP14 substrate | GPLGLKKQ |
| | motif sequence | |
| 275 | MMP14 substrate | GPLGLKQQ |
| | motif sequence | |
| 276 | MMP14 substrate | GPLGLKSQ |
| | motif sequence | |
| 277 | MMP14 substrate | GPLGLKGQ |
| | motif sequence | |
| 278 | MMP14 substrate | GPLGLKHQ |
| | motif sequence | |
| 279 | MMP14 substrate | GPLGLKPQ |
| | motif sequence | |
| 280 | MMP14 substrate | GPLGLKAG |
| | motif sequence | |
| 281 | MMP14 substrate | GPLGLKAF |
| | motif sequence | |
| 282 | MMP14 substrate | GPLGLKAP |
| | motif sequence | |
| 283 | MMP14 substrate | GPLGLKAL |
| | motif sequence | |
| 284 | MMP14 substrate | GPLGLKAE |
| | motif sequence | |
| 285 | MMP14 substrate | GPLGLKAA |
| | motif sequence | |
| 286 | MMP14 substrate | GPLGLKAH |
| | motif sequence | |
| 287 | MMP14 substrate | GPLGLKAK |
| | motif sequence | |
| 288 | MMP14 substrate | GPLGLKAS |
| | motif sequence | |
| 289 | MMP14 substrate | GPLGLFGA |
| | motif sequence | |
| 290 | MMP14 substrate | GPLGLQGA |
| | motif sequence | |
| 291 | MMP14 substrate | GPLGLVGA |
| | motif sequence | |
| 292 | MMP14 substrate | GPLGLAGA |
| | motif sequence | |
| 293 | MMP14 substrate | GPLGLLGA |
| | motif sequence | |
| 294 | MMP14 substrate | GPLGLRGA |
| | motif sequence | |
| 295 | MMP14 substrate | GPLGLYGA |
| | motif sequence | |
| 296 | CTSL1 substrate | ALFKSSPP |
| | motif sequence | |
| 297 | CTSL1 substrate | SPFRSSRQ |
| | motif sequence | |
| 298 | CTSL1 substrate | KLFKSSPP |
| | motif sequence | |
| 299 | CTSL1 substrate | HLFKSSPP |
| | motif sequence | |
| 300 | CTSL1 substrate | SLFKSSPP |
| | motif sequence | |
| 301 | CTSL1 substrate | QLFKSSPP |
| | motif sequence | |
| 302 | CTSL1 substrate | LLFKSSPP |
| | motif sequence | |
| 303 | CTSL1 substrate | PLFKSSPP |
| | motif sequence | |
| 304 | CTSL1 substrate | FLFKSSPP |
| | motif sequence | |
| 305 | CTSL1 substrate | GLFKSSPP |
| | motif sequence | |
| 306 | CTSL1 substrate | VLFKSSPP |
| | motif sequence | |
| 307 | CTSL1 substrate | ELFKSSPP |
| | motif sequence | |
| 308 | CTSL1 substrate | AKFKSSPP |
| | motif sequence | |
| 309 | CTSL1 substrate | AHFKSSPP |
| | motif sequence | |
| 310 | CTSL1 substrate | AGFKSSPP |
| | motif sequence | |
| 311 | CTSL1 substrate | APFKSSPP |
| | motif sequence | |
| 312 | CTSL1 substrate | ANFKSSPP |
| | motif sequence | |
| 313 | CTSL1 substrate | AFFKSSPP |
| | motif sequence | |
| 314 | CTSL1 substrate | AAFKSSPP |
| | motif sequence | |
| 315 | CTSL1 substrate | ASFKSSPP |
| | motif sequence | |
| 316 | CTSL1 substrate | AEFKSSPP |
| | motif sequence | |
| 317 | CTSL1 substrate | ALRKSSPP |
| | motif sequence | |
| 318 | CTSL1 substrate | ALLKSSPP |
| | motif sequence | |
| 319 | CTSL1 substrate | ALAKSSPP |
| | motif sequence | |
| 320 | CTSL1 substrate | ALQKSSPP |
| | motif sequence | |
| 321 | CTSL1 substrate | ALHKSSPP |
| | motif sequence | |
| 322 | CTSL1 substrate | ALPKSSPP |
| | motif sequence | |
| 323 | CTSL1 substrate | ALTKSSPP |
| | motif sequence | |
| 324 | CTSL1 substrate | ALGKSSPP |
| | motif sequence | |
| 325 | CTSL1 substrate | ALDKSSPP |
| | motif sequence | |
| 326 | CTSL1 substrate | ALFFSSPP |
| | motif sequence | |
| 327 | CTSL1 substrate | ALFHSSPP |
| | motif sequence | |
| 328 | CTSL1 substrate | ALFTSSPP |
| | motif sequence | |
| 329 | CTSL1 substrate | ALFASSPP |
| | motif sequence | |
| 330 | CTSL1 substrate | ALFQSSPP |
| | motif sequence | |
| 331 | CTSL1 substrate | ALFLSSPP |
| | motif sequence | |
| 332 | CTSL1 substrate | ALFGSSPP |
| | motif sequence | |
| 333 | CTSL1 substrate | ALFESSPP |
| | motif sequence | |
| 334 | CTSL1 substrate | ALFPSSPP |
| | motif sequence | |
| 335 | CTSL1 substrate | ALFKHSPP |
| | motif sequence | |
| 336 | CTSL1 substrate | ALFKLSPP |
| | motif sequence | |
| 337 | CTSLI substrate | ALFKKSPP |
| | motif sequence | |
| 338 | CTSL1 substrate | ALFKASPP |
| | motif sequence | |
| 339 | CTSL1 substrate | ALFKISPP |
| | motif sequence | |
| 340 | CTSL1 substrate | ALFKGSPP |
| | motif sequence | |
| 341 | CTSL1 substrate | ALFKNSPP |
| | motif sequence | |
| 342 | CTSL1 substrate | ALFKRSPP |
| | motif sequence | |
| 343 | CTSL1 substrate | ALFKESPP |
| | motif sequence | |
| 344 | CTSL1 substrate | ALFKFSPP |
| | motif sequence | |
| 345 | CTSL1 substrate | ALFKPSPP |
| | motif sequence | |
| 346 | CTSL1 substrate | ALFKSFPP |
| | motif sequence | |
| 347 | CTSL1 substrate | ALFKSLPP |
| | motif sequence | |
| 348 | CTSL1 substrate | ALFKSIPP |
| | motif sequence | |
| 349 | CTSL1 substrate | ALFKSKPP |
| | motif sequence | |
| 350 | CTSL1 substrate | ALFKSAPP |
| | motif sequence | |
| 351 | CTSL1 substrate | ALFKSQPP |
| | motif sequence | |
| 352 | CTSL1 substrate | ALFKSPPP |
| | motif sequence | |
| 353 | CTSLI substrate | ALFKSEPP |
| | motif sequence | |
| 354 | CTSL1 substrate | ALFKSGPP |
| | motif sequence | |
| 355 | CTSL1 substrate | ALFKSSFP |
| | motif sequence | |
| 356 | CTSL1 substrate | ALFKSSLP |
| | motif sequence | |
| 357 | CTSL1 substrate | ALFKSSGP |
| | motif sequence | |
| 358 | CTSL1 substrate | ALFKSSSP |
| | motif sequence | |
| 359 | CTSL1 substrate | ALFKSSVP |
| | motif sequence | |
| 360 | CTSLI substrate | ALFKSSHP |
| | motif sequence | |
| 361 | CTSL1 substrate | ALFKSSAP |
| | motif sequence | |
| 362 | CTSL1 substrate | ALFKSSNP |
| | motif sequence | |
| 363 | CTSL1 substrate | ALFKSSKP |
| | motif sequence | |
| 364 | CTSL1 substrate | ALFKSSEP |
| | motif sequence | |
| 365 | CTSL1 substrate | ALFKSSPF |
| | motif sequence | |
| 366 | CTSL1 substrate | ALFKSSPH |
| | motif sequence | |
| 367 | CTSL1 substrate | ALFKSSPG |
| | motif sequence | |
| 368 | CTSL1 substrate | ALFKSSPA |
| | motif sequence | |
| 369 | CTSLI substrate | ALFKSSPS |
| | motif sequence | |
| 370 | CTSL1 substrate | ALFKSSPV |
| | motif sequence | |
| 371 | CTSL1 substrate | ALFKSSPQ |
| | motif sequence | |
| 372 | CTSL1 substrate | ALFKSSPK |
| | motif sequence | |
| 373 | CTSL1 substrate | ALFKSSPL |
| | motif sequence | |
| 374 | CTSL1 substrate | ALFKSSPD |
| | motif sequence | |

1. A method for treating an advanced solid tumor, a metastatic solid tumor or a lymphoma, comprising administering to a subject in need thereof an inducible IL-12 prodrug comprising Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, or Compound 36 or an amino acid sequence variant of the foregoing.

2. The method of claim 1, wherein the administration is oral, parenteral, intravenous, intra-articular, intraperitoneal, intramuscular, subcutaneous, intracavity, transdermal, intrahepatical, intracranial, nebulization/inhalation, by installation via bronchoscopy, or intratumoral.

3. The method of claim 2, wherein the administration is intravenous.

4. The method of claim 1, wherein the inducible IL-12 prodrug is administered about twice a week or less frequently, about once a week or less frequently, or about once every 2 weeks.

5. (canceled)

6. (canceled)

7. The method of claim 1, wherein about 0.016 mg/kg, about 0.032 mg/kg, about 0.056 mg/kg, about 0.084 mg/kg, about 0.126 mg/kg, about 0.190 mg/kg, about 0.290 mg/kg, or about 0.440 mg/kg of Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, or Compound 36 or an amino acid sequence variant of the foregoing is administered every two weeks.

8-15. (canceled)

16. The method of claim 1, wherein about 1 mg, about 3 mg, about 10 mg, about 20 mg, about 30 mg, about 40 mg, about 50 mg, about 60 mg, about 100 mg, or about 200 mg of Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, or Compound 36 or an amino acid sequence variant of the foregoing is administered every two weeks.

17. (canceled)

18. The method of claim 1, wherein the subject has failed to achieve a complete response to a prior treatment or to an ongoing treatment.

19. The method of claim 16, wherein the prior or ongoing treatment comprises treatment with a checkpoint inhibitor, and the checkpoint inhibitor is an anti-PD-1 antibody, an anti-PD-L1 antibody, or an anti-CTL4-A antibody.

20. (canceled)

21. (canceled)

22. The method of claim 1, wherein the advanced solid tumor, the metastatic solid tumor or the lymphoma is adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

23. (canceled)

24. (canceled)

25. The method of claim 1, wherein Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, or Compound 36 or an amino acid sequence variant of the foregoing is administered concurrently with the anti-PD-1 antibody, or antigen binding fragment thereof.

26. A pharmaceutical composition comprising an inducible IL-12 prodrug, a citric acid and/or a citrate salt, a disaccharide and a surfactant.

27. The pharmaceutical composition of claim 26 wherein the citrate salt is sodium citrate, magnesium citrate or potassium citrate; the disaccharide is sucrose, trehalose, lactose or maltose, and the surfactant is a nonionic surfactant selected from polysorbate 80, polysorbate 20, Span-80, castor oil, or a poloxamer.

28. The pharmaceutical composition of claim 26, wherein the IL-12 prodrug comprises Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, or Compound 36 or an amino acid sequence variant of any of the foregoing.

29. The pharmaceutical composition of claim 26, wherein the composition is a liquid, a lyophilizate, or an aqueous liquid for injection or infusion.

30. (canceled)

31. The pharmaceutical composition of claim 29, wherein the composition comprises about 1 mg/mL to about 100 mg/mL IL-12 prodrug; about 5 mM to about 500 mM sodium citrate; about 20 mM to about 500 mM sucrose, about 0.001% to about 2% polysorbate 80.

32. The pharmaceutical composition of claim 31, wherein the composition comprises about 5 mg/mL IL-12 prodrug; about 50 mM sodium citrate; about 240 mM sucrose; and about 0.02% polysorbate 80.

33. The pharmaceutical composition of claim 29, wherein the pharmaceutical composition has a pH of about 5.0 to about 7.5.

34. The pharmaceutical composition of claim 33, wherein the pH is about 5.5.

33-54. (canceled)
